SEQUENCE LISTING (279 sequences)

SEQ ID NO: 1 - DNA, 336 bp, Artificial Sequence
  light chain variable region
gatgttgtgatgactcagtctccactctccctgcccgtcacccctggagagccggcctccatctcctgcaggtctagtcagagcctcctgcatagtagtggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgatcaccttcggccaagggacacgactggagattaaa

SEQ ID NO: 2 - protein, 112 aa, Artificial Sequence
  Synthetic Construct
DVVMTQSPLSLPVTPGEPASISCRSSQSLLHSSGYNYLDWYLQKPGQSPQLLIYLGSNRASGVPDRFSGSGSGTDFTLKISRVEAEDVGVYYCMQALQTPITFGQGTRLEIK

SEQ ID NO: 3 - DNA, 327 bp, Artificial Sequence
  light chain variable region
atgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgatcaccttcggccaagggacacgactggagattaaa

SEQ ID NO: 4 - protein, 109 aa, Artificial Sequence
  Synthetic Construct
MTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGQGTRLEIK

SEQ ID NO: 5 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccactcactttcggcggcgggaccaaggtggagatcaaa

SEQ ID NO: 6 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 7 - DNA, 336 bp, Artificial Sequence
  light chain variable region
gaaattgtgatgacgcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcacactttcggcggagggaccaaggtggagatcaaa

SEQ ID NO: 8 - protein, 112 aa, Artificial Sequence
  Synthetic Construct
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGGGTKVEIK

SEQ ID NO: 9 - DNA, 336 bp, Artificial Sequence
  light chain variable region
gaaattgtgctgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaacccctctcactttcggccctgggaccaaagtggatatcaaa

SEQ ID NO: 10 - protein, 112 aa, Artificial Sequence
  Synthetic Construct
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGPGTKVDIK

SEQ ID NO: 11 - DNA, 336 bp, Artificial Sequence
  light chain variable region
gatgttgtgatgactcagtctccactctccctggccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 12 - protein, 112 aa, Artificial Sequence
  Synthetic Construct
DVVMTQSPLSLAVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 13 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 14 - protein, 112 aa, Artificial Sequence; identical to SEQ ID NO: 6
  Synthetic Construct

SEQ ID NO: 15 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaagatgttggggtttattactgtatgcaagctctacaaacccccctcactttcggcggagggaccaaggtggagatcaaa

SEQ ID NO: 16 - protein, 112 aa, Artificial Sequence; identical to SEQ ID NO: 6
  Synthetic Construct

SEQ ID NO: 17 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgttcaccttcggccaagggacacgactggagattaaa

SEQ ID NO: 18 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGQGTRLEIK

SEQ ID NO: 19 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctggcgttcggccaagggaccaaggtggaaatcaaa

SEQ ID NO: 20 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLAFGQGTKVEIK

SEQ ID NO: 21 - DNA, 336 bp, Artificial Sequence
  light chain variable region
gaaattgtgctgactcagtctccactctccctgcccgtcacccctggagagccggcctccatctcctgcaggtctagtcagagcctcctgcatagtaatggatacaactatttgaattggtacctgcagaagccagggcagtctccacagctcctgatctatttgggttctaatcgggcctccggggtccctgacaggttcagtgccagtggatcaggcacagattttacactgaaaatcagcagagtggaggctgaggatgttggggtttattactgcatgcaagctctacaaactcctatcaccttcggccaagggacacgactggagattaaa

SEQ ID NO: 22 - protein, 112 aa, Artificial Sequence
  Synthetic Construct
EIVLTQSPLSLPVTPGEPASISCRSSQSLLHSNGYNYLNWYLQKPGQSPQLLIYLGSNRASGVPDRFSASGSGTDFTLKISRVEAEDVGVYYCMQALQTPITFGQGTRLEIK

SEQ ID NO: 23 - DNA, 333 bp, Artificial Sequence
  light chain variable region
aattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcagagagtgttcggcggagggaccaagctgaccgtccta

SEQ ID NO: 24 - protein, 111 aa, Artificial Sequence
  Synthetic Construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNQRVFGGGTKLTVL

SEQ ID NO: 25 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaaccccgctcactttcggcggagggaccaaggtggagatcaaa

SEQ ID NO: 26 - protein, 112 aa, Artificial Sequence; identical to SEQ ID NO: 6
  Synthetic Construct

SEQ ID NO: 27 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcttactttcggcggagggaccaaggtggagatcaaa

SEQ ID NO: 28 - protein, 112 aa, Artificial Sequence; identical to SEQ ID NO: 6
  Synthetic Construct

SEQ ID NO: 29 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcaaaagccagggcagtctccacagctcctgatctatttgggttcttatcgggcctccggggtccctgacaggttcagtgccagtggatcaggcacagattttacactgaaaatcagcagagtggaggctgaggatgttggggtttattactgcatgcaagctctacaaactccgatcaccttcggccaagggacacgactggagattaaa

SEQ ID NO: 30 - protein, 112 aa, Artificial Sequence
  Synthetic Construct
DVVMTQSPLSLPVTPGEPASISCRSSQSLLHSNGYNYLDWYLQKPGQSPQLLIYLGSYRASGVPDRFSASGSGTDFTLKISRVEAEDVGVYYCMQALQTPITFGQGTRLEIK

SEQ ID NO: 31 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattactgcatgcaaggtacacactggcctctgacgttcggccaagggaccaaggtggagatcaaa

SEQ ID NO: 32 - protein, 112 aa, Artificial Sequence [sequence omitted]

SEQ ID NO: 33 - DNA, 335 bp, Artificial Sequence
  light chain variable region
gaaattgtgatgacgcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaa

SEQ ID NO: 34 - protein, 111 aa, Artificial Sequence
  Synthetic Construct
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEI

SEQ ID NO: 35 - DNA, 321 bp, Artificial Sequence
  light chain variable region
gacatccagttgacccagtctccatcttccgtgtctgcgtctgtcggagacagagtcaccatcacttgtcgggcgagtcagggtattagcaggtggttagcctggtatcaacagaaaccagggaaagcccctagactcctgatctatgctgcgtccggtttacaaagtggggtcccatcaaggttcagcggcagtggatctgggacagatttcactctcaccatcagcaacctgcagcctgaagattttgcaacttactattgtcaacaggctagcagttttccaatcaccttcggccaagggacacgactggagactaaa

SEQ ID NO: 36 - protein, 107 aa, Artificial Sequence
  Synthetic Construct
DIQLTQSPSSVSASVGDRVTITCRASQGISRWLAWYQQKPGKAPRLLIYAASGLQSGVPSRFSGSGSGTDFTLTISNLQPEDFATYYCQQASSFPITFGQGTRLETK

SEQ ID NO: 37 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggagtttattactgcatgcaagctctacaaactccgtacacttttggccaggggaccaagctggagatcaaa

SEQ ID NO: 38 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 39 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctaacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 40 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPNRFSGSGSGTDFTLKISRVEAEDVGVYYCMQALQTPFTFGPGTKVDIK

SEQ ID NO: 41 - DNA, 336 bp, Artificial Sequence
  light chain variable region
gatgttgtgatgactcagtctccactctccctgcccgtcacccctggagagccggcctccatctcctgcaggtctagtcagagcctcctgcatagtcatggatacaactatttggattggtacctgcagaagccagggcagtctccacaacttctgatctatttgggttcttatcgggcctccggggtccctgacaggttcagtggcagtggatcaggcacagattttacactgaaaatcagcagagtggaggctgaggatgttggggtttattactgcatgcaatctctagaagttccgttcacttttggccaggggaccaagctggagatcaaa

SEQ ID NO: 42 - protein, 112 aa, Artificial Sequence
  Synthetic Construct
DVVMTQSPLSLPVTPGEPASISCRSSQSLLHSHGYNYLDWYLQKPGQSPQLLIYLGSYRASGVPDRFSGSGSGTDFTLKISRVEAEDVGVYYCMQSLEVPFTFGQGTKLEIK

SEQ ID NO: 43 - DNA, 321 bp, Artificial Sequence
  light chain variable region
tcttctgagctgactcaggaccctgctgtgtctgtggccttgggacagacagtcaggatcacatgccaaggagacagcctcagaatttattatacaggctggtaccaacagaagccaggacaggcccctgtgcttgtcctctttggtaagaacaatcggccctcagggatcccagaccgattctctggctcccactcagggaacacagcttccttgaccatcactggggctcaagcggaagatgaggctgactattactgtaactcccgggacatcactggtgtccatcgattcggcggagggaccaagctgaccgtccta

SEQ ID NO: 44 - protein, 107 aa, Artificial Sequence
  Synthetic Construct
SSELTQDPAVSVALGQTVRITCQGDSLRIYYTGWYQQKPGQAPVLVLFGKNNRPSGIPDRFSGSHSGNTASLTITGAQAEDEADYYCNSRDITGVHRFGGGTKLTVL

SEQ ID NO: 45 - DNA, 336 bp, Artificial Sequence
  light chain variable region
gaaattgtgctgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 46 - protein, 112 aa, Artificial Sequence
  Synthetic Construct
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 47 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctaacactttcggcggagggaccaaggtggagatcaaa

SEQ ID NO: 48 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPNTFGGGTKVEIK

SEQ ID NO: 49 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccaatcactttcggccctgggaccaaagtggatatcaaa

SEQ ID NO: 50 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGPGTKVDIK

SEQ ID NO: 51 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacacctatttggattggtacctgcagaagccagggcagtctccacaactcctgatctatttgggttctaatcgggcctccggggtccctgacaggttcagcggcagtggatcaggcacagattttacactgaaaatcagcagagtggagcctgaggatgttggggtctattactgcatgcaagctctagaaatgcccctcactttcggcggagggaccaaggtggagatcaaa

SEQ ID NO: 52 - protein, 112 aa, Artificial Sequence
  Synthetic Construct
DVVMTQSPLSLPVTPGEPASISCRSSQSLLHSNGYTYLDWYLQKPGQSPQLLIYLGSNRASGVPDRFSGSGSGTDFTLKISRVEPEDVGVYYCMQALEMPLTFGGGTKVEIK

SEQ ID NO: 53 - DNA, 321 bp, Artificial Sequence
  light chain variable region
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccccctcactttcggcggagggaccaaggtggagatcaaa

SEQ ID NO: 54 - protein, 107 aa, Artificial Sequence
  Synthetic Construct
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 55 - DNA, 315 bp, Artificial Sequence
  light chain variable region
tcctatgtgctgactcagccaccctcagtgtccgtgtccccaggacagacagccagcatcacctgctctggagataaattgggggataaatatgttggctggtatcagcaaaaggcaggccaagcccctgttttggtcatctatcaagacaacaagcgaccctcagggatccctgagcgattctctggctccaactctgggaacacagccagtctgaccatcagcgggacccaggctatggatgaggctgactattactgtcaggcgtgggacagcggcacggtgttcggcggagggaccaagctgaccgtccta

SEQ ID NO: 56 - protein, 105 aa, Artificial Sequence
  Synthetic Construct
SYVLTQPPSVSVSPGQTASITCSGDKLGDKYVGWYQQKAGQAPVLVIYQDNKRPSGIPERFSGSNSGNTASLTISGTQAMDEADYYCQAWDSGTVFGGGTKLTVL

SEQ ID NO: 57 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaacccccctcactttcggcggagggaccaaggtggagatcaaa

SEQ ID NO: 58 - protein, 112 aa, Artificial Sequence; identical to SEQ ID NO: 6
  Synthetic Construct

SEQ ID NO: 59 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatggaagctctacaaactccattcactttcggccctgggaccaaggtggaaatcaaa

SEQ ID NO: 60 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMEALQTPFTFGPGTKVEIK

SEQ ID NO: 61 - DNA, 321 bp, Artificial Sequence
  light chain variable region
gacatccagttgacccagtctccatcctccctgtctgcgtctgtgggagacagagtcaccatcacttgccggtcaagtcaaggcattggttacttcttaaattggtatcagcaggaaccagggaaagccccaaagatcctgatctctgctgcatccactttgcaaagtggggtcccatcaaggttcagtggcagtggatctgggacagatttcacactctccatcaacaatctgcaacccgcagattttgcgacatactactgtcaacagagtcacagtcccccgtacactttcggccaggggaccaaggtggagatcaaa

SEQ ID NO: 62 - protein, 107 aa, Artificial Sequence
  Synthetic Construct
DIQLTQSPSSLSASVGDRVTITCRSSQGIGYFLNWYQQEPGKAPKILISAASTLQSGVPSRFSGSGSGTDFTLSINNLQPADFATYYCQQSHSPPYTFGQGTKVEIK

SEQ ID NO: 63 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 64 - protein, 112 aa, Artificial Sequence; identical to SEQ ID NO: 6
  Synthetic Construct

SEQ ID NO: 65 - DNA, 336 bp, Artificial Sequence
  light chain variable region
gaaattgtgctgactcagtctccactctccctgcccgtcacccctggagagccggcctccatctcctgcaggtctagtcagagcctcctgcatagtaatggatacaactatttggattggtacctgcagaagccagggcagtctccacagctcctgatgtatttggtttctaatcgggcctccggggtccctgagaggttcagtggcagtggatcaggcacagattttacactgaaaatcagcagagtggaggctgaggatgttggggtttattactgcatgcaaactctacaaactcctctcagttttggccaggggaccaagctggagatcaaa

SEQ ID NO: 66 - protein, 112 aa, Artificial Sequence
  Synthetic Construct
EIVLTQSPLSLPVTPGEPASISCRSSQSLLHSNGYNYLDWYLQKPGQSPQLLMYLVSNRASGVPERFSGSGSGTDFTLKISRVEAEDVGVYYCMQTLQTPLSFGQGTKLEIK

SEQ ID NO: 67 - DNA, 336 bp, Artificial Sequence; identical to SEQ ID NO: 63
  light chain variable region

SEQ ID NO: 68 - protein, 112 aa, Artificial Sequence; identical to SEQ ID NO: 6
  Synthetic Construct

SEQ ID NO: 69 - DNA, 330 bp, Artificial Sequence
  light chain variable region
aattttatgctgactcagccccactctgtgtcggcgtctccggggaagacggttaccatctcctgcacccgcagcagtggcgacattgacaacaactatgtgcagtggtaccagcagcgcccgggcaattcccccaccaatgtgatttatgaggataaccgaagaccctctggggtcccggatcgcttctctggctccatcgacagctcctccaactctgcctccctcaccatctctggactgcagcctgaggacgaggctgactactattgtcagtcttatcaaagcgacaattgggtgttcggcggagggaccaaggtgaccgtccta

SEQ ID NO: 70 - protein, 110 aa, Artificial Sequence
  Synthetic Construct
NFMLTQPHSVSASPGKTVTISCTRSSGDIDNNYVQWYQQRPGNSPTNVIYEDNRRPSGVPDRFSGSIDSSSNSASLTISGLQPEDEADYYCQSYQSDNWVFGGGTKVTVL

SEQ ID NO: 71 - DNA, 330 bp, Artificial Sequence
  light chain variable region
aattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcgattctctggctccatcgacagctcctccaactctgcctccctcaccatctctggactgaagactgaggacgaggctgactactactgtcagtcttatgatagcagcaatgtggtgttcggcggagggaccaagctgaccgtccta

SEQ ID NO: 72 - protein, 110 aa, Artificial Sequence
  Synthetic Construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVL

SEQ ID NO: 73 - DNA, 336 bp, Artificial Sequence
  light chain variable region
gatgttgtgatgactcagtctccactctccctgcccgtcacccctggggagccggcctccatctcctgcaggtctagtcagagcctcctgcatagtaatggatacaactatttggattggtacctgcagaagccagggcagtctccacagctcctgatctatttgggttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaggctggagatcaaa

SEQ ID NO: 74 - protein, 112 aa, Artificial Sequence
  Synthetic Construct
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPYTFGQGTRLEIK

SEQ ID NO: 75 - DNA, 336 bp, Artificial Sequence
  light chain variable region
gatgttgtgatgactcagtctccactctccctgcccgtcacccctggagagtcggcctccatctcctgcaggtctagtcagagcctcctgcatagtaatggatacaactttttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 76 - protein, 112 aa, Artificial Sequence
  Synthetic Construct
DVVMTQSPLSLPVTPGESASISCRSSQSLLHSNGYNFLDWYLQKPGQSPQLLIYLGSNRASGVPDRFSGSGSGTDFTLKISRVEAEDVGVYYCMQALQTPLTFGGGTKVEIK

SEQ ID NO: 77 - DNA, 336 bp, Artificial Sequence; identical to SEQ ID NO: 57
  light chain variable region

SEQ ID NO: 78 - protein, 112 aa, Artificial Sequence; identical to SEQ ID NO: 6
  Synthetic Construct

SEQ ID NO: 79 - DNA, 321 bp, Artificial Sequence
  light chain variable region
gaaacgacactcacgcagtctccagccaccctgtctttgtctccagggcaaagagccaccctctcctgcagggccagtcagagtgtctacaactacttagcctggtaccaacagaagcctggccaggctcccaggctcctcatctatgatgcatccagaagggcaactggcatcccagccaggttcagtggcagtgggtctgggacagacttcactctcaccatcagcagcctagagcctgaagattttgcagtttattactgtcagcagcgtaacaactggccgctcactttcggtggagggaccaaggtggagatcaaa

SEQ ID NO: 80 - protein, 107 aa, Artificial Sequence
  Synthetic Construct
ETTLTQSPATLSLSPGQRATLSCRASQSVYNYLAWYQQKPGQAPRLLIYDASRRATGIPARFSGSGSGTDFTLTISSLEPEDFAVYYCQQRNNWPLTFGGGTKVEIK

SEQ ID NO: 81 - DNA, 321 bp, Artificial Sequence
  light chain variable region
gacatccagttgacccagtctccatcctccctgtctgcttctgttggagacagcgtcaccatctcttgccgggcaagtcagagtcctggcatctttttaaattggtatcagcagataccagggaaagcccctaaactcctgatctacgctacatccactctggaaagtggggtcccccccaggttcaccggcagtggatctgggacagatttcactctcaccatcagcagtctgcaacctgaggactttgcaacttactactgtcaacagagtaacagtgttccgctcactttcggcggcgggaccaaggtggagatcaaa

SEQ ID NO: 82 - protein, 107 aa, Artificial Sequence
  Synthetic Construct
DIQLTQSPSSLSASVGDSVTISCRASQSPGIFLNWYQQIPGKAPKLLIYATSTLESGVPPRFTGSGSGTDFTLTISSLQPEDFATYYCQQSNSVPLTFGGGTKVEIK

SEQ ID NO: 83 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactaaaaatcagcagagtggaggctgaggatgttggggtttattactgcatgcaagctctacaaactcctctaaccttcggccaagggacacgactggagattaaa

SEQ ID NO: 84 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGQGTRLEIK

SEQ ID NO: 85 - DNA, 321 bp, Artificial Sequence
  light chain variable region
gaaattgtgatgacgcagtctccagccaccctgtctgtgtctccaggggaaagagccaccttctcctgtagggccagtcagagtgttggcagcaacttagcctggtaccagcagaaacctggccaggctcccaggctcctcatctatgatgcatccaacagggccactggcatcccagccaggttcagtggcagtgggtctgggacagacttcactctcaccatcagcagactggagcctgaagattttgcagtgtattactgtcagcagcgtagcaactggcccctcactttcggcggagggaccaaggtggagatcaaa

SEQ ID NO: 86 - protein, 107 aa, Artificial Sequence
  Synthetic Construct
EIVMTQSPATLSVSPGERATFSCRASQSVGSNLAWYQQKPGQAPRLLIYDASNRATGIPARFSGSGSGTDFTLTISRLEPEDFAVYYCQQRSNWPLTFGGGTKVEIK

SEQ ID NO: 87 - DNA, 336 bp, Artificial Sequence; identical to SEQ ID NO: 63
  light chain variable region

SEQ ID NO: 88 - protein, 112 aa, Artificial Sequence; identical to SEQ ID NO: 6
  Synthetic Construct

SEQ ID NO: 89 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctacttgggttctactcgggcctccggcgtccctgacaggttcagtggcagtggatcaggcacagattttacactgaaaatcagcagagtggaggctgaggatgttggggtttattactgcatgcaagctctacaaactccttacactttcggcggagggaccaaggtggagatcaaa

SEQ ID NO: 90 - protein, 112 aa, Artificial Sequence
  Synthetic Construct
DVVMTQSPLSLPVTPGEPASISCRSSQSLLHSNGYNYLDWYLQKPGQSPQLLIYLGSTRASGVPDRFSGSGSGTDFTLKISRVEAEDVGVYYCMQALQTPYTFGGGTKVEIK

SEQ ID NO: 91 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcccctcactttcggcggagggaccaaggtggagatcaaa

SEQ ID NO: 92 - protein, 112 aa, Artificial Sequence; identical to SEQ ID NO: 6
  Synthetic Construct

SEQ ID NO: 93 - DNA, 336 bp, Artificial Sequence
  light chain variable region
gatgttgtgatgactcagtctccactctccctgcccgtcacccctggagagccggcctccatctcctgcaggtctagtcagagcctcctgcatactaatggatacaactatttggattggtacctgcagaagccagggcagtctccacggctcctgatctatttgggttttaatcgggcctccggggtccctgacaggttcagtggcagtggatcaggcacagattttacactgaaaatcagcagagtggaggctgaggatgttggggtttattactgtatgcaaggtctacaaactcccctcactttcggcggagggaccaaggtggagatcaaa

SEQ ID NO: 94 - protein, 112 aa, Artificial Sequence
  Synthetic Construct
DVVMTQSPLSLPVTPGEPASISCRSSQSLLHTNGYNYLDWYLQKPGQSPRLLIYLGFNRASGVPDRFSGSGSGTDFTLKISRVEAEDVGVYYCMQGLQTPLTFGGGTKVEIK

SEQ ID NO: 95 - DNA, 336 bp, Artificial Sequence
  light chain variable region
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattattgcatgcaagctacacactggccgtacacttttggccaggggaccaagctggagatcaaa

SEQ ID NO: 96 - protein, 112 aa, Artificial Sequence
  Synthetic Construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQATHWPYTFGQGTKLEIK

SEQ ID NO: 97 - DNA, 330 bp, Artificial Sequence
  light chain variable region
aattttatgctgactcagccccactctgtgtcggagtctccggggaagacggtaagcatctcctgcacccgcaacagtggcagcattgccagcaactttgtgcagtggtaccagcagcgcccgggcagtgcccccaccattgtaatctatgaggataaccaaagaccctctgcggtccctactcggttctctggctccatcgacaggtcctccaactctgcctccctcaccatctctggactgacgactgaggacgaggctgactactactgtcagtcttatgatagcgccaatgtcattttcggcggggggaccaagctgaccgtccta

SEQ ID NO: 98 - protein, 110 aa, Artificial Sequence
  Synthetic Construct
NFMLTQPHSVSESPGKTVSISCTRNSGSIASNFVQWYQQRPGSAPTIVIYEDNQRPSAVPTRFSGSIDRSSNSASLTISGLTTEDEADYYCQSYDSANVIFGGGTKLTVL

SEQ ID NO: 99 - DNA, 324 bp, Artificial Sequence
  light chain variable region
gaaacgacactcacgcagtctccaggcaccctgtctttgtctccaggggagagagccaccctctcctgcagggccagtcagactatcagcagcagccacttagcctggtaccagcagaaacctggccagtctcccaggctcctcatctatggtgcgggctacagggccaccggcattccagacaggttcagtggcagtgggtctggcacagacttcactctcaccatcagcagactggagcctgaagattttgcagtgtattactgtcagcactatggtagttcactccggacgttcggccaagggaccaaggtggaaatcaaa

SEQ ID NO: 100 - protein, 108 aa, Artificial Sequence
  Synthetic Construct
ETTLTQSPGTLSLSPGERATLSCRASQTISSSHLAWYQQKPGQSPRLLIYGAGYRATGIPDRFSGSGSGTDFTLTISRLEPEDFAVYYCQHYGSSLRTFGQGTKVEIK

SEQ ID NO: 101 - DNA, 330 bp, Artificial Sequence
  light chain variable region
aattttatgctgactcagccccactctgtgtcggagtctccggggaagacggtaaccatctcctgcaccggcagcggtggcaacattgccagcaattatgtgcagtggtaccagcagcgcccgggcagggcccccaccactgtgatctatgaggataatcgaagaccctctggggtccctgatcggttctctggctccatcgacagctcctccaactctgcctccctcaccatctctggactgaagactgaagacgaggctgactactactgtcagtcttatgatccctacaatcgagtgttcggcggagggaccaagctgaccgtccta

SEQ ID NO: 102 - protein, 110 aa, Artificial Sequence
  Synthetic Construct
NFMLTQPHSVSESPGKTVTISCTGSGGNIASNYVQWYQQRPGRAPTTVIYEDNRRPSGVPDRFSGSIDSSSNSASLTISGLKTEDEADYYCQSYDPYNRVFGGGTKLTVL

SEQ ID NO: 103 - DNA, 336 bp, Artificial Sequence
  light chain variable region
gaaattgtgatgacgcagtctccactctccctgcccgtcacccctggagagccggcctccatctcctgcaggtctagtcagagcctcctgcatactaatggatacgactatttggattggtacctgcagaagccagggcagtctccacagcttctgatctatttgggttctactcgggcctccggggtccctgacaggttcagtggcagtggatcgggcacagattttacactgaaaatcagcagagtggaggctgaggatgttggggtttattactgcatgcaagcttttcaaactccgctcactttcggcggagggaccaagatggagatcaaa

SEQ ID NO: 104 - protein, 112 aa, Artificial Sequence
  Synthetic Construct
EIVMTQSPLSLPVTPGEPASISCRSSQSLLHTNGYDYLDWYLQKPGQSPQLLIYLGSTRASGVPDRFSGSGSGTDFTLKISRVEAEDVGVYYCMQAFQTPLTFGGGTKMEIK

SEQ ID NO: 105 - DNA, 351 bp, Artificial Sequence
  heavy chain variable region
gaggtgcagctggtggagaccggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagatttaattactatgatagtagtgtctggggccagggaaccctggtcaccgtctcaagc

SEQ ID NO: 106 - protein, 117 aa, Artificial Sequence
  Synthetic Construct
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCARFNYYDSSVWGQGTLVTVSS

SEQ ID NO: 107 - DNA, 348 bp, Artificial Sequence
  heavy chain variable region
gaggtgcagctggtggagaccggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagaggggttgagcagattgactactggggccagggaaccctggtcaccgtctcaagc

SEQ ID NO: 108 - protein, 116 aa, Artificial Sequence
  Synthetic Construct
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCARGVEQIDYWGQGTLVTVSS

SEQ ID NO: 109 - DNA, 354 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgactgccgcggacacggccgtgtattactgtgcgaaaaatttagcagcaggggcggttgcctactggggccagggcaccctggtcaccgtctcaagc

SEQ ID NO: 110 - protein, 118 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAKNLAAGAVAYWGQGTLVTVSS

SEQ ID NO: 111 - DNA, 351 bp, Artificial Sequence
  heavy chain variable region
caggtgcagctacagcagtggggcgcaggactgttgaagccttcggagaccctgtccctcacctgcgctgtctctggtgggtccttcagtggttactactggagctggatccgtcagcccccagggaaggggctggagtggattggggaaatcaatcatagtggaagtaccaactacaaccggtccctcaagagtcgagtcaccatatcagtagacacgtccaagaaccagttctccctgaagctgagctctgtgaccgccgcggacacggctgtgtattactgtgcgagactttcatatggttcgggcgttgactactggggccagggcaccctggtcaccgtctcaagc

SEQ ID NO: 112 - protein, 117 aa, Artificial Sequence
  Synthetic Construct
QVQLQQWGAGLLKPSETLSLTCAVSGGSFSGYYWSWIRQPPGKGLEWIGEINHSGSTNYNRSLKSRVTISVDTSKNQFSLKLSSVTAADTAVYYCARLSYGSGVDYWGQGTLVTVSS

SEQ ID NO: 113 - DNA, 360 bp, Artificial Sequence
  heavy chain variable region
cagctgcagctgcaggagtcgggcccaggactggtgaagccttcacagaccctgtccctcacctgcactgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgaggtatagcagcagccgcaatgatgcttttgatatctggggccaagggacaatggtcaccgtctcaagc

SEQ ID NO: 114 - protein, 120 aa, Artificial Sequence
  Synthetic Construct
QLQLQESGPGLVKPSQTLSLTCTVSGGSISSSNWWSWVRQPPGKGLEWIGEIYHSGSTNYNPSLKSRVTISVDKSKNQFSLKLSSVTAADTAVYYCARYSSSRNDAFDIWGQGTMVTVSS

SEQ ID NO: 115 - DNA, 354 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagatgggcagctggatgcttttgatatctggggccaagggacaatggtcaccgtctcaagc

SEQ ID NO: 116 - protein, 118 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARDGQLDAFDIWGQGTMVTVSS

SEQ ID NO: 117 - DNA, 354 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagattttgggactactacggtatggacgtctggggccaagggaccacggtcaccgtctcaagc

SEQ ID NO: 118 - protein, 118 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARFWDYYGMDVWGQGTTVTVSS

SEQ ID NO: 119 - DNA, 351 bp, Artificial Sequence
  heavy chain variable region
caggtgcagctacagcagtggggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcgagagtcgagtcaccatatcagtagacaagtccaagaaccagttctccctgaagctgagctctgtgaccgccgcagacacggccgtgtattactgtgcgagagatcggtactacggtatggacgtctggggccaagggaccacggtcaccgtctcaagc

SEQ ID NO: 120 - protein, 117 aa, Artificial Sequence
  Synthetic Construct
QVQLQQWGPGLVKPSGTLSLTCAVSGGSISSSNWWSWVRQPPGKGLEWIGEIYHSGSTNYNPSLESRVTISVDKSKNQFSLKLSSVTAADTAVYYCARDRYYGMDVWGQGTTVTVSS

SEQ ID NO: 121 - DNA, 354 bp, Artificial Sequence
  heavy chain variable region
gaggtgcagctggtcgagtctggcccaggactggtgaagccttcggggaccctgtccctcacctgcgctgtctctggtggctccatcagcagtagtaactggtggagttgggtccgccagcccccagggaaggggctggagtggattgggtacatctattatagtgggagcacctactacaacccgtccctcaagagtcgagtcaccatgtcagtagacacgtccaagaaccagttctccctgaagctgagctctgtgaccgccgcagacacggccgtgtattactgtgcgagatggagctacttggatgcttttgatatctggggccaagggacaatggtcaccgtctcaagc

SEQ ID NO: 122 - protein, 118 aa, Artificial Sequence
  Synthetic Construct
EVQLVESGPGLVKPSGTLSLTCAVSGGSISSSNWWSWVRQPPGKGLEWIGYIYYSGSTYYNPSLKSRVTMSVDTSKNQFSLKLSSVTAADTAVYYCARWSYLDAFDIWGQGTMVTVSS

SEQ ID NO: 123 - DNA, 354 bp, Artificial Sequence
  heavy chain variable region
gaggtgcagctggtggagtctggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagattacgatattttcggtatggacgtctggggccaagggaccacggtcaccgtctcaagc

SEQ ID NO: 124 - protein, 118 aa, Artificial Sequence
  Synthetic Construct
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARDYDIFGMDVWGQGTTVTVSS

SEQ ID NO: 125 - DNA, 354 bp, Artificial Sequence
  heavy chain variable region
cagctgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagtcctccctgaagctgagctctgtgaccgccgcggacacggccgtgtattactgtgcgagagccaacagagatgatgcttttgatatctggggccaagggacaatggtcaccgtctcaagc

SEQ ID NO: 126 - protein, 118 aa, Artificial Sequence
  Synthetic Construct
QLQLQESGPGLVKPSGTLSLTCAVSGGSISSSNWWSWVRQPPGKGLEWIGEIYHSGSTNYNPSLKSRVTISVDKSKNQSSLKLSSVTAADTAVYYCARANRDDAFDIWGQGTMVTVSS

SEQ ID NO: 127 - DNA, 357 bp, Artificial Sequence
  heavy chain variable region
gaggtgcagctggtggagtctgggggaggcttggtacagccgggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagtctgagcgccgacgacacggccgtatatttctgtgcgtcgggtggctggtacggggactactttgactactggggccagggaaccctggtcaccgtctcaagc

SEQ ID NO: 128 - protein, 119 aa, Artificial Sequence
  Synthetic Construct
EVQLVESGGGLVQPGGSLRLSCAASGFTFSSYAMSWVRQAPGKGLEWVSAISGSGGSTYYADSVKGRFTISRDNSKNTLYLQMNSLSADDTAVYFCASGGWYGDYFDYWGQGTLVTVSS

SEQ ID NO: 129 - DNA, 363 bp, Artificial Sequence
  heavy chain variable region
caggtgcagctgcaggagtccggcccaggactggtgaagccttcggagaccctgtccctcacctgcactgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagaagggaaccgaacggtgactagtgcttttgatatctggggccaagggacaatggtcaccgtctcaagc

SEQ ID NO: 130 - protein, 121 aa, Artificial Sequence
  Synthetic Construct
QVQLQESGPGLVKPSETLSLTCTVSGGSISSSNWWSWVRQPPGKGLEWIGEIYHSGSTNYNPSLKSRVTISVDKSKNQFSLKLSSVTAADTAVYYCAREGNRTVTSAFDIWGQGTMVTVSS

SEQ ID NO: 131 - DNA, 357 bp, Artificial Sequence
  heavy chain variable region
caggtgcagctgcaggagtccggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgctgcggacacggccgtgtactactgtgcgagagggctgggggatagtagtggttatatcctttggggccaagggacaatggtcaccgtctcaagc

SEQ ID NO: 132 - protein, 119 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGLGDSSGYILWGQGTMVTVSS

SEQ ID NO: 133 - DNA, 357 bp, Artificial Sequence; identical to SEQ ID NO: 131
  heavy chain variable region

SEQ ID NO: 134 - protein, 119 aa, Artificial Sequence; identical to SEQ ID NO: 132
  Synthetic Construct

SEQ ID NO: 135 - DNA, 357 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagatggaccgggcgtactgatgcttttgatatctggggccaagggacaatggtcaccgtctcaagc

SEQ ID NO: 136 - protein, 119 aa, Artificial Sequence [sequence omitted]

SEQ ID NO: 137 - DNA, 354 bp, Artificial Sequence
  heavy chain variable region
caggtgcagctgcaggagtccggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagacaaggggcgttagatgcttttgatatctggggccaagggaccacggtcaccgtctcaagc

SEQ ID NO: 138 - protein, 118 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARQGALDAFDIWGQGTTVTVSS

SEQ ID NO: 139 - DNA, 366 bp, Artificial Sequence
  heavy chain variable region
caggtgcagctggtggagtccgggggaggcgtggtccgacctggggggtccctgagactctcctgtgcagcgtctggattcacctttagcagctatgccatgagctgggtccgccaggctccagggaaggggctggagtgggtctcaactattagtggtagtggtggtagcacatactacgcagactccgtgaagggccggttcaccatctccagagacaattccaagaacacgctgtatctgcagatgaacagcctgagagccgaggacacggccgtatattactgtgcgaaagagcgtggcagtggctggtccttagacaatatggacgtctggggccaagggaccacggtcaccgtctcaagc

SEQ ID NO: 140 - protein, 122 aa, Artificial Sequence
  Synthetic Construct
QVQLVESGGGVVRPGGSLRLSCAASGFTFSSYAMSWVRQAPGKGLEWVSTISGSGGSTYYADSVKGRFTISRDNSKNTLYLQMNSLRAEDTAVYYCAKERGSGWSLDNMDVWGQGTTVTVSS

SEQ ID NO: 141 - DNA, 357 bp, Artificial Sequence
  heavy chain variable region
caggtgcagctggtggagtctggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgctgcggacacggccgtgtattactgtgcgagagatagcagtgggttctacggtatggacgtctggggccaagggaccacggtcaccgtctcaagc

SEQ ID NO: 142 - protein, 119 aa, Artificial Sequence
  Synthetic Construct
QVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARDSSGFYGMDVWGQGTTVTVSS

SEQ ID NO: 143 - DNA, 360 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgactgccgcggacacggccgtgtattactgtgcgagaagcagcagctggtactggaatgcttttgatatctggggccaagggacaatggtcaccgtctcaagc

SEQ ID NO: 144 - protein, 120 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARSSSWYWNAFDIWGQGTMVTVSS

SEQ ID NO: 145 - DNA, 351 bp, Artificial Sequence
  heavy chain variable region
caggtgcagctacagcagtggggcccagcactggtgaagccttcggggaccctgtccctcacctgctctgtctctggtgtctccatcaccagtaatatctggtggagttgggtccgccagtccccagggaaggggctggagtggattggggaagtctatcatagtgggagcaccaactacaacccgtccctcaagagtcgagtcaccatatcagtagacaagtccaagaaccagttctccctgaagctgagctctgtgaccgccgcggacacggctgtgtattactgtgcggggtaccgtagcttcggggagtcctactggggccagggaaccctggtcaccgtctcaagc

SEQ ID NO: 146 - protein, 117 aa, Artificial Sequence
  Synthetic Construct
QVQLQQWGPALVKPSGTLSLTCSVSGVSITSNIWWSWVRQSPGKGLEWIGEVYHSGSTNYNPSLKSRVTISVDKSKNQFSLKLSSVTAADTAVYYCAGYRSFGESYWGQGTLVTVSS

SEQ ID NO: 147 - DNA, 366 bp, Artificial Sequence
  heavy chain variable region
caggtgcagctacagcagtggggcgcagggctgttgaagccttcggagaccctgtctctcacctgcgttgtctatggtgggtccttcagcgatttctactggagctggatccgccagcccccagggaaggggccagagtggattggggaagtcaatcctagaggaagcaccaactacaacccgtccctcaagagtcgagccaccatatcactagacacgtccaagaaccagttctccctgaagctgagttctgtgaccgccgcggacacggctgtgtatttctgtgcgagaggtcctcggcccgggagagatggctacaattactttgacaactggggccagggcaccctggtcaccgtctcaagc

SEQ ID NO: 148 - protein, 122 aa, Artificial Sequence
  Synthetic Construct
QVQLQQWGAGLLKPSETLSLTCVVYGGSFSDFYWSWIRQPPGKGPEWIGEVNPRGSTNYNPSLKSRATISLDTSKNQFSLKLSSVTAADTAVYFCARGPRPGRDGYNYFDNWGQGTLVTVSS

SEQ ID NO: 149 - DNA, 357 bp, Artificial Sequence
  heavy chain variable region
caggtgcagctgcaggagtcgggcccaggactggtgaagccttcggagaccctgtccctcacctgcactgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagaggtatagcagcagctggtcaaggtgactactggggccagggaaccctggtcaccgtctcaagc

SEQ ID NO: 150 - protein, 119 aa, Artificial Sequence
  Synthetic Construct
QVQLQESGPGLVKPSETLSLTCTVSGGSISSSNWWSWVRQPPGKGLEWIGEIYHSGSTNYNPSLKSRVTISVDKSKNQFSLKLSSVTAADTAVYYCARGIAAAGQGDYWGQGTLVTVSS

SEQ ID NO: 151 - DNA, 363 bp, Artificial Sequence
  heavy chain variable region
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcggacacggccgtgtattactgtgcgagagatgggggatactactactacggtatggacgtctggggccaagggaccacggtcaccgtctcaagc

SEQ ID NO: 152 - protein, 121 aa, Artificial Sequence
  Synthetic Construct
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDGGYYYYGMDVWGQGTTVTVSS

SEQ ID NO: 153 - DNA, 351 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagtagtggttatgatgcttttgatatctggggccaagggaccacggtcaccgtctcaagc

SEQ ID NO: 154 - protein, 117 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCASSGYDAFDIWGQGTTVTVSS

SEQ ID NO: 155 - DNA, 357 bp, Artificial Sequence
  heavy chain variable region
caggtgcagctgcaggagtcgggcccaggactggtgaagccttcggggaccctgtccctcacctgcgctgtctctggtggctccatcagcagtagtaattggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcacgatacagctatggaacggtaggaattgactactggggccagggaaccctggtcaccgtctcaagc

SEQ ID NO: 156 - protein, 119 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARYSYGTVGIDYWGQGTLVTVSS

SEQ ID NO: 157 - DNA, 351 bp, Artificial Sequence
  heavy chain variable region
gaggtgcagctggtgcagtctgggggaggcgtggtccagcctgggacgtccctgagactctcctgtgcagcctctggattcagcttcagaagtcatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgactatagggccggggggatttgactactggggccagggcaccctggtcaccgtctcaagc

SEQ ID NO: 158 - protein, 117 aa, Artificial Sequence
  Synthetic Construct
EVQLVQSGGGVVQPGTSLRLSCAASGFSFRSHGMHWVRQAPGKGLEWVAVISYDGSNKYYADSVKGRFTISRDNSKNTLYLQMNSLRAEDTAVYYCATIGPGGFDYWGQGTLVTVSS

SEQ ID NO: 159 - DNA, 357 bp, Artificial Sequence
  heavy chain variable region
caggtgcagctgcaggagtccggcccaggactggtgaagccttcggagaccctgtccctcacctgcactgtctctggtggctccattagaaattactactggagttggatccggcagcccccagggaagggactggagtggattgggtatatttctgacagtgggaataccaactacaatccctccctcaagagtcgagtcaccatatcagtagacacgtccaagaaccagttctccctaaagctgacctctgtgaccgccacagacacggctgcgtatttctgtgcgagacatcgaagcagctgggcatggtacttcgatctctggggccgtggcaccctggtcaccgtctcaagc

SEQ ID NO: 160 - protein, 119 aa, Artificial Sequence
  Synthetic Construct
QVQLQESGPGLVKPSETLSLTCTVSGGSIRNYYWSWIRQPPGKGLEWIGYISDSGNTNYNPSLKSRVTISVDTSKNQFSLKLTSVTATDTAAYFCARHRSSWAWYFDLWGRGTLVTVSS

SEQ ID NO: 161 - DNA, 354 bp, Artificial Sequence
  heavy chain variable region
caggtgcagctgcaggagtcgggcccaggactggtgaagccttcggagaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagtgggcagtggctggtacgttgactactggggccagggaaccctggtcaccgtctcaagc

SEQ ID NO: 162 - protein, 118 aa, Artificial Sequence
  Synthetic Construct
QVQLQESGPGLVKPSETLSLTCAVSGGSISSSNWWSWVRQPPGKGLEWIGEIYHSGSTNYNPSLKSRVTISVDKSKNQFSLKLSSVTAADTAVYYCARVGSGWYVDYWGQGTLVTVSS

SEQ ID NO: 163 - DNA, 360 bp, Artificial Sequence
  heavy chain variable region
caggtgcagctgcaggagtccggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagtttctggctactactactacggtatggacgtctggggccaagggaccacggtcaccgtctcaagc

SEQ ID NO: 164 - protein, 120 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARVSGYYYYGMDVWGQGTTVTVSS

SEQ ID NO: 165 - DNA, 369 bp, Artificial Sequence
  heavy chain variable region
gaggtccagctggtacagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagcgtatagcagtggctggtacgactactacggtatggacgtctggggccaagggaccacggtcaccgtctcaagc

SEQ ID NO: 166 - protein, 123 aa, Artificial Sequence
  Synthetic Construct
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKAYSSGWYDYYGMDVWGQGTTVTVSS

SEQ ID NO: 167 - DNA, 351 bp, Artificial Sequence
  heavy chain variable region
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagccagcgttgatgcttttgatatctggggccaagggacaatggtcaccgtctcaagc

SEQ ID NO: 168 - protein, 117 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARASVDAFDIWGQGTMVTVSS

SEQ ID NO: 169 - DNA, 357 bp, Artificial Sequence; identical to SEQ ID NO: 131
  heavy chain variable region

SEQ ID NO: 170 - protein, 119 aa, Artificial Sequence; identical to SEQ ID NO: 132
  Synthetic Construct

SEQ ID NO: 171 - DNA, 348 bp, Artificial Sequence
  heavy chain variable region
caggtacagctgcagcagtcaggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgactcccgaggacacggctgtgtattactgtgcaagagatcacggcccctttgactactggggccggggaaccctggtcaccgtctcaagc

SEQ ID NO: 172 - protein, 116 aa, Artificial Sequence
  Synthetic Construct
QVQLQQSGPGLVKPSGTLSLTCAVSGGSISSSNWWSWVRQPPGKGLEWIGEIYHSGSTNYNPSLKSRVTISVDKSKNQFSLKLSSVTPEDTAVYYCARDHGPFDYWGRGTLVTVSS

SEQ ID NO: 173 - DNA, 360 bp, Artificial Sequence
  heavy chain variable region
caggtgcagctggtgcaatctgggggaggcgtggtccagcctgggaggtccctgagactctcctgtgcagcctctggattcgccttcagtagctatggcatgcactgggtccgccaggctccagggaaggggctggagtgggtttcatacattagtagtagtagtagtaccatatactacgcagactctgtgaagggccgattcaccatctccagagacaattccaagaacacgctgtatctgcaaatgaacagcctgagagccgaggacacggctgtgtattactgtgcgagagatcgatttgggtcggggcacttgcccgactactggggccagggaaccctggtcaccgtctcaagc

SEQ ID NO: 174 - protein, 120 aa, Artificial Sequence
  Synthetic Construct
QVQLVQSGGGVVQPGRSLRLSCAASGFAFSSYGMHWVRQAPGKGLEWVSYISSSSSTIYYADSVKGRFTISRDNSKNTLYLQMNSLRAEDTAVYYCARDRFGSGHLPDYWGQGTLVTVSS

SEQ ID NO: 175 - DNA, 357 bp, Artificial Sequence
  heavy chain variable region
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagagttgggtatagcagtggccgtgacgttgactactggggccagggcaccctggtcaccgtctcaagc

SEQ ID NO: 176 - protein, 119 aa, Artificial Sequence
  Synthetic Construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVGYSSGRDVDYWGQGTLVTVSS

SEQ ID NO: 177 - DNA, 360 bp, Artificial Sequence
  heavy chain variable region
gaggtccagctggtggagtctggcccaggactggtgaagccttcggggaccctgtccctcacctgcgctgtctctggtggctccatcagcagtagtaactggtggagttggatccggcagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagatagcagcagctggtactacggtatggacgtctggggccaagggaccacggtcaccgtctcaagc

SEQ ID NO: 178 - protein, 120 aa, Artificial Sequence
  Synthetic Construct
EVQLVESGPGLVKPSGTLSLTCAVSGGSISSSNWWSWIRQPPGKGLEWIGEIYHSGSTNYNPSLKSRVTISVDKSKNQFSLKLSSVTAADTAVYYCARDSSSWYYGMDVWGQGTTVTVSS

SEQ ID NO: 179 - DNA, 348 bp, Artificial Sequence
  heavy chain variable region
gaggtccagctggtggagtccggcccaggactggtgaagccttcggagaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgctgcggacacggccgtatattattgtgcgagatcgacgtggtcccttgactactggggccagggcaccctggtcaccgtctcaagc

SEQ ID NO: 180 - protein, 116 aa, Artificial Sequence
  Synthetic Construct
EVQLVESGPGLVKPSETLSLTCAVSGGSISSSNWWSWVRQPPGKGLEWIGEIYHSGSTNYNPSLKSRVTISVDKSKNQFSLKLSSVTAADTAVYYCARSTWSLDYWGQGTLVTVSS

SEQ ID NO: 181 - DNA, 354 bp, Artificial Sequence
  heavy chain variable region
gaggtccagctggtggagtctggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgctgcggacacggccgtatattactgtgcgagactctcgtttgccgatccttttgatatctggggccaagggacaatggtcaccgtctcaagc

SEQ ID NO: 182 - protein, 118 aa, Artificial Sequence
  Synthetic Construct
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARLSFADPFDIWGQGTMVTVSS

SEQ ID NO: 183 - DNA, 366 bp, Artificial Sequence
  heavy chain variable region
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccccatccttggtatagcaaactacgcacagaagttccagggcagagtcacgattaccgcggacaaatccacgagcacagcctacatggagctgagcagcctgagatctgaggacacggccgtgtattactgtgcatatggttcggggagttattacgactactactacatggacgtctggggcaaagggaccacggtcaccgtctcaagc

SEQ ID NO: 184 - protein, 122 aa, Artificial Sequence
  Synthetic Construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAYGSGSYYDYYYMDVWGKGTTVTVSS

SEQ ID NO: 185 - DNA, 357 bp, Artificial Sequence
  heavy chain variable region
gaggtccagctggtgcagtctgggggaggcttggtccagcctggggggtccctgagactctcctgttcagcctccggattcaccttcagtagctatgctatgcactgggtccgccaggctccagggaagggactggaatatgtttcaactattagtagtaatggggatagcacatactacgcagactccgtgaagggcagattcaccatctccagagacaattccaagaacacgctgtatctgcaaatgaacagcctgagagctgaggacacggctgtgtattactgtgcgaaagaagaagtatggctacaggcttttgatatctggggccaagggacaatggtcaccgtctcaagc

SEQ ID NO: 186 - protein, 119 aa, Artificial Sequence
  Synthetic Construct
EVQLVQSGGGLVQPGGSLRLSCSASGFTFSSYAMHWVRQAPGKGLEYVSTISSNGDSTYYADSVKGRFTISRDNSKNTLYLQMNSLRAEDTAVYYCAKEEVWLQAFDIWGQGTMVTVSS

SEQ ID NO: 187 - DNA, 345 bp, Artificial Sequence
  heavy chain variable region
cagctgcagctgcaggagtcgggcccaggactggtgaagccttcggagaccctgtccctcacctgcactgtctctggtggctccatcagtagtaactggtggagttgggtccgccagcccccagggaaggggctggagtggattggggaaatctatcatagtgggagcaccaactacaacccctccctcaagagtcgagtcaccatctcagtagacacgtccaagaaccagttctccctgaagctgagctctgtgaccgctgcggacacggccgtgtattactgtgcgagagataagggatacatggacgtctggggcaaagggaccacggtcaccgtctcaagc

SEQ ID NO: 188 - protein, 115 aa, Artificial Sequence
  Synthetic Construct
QLQLQESGPGLVKPSETLSLTCTVSGGSISSNWWSWVRQPPGKGLEWIGEIYHSGSTNYNPSLKSRVTISVDTSKNQFSLKLSSVTAADTAVYYCARDKGYMDVWGKGTTVTVSS

SEQ ID NO: 189 - DNA, 363 bp, Artificial Sequence
  heavy chain variable region
caggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagatcataggttcgactacgcctggtacttcgatctctggggccgtggcaccctggtcaccgtctcaagc

SEQ ID NO: 190 - protein, 121 aa, Artificial Sequence
  Synthetic Construct
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARDHRFDYAWYFDLWGRGTLVTVSS

SEQ ID NO: 191 - DNA, 351 bp, Artificial Sequence
  heavy chain variable region
caggtgcagctgcaggagtcgggcccaggactgctgaagccttcggggaccctgtccctcacctgcgctgtctctggtggctccatcagcagtagcaactggtggagttgggtccgccagcccccaggggaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtctattactgtgcgagagatctaacggggagtcttgactactggggccagggaaccctggtcaccgtctcaagc

SEQ ID NO: 192 - protein, 117 aa, Artificial Sequence
  Synthetic Construct
QVQLQESGPGLLKPSGTLSLTCAVSGGSISSSNWWSWVRQPPGEGLEWIGEIYHSGSTNYNPSLKSRVTISVDKSKNQFSLKLSSVTAADTAVYYCARDLTGSLDYWGQGTLVTVSS

SEQ ID NO: 193 - DNA, 351 bp, Artificial Sequence
  heavy chain variable region
caggtgcagctgcaggagtccggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagaatacgctatgatgcttttgatatctggggccaagggacaatggtcaccgtctcaagc

SEQ ID NO: 194 - protein, 117 aa, Artificial Sequence
  Synthetic Construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARIRYDAFDIWGQGTMVTVSS

SEQ ID NO: 195 - DNA, 354 bp, Artificial Sequence
  heavy chain variable region
caggtgcagctgcaggagtcgggcccaggactggtgaagccttcggagaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgctgcggacacggccgtgtattactgtgccgtgacggcagcccatgatgcttttgatatctggggccaagggacaatggtcaccgtctcaagc

SEQ ID NO: 196 - protein, 118 aa, Artificial Sequence
  Synthetic Construct
QVQLQESGPGLVKPSETLSLTCAVSGGSISSSNWWSWVRQPPGKGLEWIGEIYHSGSTNYNPSLKSRVTISVDKSKNQFSLKLSSVTAADTAVYYCAVTAAHDAFDIWGQGTMVTVSS

SEQ ID NO: 197 - DNA, 357 bp, Artificial Sequence
  heavy chain variable region
caggtgcagctacagcagtggggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagacagcagtggccaagggtactttgactactggggccagggcaccctggtcaccgtctcaagc

SEQ ID NO: 198 - protein, 119 aa, Artificial Sequence
  Synthetic Construct
QVQLQQWGPGLVKPSGTLSLTCAVSGGSISSSNWWSWVRQPPGKGLEWIGEIYHSGSTNYNPSLKSRVTISVDKSKNQFSLKLSSVTAADTAVYYCARDSSGQGYFDYWGQGTLVTVSS

SEQ ID NO: 199 - DNA, 354 bp, Artificial Sequence
  heavy chain variable region
gaggtgcagctggtgcagtctggggctgaggtgaagaagcctggggcctcagtgaaggtctcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccatgaccagggacacgtccacgagcacagtctacatggagctgagcagcctgagatctgaggacacggccgtgtattactgtgctagacactcgtactactacggtatggacgtctggggccaaggcaccctggtcaccgtctcaagc

SEQ ID NO: 200 - protein, 118 aa, Artificial Sequence
  Synthetic Construct
EVQLVQSGAEVKKPGASVKVSCKASGYTFTSYAMHWVRQAPGQRLEWMGWINAGNGNTKYSQKFQGRVTMTRDTSTSTVYMELSSLRSEDTAVYYCARHSYYYGMDVWGQGTLVTVSS

SEQ ID NO: 201 - DNA, 360 bp, Artificial Sequence
  heavy chain variable region
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcggtagacacgtccaagaaccagttctccctgaagctgagctctgtgaccgccgcggacacggctgtgtattactgtgcgagagtcgggtatagccacggcgaagaagtcctggacgtctggggcaaagggaccacggtcaccgtctcaagc

SEQ ID NO: 202 - protein, 120 aa, Artificial Sequence
  Synthetic Construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVGYSHGEEVLDVWGKGTTVTVSS

SEQ ID NO: 203 - DNA, 354 bp, Artificial Sequence
  heavy chain variable region
caggtgcagctgcaggagtcgggcccaggactggtgaagccttcggagaccctgtccctcacctgcactgtctctggtggctccatcggcaattatgactggagttggatccggcagcccccagggaagggactggagtggattgggactatctactctagtgggagtacgtactacagtccgtccctcaagagtcgactcaccatatcagtagacaagtccaagaaccggttctccctgaagctgagctctgtgaccgccgcggacacggccgtgtattactgtgcgagagcacgagggtatagcagccccttcgacccctggggccagggcaccctggtcaccgtctcaagc

SEQ ID NO: 204 - protein, 118 aa, Artificial Sequence
  Synthetic Construct
QVQLQESGPGLVKPSETLSLTCTVSGGSIGNYDWSWIRQPPGKGLEWIGTIYSSGSTYYSPSLKSRLTISVDKSKNRFSLKLSSVTAADTAVYYCARARGYSSPFDPWGQGTLVTVSS

SEQ ID NO: 205 - DNA, 357 bp, Artificial Sequence
  heavy chain variable region
caggtccagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggaataatcaaccctagtggtggtagcacaagctacgcacagaagttccagggcagagtcaccattaccagggacacatccgcgagcacagcctacatggagctgagcagcctgagatctgaagacacggctgtgtattactgtgcgagagatcggtggaggtacgatgcttttgatatctggggccaagggacaatggtcaccgtctcaagc

SEQ ID NO: 206 - protein, 119 aa, Artificial Sequence
  Synthetic Construct
QVQLVQSGAEVKKPGSSVKVSCKASGGTFSSYAISWVRQAPGQGLEWMGIINPSGGSTSYAQKFQGRVTITRDTSASTAYMELSSLRSEDTAVYYCARDRWRYDAFDIWGQGTMVTVSS

SEQ ID NO: 207 - DNA, 348 bp, Artificial Sequence
  heavy chain variable region
gaggtgcagctggtggagtctggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagaaaaatcgggtatggacgtctggggccaagggaccacggtcaccgtctcaagc

SEQ ID NO: 208 - protein, 116 aa, Artificial Sequence
  Synthetic Construct
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCAREKSGMDVWGQGTTVTVSS

SEQ ID NO: 209 - DNA, 321 bp, Artificial Sequence
  light chain constant region
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 210 - protein, 107 aa, Artificial Sequence [sequence omitted]

SEQ ID NO: 211 - DNA, 990 bp, Artificial Sequence
  heavy chain constant region
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 212 - protein, 330 aa, Artificial Sequence [sequence omitted]

SEQ ID NO: 213 - protein, 9 aa, Artificial Sequence
  Light chain CDR3
MQALQTPXT

SEQ ID NO: 214 - protein, 9 aa, Artificial Sequence
  Light chain CDR3
QQXXXXPLT

SEQ ID NO: 215 - protein, 10 aa, Artificial Sequence
  Light chain CDR3
QSYDSSNXXV

SEQ ID NO: 216 - protein, 8 aa, Artificial Sequence
  Heavy chain CDR3
SRLDAFDI

SEQ ID NO: 217 - protein, 10 aa, Artificial Sequence
  Heavy chain CDR3
SXYDYYGMDV

SEQ ID NO: 218 - protein, 11 aa, Artificial Sequence
  Heavy chain CDR3
HRXDXAWYFDL

SEQ ID NO: 219 - protein, 4 aa, Artificial Sequence
  Heavy chain CDR3
DSSG

SEQ ID NO: 220 - protein, 16 aa, Artificial Sequence
  Light chain CDR1
RSSQSLLHSNGYNYLD

SEQ ID NO: 221 - protein, 11 aa, Artificial Sequence
  Light chain CDR1
RASQXXXXXLX

SEQ ID NO: 222 - protein, 11 aa, Artificial Sequence
  Light chain CDR1
RSSQSXXXXXX

SEQ ID NO: 223 - protein, 7 aa, Artificial Sequence
  Light chain CDR2
LGSNRAS

SEQ ID NO: 224 - protein, 7 aa, Artificial Sequence
  Light chain CDR2
AASTLQS

SEQ ID NO: 225 - protein, 7 aa, Artificial Sequence
  Light chain CDR2
EDNXRPS

SEQ ID NO: 226 - protein, 6 aa, Artificial Sequence
  Heavy chain CDR1
SSNWWS

SEQ ID NO: 227 - protein, 5 aa, Artificial Sequence
  Heavy chain CDR1
XYYWS

SEQ ID NO: 228 - protein, 5 aa, Artificial Sequence
  Heavy chain CDR1
SYAMX

SEQ ID NO: 229 - protein, 16 aa, Artificial Sequence
  Heavy chain CDR2
XXXXSGSTXYNPSLKS

SEQ ID NO: 230 - protein, 17 aa, Artificial Sequence
  Heavy chain CDR2
XISXSGXSTYYADSVKG

SEQ ID NO: 231 - protein, 1162 aa, Artificial Sequence
  huIGF-1RFc
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLDEVDGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 232 - protein, 1180 aa, Artificial Sequence
  hu INSRfC
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNHIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTTTQGKLFFHYNPKLCLSEIHKMEEVSGTKGRQERNDIALKTNGDKASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKVDGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 233 - protein, 1062 aa, Artificial Sequence
  hu IGF-1Ravidin
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYEAAAARKCSLTGKWTNDLGSNMTIGAVNSKGEFTGTYTTAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 234 - protein, 107 aa, Artificial Sequence [sequence omitted]

SEQ ID NO: 235 - protein, 330 aa, Artificial Sequence [sequence omitted]

SEQ ID NO: 236 - protein, 16 aa, Artificial Sequence
  light chain CDR1
RSSQSLLHXXGYNXLX

SEQ ID NO: 237 - protein, 13 aa, Artificial Sequence
  light chain CDR1
TRSSGXIXXNYVQ

SEQ ID NO: 238 - protein, 11 aa, Artificial Sequence
  light chain CDR1
RASQXXXXXLX

SEQ ID NO: 239 - protein, 7 aa, Artificial Sequence
  light chain CDR2
LXXXRXS

SEQ ID NO: 240 - protein, 7 aa, Artificial Sequence
  light chain CDR2
AXSXLXS

SEQ ID NO: 241 - protein, 7 aa, Artificial Sequence
  light chain CDR2
XXNXRPS

SEQ ID NO: 242 - protein, 9 aa, Artificial Sequence
  light chain CDR3
MXXXXXPXX

SEQ ID NO: 243 - protein, 9 aa, Artificial Sequence
  light chain CDR3
QQXXXXPXT

SEQ ID NO: 244 - protein, 10 aa, Artificial Sequence
  light chain CDR3
QSYXSXNXXV

SEQ ID NO: 245 - protein, 6 aa, Artificial Sequence
  heavy chain CDR1
XXXWWS

SEQ ID NO: 246 - protein, 5 aa, Artificial Sequence
  heavy chain CDR1
XXYWS

SEQ ID NO: 247 - protein, 5 aa, Artificial Sequence
  heavy chain CDR1
SYXXX

SEQ ID NO: 248 - protein, 16 aa, Artificial Sequence
  heavy chain CDR2
XXXXXGXTXYNPSLXS

SEQ ID NO: 249 - protein, 17 aa, Artificial Sequence
  heavy chain CDR2
XISXXXXXXYYADSVKG

SEQ ID NO: 250 - protein, 12 aa, Artificial Sequence
  heavy chain CDR3
XXXXXXXXXFDI

SEQ ID NO: 251 - protein, 14 aa, Artificial Sequence
  heavy chain CDR3
XXXXXXXXXXXMDV

SEQ ID NO: 252 - protein, 11 aa, Artificial Sequence
  heavy chain CDR3
XXXXXXXXXXY

SEQ ID NO: 253 - protein, 14 aa, Artificial Sequence
  Heavy chain CDR3
XXXXXXXXXXYFDX

SEQ ID NO: 254 - protein, 15 aa, Artificial Sequence
  heavy chain CDR3
XXXXDSSXXXXXXXX

SEQ ID NO: 255 - DNA, 8 bp, Artificial Sequence
  polypeptide
dykddddk

SEQ ID NO: 256 - DNA, 37 bp, Artificial Sequence
  nucleic acid
agcaagcttccaccatgaagtctggctccggaggagg

SEQ ID NO: 257 - DNA, 36 bp, Artificial Sequence
  nucleic acid
atttgtcgacttcgtccagatggatgaagttttcat

SEQ ID NO: 258 - DNA, 3486 bp, Artificial Sequence
  nucleic acid
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcactgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctggacgaagtcgacggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtaaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 259 - DNA, 32 bp, Artificial Sequence
  nucleic acid
agcaagcttccaccatgggcaccgggggccgg

SEQ ID NO: 260 - DNA, 37 bp, Artificial Sequence
  nucleic acid
atttgtcgacttttgcaatatttgacgggacgtctaa

SEQ ID NO: 261 - DNA, 3540 bp, Artificial Sequence
  nucleic acid
atgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataatcacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccaccactcaggggaaactcttcttccactataaccccaaactctgcttgtcagaaatccacaagatggaagaagtttcaggaaccaaggggcgccaggagagaaacgacattgccctgaagaccaatggggacaaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgatgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaagtcgacggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtaaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 262 - DNA, 1409 bp, Artificial Sequence
  nucleic acid
tctagaccaccatggacatcaggctcagcttagttttccttgtccttttcataaaaggtgtccagtgtgaggtagaactggtggagtctgggggcggcttagtacaacctggaaggtccatgacactctcctgtgcagcctcgggattcactttcagaacctatggcatggcctgggtccgccaggccccaacgaagggtctggagtgggtctcatcaattactgctagtggtggtaccacctactatcgagactccgtgaagggccgcttcactatttttagggataatgcaaaaagtaccctatacctgcagatggacagtccgaggtctgaggacacggccacttatttctgtacatcaatttcggaatactggggccacggagtcatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgataagtcgac

SEQ ID NO: 263 - protein, 22 aa, Artificial Sequence
  protein
MDMRVPAQLLGLLLLWLRGARC

SEQ ID NO: 264 - protein, 22 aa, Artificial Sequence
  protein
MDMRVPAQLLGLLLLWLRGARC

SEQ ID NO: 265 - DNA, 28 bp, Artificial Sequence
  nucleic acid
gcaagcttgggagaaatctgcgggccag

SEQ ID NO: 266 - DNA, 32 bp, Artificial Sequence
  nucleic acid
attgcggccgcttcatatcctgttttggcctg

SEQ ID NO: 267 - DNA, 30 bp, Artificial Sequence
  nucleic acid
attgcggccgccccacattcctttgggggc

SEQ ID NO: 268 - DNA, 28 bp, Artificial Sequence
  nucleic acid
agcaagcttggacctgtgtccagggacc

SEQ ID NO: 269 - DNA, 30 bp, Artificial Sequence
  nucleic acid
attgcggccgcgcaaggaccttcacaaggg

SEQ ID NO: 270 - DNA, 29 bp, Artificial Sequence
  nucleic acid
agcaagcttgccgaaggtctgtgaggaag

SEQ ID NO: 271 - DNA, 30 bp, Artificial Sequence
  nucleic acid
attgcggccgcactttcacaggaggctctc

SEQ ID NO: 272 - DNA, 30 bp, Artificial Sequence
  nucleic acid
agcaagcttggacgtcctgcatttcacctc

SEQ ID NO: 273 - DNA, 32 bp, Artificial Sequence
  nucleic acid
attgcggccgcggtgcgaatgtacaagatctc

SEQ ID NO: 274 - DNA, 32 bp, Artificial Sequence
  nucleic acid
agcaagcttgaatgcttcagttccttccattc

SEQ ID NO: 275 - DNA, 33 bp, Artificial Sequence
  nucleic acid
attgcggccgcagtccttgcaaagacgaagttg

SEQ ID NO: 276 - DNA, 29 bp, Artificial Sequence
  nucleic acid
agcaagcttgatgcccgcagaaggagcag

SEQ ID NO: 277 - DNA, 32 bp, Artificial Sequence
  nucleic acid
attgcggccgctttaatggccactctggtttc

SEQ ID NO: 278 - DNA, 29 bp, Artificial Sequence
  nucleic acid
agcaagcttgggagaaatctgcgggccag

SEQ ID NO: 279 - DNA, 29 bp, Artificial Sequence
  nucleic acid
agcaagcttgggagaaatctgcgggccag

(19) United States
(12) Patent Application Publication
(10) Pub. No.: US 2014/0037642 A1
(43) Pub. Date: Feb. 6, 2014

(54) METHODS AND COMPOSITIONS RELATING TO INHIBITION OF IGF-1R

(71) Applicants: Ian McCaffery, Oakland, CA (US); Jian-Feng Lu, Oak Park, CA (US)

(72) Inventors: Ian McCaffery, Oakland, CA (US); Jian-Feng Lu, Oak Park, CA (US)

(73) Assignee: AMGEN INC., Thousand Oaks, CA (US)

(21) Appl. No.: 13/983,460

(22) PCT Filed: Feb. 2, 2012
(86) PCT No.: PCT/US2012/023691

§ 371 Date: Oct. 18, 2013

Related U.S. Application Data
(60) Provisional application No. 61/438,918, filed on Feb. 2, 2011.

(51) Int. Cl. G01N 33/574

(52) U.S. Cl. CPC G01N 33/57488
USPC 424/142.1; 436/501

(57) ABSTRACT

The present invention provides, in part, methods for treating a tumor in a human subject comprising inhibiting IGF-1 receptor signaling, methods of determining whether a tumor is more or less likely to respond to such treatment, and compositions for practicing such methods. In particular embodiments, the invention provides fully human, humanized, or chimeric anti-IGF-1R antibodies that bind human IGF-1R, IGF-1R-binding fragments and derivatives of such antibodies, and IGF-1R-binding polypeptides comprising such fragments. Other embodiments provide nucleic acids encoding such antibodies, antibody fragments and derivatives and polypeptides, cells comprising such polynucleotides, methods of making such antibodies, antibody fragments and derivatives and polypeptides, and methods of using such antibodies, antibody fragments and derivatives and polypeptides, including methods of treating or diagnosing subjects having IGF-1R-related disorders or conditions, and kits for practicing the foregoing.

### CROSS-REFERENCE TO RELATED APPLICATIONS

This application claims the benefit of U.S. Provisional Application No. 61/438,918, filed Feb. 2, 2011, which is hereby incorporated by reference.

### REFERENCE TO THE SEQUENCE LISTING

The present application is being filed along with a Sequence Listing in electronic format via EFS-Web. The Sequence Listing is provided as a text file entitled A1610WOPCTst25.txt, created Jan. 26, 2012, which is 388,961 bytes in size. The information in the electronic format of the Sequence Listing is incorporated herein by reference in its entirety.

### FIELD OF THE INVENTION

This application provides methods and compositions relating to the treatment of tumor diseases such as pancreatic cancer and other cancers and proliferative diseases. This application also provides methods and compositions for determining whether a subject with a cancer condition such as pancreatic cancer is likely to respond to treatment with an inhibitor of IGF-1 and/or IGF-2-mediated signaling.

### BACKGROUND OF THE INVENTION

Pancreatic cancer is one of the most aggressive and deadly forms of cancer. With the current standard of care median overall survival is about six months from diagnosis. More and better treatment options are urgently needed, as are methods of determining which patients are more likely to benefit from such treatments.

### BRIEF DESCRIPTION OF THE DRAWINGS

FIG. 1 provides nucleotide sequences encoding light chain variable domains L1 through L52 and heavy chain variable domains H1 through H52.

FIG. 2 provides amino acid sequences of light chain variable domains L1 through L52. CDR and FR regions are indicated.

FIG. 3 provides amino acid sequences of heavy chain variable domains H1 through H52. CDR and FR regions are indicated.

FIG. 4 provides amino acid sequences of the light chain CDR1 regions of light chain variable domains L1 through L52. Consensus sequences for groups of related CDR sequences are also provided.

FIG. 5 provides amino acid sequences of the light chain CDR2 regions of light chain variable domains L1 through L52. Consensus sequences for groups of related CDR sequences are also provided.

FIG. 6 provides amino acid sequences of the light chain CDR3 regions of light chain variable domains L1 through L52. Consensus sequences for groups of related CDR sequences are also provided.

FIG. 7 provides amino acid sequences of the heavy chain CDR1 regions of heavy chain variable domains H1 through H52. Consensus sequences for groups of related CDR sequences are also provided.

FIG. 8 provides amino acid sequences of the heavy chain CDR2 regions of heavy chain variable domains H1 through H52. Consensus sequences for groups of related CDR sequences are also provided.

FIG. 9 provides amino acid sequences of the heavy chain CDR3 regions of heavy chain variable domains H1 through H52. Consensus sequences for groups of related CDR sequences are also provided.

FIG. 10 provides the amino acid sequence of a human IGF-1R extracellular domain fused to a human IgG1 Fc region (underlined) with an intervening caspace-3 cleavage site (bold).

FIG. 11 provides the amino acid sequence of a human insulin receptor extracellular domain fused to a human IgG1 Fc region (underlined).

FIG. 12 provides the protein sequence of a human IGF-1R extracellular domain (including signal peptide) fused at the C-terminus with chicken avidin. The initiating met in the IGF-1R ECD is designated position 1 in this figure.

FIG. 13 provides the polypeptide sequence of a human kappa light chain antibody constant region and a human IgG1 heavy chain antibody constant region.

FIG. 14 provides a graph illustrating that four phage-displayed antibodies bind significantly better to an IGF-1R-Fc molecule than they bind to an insulin-receptor-Fc or a murine Fc.

FIG. 15 provides graphs illustrating the ability of certain antibodies to compete for binding to IGF-1R with IGF-1 and IGF-2.

FIG. 16 provides graphs illustrating the ability of certain antibodies to inhibit the growth of 32D hu IGF-1R+IRS-1 cells.

FIG. 17 provides graphs illustrating the ability of certain antibodies to inhibit the growth of Balb/C 3T3 hu IGF-1R cells.

FIG. 18 provides the amino acid sequences of ganitumab (AMG 479).

FIG. 19 provides the amino acid sequences of conatumumab (AMG 655).

FIG. 20 provides the study schema for a placebo-controlled, randomized phase 2 study of conatumumab (AMG 655) or ganitumab (AMG 479) or placebo plus gemcitabine in patients with metastatic pancreatic cancer.

FIG. 21 provides overall survival data for the study of FIG. 20.

FIG. 22 provides progression free survival data for the study of FIG. 20.

FIG. 23 provides a graph illustrating the effect of ganitumab exposure on overall survival.

FIG. 24 provides a graph illustrating the effect of ganitumab exposure on progression free survival.

FIG. 25 provides a graph illustrating the observed ganitumab AUC_{SS} distributions at 20 mg/kg in patients with non-pancreatic tumors and 12 mg/kg in patients with pancreatic or non-pancreatic tumors and a predicted AUC_{SS} distribution at 20 mg/kg for patients with pancreatic tumors.

FIG. 26 provides projected progression free survival profiles for placebo and 12 and 20 mg/kg ganitumab.

FIG. 27 provides an analysis of the relationship between overall survival and baseline levels of the IGF biomarkers by Cox proportional hazard model. Data are tabulated and FIR shown graphically as a Forest plot with error bars representing 95% confidence intervals.

FIGS. 28 A and B provide a comparison of the overall survival of subgroups having higher-than-median and lower-than-median baseline levels of the IGF biomarkers, treated with either ganitumab or placebo, by Cox proportional hazard model. Data are tabulated and HR shown graphically as a Forest plot with error bars representing 95% confidence intervals.

FIG. 29 provides a Kaplan-Maier plot illustrating the difference in overall survival between high and low total IGF-1 patient groups. The arrow indicates the line corresponding to the ganitumab-treated high total IGF-1 subgroup.

FIG. 30 provides a Kaplan-Maier plot illustrating the difference in overall survival between high and low free IGF-1 patient groups. The arrow indicates the line corresponding to the ganitumab-treated high free IGF-1 subgroup.

FIG. 31 provides a Kaplan-Maier plot illustrating the difference in overall survival between high and low total IGF-2 patient groups. The arrow indicates the line corresponding to the ganitumab-treated high IGF-2 subgroup.

FIG. 32 provides a Kaplan-Maier plot illustrating the difference in overall survival between high and low total IGFBP-2 patient groups. The arrow indicates the line corresponding to the ganitumab-treated low IGFBP-2 subgroup.

FIG. 33 provides a Kaplan-Maier plot illustrating the difference in overall survival between high and low IGFBP-3 patient groups. The arrow indicates the line corresponding to the ganitumab-treated high IGFBP-3 subgroup.

FIG. 34 provides a graph illustrating the best tumor response achieved for each of twelve human subjects treated with an inhibitor of IGF-1 receptor signaling.

FIG. 35 provides a scatter plot for pairs of biomarkers.

### SUMMARY OF THE INVENTION

In one aspect, the present invention provides a method of determining whether a cancerous condition in a patient is likely to respond to a treatment that reduces signaling mediated by IGF-1 or IGF-2, comprising determining the concentration of a circulating biomarker in said patient's serum, wherein the amount of said circulating biomarker is predictive of a response to said treatment. In one embodiment, said treatment comprises administering an inhibitor of signaling through an IGF-1R signaling pathway. In another embodiment, said inhibitor inhibits signaling upstream of IGF-1R. In another embodiment, said inhibitor inhibits IGF-1 or IGF-2. In another embodiment, said inhibitor inhibits the transcription or translation of IGF-1 or IGF-2. In another embodiment, said inhibitor inhibits the processing or secretion of IGF-1 or IGF-2. In another embodiment, said inhibitor binds to IGF-1 or IGF-2. In another embodiment, said inhibitor inhibits the binding of IGF-1 or IGF-2 to IGF-1R. In another embodiment, said inhibitor is an anti-IGF-1 or anti-IGF-2 antibody, antigen binding fragment of said antibody, isolated IGF-1 or IGF-2 binding protein, recombinant human IGF-1 or IGF-2 binding protein, IGFBP1, IGFBP2, IGFBP3, IGFBP4, IGFBP5, IGFBP6, or IGFBP7. In another embodiment, said inhibitor is an inhibitor of IGF-1R. In another embodiment, said inhibitor is a small molecule inhibitor of IGF-1R. In another embodiment, said small molecule inhibitor of IGF-1R is a kinase inhibitor. In another embodiment, said small molecule inhibitor is OSI-906, linsitinib, BMS-754807, INSM-18, XL228, AXL1717, BMS-536924, NVP-ADW742, GSK621659A, GSK1838705A, A-928605, AZD4253, TAE226, or AG1024. In another embodiment, said inhibitor is an anti-IGF-1R antibody, or an antigen binding fragment of said antibody. In another embodiment, said antibody inhibits binding of IGF-1 or IGF-2 to IGF-1R. In another embodiment, said antibody inhibits binding of IGF-1 and IGF-2 to IGF-1R. In another embodiment, said antibody downregulates IGF-1R. In another embodiment, said antibody is fully human, humanized, or chimeric. In another embodiment, said anti-IGF-1R antibody is ganitumab, AMG 479, figitumumab, CP-751,871, cixutumumab, IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52. In another embodiment, said inhibitor affects an IGF-1 or IGF-2 binding protein. In another embodiment, said inhibitor affects IGFBP1, IGFBP2, IGFBP3, IGFBP4, IGFBP5, IGFBP6, or IGFBP7. In another embodiment, said inhibitor inhibits downstream signaling of IGF-1R. In another embodiment, said inhibitor inhibits the Ras/Raf signaling pathway or the PI3K signaling pathway. In another embodiment, said inhibitor inhibits Shc, Grb2, SOS, Ras, Raf, MEK, ERK, Elk-1, IRS1, PI3K, or AKT/PKB. In another embodiment, said inhibitor is an mTOR inhibitor. In another embodiment, said mTOR inhibitor is everolimus. In another embodiment, said cancerous condition is characterized by IGF-1R signaling activity. In another embodiment, said cancerous condition is of a type that responds to treatment using an inhibitor of IGF-1R signaling. In another embodiment, said patient has a tumor. In another embodiment, said tumor is a solid tumor. In another embodiment, said tumor is a primary tumor. In another embodiment, said tumor is a metastatic tumor. In another embodiment, said tumor is a pancreatic cancer tumor, a sarcoma tumor, a Ewing's sarcoma tumor, an ovarian tumor, a breast cancer tumor, a small cell lung cancer tumor, a non-small cell lung cancer tumor, a colorectal cancer tumor with an activating KRAS mutation, a colorectal cancer tumor with a wild-type KRAS allele, a prostate cancer tumor, a hepatic cancer tumor, a head and neck cancer tumor, a carcinoid, tumor, a gastric cancer tumor, a multiple myeloma tumor, a neuroendocrine cancer tumor, an adrenal cell carcinoma tumor, or a hepatocellular carcinoma tumor. In another embodiment, said pancreatic cancer tumor is a metastatic pancreatic cancer tumor. In another embodiment, said pancreatic cancer tumor is a locally advanced pancreatic cancer tumor. In another embodiment, said cancerous condition is Acute Lymphoblastic Leukemia, Adrenocortical Carcinoma, an AIDS-Related Cancer, AIDS-Related Lymphoma, Anal Cancer, Childhood Cerebellar Astrocytoma, Childhood Cerebral Astrocytoma, Basal Cell Carcinoma, Extrahepatic Bile Duct Cancer, Bladder Cancer, Osteosarcoma/Malignant Fibrous Histiocytoma Bone Cancer, a Brain Tumor (e.g., Brain Stem Glioma, Cerebellar Astrocytoma, Cerebral Astrocytoma/Malignant Glioma, Ependymoma, Medulloblastoma, a Supratentorial Primitive Neuroectodermal Tumor, Visual Pathway or Hypothalamic Glioma), Breast Cancer, a Bronchial Adenoma/Carcinoid, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, a Childhood Cancer, Chronic Lymphocytic Leukemia, Chronic Myelogenous Leukemia, a Chronic Myeloproliferative Disorder, Colon Cancer, Colorectal Cancer, Cutaneous T-Cell Lymphoma, Endometrial Cancer, Ependymoma, Esophageal Cancer, a Ewing's Family Tumor, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, a Germ Cell Tumor (e.g., Extracranial, Extragonadal, or Ovarian), Gestational Trophoblastic Tumor, Glioma (e.g., Adult, Childhood Brain Stem, Childhood Cerebral Astrocytoma, Childhood Visual Pathway or Hypothalamic), Hairy Cell Leukemia, Head and Neck Cancer, Hepatocellular (Liver) Cancer, Hodgkin's Lymphoma, Hypopharyngeal Cancer, Hypothalamic or Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocytic, Chronic Myelogenous, or Hairy Cell), Lip or Oral Cavity Cancer, Liver Cancer, Non-Small Cell Lung Cancer, Small Cell Lung Cancer, Lymphoma (e.g., AIDS-Related, Burkitt's, Cutaneous T-Cell, Hodgkin's, Non-Hodgkin's, or Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, a Myelodysplastic Syndrome, a Myelodysplastic/Myeloproliferative Disease, Myelogenous Leukemia, Chronic Myeloid Leukemia, Multiple Myeloma, a Chronic Myeloproliferative Disorder, Nasal Cavity or Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus or Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis or Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-Cell Lymphoma, Testicular Cancer, Thymoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway or Hypothalamic Glioma, Vulvar Cancer, Waldenstrom's Macroglobulinemia, or Wilms' Tumor. In another embodiment, said circulating biomarker is total IGF-1, free IGF-1, total IGF-2, free IGF-2, IGFBP-1, IGFBP-2, IGFBP-3, IGFBP-4, IGFBP-5, IGFBP-6, or IGFBP-7. In another embodiment, said circulating biomarker is total IGF-1, and wherein a higher total IGF-1 concentration indicates that said patient is more likely to respond to said treatment than a lower total IGF-1 concentration. In another embodiment, said patient's total IGF-1 concentration is higher if it is greater than about 118 ng/mL and lower if it is less than about 118 ng/mL. In another embodiment, said circulating biomarker is total IGF-2, and wherein a higher total IGF-2 concentration indicates that said patient is more likely to respond to said treatment than a lower total IGF-2 concentration. In another embodiment, said patient's total IGF-2 concentration is higher if it is greater than about 1734 ng/mL and lower if it is less than about 1734 ng/mL. In another embodiment, said circulating biomarker is IGFBP-1, and wherein a lower IGFBP-1 concentration indicates that said patient is more likely to respond to said treatment than a higher IGFBP-1 concentration. In another embodiment, said patient's total IGFBP-1 concentration is higher if it is greater than about 30 ng/mL and lower if it is less than about 30 ng/mL. In another embodiment, said circulating biomarker is IGFBP-2, and wherein a lower IGFBP-2 concentration indicates that said patient is more likely to respond to said treatment than a higher total IGFBP-2 concentration. In another embodiment, said patient's total IGFBP-2 concentration is higher if it is greater than about 170 ng/mL and lower if it is less than about 170 ng/mL. In another embodiment, said circulating biomarker is IGFBP-3, and wherein a higher IGFBP-3 concentration indicates that said patient is more likely to respond to said treatment than a lower total IGFBP-3 concentration. In another embodiment, said patient's total IGFBP-3 concentration is higher if it is greater than about 1.9 μg/mL and lower if it is less than about 1.9 μg/mL. In another embodiment, said circulating biomarker is IGFBP-4, and wherein a higher IGFBP-4 concentration indicates that said patient is more likely to respond to said treatment than a lower total IGFBP-4 concentration. In another embodiment, said patient's total IGFBP-4 concentration is higher if it is greater than about 40 ng/mL and lower if it is less than about 40 ng/mL. In another embodiment, said method further comprises treating said patient with said treatment. In another embodiment, said method further comprises treating said patient with said treatment if said patient's circulating biomarker concentration indicates that said patient is more likely to respond to said treatment. In another embodiment, said treatment comprises administering an inhibitor of signaling through an IGF-1R signaling pathway. In another embodiment, said inhibitor inhibits signaling upstream of IGF-1R. In another embodiment, said inhibitor inhibits IGF-1 or IGF-2. In another embodiment, said inhibitor inhibits the transcription or translation of IGF-1 or IGF-2. In another embodiment, said inhibitor inhibits the processing or secretion of IGF-1 or IGF-2. In another embodiment, said inhibitor binds to IGF-1 or IGF-2. In another embodiment, said inhibitor inhibits the binding of IGF-1 or IGF-2 to IGF-1R. In another embodiment, said inhibitor is an anti-IGF-1 or anti-IGF-2 antibody, antigen binding fragment of said antibody, isolated IGF-1 or IGF-2 binding protein, recombinant human IGF-1 or IGF-2 binding protein, IGFBP1, IGFBP2, IGFBP3, IGFBP4, IGFBP5, IGFBP6, or IGFBP7. In another embodiment, said inhibitor is an inhibitor of IGF-1R. In another embodiment, said inhibitor is a small molecule inhibitor of IGF-1R. In another embodiment, said small molecule inhibitor of IGF-1R is a kinase inhibitor. In another embodiment, said small molecule inhibitor is OSI-906, linsitinib, BMS-754807, INSM-18, XL228, AXL1717, BMS-536924, NVP-ADW742, GSK621659A, GSK1838705A, A-928605, AZD4253, TAE226, or AG1024. In another embodiment, said inhibitor is an anti-IGF-1R antibody, or an antigen binding fragment of said antibody. In another embodiment, said antibody inhibits binding of IGF-1 or IGF-2 to IGF-1R. In another embodiment, said antibody inhibits binding of IGF-1 and IGF-2 to IGF-1R. In another embodiment, said antibody downregulates IGF-1R. In another embodiment, said antibody is fully human, humanized, or chimeric. In another embodiment, said anti-IGF-1R antibody is ganitumab, AMG 479, figitumumab, CP-751,871, cixutumumab, IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52. In another embodiment, said inhibitor affects an IGF-1 or IGF-2 binding protein. In another embodiment, said inhibitor affects IGFBP1, IGFBP2, IGFBP3, IGFBP4, IGFBP5, IGFBP6, or IGFBP1. In another embodiment, said inhibitor inhibits downstream signaling of IGF-1R. In another embodiment, said inhibitor inhibits the Ras/Raf signaling pathway or the PI3K signaling pathway. In another embodiment, said inhibitor inhibits Shc, Grb2, SOS, Ras, Raf, MEK, ERK, Elk-1, IRS1, PI3K, or AKT/PKB. In another embodiment, said inhibitor is an mTOR inhibitor. In another embodiment, said mTOR inhibitor is everolimus. In another embodiment, said response is an increase in survival. In another embodiment, said response is an increase in progression free survival. In another embodiment, said response is an objective response. In another embodiment, said objective response is stable disease. In another embodiment, said objective response is a partial response. In another embodiment, said objective response is a complete response. In another embodiment, said complete response is a durable complete response.

In another aspect, the present invention provides a method of determining whether a cancer condition in a patient is likely to respond to a treatment with an inhibitor of signaling through an IGF-1R signaling pathway, comprising determining the exposure of said patient to said inhibitor, wherein said cancer condition is likely to respond to said treatment if said exposure of said patient is about equal to or greater than the median exposure of patients with said cancer condition treated with said inhibitor. In one embodiment, said inhibitor inhibits signaling upstream of IGF-1R. In another embodiment, said inhibitor inhibits IGF-1 or IGF-2. In another embodiment, said inhibitor inhibits the transcription or translation of IGF-1 or IGF-2. In another embodiment, said inhibitor inhibits the processing or secretion of IGF-1 or IGF-2. In another embodiment, said inhibitor binds to IGF-1 or IGF-2. In another embodiment, said inhibitor inhibits the binding of IGF-1 or IGF-2 to IGF-1R. In another embodiment, said inhibitor is an anti-IGF-1 or anti-IGF-2 antibody, antigen binding fragment of said antibody, isolated IGF-1 or IGF-2 binding protein, recombinant human IGF-1 or IGF-2 binding protein, IGFBP1, IGFBP2, IGFBP3, IGFBP4, IGFBP5, IGFBP6, or IGFBP7. In another embodiment, said inhibitor is an inhibitor of IGF-1R. In another embodiment, said inhibitor is a small molecule inhibitor of IGF-1R. In another embodiment, said small molecule inhibitor of IGF-1R is a kinase inhibitor. In another embodiment, said small molecule inhibitor is OSI-906, linsitinib, BMS-754807, INSM-18, XL228, AXL1717, BMS-536924, NVP-ADW742, GSK621659A, GSK1838705A, A-928605, AZD4253, TAE226, or AG1024. In another embodiment, said inhibitor is an anti-IGF-1R antibody, or an antigen binding fragment of said antibody. In another embodiment, said antibody or fragment inhibits binding of IGF-1 or IGF-2 to IGF-1R. In another embodiment, said antibody or fragment inhibits binding of IGF-1 and IGF-2 to IGF-1R. In another embodiment, said antibody or fragment downregulates IGF-1R. In another embodiment, said antibody or fragment is fully human, humanized, or chimeric. In another embodiment, said anti-IGF-1R antibody or fragment is ganitumab, AMG 479, figitumumab, CP-751,871, cixutumumab, IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52. In another embodiment, said inhibitor affects an IGF-1 or IGF-2 binding protein. In another embodiment, said inhibitor affects IGFBP1, IGFBP2, IGFBP3, IGFBP4, IGFBP5, IGFBP6, or IGFBP7. In another embodiment, said inhibitor inhibits downstream signaling of IGF-1R. In another embodiment, said inhibitor inhibits the Ras/Raf signaling pathway or the PI3K signaling pathway. In another embodiment, said inhibitor inhibits Shc, Grb2, SOS, Ras, Raf, MEK, ERK, Elk-1, IRS1, PI3K, or AKT/PKB. In another embodiment, said inhibitor is an mTOR inhibitor. In another embodiment, said mTOR inhibitor is everolimus. In another embodiment, said cancerous condition is characterized by IGF-1R signaling activity. In another embodiment, said cancerous condition is of a type that responds to treatment using an inhibitor of IGF-1R signaling. In another embodiment, said patient has a tumor. In another embodiment, said tumor is a solid tumor. In another embodiment, said tumor is a primary tumor. In another embodiment, said tumor is a metastatic tumor. In another embodiment, said tumor is a pancreatic cancer tumor, a sarcoma tumor, a Ewing's sarcoma tumor, an ovarian tumor, a breast cancer tumor, a small cell lung cancer tumor, a non-small cell lung cancer tumor, a colorectal cancer tumor with an activating KRAS mutation, a colorectal cancer tumor with a wild-type KRAS allele, a prostate cancer tumor, a hepatic cancer tumor, a head and neck cancer tumor, a carcinoid tumor, a gastric cancer tumor, a multiple myeloma tumor, a neuroendocrine cancer tumor, an adrenal cell carcinoma tumor, or a hepatocellular carcinoma tumor. In another embodiment, said pancreatic cancer tumor is a metastatic pancreatic cancer tumor. In another embodiment, said pancreatic cancer tumor is a locally advanced pancreatic cancer tumor. In another embodiment, said method further comprises administering to said patient a dose of an anti-IGF-1R antibody. In another embodiment, said dose is a 12 mg/kg dose. In another embodiment, said dose is a 20 mg/kg dose. In another embodiment, said antibody is ganitumab. In another embodiment, said cancerous condition is pancreatic cancer. In another embodiment, said pancreatic cancer is metastatic. In another embodiment, said pancreatic cancer is locally advanced. In another embodiment, said method further comprises administering to said patient a second dose of said anti-IGF-1R antibody. In another embodiment, said second dose is administered two weeks after the first dose. In another embodiment, said patient receives at least one additional dose of said anti-IGF-1R antibody. In another embodiment, said doses are administered once every two weeks. In another embodiment, the method further comprises determining the concentration of a second circulating biomarker. In another embodiment, the method further comprises determining the concentration of a third circulating biomarker. In another embodiment, the method further comprises determining the concentration of a fourth circulating biomarker. In another embodiment, said biomarkers are IGFBP-2 and total IGF-1. In another embodiment, said biomarkers are IGFBP-2 and IGF-2. In another embodiment, said biomarkers are IGFBP-2 and IGFBP-3. In another embodiment, said biomarkers are total IGF-1 and IGF-2. In another embodiment, said biomarkers are total IGF-1 and IGFBP-3. In another embodiment, said biomarkers are IGF-2 and IGFBPF-3. In another embodiment, said circulating biomarker is total IGF-1, free IGF-1, total IGF-2, free IGF-2, IGFBP-1, IGFBP-3, IGFBP-4, IGFBP-5, IGFBP-6, or IGFBP-7, and wherein said concentration of said circulating biomarker is greater than the median value from a reference range derived from an analysis of at least 100 patients having said cancerous condition. In another embodiment, said circulating biomarker is IGFBP-2 and wherein said concentration of said circulating biomarker is less than the median value from a reference range derived from an analysis of at least 100 patients having said cancerous condition. In another embodiment, said cancer condition is likely to respond to said treatment if said patient's AUC_{SS} is greater than or equal to the median value. In another embodiment, said median value is 19.2 mg·h/mL. In another embodiment, it is the concentration of said circulating biomarker before treatment that is determined. In another aspect, the present invention provides a method for determining a prognosis for a patient comprising determining the amount of a circulating biomarker, wherein said patient has a cancerous condition and the amount of said circulating biomarker indicates whether said patient has a better prognosis. In one embodiment, said cancerous condition is characterized by IGF-1R signaling activity. In another embodiment, said cancerous condition is of a type that responds to treatment using an inhibitor of IGF-1R signaling. In another embodiment, said patient has a tumor. In another embodiment, said tumor is a solid tumor. In another embodiment, said tumor is a primary tumor. In another embodiment, said tumor is a metastatic tumor. In another embodiment, said tumor is a pancreatic cancer tumor, a sarcoma tumor, a Ewing's sarcoma tumor, an ovarian tumor, a breast cancer tumor, a small cell lung cancer tumor, a non-small cell lung cancer tumor, a colorectal cancer tumor with an activating KRAS mutation, a colorectal cancer tumor with a wild-type KRAS allele, a prostate cancer tumor, a hepatic cancer tumor, a head and neck cancer tumor, a carcinoid tumor, a gastric cancer tumor, a multiple myeloma tumor, a neuroendocrine cancer tumor, an adrenal cell carcinoma tumor, or a hepatocellular carcinoma tumor. In another embodiment, said pancreatic cancer tumor is a metastatic pancreatic cancer tumor. In another embodiment, said pancreatic cancer tumor is a locally advanced pancreatic cancer tumor. In another embodiment, said cancerous condition is Acute Lymphoblastic Leukemia, Adrenocortical Carcinoma, an AIDS-Related Cancer, AIDS-Related Lymphoma, Anal Cancer, Childhood Cerebellar Astrocytoma, Childhood Cerebral Astrocytoma, Basal Cell Carcinoma, Extrahepatic Bile Duct Cancer, Bladder Cancer, Osteosarcoma/Malignant Fibrous Histiocytoma Bone Cancer, a Brain Tumor (e.g., Brain Stem Glioma, Cerebellar Astrocytoma, Cerebral Astrocytoma/Malignant Glioma, Ependymoma, Medulloblastoma, a Supratentorial Primitive Neuroectodermal Tumor, Visual Pathway or Hypothalamic Glioma), Breast Cancer, a Bronchial Adenoma/Carcinoid, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, a Childhood Cancer, Chronic Lymphocytic Leukemia, Chronic Myelogenous Leukemia, a Chronic Myeloproliferative Disorder, Colon Cancer, Colorectal Cancer, Cutaneous T-Cell Lymphoma, Endometrial Cancer, Ependymoma, Esophageal Cancer, a Ewing's Family Tumor, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, a Germ Cell Tumor (e.g., Extracranial, Extragonadal, or Ovarian), Gestational Trophoblastic Tumor, Glioma (e.g., Adult, Childhood Brain Stem, Childhood Cerebral Astrocytoma, Childhood Visual Pathway or Hypothalamic), Hairy Cell Leukemia, Head and Neck Cancer, Hepatocellular (Liver) Cancer, Hodgkin's Lymphoma, Hypopharyngeal Cancer, Hypothalamic or Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocytic, Chronic Myelogenous, or Hairy Cell), Lip or Oral Cavity Cancer, Liver Cancer, Non-Small Cell Lung Cancer, Small Cell Lung Cancer, Lymphoma (e.g., AIDS-Related, Burkitt's, Cutaneous T-Cell, Hodgkin's, Non-Hodgkin's, or Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, a Myelodysplastic Syndrome, a Myelodysplastic/Myeloproliferative Disease, Myelogenous Leukemia, Chronic Myeloid Leukemia, Multiple Myeloma, a Chronic Myeloproliferative Disorder, Nasal Cavity or Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus or Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis or Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-Cell Lymphoma, Testicular Cancer, Thymoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway or Hypothalamic Glioma, Vulvar Cancer, Waldenstrom's Macroglobulinemia, or Wilms' Tumor. In another embodiment, said circulating biomarker is total IGF-1, free IGF-1, total IGF-2, free IGF-2, IGFBP-1, IGFBP-2, IGFBP-3, IGFBP-4, IGFBP-5, IGFBP-6, or IGFBP-7. In another embodiment, said circulating biomarker is total IGF-1, and wherein a higher total IGF-1 concentration indicates that said patient has a better prognosis. In another embodiment, said patient's total IGF-1 concentration is higher if it is greater than about 118 ng/mL and lower if it is less than about 118 ng/mL. In another embodiment, said circulating biomarker is total IGF-2, and wherein a lower IGF-2 concentration indicates that said patient has a better prognosis. In another embodiment, said patient's total IGF-2 concentration is higher if it is greater than about 1734 ng/mL and lower if it is less than about 1734 ng/mL. In another embodiment, said circulating biomarker is IGFBP-1, and wherein a lower IGFBP-1 concentration indicates that said patient has a better prognosis. In another embodiment, said patient's total IGFBP-1 concentration is higher if it is greater than about 30 ng/mL and lower if it is less than about 30 ng/mL. In another embodiment, said circulating biomarker is IGFBP-2, and wherein a lower IGFBP-2 concentration indicates that said patient has a better prognosis. In another embodiment, said patient's total IGFBP-2 concentration is higher if it is greater than about 170 ng/mL and lower if it is less than about 170 ng/mL. In another embodiment, said circulating biomarker is IGFBP-3, and wherein a higher IGFBP-3 concentration indicates that said patient has a better prognosis. In another embodiment, said patient's total IGFBP-3 concentration is higher if it is greater than about 1.9 μg/mL and lower if it is less than about 1.9 μg/mL. In another embodiment, said circulating biomarker is IGFBP-4, and wherein a lower IGFBP-4 concentration indicates that said patient has a better prognosis. In another embodiment, said patient's total IGFBP-4 concentration is higher if it is greater than about 40 ng/mL and lower if it is less than about 40 ng/mL. In another embodiment, said method further comprises determining the concentration of a second circulating biomarker. In another embodiment, said method further comprises determining the concentration of a third circulating biomarker. In another embodiment, said method further comprises determining the concentration of a fourth circulating biomarker. In another embodiment, said biomarkers are IGFBP-2 and total IGF-1. In another embodiment, said biomarkers are IGFBP-2 and IGF-2. In another embodiment, said biomarkers are IGFBP-2 and IGFBP-3. In another embodiment, said biomarkers are total IGF-1 and IGF-2. In another embodiment, said biomarkers are total IGF-1 and IGFBP-3. In another embodiment, said biomarkers are IGF-2 and IGFBPF-3.

In another aspect, the present invention provides a kit for practicing any of the foregoing methods.

### DETAILED DESCRIPTION OF THE INVENTION

The present invention provides compositions, kits, and methods relating to molecules that bind to the Insulin-Like Growth Factor Receptor (“IGF-1R”), including molecules that agonize or antagonize IGF-1R, such as anti-IGF-1R antibodies, antibody fragments, and antibody derivatives, e.g., antagonistic anti-IGF-1R antibodies, antibody fragments, or antibody derivatives. Also provided are nucleic acids, and derivatives and fragments thereof, comprising a sequence of nucleotides that encodes all or a portion of a polypeptide that binds to IGF-1R, e.g., a nucleic acid encoding all or part of an anti-IGF-1R antibody, antibody fragment, or antibody derivative, plasmids and vectors comprising such nucleic acids, and cells or cell lines comprising such nucleic acids and/or vectors and plasmids. The provided methods include, for example, methods of making, identifying, or isolating molecules that bind to IGF-1R, such as anti-IGF-1R antibodies, methods of determining whether a molecule binds to IGF-1R, methods of determining whether a molecule agonizes or antagonizes IGF-1R, methods of making compositions, such as pharmaceutical compositions, comprising a molecule that binds to IGF-1R, and methods for administering a molecule that binds IGF-1R to a subject, for example, methods for treating a condition mediated by IGF-1R, and for agonizing or antagonizing a biological activity of IGF-1R, IGF-1, and/or IGF-2 in vivo or in vitro.

Polynucleotide and polypeptide sequences are indicated using standard one- or three-letter abbreviations. Unless otherwise indicated, polypeptide sequences have their amino termini at the left and their carboxy termini at the right and single-stranded nucleic acid sequences, and the top strand of double-stranded nucleic acid sequences, have their 5′ termini at the left and their 3′ termini at the right. A particular polypeptide or polynucleotide sequence also can be described by explaining how it differs from a reference sequence.

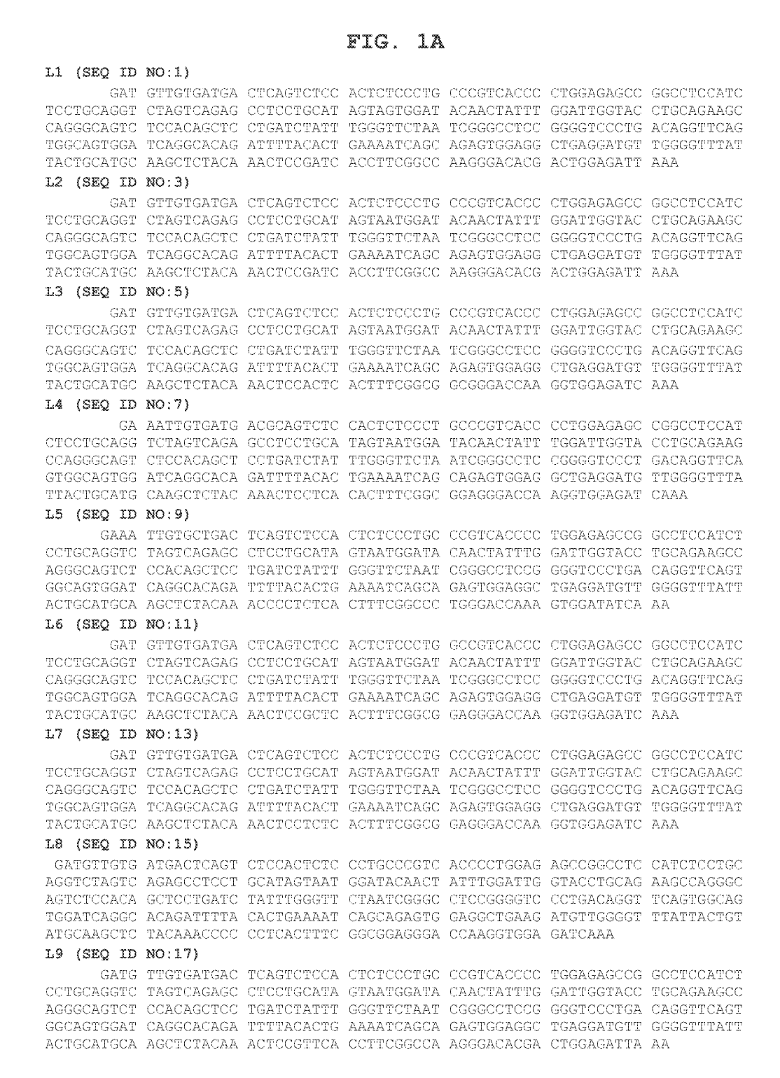

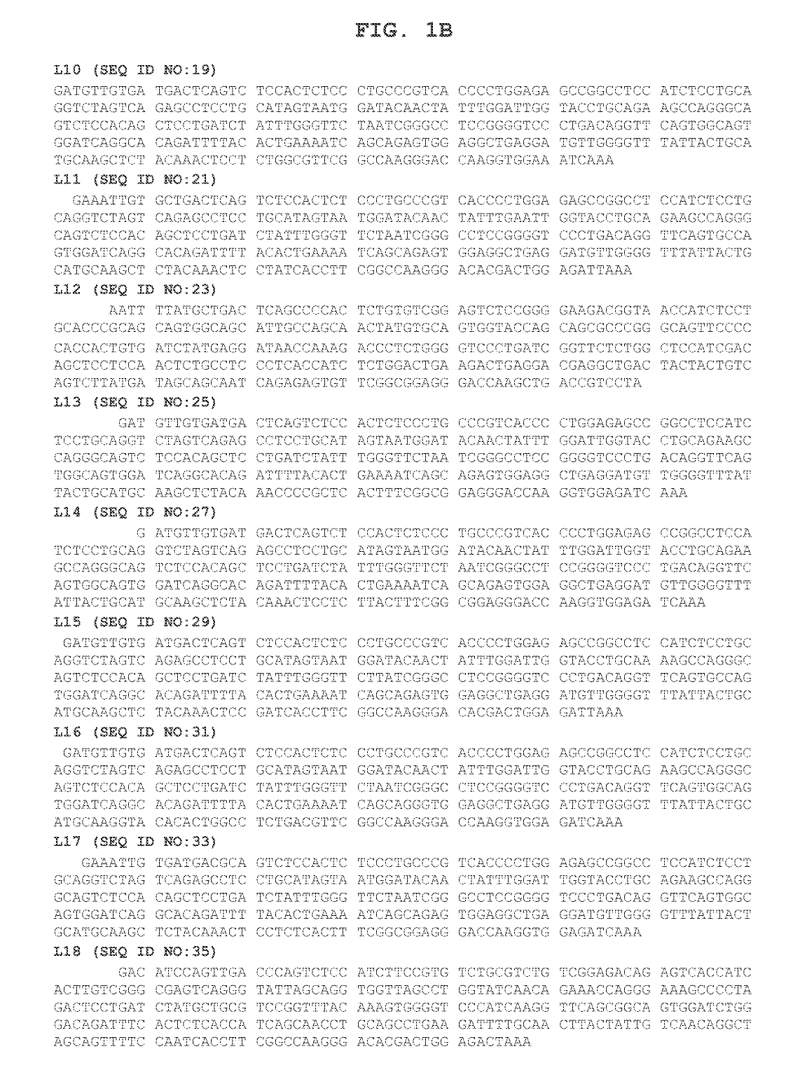

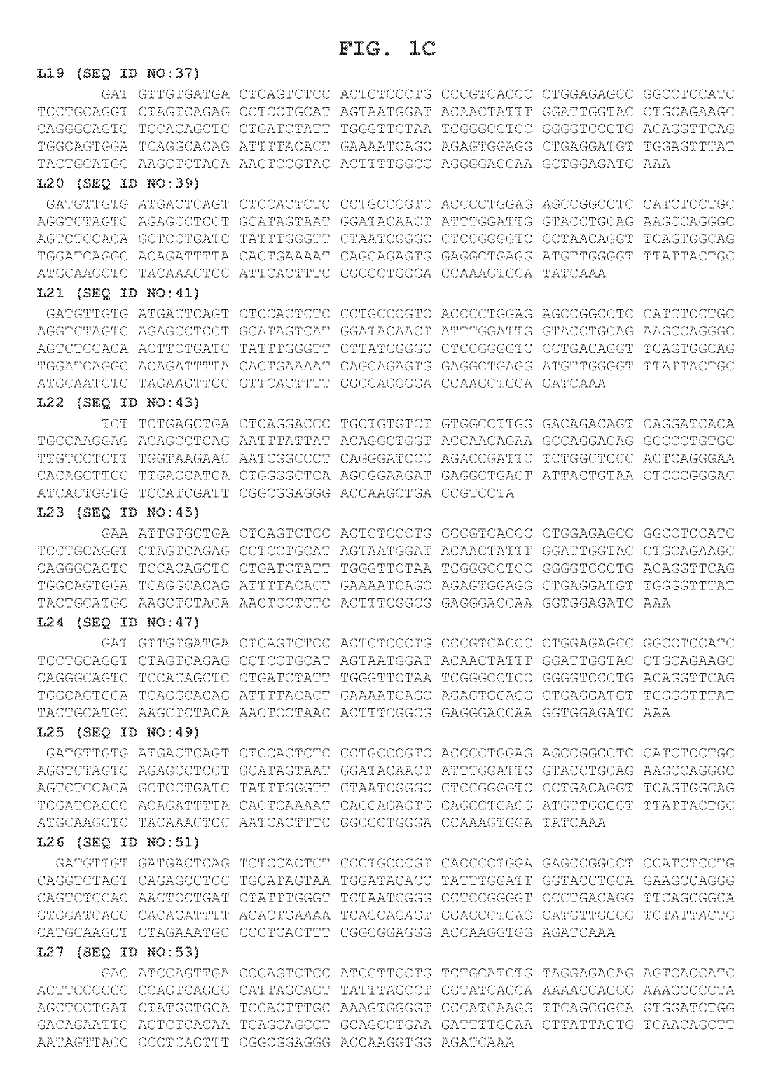

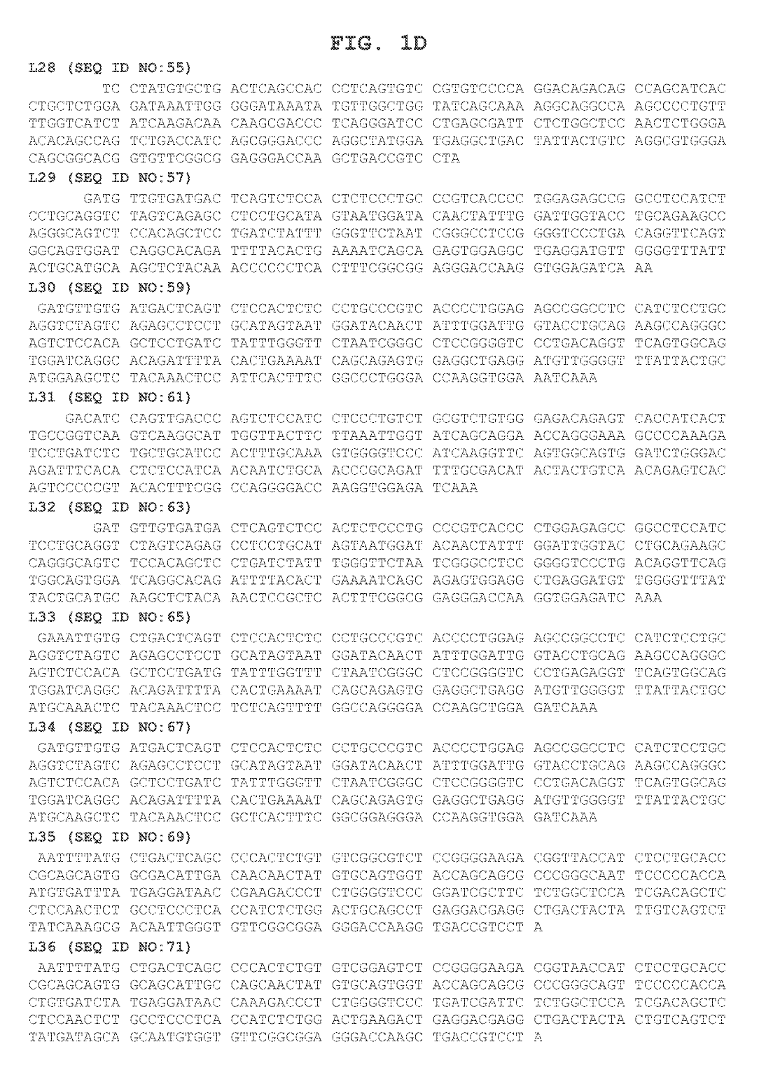

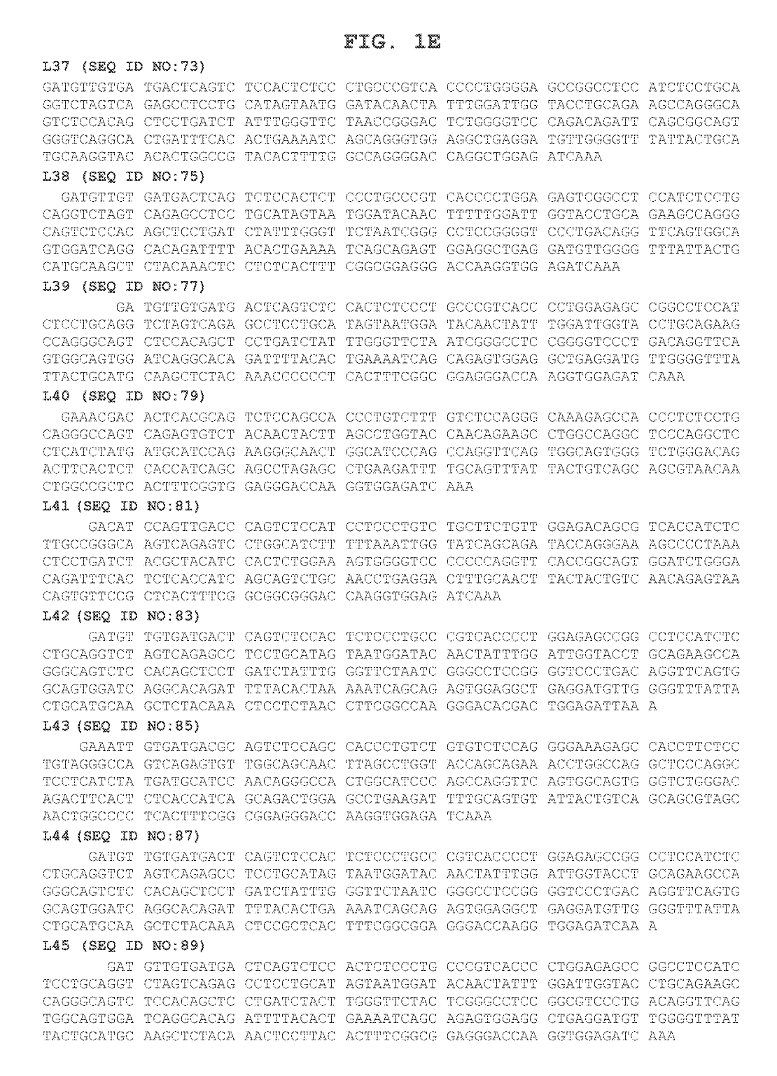

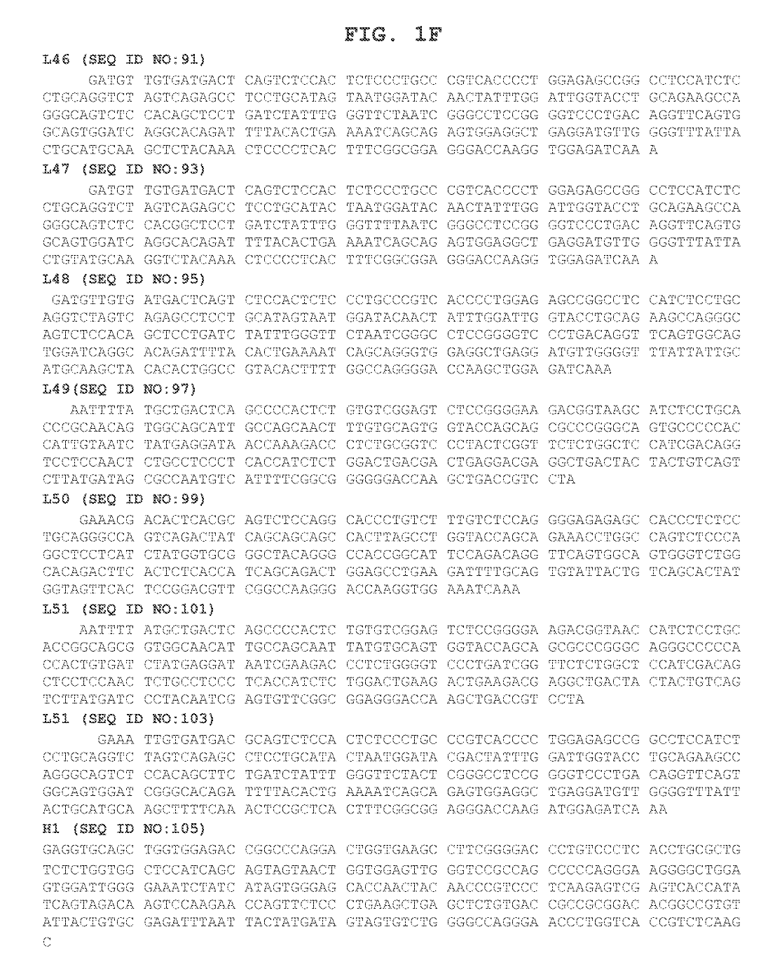

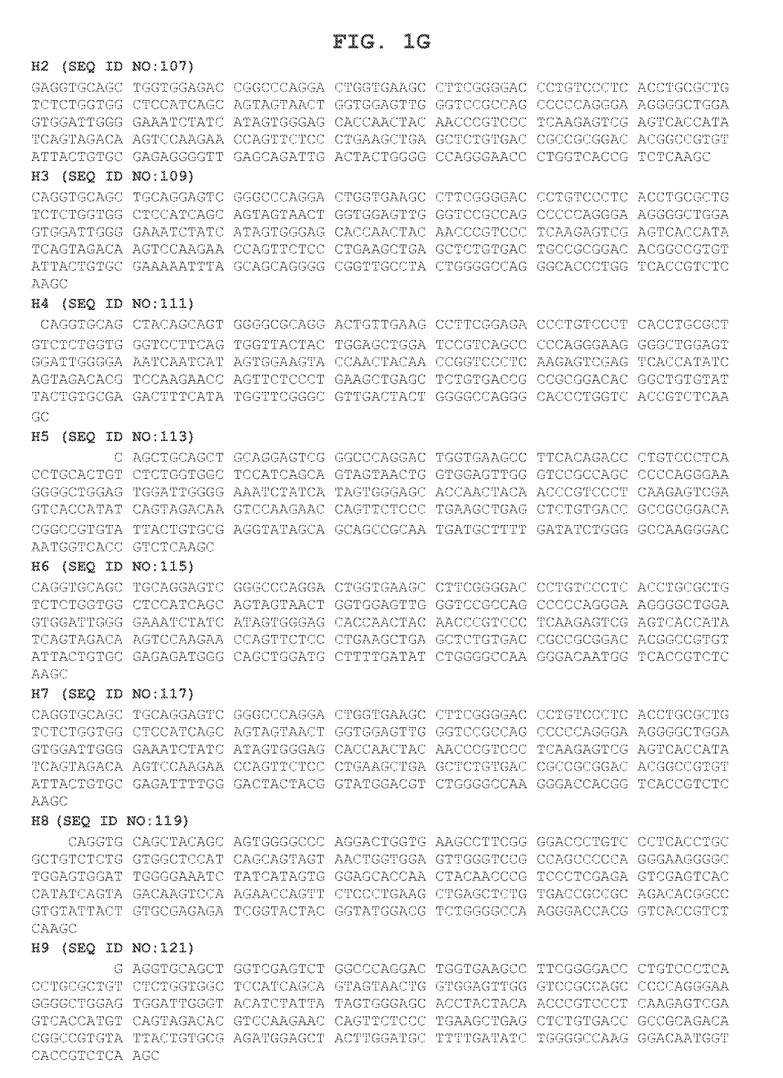

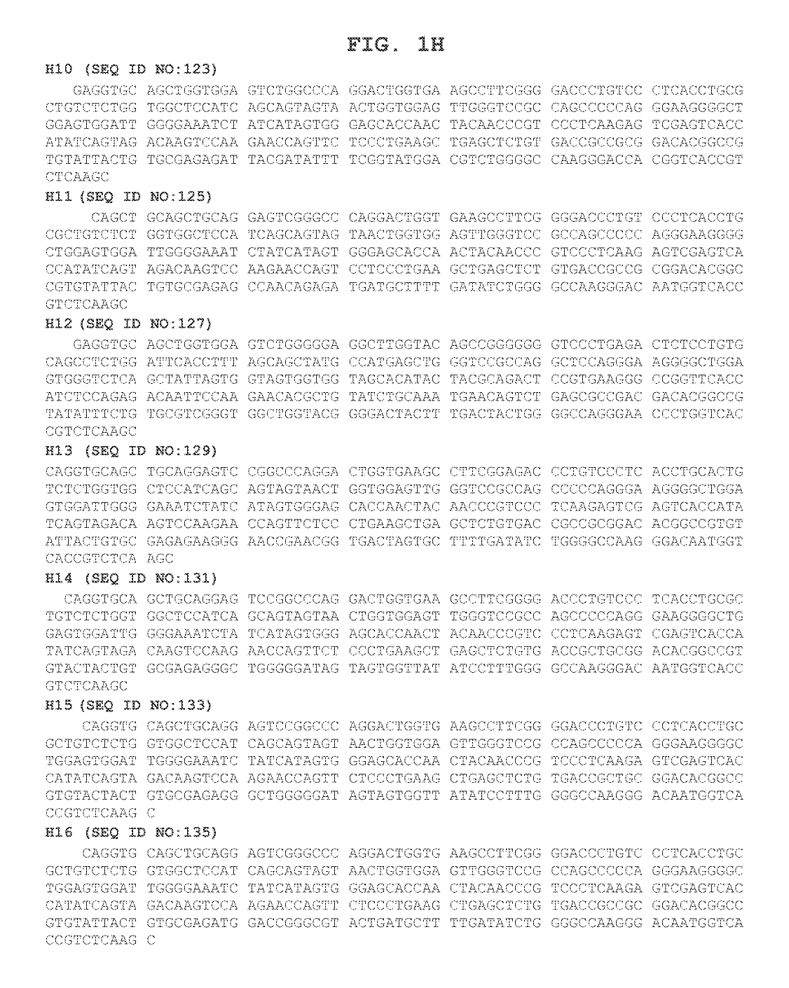

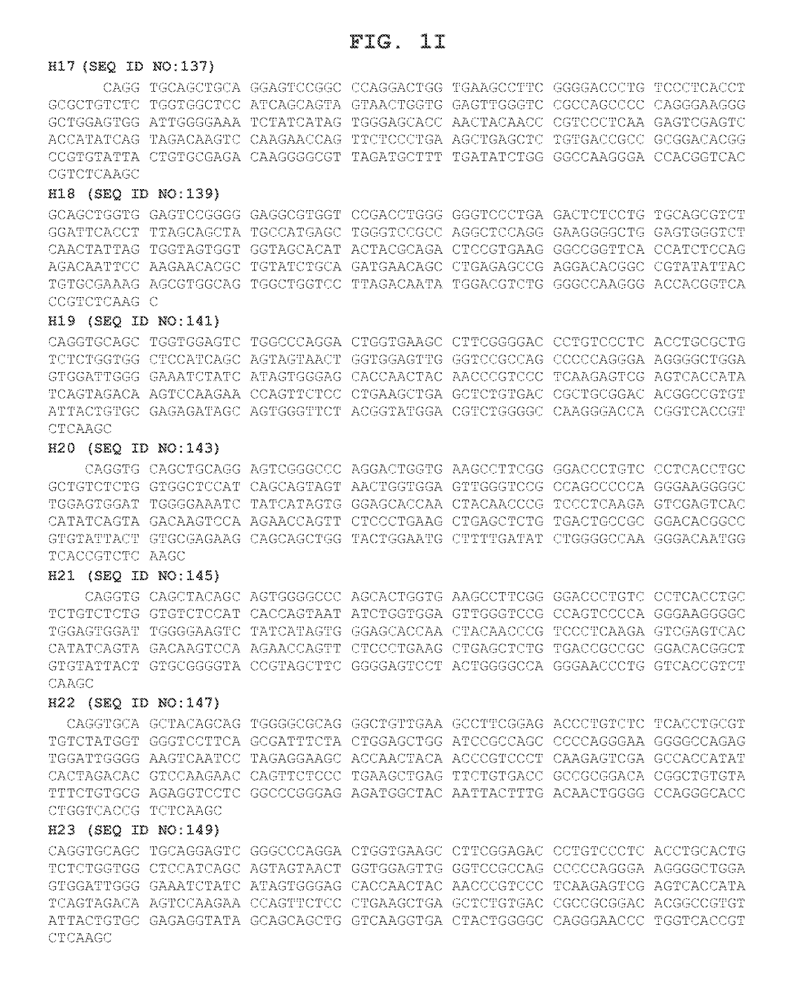

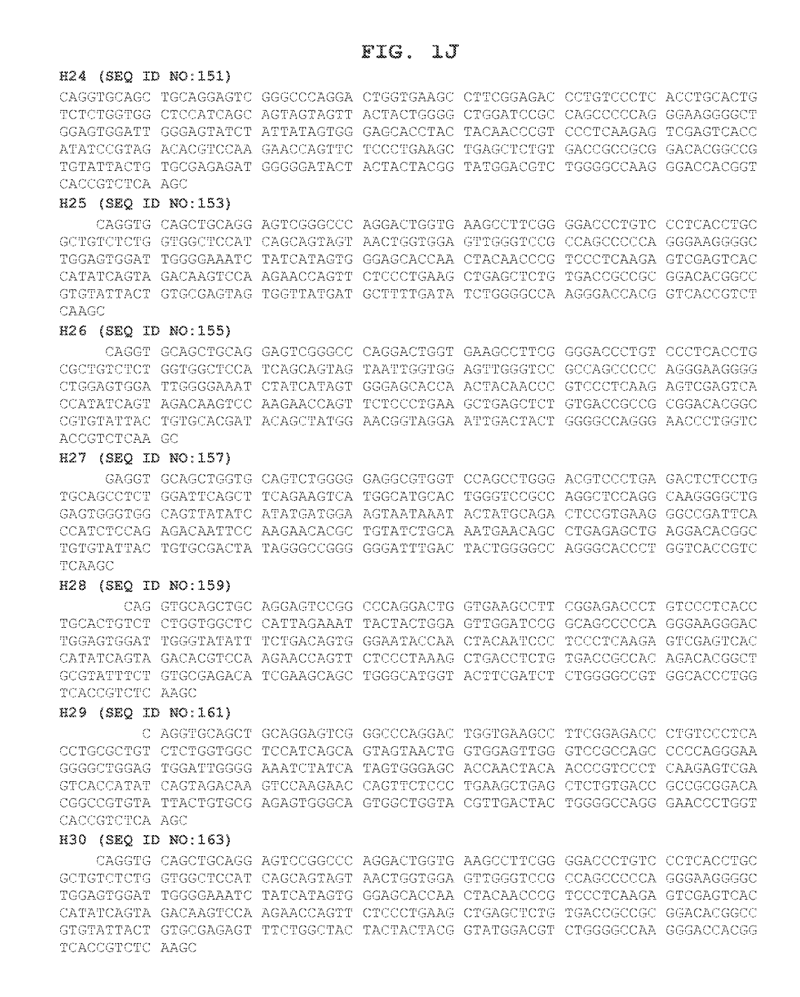

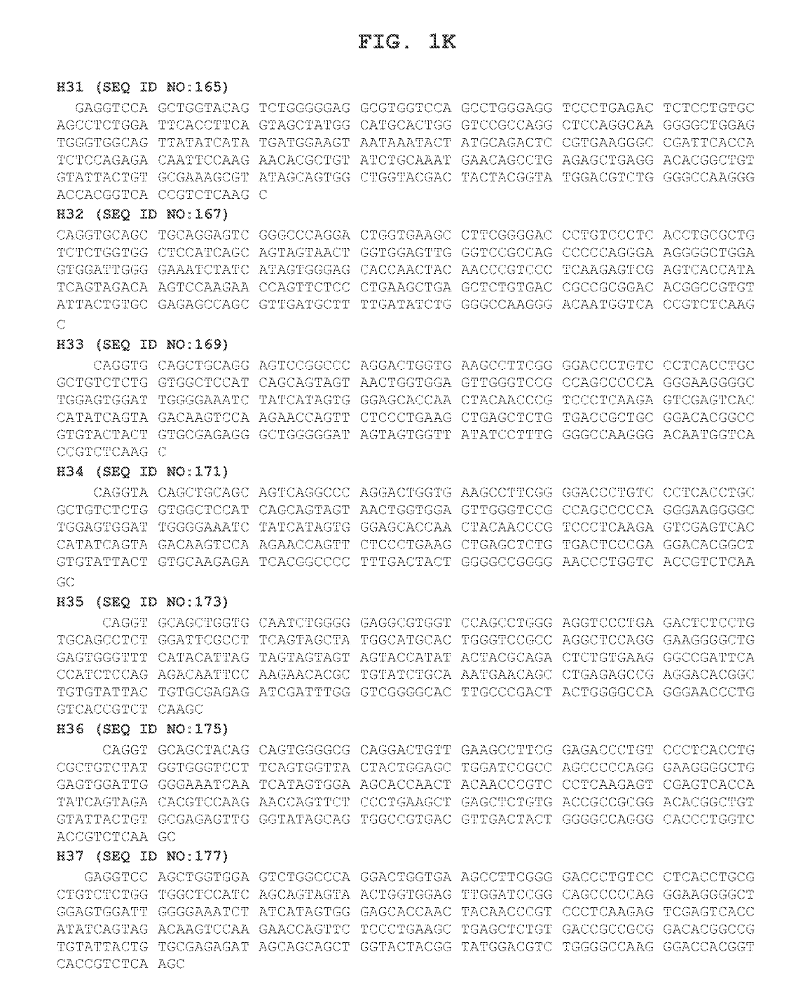

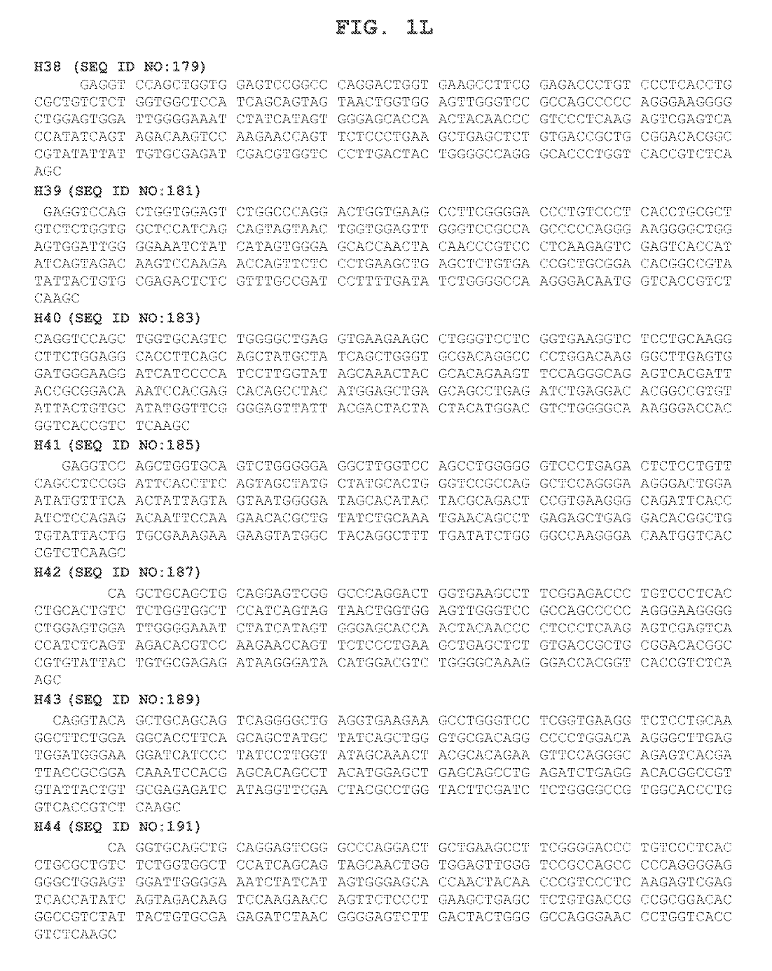

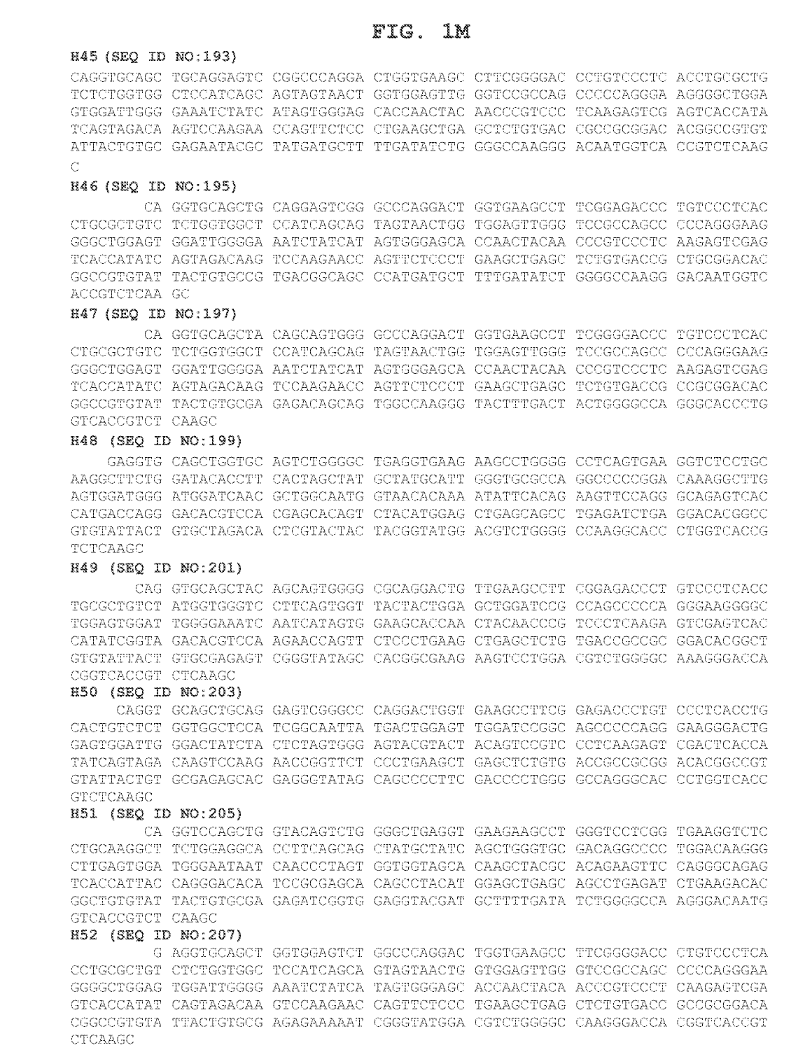

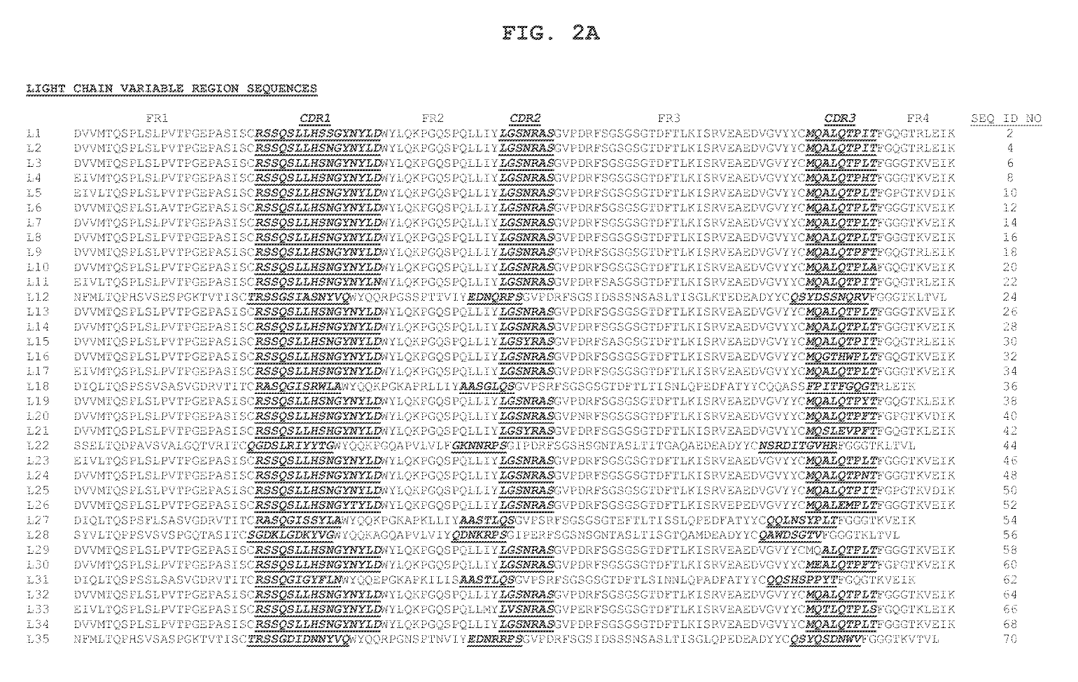

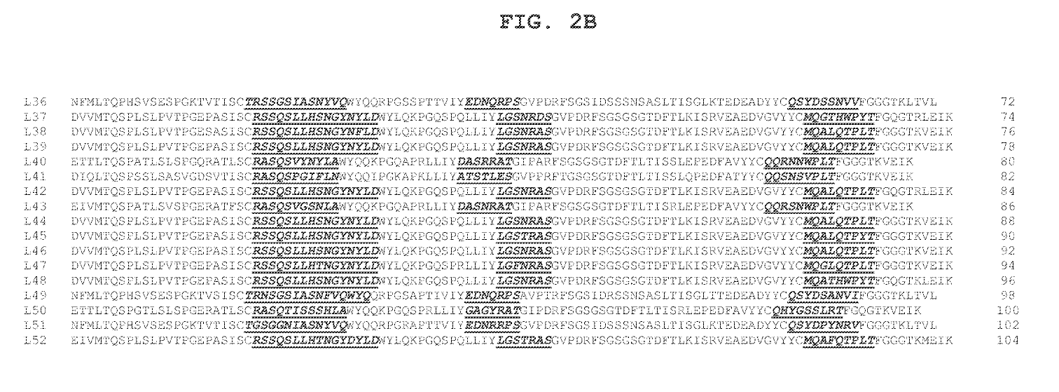

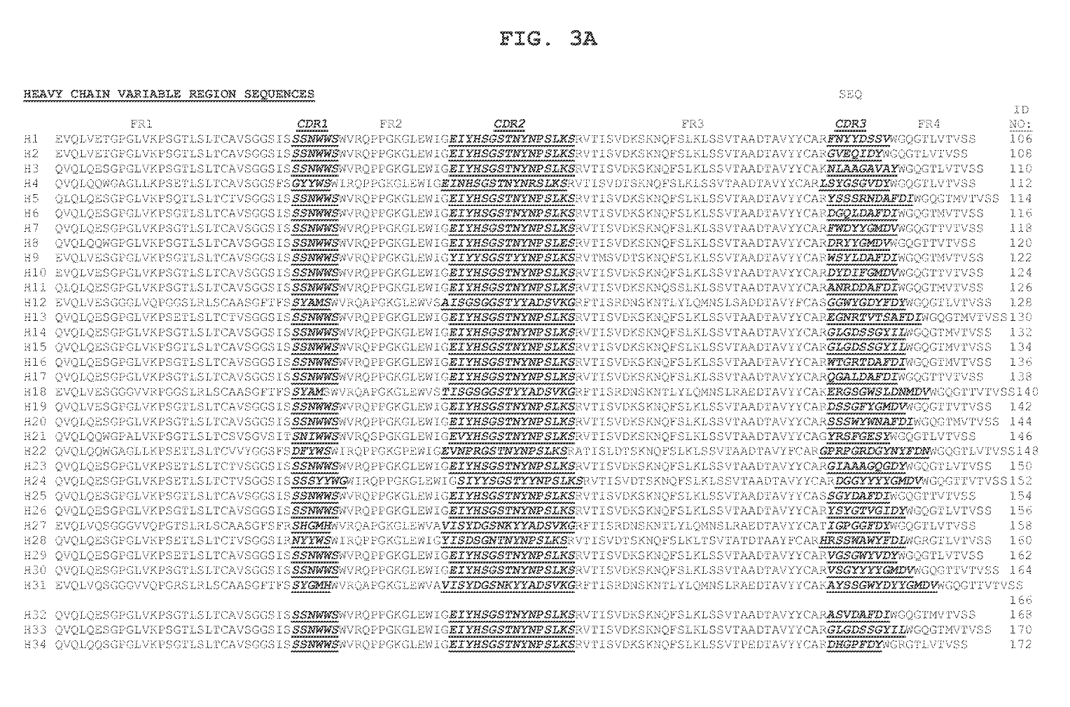

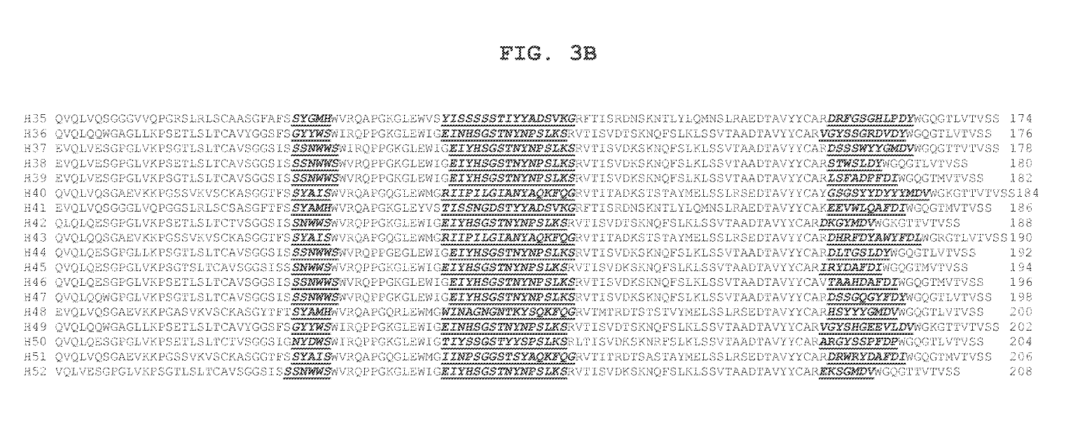

Polynucleotide and polypeptide sequences of particular light and heavy chain variable domains are shown in FIGS. 1, 2 and 3, where they are labeled, for example, L1 (“light chain variable domain 1”), H1 (“heavy chain variable domain 1”), etc. Antibodies comprising a light chain and heavy chain from FIGS. 2 and 3 are indicated by combining the name of the light chain and the name of the heavy chain variable domains. For example, “L4H7,” indicates an antibody comprising the light chain variable domain of L4 and the heavy chain variable domain of H7.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or synthesized in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The terms “IGF-1R inhibitor” and “IGF-1R antagonist” are used interchangeably. Each is a molecule that detectably inhibits at least one function of IGF-1R. Conversely, an “IGF-1R agonist” is a molecule that detectably increases at least one function of IGF-1R. The inhibition caused by an IGF-1R inhibitor need not be complete so long as it is detectable using an assay. Any assay of a function of IGF-1R can be used, examples of which are provided herein. Examples of functions of IGF-1R that can be inhibited by an IGF-1R inhibitor, or increased by an IGF-1R agonist, include binding to IGF-1, IGF-12, and/or another IGF-1R-activating molecule, kinase activity, downstream signaling, and so on. Examples of types of IGF-1R inhibitors and IGF-1R agonists include, but are not limited to, IGF-1R binding polypeptides such as antigen binding proteins (e.g., IGF-1R inhibiting antigen binding proteins), antibodies, antibody fragments, and antibody derivatives.

The terms “peptide,” “polypeptide” and “protein” each refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric.

The term “polypeptide fragment” as used herein refers to a polypeptide that has an amino-terminal and/or carboxy-terminal deletion as compared to a corresponding full-length protein. Fragments can be, for example, at least 5, 6, 7, 8, 9, 10, 11, 12, 13, 14, 15, 20, 50, 70, 80, 90, 100, 150 or 200 amino acids in length. Fragments can also be, for example, at most 1,000, 750, 500, 250, 200, 175, 150, 125, 100, 90, 80, 70, 60, 50, 40, 30, 20, 15, 14, 13, 12, 11, or 10 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

Polypeptides of the invention include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties. Analogs include muteins of a polypeptide. For example, single or multiple amino acid substitutions (e.g., conservative amino acid substitutions) may be made in the naturally occurring sequence (e.g., in the portion of the polypeptide outside the domain(s) forming intermolecular contacts. A “conservative amino acid substitution” is one that does not substantially change the structural characteristics of the parent sequence (e.g., a replacement amino acid should not tend to break a helix that occurs in the parent sequence, or disrupt other types of secondary structure that characterize the parent sequence or are necessary for its functionality). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The present invention also provides non-peptide analogs of IGF-1R binding polypeptides. Non-peptide analogs are commonly used in the pharmaceutical industry as drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference), for example, by adding internal cysteine residues capable of forming intramolecular disulfide bridges which cyclize the peptide.

A “variant” of a polypeptide (e.g., an antibody) comprises an amino acid sequence wherein one or more amino acid residues are inserted into, deleted from and/or substituted into the amino acid sequence relative to another polypeptide sequence. Variants of the invention include fusion proteins.

A “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

An “antigen binding protein” is a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991.

An “antibody” refers to an intact immunoglobulin or to an antigen binding portion thereof that competes with the intact antibody for specific binding, unless otherwise specified. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546, 1989).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Complementarity determining regions (CDRs) and framework regions (FR) of a given antibody may be identified using the system described by Kabat et al. in Sequences of Proteins of Immunological Interest, 5th Ed., US Dept. of Health and Human Services, PHS, NIH, NIH Publication no. 91-3242, 1991. One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

An antigen binding protein may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-IGF-1R antibody. In another embodiment, all of the CDRs are derived from a human anti-IGF-1R antibody. In another embodiment, the CDRs from more than one human anti-IGF-1R antibodies are mixed and matched in a chimeric antibody. For instance, a chimeric antibody may comprise a CDR1 from the light chain of a first human anti-IGF-1R antibody, a CDR2 and a CDR3 from the light chain of a second human anti-IGF-1R antibody, and the CDRs from the heavy chain from a third anti-IGF-1R antibody. Further, the framework regions may be derived from one of the same anti-IGF-1R antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind IGF-1R). See, e.g., U.S. Pat. No. 4,816,567 and Morrison, 1985, Science 229:1202-07.

A “neutralizing antibody” or “an inhibitory antibody” is an antibody that inhibits the binding of IGF-1R to IGF-I and/or IGF-2 when an excess of the anti-IGF-1R antibody reduces the amount of IGF-I and/or IGF-2 bound to IGF-1R by at least about 20% using the assay described in Example 9. In various embodiments, the antibody reduces the amount of IGF-I and/or IGF-2 bound to IGF-1R by at least 30%, 40%, 50%, 60%, 70%, 75%, 80%, 85%, 90%, 95%, 97%, 99%, and 99.9%.

An “activating antibody” is an antibody that activates IGF-1R by at least about 20% when added to a cell, tissue or organism expressing IGF-1R, where “100% activation” is the level of activation achieved under physiological conditions by the same molar amount of IGF-1 and/or IGF-2. In various embodiments, the antibody activates IGF-1R activity by at least 30%, 40%, 50%, 60%, 70%, 80%, 90%, 100%, 125%, 150%, 175%, 200%, 250%, 300%, 350%, 400%, 450%, 500%, 750%, or 1000%.

Fragments or analogs of antibodies can be readily prepared by those of ordinary skill in the art following the teachings of this specification and using techniques well-known in the art. Preferred amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., 1991, Science 253:164.

A “CDR grafted antibody” is an antibody comprising one or more CDRs derived from an antibody of a particular species or isotype and the framework of another antibody of the same or different species or isotype.

A “multi-specific antibody” is an antibody that recognizes more than one epitope on one or more antigens. A subclass of this type of antibody is a “bi-specific antibody” which recognizes two distinct epitopes on the same or different antigens.

An antigen binding protein “specifically binds” to an antigen (e.g., human IGF-1R) if it binds to the antigen with a dissociation constant of 1 nanomolar or less.

An “antigen binding domain,” “antigen binding region,” or “antigen binding site” is a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

An “epitope” is the portion of a molecule that is bound by an antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein).

The “percent identity” of two polynucleotide or two polypeptide sequences is determined by comparing the sequences using the GAP computer program (a part of the GCG Wisconsin Package, version 10.3 (Accelrys, San Diego, Calif.)) using its default parameters.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one embodiment, the nucleic acid molecules of the invention comprise a contiguous open reading frame encoding an antibody, or a fragment, derivative, mutein, or variant thereof, of the invention.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293,293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

### IGF-1R

IGF-1R is a transmembrane receptor tyrosine kinase (Blume-Jensen et al., 2001, Nature 411:355-65). The human IGF-1R is synthesized as a 1367 amino acid precursor polypeptide that includes a 30 amino acid signal peptide removed during translocation into the endoplasmic reticulum (Swiss-Prot: P08069). The IGF-1R proreceptor is glycosylated and cleaved by a protease at positions 708-711 (counting from the first amino acid following the signal peptide sequence) during maturation in the ER-golgi resulting in the formation of an α-chain (1-707) and a β-chain (712-1337) that remain linked by disulfide bonds (Bhaumick et al., 1981, Proc Natl Acad Sci USA 78:4279-83, Chemausek et al., 1981, Biochemistry 20:7345-50, Jacobs et al., 1983, Proc Natl Acad Sci USA 80:1228-31, LeBon et al., 1986, J Biol Chem 261:7685-89, Elleman, et al., 2000, Biochem J 347:771-79). The predominant form of the IGF-1R (and INSR) that exists on the cell-surface is a proteolytically processed and glycosylated (αβ)₂ dimer joined covalently by one or more disulfide bonds.

The extracellular portion of the IGF-1R consists of the α-chain and 191 amino acids of the β-chain (712-905). The receptor contains a single transmembrane spanning sequence (906-929) and a 408-residue cytoplasmic domain that includes a functional tyrosine kinase (Rubin et al., 1983, Nature 305:438-440). Comparative sequence analysis has revealed that the IGF-1R is composed of 11 distinct structural motifs (reviewed by Adams et al., 2000, Cell Mol Life Sci 57:1050-93, Marino-Buslje et al., 1998, FEBS Ltrs 441:331-36, Ward et al., 2001, BMC Bioinformatics 2:4). The N-terminal half of the extracellular domain contains two homologous domains referred to as L1 (1-151) and L2 (299-461) (Ward et al., 2001, supra) separated by a cysteine-rich (CR) region (152-298) consisting of several structural modules with disulfide linkages that align with repeating units present in the TNF receptor and laminin (Ward et al., 1995, Proteins 22:141-53). The crystal structure of the L1-CR-L2 domain has been solved (Garrett et al., 1998, Nature 394:395-99). The L2 domain is followed by three fibronectin type III domains (Marino-Buslje et al., 1998, supra, Mulhem et al., 1998, Trends Biochem Sci 23:465-66, Ward et al., 1999, Growth Factors 16:315-22). The first FnIII domain (FnIII-1, 461-579) is 118 amino acids in length. The second FnIII domain (FnIII-2, 580-798) is disrupted by a major insert sequence (ID) of about 120 amino acids in length. The ID domain includes a furin protease cleavage site that separates the α and β chains of the mature receptor. The third FnIII domain (FnIII-3) is located entirely in the β-chain (799-901) terminating several residues before the transmembrane sequence. The catalytic domain of the IGF-1R tyrosine kinase is located between amino acids positions 973-1229, and its structure has been solved (Favelyukis et al., 2001, Nature Structural Biol 8:1058-63, Pautsch et al., 2001, Structure 9:955-65). The kinase is flanked by two regulatory regions, the juxtamembrane region (930-972) and a 108 amino acid C-terminal tail (1220-1337) (Surmacz et al., 1995, Experimental Cell Res 218:370-80, Hongo et al., 1996, Oncogene 12:1231-38). The two regulatory regions contain tyrosine residues that serve as docking sites for signal transducing proteins when phosphorylated by the activated IGF-1R tyrosine kinase (reviewed by Baserga (ed.), 1998 The IGF-1 Receptor in Normal and Abnormal Growth, Hormones and Growth Factors in Development and Neoplasia, Wiley-Liss, Inc., Adams et al., 2000, Cell Mol Life Sci 57:1050-93).

The IGF-1R amino acid sequence is about 70% identical to the insulin receptor (INSR; Swiss-Prot: P06213). The highest homology between the receptors is located in the tyrosine kinase domain (84%); the lowest identity is in the CR region and the C-terminus. The IGF-1R is also highly related (˜55% identical) to the insulin related receptor (IRR; Swiss-Prot: P14616).

Human IGF-1R can be activated by the insulin-like growth factors, IGF-1 and IGF-2 and insulin (INS) (Hill et al., 1985, Pediatric Research 19:879-86). IGF-1 and IGF-2 are encoded nonallelic genes (Brissenden et al., 1984, Nature 310: 781-8, Bell et al., 1985, Proceedings of the National Academy of Sciences of the United States of America 82: 6450-54), and both genes express alternative proteins related by differential RNA splicing and protein processing. The most common and well-studied mature forms of IGF-1 and IGF-2 are respectively 70 and 67 amino acids in length (Jansen et al., 1983, Nature 306:609-11, Dull et al., 1984, Nature 310: 777-81). These proteins (and their isoforms) are identical at 11/21 positions to the insulin A-peptide, and identical at 12/30 positions with the insulin B-peptide.

Both IGF-1 and IGF-2 are secreted in large quantities by the liver under tight regulation by growth hormone secreted by the pituitary. In response to growth hormone (GH) which is in turn regulated by somatostatin (SMS) and GH releasing hormone. IGF-binding proteins (IGFBPs) are also secreted by the liver. IGF-1 and IGF-2 are both bound in circulation and sequestered in an inactive form by a series of seven IGF binding proteins (IGFBP 1-7) that bind to IGF1 and IGF2 with an affinity that is in the same order of magnitude as IGF-1R. Therefore IGF-1 and IGF-2 are only active when unbound and “bioavailable.” These binding proteins are also secreted by the liver and numerous other tissues and serve a number of functions with respect to regulating levels of free IGFs and in turn activity of IGF-1R in tumor tissue. IGFBP-3 provides most of the IGF binding capacity of serum and greatly prolongs the circulating half-life of the IGFs while competing with receptor for its ligands. The ternary complex of IGF-1 or IGF-2 and IGFBP-3, together with the acid labile subunit, is stable until dissociation through proteolysis of IGFBP-3 by proteolytic enzymes that are expressed at sites of IGF ligand activity. The functions of the other six IGFBPs are not well characterized apart from there IGF-1 and IGF-2 binding activities.

IGF-1R is expressed in all cells types in the normal adult animal except for liver hepatocytes and mature B-cells. Human blood plasma contains high concentrations of IGF-1 and IGF-2, and IGF-1 can be detected in most tissues. The receptor is an integral component of the physiological mechanism controlling organ size and homeostasis. Without being bound to a particular theory, the “Somatomedin Hypothesis” states that Growth Hormone (GH) mediated somatic growth that occurs during childhood and adolescence is dependent on the endocrine form of IGF-1 that is mainly produced and secreted by the liver (Daughaday, 2000, Pediatric Nephrology 14: 537-40). The synthesis of hepatic IGF-1 is stimulated by GH release in the pituitary in response to hypothalamic GHRH (GH releasing hormone). The serum concentration of IGF-1 increases over 100 fold between ages 5-15 in humans. The bioavailability of IGF-1 is regulated by IGF binding protein 3 (IGFBP3) with approximately 99% of the growth factor compartmentalized in the bound state. Primary IGF-1 deficiency arising form partial gene deletions, and secondary IGF-1 deficiency resulting from defects in GH production or signaling are not lethal (Woods, 1999, IGF Deficiency in Contemporary Endocrinology: The IGF System, R. a. R. Rosenfeld, C. Jr. Totowa, ed.s, Humana Press, NJ: 651-74). The affected individuals exhibit growth retardation at birth, grow slowly and can face certain CNS abnormalities.

IGF-1R signaling promotes cell growth and survival through the IRS adapter protein-dependent activation of the PI3Kinase/Akt pathway. IGF-1R transmits a signal to its major substrates, IRS-1 through IRS-4 and the Shc proteins (Blakesley et al., 1999, IGF-1 receptor function: transducing the IGF-1 signal into intracellular events in The IGF System, R. G. a. R. Rosenfeld, Jr. C. T. Totowa, ed.s, Humana Press, NJ: 143-63). This results in activation of the Ras/Raf/MAP kinase and PI3 Kinase/Akt signaling pathways. However, induction of Akt-mediated cell survival via IRS is the dominant pathway response upon IGF stimulation of most cells. See FIG. 10.

### Antigen Binding Proteins

In one aspect, the present invention provides antigen binding proteins (e.g., antibodies, antibody fragments, antibody derivatives, antibody muteins, and antibody variants), that bind to IGF-1R, e.g., human IGF-1R.

Antigen binding proteins in accordance with the present invention include antigen binding proteins that inhibit a biological activity of IGF-1R. Examples of such biological activities include binding a signaling molecule (e.g., IGF-1 and/or IGF-2), and transducing a signal in response to binding a signaling molecule.

Different antigen binding proteins may bind to different domains or epitopes of IGF-1R or act by different mechanisms of action. Examples include but are not limited to antigen binding proteins that interfere with binding of IGF-1 and/or IGF-2 to IGF-1R or that inhibit signal transduction. The site of action may be, for example, intracellular (e.g., by interfering with an intracellular signaling cascade) or extracellular. An antigen binding protein need not completely inhibit an IGF-1 and/or IGF-2 induced activity to find use in the present invention; rather, antigen binding proteins that reduce a particular activity of IGF-1 and/or IGF-2 are contemplated for use as well. (Discussions herein of particular mechanisms of action for IGF-1R-binding antigen binding proteins in treating particular diseases are illustrative only, and the methods presented herein are not bound thereby.)

It has been observed that IGF-1 and IGF-2 each exhibits biphasic binding to IGF-1R. High affinity binding has been reported to have a K_{D} in the range of 0.2 nM; high affinity binding, about ten fold higher. Thus, in one embodiment, the present invention provides an IGF-1R inhibitor that inhibits both the high and low affinity binding of IGF-1 and/or IGF-2 to IGF-R. It has been suggested that the high affinity binding, rather than the low affinity binding, of IGF-1 and/or IGF-2 to IGF-1R is required for the conformation change that activates the tyrosine kinase activity of IGF-1R. Thus, in another embodiment, the IGF-1R inhibitor preferentially inhibits the high affinity binding of IGF-1 and/or IGF-2 to IGF-1R as compared to the low affinity binding.

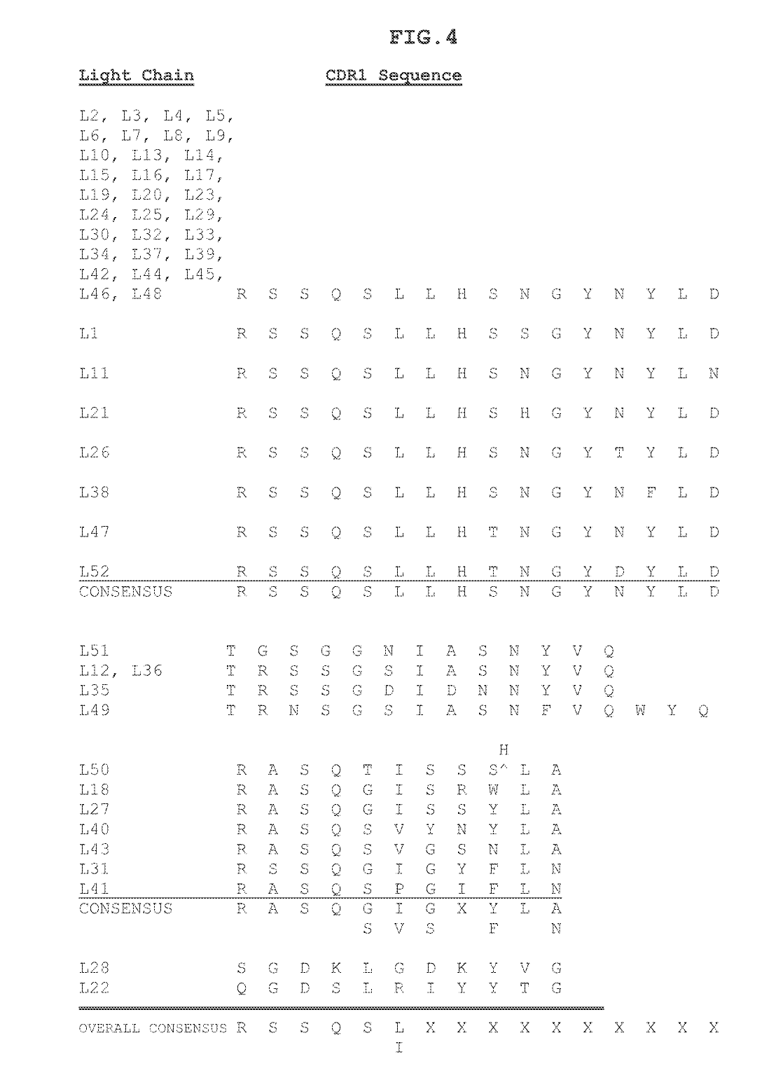

In another aspect, the present invention provides antigen binding proteins that comprise a light chain variable region selected from the group consisting of L1 through L52 and/or a heavy chain variable region selected from the group consisting of H1 through H52, and fragments, derivatives, muteins, and variants thereof (see FIGS. 2 and 3). Such an antigen binding protein can be denoted using the nomenclature “LxHy”, wherein “x” corresponds to the number of the light chain variable region and “y” corresponds to the number of the heavy chain variable region as they are labeled in FIGS. 2 and 3. For example, L2H1 refers to an antigen binding protein with a light chain variable region comprising the amino acid sequence of L2 and a heavy chain variable region comprising the amino acid sequence of H1, as shown in FIGS. 2 and 3. FIGS. 2 and 3 also indicate the location of the CDR and framework regions of each of these variable domain sequences. The CDR regions of each light and heavy chain also are grouped by type and by sequence similarity in FIGS. 4 through 9. Antigen binding proteins of the invention include, for example, antigen binding proteins having a combination of light chain and heavy chain variable domains selected from the group of combinations consisting of L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52.

In one embodiment, the present invention provides an antigen binding protein comprising a light chain variable domain comprising a sequence of amino acids that differs from the sequence of a light chain variable domain selected from the group consisting of L1 through L52 only at 15, 14, 13, 12, 11, 10, 9, 8, 7, 6, 5, 4, 3, 2, or 1 residues, wherein each such sequence difference is independently either a deletion, insertion, or substitution of one amino acid residue. In another embodiment, the light-chain variable domain comprises a sequence of amino acids that is at least 70%, 75%, 80%, 85%, 90%, 95%, 97%, or 99% identical to the sequence of a light chain variable domain selected from the group consisting of L1 through L52. In another embodiment, the light chain variable domain comprises a sequence of amino acids that is encoded by a nucleotide sequence that is at least 70%, 75%, 80%, 85%, 90%, 95%, 97%, or 99% identical to a nucleotide sequence that encodes a light chain variable domain selected from the group consisting of L1 through L52. In another embodiment, the light chain variable domain comprises a sequence of amino acids that is encoded by a polynucleotide that hybridizes under moderately stringent conditions to the complement of a polynucleotide that encodes a light chain variable domain selected from the group consisting of L1 through L52. In another embodiment, the light chain variable domain comprises a sequence of amino acids that is encoded by a polynucleotide that hybridizes under moderately stringent conditions to the complement of a polynucleotide that encodes a light chain variable domain selected from the group consisting of L1 through L52. In another embodiment, the light chain variable domain comprises a sequence of amino acids that is encoded by a polynucleotide that hybridizes under moderately stringent conditions to a complement of a light chain polynucleotide selected from FIG. 1.

In another embodiment, the present invention provides an antigen binding protein comprising a heavy chain variable domain comprising a sequence of amino acids that differs from the sequence of a heavy chain variable domain selected from the group consisting of H1 through H52 only at 15, 14, 13, 12, 11, 10, 9, 8, 7, 6, 5, 4, 3, 2, or 1 residue(s), wherein each such sequence difference is independently either a deletion, insertion, or substitution of one amino acid residue. In another embodiment, the heavy chain variable domain comprises a sequence of amino acids that is at least 70%, 75%, 80%, 85%, 90%, 95%, 97%, or 99% identical to the sequence of a heavy chain variable domain selected from the group consisting of H1 through H52. In another embodiment, the heavy chain variable domain comprises a sequence of amino acids that is encoded by a nucleotide sequence that is at least 70%, 75%, 80%, 85%, 90%, 95%, 97%, or 99% identical to a nucleotide sequence that encodes a heavy chain variable domain selected from the group consisting of H1 through H52. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of H1 through H52. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of H1 through H52. In another embodiment, the heavy chain variable domain comprises a sequence of amino acids that is encoded by a polynucleotide that hybridizes under moderately stringent conditions to a complement of a heavy chain polynucleotide selected from FIG. 1.

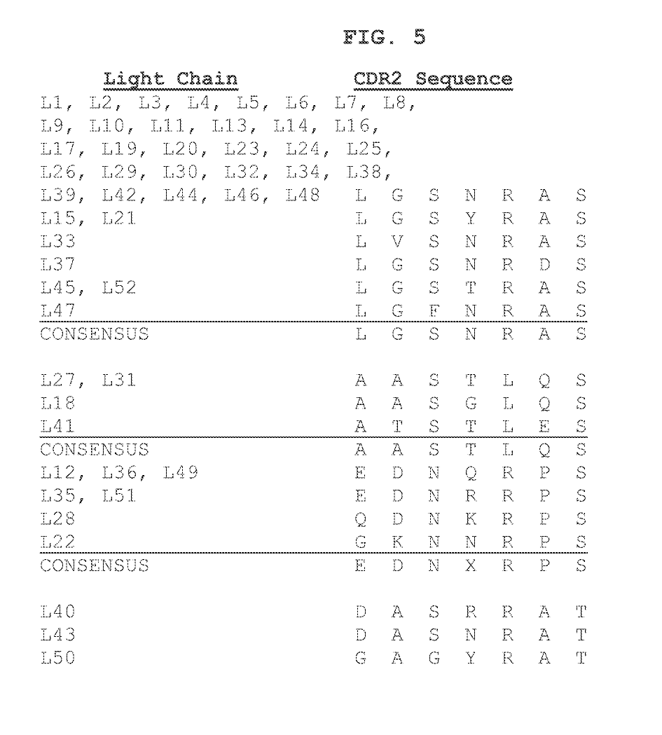

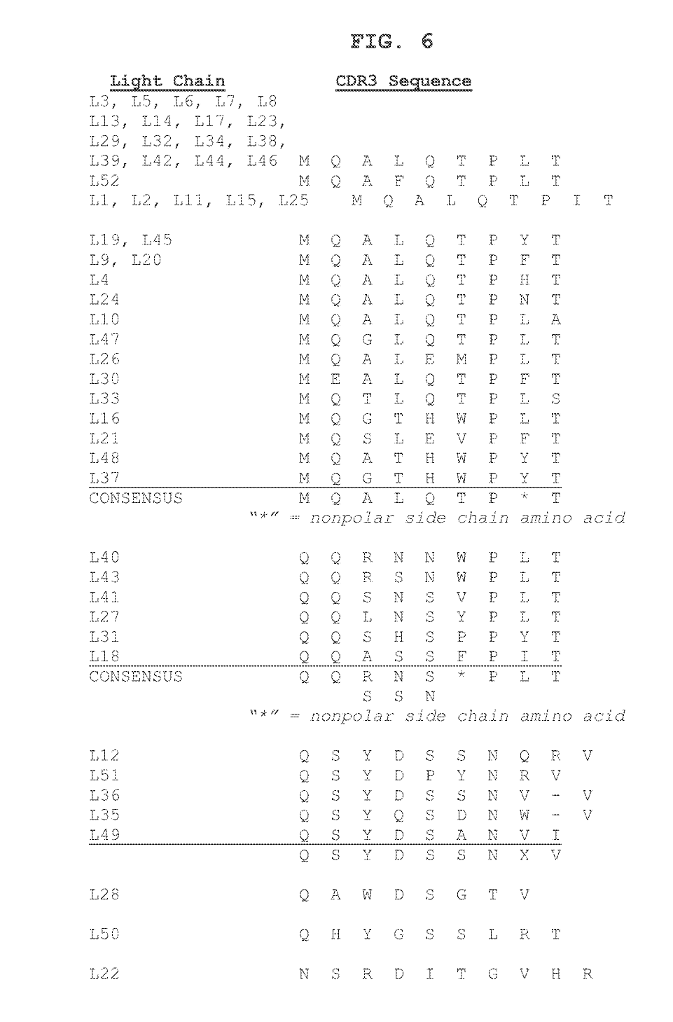

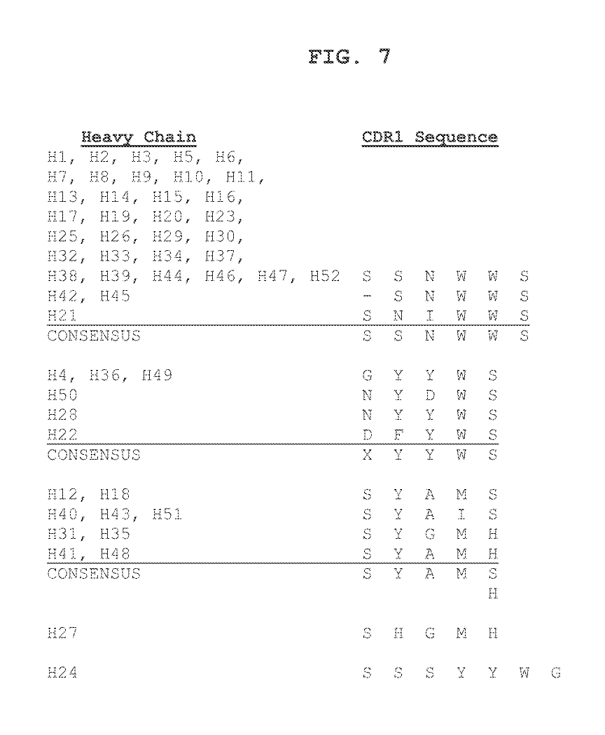

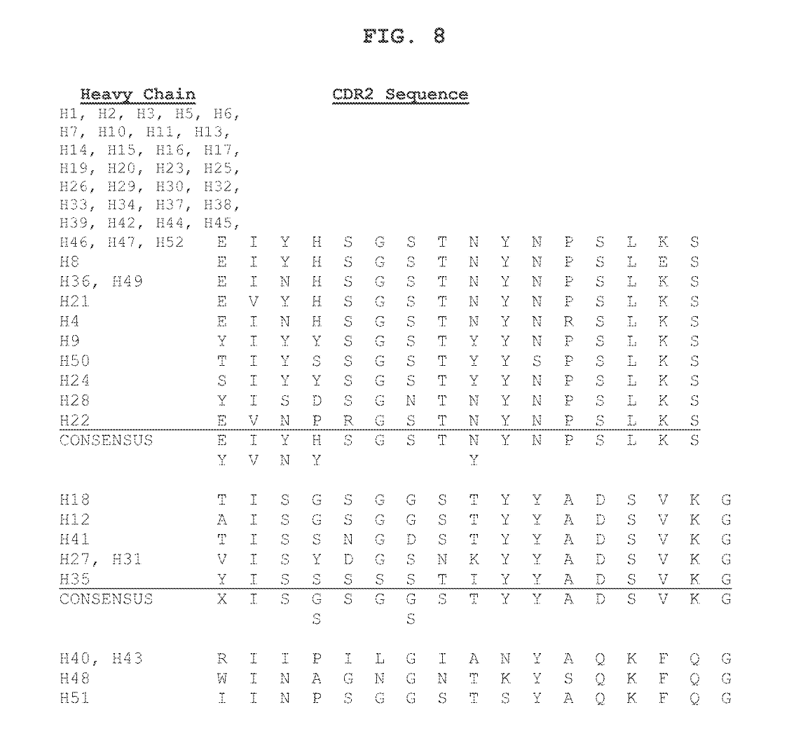

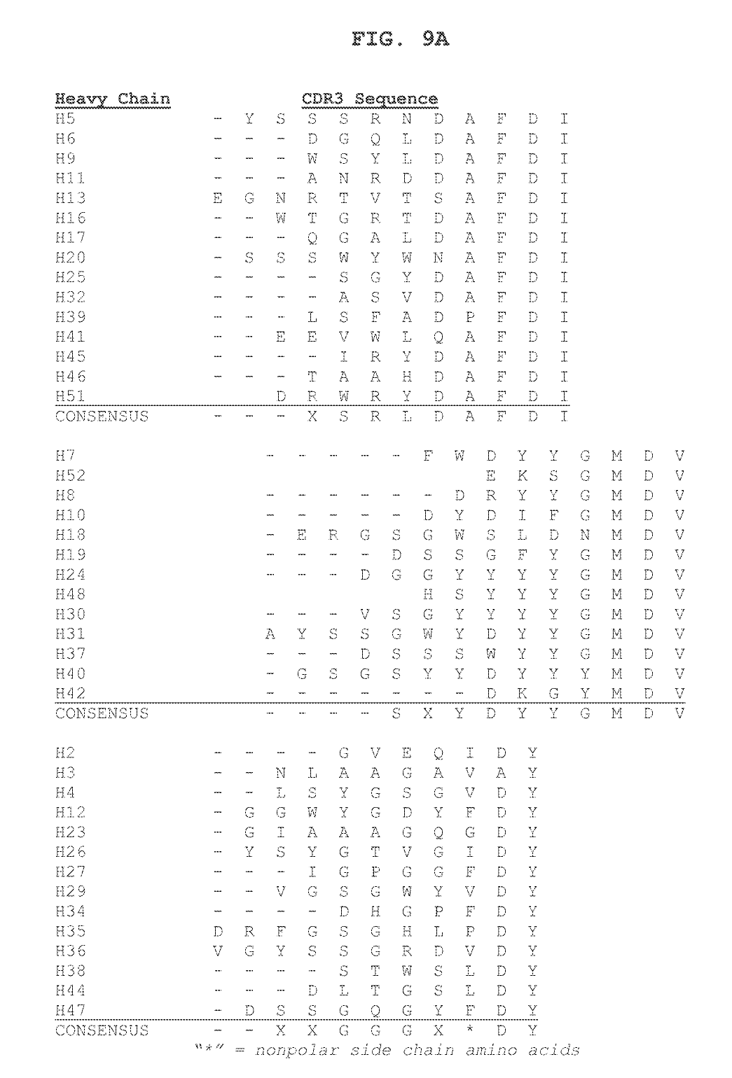

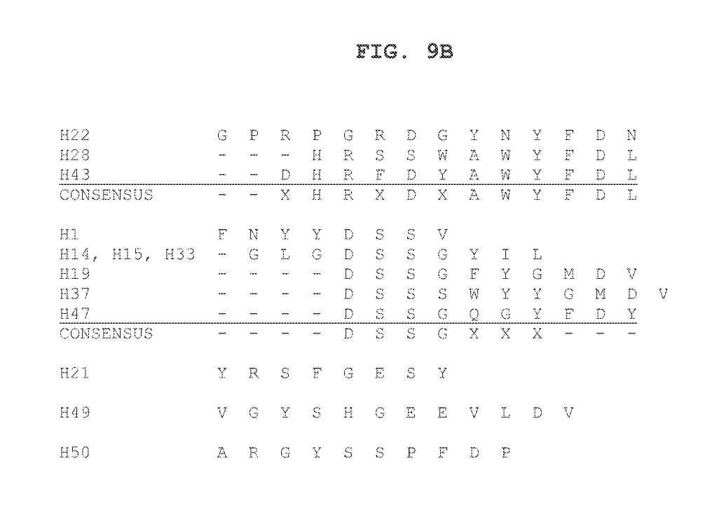

Particular embodiments of antigen binding proteins of the present invention comprise one or more amino acid sequences that are identical to the amino acid sequences of one or more of the CDRs and/or FRs illustrated in FIGS. 2 through 9. In one embodiment, the antigen binding protein comprises a light chain CDR1 sequence illustrated in FIG. 4. In another embodiment, the antigen binding protein comprises a light chain CDR2 sequence illustrated in FIG. 5. In another embodiment, the antigen binding protein comprises a light chain CDR3 sequence illustrated in FIG. 6. In another embodiment, the antigen binding protein comprises a heavy chain CDR1 sequence illustrated in FIG. 7. In another embodiment, the antigen binding protein comprises a heavy chain CDR2 sequence illustrated in FIG. 8. In another embodiment, the antigen binding protein comprises a heavy chain CDR3 sequence illustrated in FIG. 9. In another embodiment, the antigen binding protein comprises a light chain FR1 sequence illustrated in FIG. 2. In another embodiment, the antigen binding protein comprises a light chain FR2 sequence illustrated in FIG. 2. In another embodiment, the antigen binding protein comprises a light chain FR3 sequence illustrated in FIG. 2. In another embodiment, the antigen binding protein comprises a light chain FR4 sequence illustrated in FIG. 2. In another embodiment, the antigen binding protein comprises a heavy chain FR1 sequence illustrated in FIG. 3. In another embodiment, the antigen binding protein comprises a heavy chain FR2 sequence illustrated in FIG. 3. In another embodiment, the antigen binding protein comprises a heavy chain FR3 sequence illustrated in FIG. 3. In another embodiment, the antigen binding protein comprises a heavy chain FR4 sequence illustrated in FIG. 3.

In one embodiment, the present invention provides an antigen binding protein that comprises one or more CDR sequences that differ from a CDR sequence shown in FIGS. 2 through 9 by no more than 5, 4, 3, 2, or 1 amino acid residues.

In one embodiment, the present invention provides an antigen binding protein that comprises at least one CDR from L1-L52 and/or H1-H52, as shown in FIGS. 2 through 9, and at least one CDR sequence from an anti-IGF-1R antibody described in US Pat. App. Pub. Nos. 03/0235582, 04/0228859, 04/0265307, 04/0886503, 05/0008642, 05/0084906, 05/0186203, 05/0244408, PCT Pub. Nos. WO 03/059951, WO 03/100008, WO 04/071529A2, WO 04/083248, WO 04/087756, WO 05/016967, WO 05/016970, or WO 05/058967 (each of which is incorporated herein by reference in its entirety for all purposes) wherein the antigen binding protein binds to IGF-1 receptor. In another embodiment, the antigen binding protein comprises 2, 3, 4, or 5 CDR sequences from L1-L52 and/or H1-H52, as shown in FIGS. 2 through 9. In another embodiment, the antigen binding protein comprises 2, 3, 4, or 5 CDR sequences from an anti-IGF-1R antibody described in US Pat. App. Pub. Nos. 03/0235582, 04/0228859, 04/0265307, 04/0886503, 05/0008642, 05/0084906, 05/0186203, 05/0244408, PCT Pub. Nos. WO 03/059951, WO 03/100008, WO 04/071529A2, WO 04/083248, WO 04/087756, WO 05/016967, WO 05/016970, or WO 05/058967. In another embodiment, at least one of the antigen binding protein's CDR3 sequences is a CDR3 sequence from L1-L52 and/or H1-H52, as shown in FIGS. 2, 3, 6, and 9. In another embodiment, the antigen binding protein's light chain CDR3 sequence is a light chain CDR3 sequence from L1-L52 as shown in FIGS. 2 and 6 and the antigen binding protein's heavy chain CDR3 sequence is a heavy chain sequence from H1-H52 as shown in FIGS. 3 and 9. In another embodiment, the antigen binding protein comprises 1, 2, 3, 4, or 5 CDR sequences that each independently differs by 6, 5, 4, 3, 2, 1, or 0 single amino acid additions, substitutions, and/or deletions from a CDR sequence of L1-L52 and/or H1-H52, and the antigen binding protein further comprises 1, 2, 3, 4, or 5 CDR sequences that each independently differs by 6, 5, 4, 3, 2, 1, or 0 single amino acid additions, substitutions, and/or deletions from a CDR sequence of US Pat. App. Pub. Nos. 03/0235582, 04/0228859, 04/0265307, 04/0886503, 05/0008642, 05/0084906, 05/0186203, 05/0244408, PCT Pub. Nos. WO 03/059951, WO 03/100008, WO 04/071529A2, WO 04/083248, WO 04/087756, WO 05/016967, WO 05/016970, or WO 05/058967. In another embodiment, the CDR sequence(s) from US Pat. App. Pub. Nos. 03/0235582, 04/0228859, 04/0265307, 04/0886503, 05/0008642, 05/0084906, 05/0186203, 05/0244408, PCT Pub. Nos. WO 03/059951, WO 03/100008, WO 04/071529A2, WO 04/083248, WO 04/087756, WO 05/016967, WO 05/016970, or WO 05/058967. In another embodiment, the CDR sequence(s) are from (an) antibody(-ies) that bind(s) to the L2 portion of the extracellular domain of IGF-1 receptor. In another embodiment, the antigen binding protein does not comprise a light chain CDR3 sequence and/or a heavy chain CDR3 sequence from an anti-IGF-1R antibody from US Pat. App. Pub. Nos. 03/0235582, 04/0228859, 04/0265307, 04/0886503, 05/0008642, 05/0084906, 05/0186203, 05/0244408, PCT Pub. Nos. WO 03/059951, WO 03/100008, WO 04/071529A2, WO 04/083248, WO 04/087756, WO 05/016967, WO 05/016970, or WO 05/058967.

In one embodiment, the present invention provides an antigen binding protein that comprises a light chain CDR1 comprising the sequence RSSQSLLHX₁X₂GYNX₃LX₄ (SEQ ID NO:236), wherein X₁ is a serine or a threonine residue, X₂ is an asparagine, serine, or histidine residue, X₃ is a tyrosine or a phenylalanine residue, and X₄ is an aspartate or an asparagine residue. In another embodiment, the light chain CDR1 comprises the sequence TRSSGX₁IX₂X₃NYVQ (SEQ ID NO:237), wherein X₁ is a serine or an aspartate residue, X₂ is an alanine or an aspartate residue, and X₃ is a serine or an asparagine residue. In another embodiment, the light chain CDR1 comprises the sequence RASQX₁X₂X₃X₄X₅LX₆ (SEQ ID NO:238), wherein X₁ is a glycine or a serine residue, X₂ is an isoleucine, valine, or proline residue, and X₃ is a serine, glycine, or tyrosine residue, X₄ is any amino acid residue, X₅ is a phenylalanine, tyrosine, asparagine, or tryptophan residue, and X₆ is an alanine or an asparagine residue. In another embodiment, X₂ is an isoleucine or valine residue, X₃ is a glycine or serine residue, X₄ is an arginine, serine, asparagine, serine, tyrosine, or isoleucine residue, and X₅ is a phenylalanine or a tyrosine residue.

In one embodiment, the present invention provides an antigen binding protein that comprises a light chain CDR2 comprising the sequence LX₁X₂X₃RX₄S (SEQ ID NO:239), wherein X₁ is a glycine or a valine residue, X₂ is a serine or a phenylalanine residue, X₃ is an asparagine, tyrosine, or threonine residue, and X₄ is an alanine or an aspartate residue. In another embodiment, the CDR2 comprises the sequence AX₁SX₂LX₃S (SEQ ID NO:240), wherein X₁ is an alanine or a threonine residue, X₂ is a threonine or a glycine residue, and X₃ is a glutamine or a glutamate residue. In another embodiment, the CDR2 comprises the sequence X₁X₂NX₃RPS (SEQ ID NO:241), wherein X₁ is a glutamate, glutamine, or glycine residue, X₂ is an aspartate or lysine residue, and X₃ is any amino acid residue.

In one embodiment, the present invention provides an antigen binding protein that comprises a light chain CDR3 comprising the sequence MX₁X₂X₃X₄X₅PX₆X₇ (SEQ ID NO:242), wherein X₁ is a glutamine or glutamate residue, X₂ is an alanine, glycine, serine, or threonine residue, X₃ is a leucine or threonine residue, X₄ is a glutamine, glutamate, or histidine residue, X₅ is a threonine, tryptophan, methionine, or valine residue, X₆ is a nonpolar side chain residue, and X₇ is a threonine, serine, or alanine residue. In another embodiment, the CDR3 comprises the sequence QQX₁X₂X₃X₄PX₅T (SEQ ID NO:243), wherein X₁ is an arginine, serine, leucine, or alanine residue, X₂ is an asparagine, serine, or histidine residue, X₃ is a serine or an asparagine residue, X₄ is a nonpolar side chain residue, and X₅ is a leucine, isoleucine, tyrosine, or tryptophan residue. In another embodiment, the CDR3 comprises the sequence QSYX₁SX₂NX₃X₄V (SEQ ID NO:244), wherein X₁ is an aspartate or a glutamine residue, X₂ is a serine or an aspartate residue, X₃ is a glutamine, valine, or tryptophan residue, and X₄ is an arginine residue or no residue.

In one embodiment, the present invention provides an antigen binding protein that comprises a heavy chain CDR1 comprising the sequence X₃X₂X₃WWS (SEQ ID NO:245), wherein X₁ is a serine residue or no residue, X₂ is a serine or asparagine residue, and X₃ is an asparagine residue and an isoleucine residue. In another embodiment, the heavy chain CDR1 comprises the sequence X₁X₂YWS (SEQ ID NO:246), wherein X₁ is a glycine, asparagine, or aspartate residue, and X₂ is a tyrosine or phenylalanine residue. In another embodiment, the heavy chain CDR1 comprises the sequence SYX₁X₂X₃ (SEQ ID NO:247), wherein X₁ is an alanine or glycine residue, X₂ is a methionine or isoleucine residue, and X₃ is a serine or histidine residue.

In one embodiment, the present invention provides an antigen binding protein that comprises a heavy chain CDR2 comprising the sequence X₁X₂X₃X₄X₅GX₆TX₇YNPSLX₈S (SEQ ID NO:248), wherein X₁ is a glutamate, tyrosine, or serine residue, X₂ is a isoleucine or valine residue, X₃ is a tyrosine, asparagine, or serine residue, X₄ is a histidine, tyrosine, aspartate, or proline residue, X₅ is a serine or arginine residue, X₆ is a serine or asparagine residue, X₇ is an asparagine or tyrosine residue, and X₈ is a lysine or glutamate residue. In another embodiment, the heavy chain CDR2 comprises the sequence X₁ISX₂X₃X₄X₅X₆X₇YYADSVKG (SEQ ID NO:249), wherein X₁ is a threonine, alanine, valine, or tyrosine residue, X₂ is a glycine, serine, or tyrosine residue, X₃ is a serine, asparagine, or aspartate residue, X₄ is a glycine or serine residue, X₅ is a glycine, serine, or aspartate residue, X₆ is a serine, threonine, or asparagine residue, and X₇ is a threonine, lysine, or isoleucine residue.

In one embodiment, the present invention provides an antigen binding protein that comprises a heavy chain CDR3 comprising the sequence X₁X₂X₃X₄X₅X₆X₇X₈X₉FDI (SEQ ID NO:250), wherein X₁ is a glutamate residue or no residue, X₂ is tyrosine, glycine, or serine residue or no residue, X₃ is a serine, asparagine, tryptophan, or glutamate residue, or no residue, X₄ is a serine, aspartate, tryptophan, alanine, arginine, threonine, glutamine, leucine, or glutamate residue, or no residue, X₅ is a serine, glycine, asparagine, threonine, tryptophan, alanine, valine, or isoleucine residue, X₆ is an arginine, glutamine, tyrosine, valine, alanine, glycine, serine, phenylalanine, or tryptophan residue, X₇ is a leucine, asparagine, aspartate, threonine, tryptophan, tyrosine, valine, alanine, or histidine residue, X₈ is an aspartate, serine, asparagine, or glutamine residue, and X₉ is an alanine or a proline residue. In another embodiment, the heavy chain CDR3 comprises the sequence X₁X₂X₃X₄X₅X₆X₇X₈X₉X₁₀X₁₁MDV (SEQ ID NO:251), wherein X_{I} is an alanine residue, or no residue, X₂ is a glutamate, tyrosine, or glycine residue, or no residue, X₃ is a serine or arginine residue, or no residue, X₄ is an aspartate, glycine, serine, or valine residue, or no residue, X₅ is a serine, glycine, or aspartate residue, or no residue, X₆ is a glycine, phenylalanine, aspartate, serine, tryptophan, or tyrosine residue, or no residue, X₇ is a tyrosine, tryptophan, serine, or aspartate residue, or no residue, X₈ is an aspartate, arginine, serine, glycine, tyrosine, or tryptophan residue, X₉ is a tyrosine, isoleucine, leucine, phenylalanine, or lysine residue, X₁₀ is a tyrosine, phenylalanine, aspartate, or glycine residue, and X₁₁ is a glycine, tyrosine, or asparagine residue. In another embodiment, the heavy chain CDR3 comprises the sequence X₁X₂X₃X₄X₅X₆X₇X₈X₉X₁₀Y (SEQ ID NO:252), wherein X₁ is an aspartate or valine residue, or no residue, X₂ is a glycine, tyrosine, arginine, or aspartate residue, or no residue, X₃ is an asparagine, leucine, glycine, isoleucine, serine, valine, phenylalanine, or tyrosine residue, or no residue, X₄ is a leucine, serine, tryptophan, alanine, tyrosine, isoleucine, glycine, or aspartate residue, or no residue, X₅ is a glycine, alanine, tyrosine, serine, aspartate, or leucine residue, X₆ is a valine, alanine, glycine, threonine, proline, histidine, or glutamine residue, X₇ is a glutamate, glycine, serine, aspartate, glycine, valine, tryptophan, histidine, or arginine residue, X₈ is a glutamine, alanine, glycine, tyrosine, proline, leucine, aspartate, or serine residue, X₉ is a nonpolar side chain residue, and X₁₀ is an aspartate or alanine residue. In another embodiment, the heavy chain CDR3 comprises the sequence X_{I}X₂X₃X₄X₅X₆X₇X₈X₉X₁₀YFDX₁₁ (SEQ ID NO:253), wherein X₁ is a glycine residue, or no residue, X₂ is a proline residue, or no residue, X₃ is an arginine or aspartate residue, or no residue, X₄ is a histidine or proline residue, X₅ is an arginine or glycine residue, X₆ is an arginine, serine, or phenylalanine residue, X₇ is an aspartate or serine residue, X₈ is a glycine, tryptophan, or tyrosine residue, X₉ is a tyrosine or alanine residue, X₁₀ is an asparagine or tryptophan residue, and X₁₁ is an asparagine or leucine residue. In another embodiment, the heavy chain CDR3 comprises the sequence X₁X₂X₃X₄DSSX₅X₆X₇X₈X₉X₁₀X₁₁X₁₂ (SEQ ID NO:254), wherein X₁ is a phenylalanine residue, or no residue, X₂ is an asparagine or glycine residue, or no residue, X₃ is a tyrosine or a leucine residue, or no residue, X₄ is a tyrosine or glycine residue, or no residue, X₅ is a glycine, serine, or valine residue, X₆ is a tyrosine, phenylalanine, tryptophan, or glutamine residue, or no residue, X₇ is a tyrosine, glycine, or isoleucine residue, or no residue, X₈ is a tyrosine, leucine, or glycine residue, or no residue, X₉ is a methionine, glycine, or phenylalanine residue, or no residue, X₁₀ is an aspartate or methionine residue, or no residue, X₁₁ is a valine, aspartate, or tyrosine residue, or no residue, and X₁₂ is a valine residue, or no residue.

In one embodiment, the present invention provides an isolated antigen binding protein, comprising either: a. a light chain CDR3 comprising a sequence selected from the group consisting of: i. a light chain CDR3 sequence selected from the group consisting of the light chain CDR3 sequences of L1-L52 as shown in FIG. 6; ii. MQALQTPZT; QQ(R/S)(N/S)(S/N)ZPLT; and iv. QSYDSSNXJV; b. a heavy chain CDR3 comprising a sequence selected from the group consisting of: i. a heavy chain CDR3 sequence that differs by no more than a total of three amino acid additions, substitutions, or deletions from a CDR3 sequence selected from the group consisting of the heavy chain CDR3 sequences of H1-H52 as shown in FIG. 9; ii. SRLDAFDI; iii. SXYDYYGMDV; iv. HRXDXAWYFDL; and v. DSSG; or c. the light chain CDR3 sequence of (a) and the heavy chain CDR3 sequence of (b); wherein amino acid residue symbols enclosed in parentheses identify alternative residues for the same position in a sequence, each X is independently any amino acid residue, each Z is independently a glycine residue, an alanine residue, a valine residue, a leucine residue, an isoleucine residue, a proline residue, a phenylalanine residue, a methionine residue, a tryptophan residue, or a cysteine residue, each J is independently a glutamine residue, an arginine residue, a valine residue, or a tryptophan residue, and the antigen binding protein binds to human IGF-1R.

The nucleotide sequences of FIG. 1, or the amino acid sequences of FIGS. 2 through 9, can be altered, for example, by random mutagenesis or by site-directed mutagenesis (e.g., oligonucleotide-directed site-specific mutagenesis) to create an altered polynucleotide comprising one or more particular nucleotide substitutions, deletions, or insertions as compared to the non-mutated polynucleotide. Examples of techniques for making such alterations are described in Walder et al., 1986, Gene 42:133; Bauer et al. 1985, Gene 37:73; Craik, BioTechniques, January 1985, 12-19; Smith et al., 1981, Genetic Engineering: Principles and Methods, Plenum Press; and U.S. Pat. Nos. 4,518,584 and 4,737,462. These and other methods can be used to make, for example, derivatives of anti-IGF-1R antibodies that have a desired property, for example, increased affinity, avidity, or specificity for IGF-1R, increased activity or stability in vivo or in vitro, or reduced in vivo side-effects as compared to the underivatized antibody.

Other derivatives of anti-IGF-1R antibodies within the scope of this invention include covalent or aggregative conjugates of anti-IGF-1R antibodies, or fragments thereof, with other proteins or polypeptides, such as by expression of recombinant fusion proteins comprising heterologous polypeptides fused to the N-terminus or C-terminus of an anti-IGF-1R antibody polypeptide. For example, the conjugated peptide may be a heterologous signal (or leader) polypeptide, e.g., the yeast alpha-factor leader, or a peptide such as an epitope tag. Antigen binding protein-containing fusion proteins can comprise peptides added to facilitate purification or identification of antigen binding protein (e.g., poly-His). An antigen binding protein also can be linked to the FLAG peptide Asp-Tyr-Lys-Asp-Asp-Asp-Asp-Lys (DYKDDDDK) (SEQ ID NO:255) as described in Hopp et al., Bio/Technology 6:1204, 1988, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

Oligomers that contain one or more antigen binding proteins may be employed as IGF-1R antagonists. Oligomers may be in the form of covalently-linked or non-covalently-linked dimers, trimers, or higher oligomers. Oligomers comprising two or more antigen binding protein are contemplated for use, with one example being a homodimer. Other oligomers include heterodimers, homotrimers, heterotrimers, homotetramers, heterotetramers, etc.

One embodiment is directed to oligomers comprising multiple antigen binding proteins joined via covalent or non-covalent interactions between peptide moieties fused to the antigen binding proteins. Such peptides may be peptide linkers (spacers), or peptides that have the property of promoting oligomerization. Leucine zippers and certain polypeptides derived from antibodies are among the peptides that can promote oligomerization of antigen binding proteins attached thereto, as described in more detail below.

In particular embodiments, the oligomers comprise from two to four antigen binding proteins. The antigen binding proteins of the oligomer may be in any form, such as any of the forms described above, e.g., variants or fragments. Preferably, the oligomers comprise antigen binding proteins that have IGF-1R binding activity.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, PNAS USA 88:10535; Byrn et al., 1990, Nature 344:677; and Hollenbaugh et al., 1992 “Construction of Immunoglobulin Fusion Proteins”, in Current Protocols in Immunology, Suppl. 4, pages 10.19.1-10.19.11.

One embodiment of the present invention is directed to a dimer comprising two fusion proteins created by fusing an IGF-1R binding fragment of an anti-IGF-1R antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, the variable portion of the heavy and/or light chains of an anti-IGF-1R antibody may be substituted for the variable portion of an antibody heavy and/or light chain.

Alternatively, the oligomer is a fusion protein comprising multiple antigen binding proteins, with or without peptide linkers (spacer peptides). Among the suitable peptide linkers are those described in U.S. Pat. Nos. 4,751,180 and 4,935,233.

Another method for preparing oligomeric antigen binding proteins involves use of a leucine zipper. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191, hereby incorporated by reference. The use of a modified leucine zipper that allows for stable trimerization of a heterologous protein fused thereto is described in Fanslow et al., 1994, Semin. Immunol. 6:267-78. In one approach, recombinant fusion proteins comprising an anti-IGF-1R antibody fragment or derivative fused to a leucine zipper peptide are expressed in suitable host cells, and the soluble oligomeric anti-IGF-1R antibody fragments or derivatives that form are recovered from the culture supernatant.

In one aspect, the present invention provides antigen binding proteins that interfere with the binding of IGF-1 and/or IGF-2 to an IGF-1R. Such antigen binding proteins can be made against IGF-1R, or a fragment, variant or derivative thereof, and screened in conventional assays for the ability to interfere with binding of IGF-1 and/or IGF-2 to IGF-1R. Examples of suitable assays are assays that test the antigen binding proteins for the ability to inhibit binding of IGF-1 and/or IGF-2 to cells expressing IGF-1R, or that test antigen binding proteins for the ability to reduce a biological or cellular response that results from the binding of IGF-1 and/or IGF-2 to cell surface IGF-1R receptors.

In another aspect, the present invention provides an antigen binding protein that blocks the binding of IGF-1 and/or IGF-2 to IGF-1R but does not significantly block the binding of insulin to insulin receptor (INS-R). In one embodiment, the antigen binding protein does not bind to INS-R. In another embodiment, the antigen binding protein binds to the INS-R with such a low affinity that it does not effectively block the binding of insulin to INS-R. In another embodiment, the antigen binding protein binds to INS-R, but antigen binding protein-bound INS-R can still bind to insulin. In another embodiment, the antigen binding protein's selectivity for IGF-1R is at least 50 times greater than its selectivity for insulin receptor. In another embodiment, the selectivity of the antigen binding protein is more than 100 times greater than its selectivity for insulin receptor.

In another aspect, the present invention provides an antigen binding protein that demonstrates species selectivity. In one embodiment, the antigen binding protein binds to one or more mammalian IGF-1R, for example, to human IGF-1R and one or more of mouse, rat, guinea pig, hamster, gerbil, cat, rabbit, dog, goat, sheep, cow, horse, camel, and non-human primate IGF-1R. In another embodiment, the antigen binding protein binds to one or more primate IGF-1R, for example, to human IGF-1R and one or more of cynomologous, marmoset, rhesus, and chimpanzee IGF-1R. In another embodiment, the antigen binding protein binds specifically to human, cynomologous, marmoset, rhesus, or chimpanzee IGF-1R. In another embodiment, the antigen binding protein does not bind to one or more of mouse, rat, guinea pig, hamster, gerbil, cat, rabbit, dog, goat, sheep, cow, horse, camel, and non-human primate IGF-1R. In another embodiment, the antigen binding protein does not bind to a New World monkey species such as a marmoset. In another embodiment, the antigen binding protein does not exhibit specific binding to any naturally occurring protein other than IGF-1R. In another embodiment, the antigen binding protein does not exhibit specific binding to any naturally occurring protein other than mammalian IGF-1R. In another embodiment, the antigen binding protein does not exhibit specific binding to any naturally occurring protein other than primate IGF-1R. In another embodiment, the antigen binding protein does not exhibit specific binding to any naturally occurring protein other than human IGF-1R. In another embodiment, the antigen binding protein specifically binds to mouse, rat, cynomolgus monkey, and human IGF-1R. In another embodiment, the antigen binding protein specifically binds to mouse, rat, cynomolgus monkey, and human IGF-1R with a similar binding affinity. In another embodiment, the antigen binding protein blocks binding of human IGF-1 and IGF-2 with mouse, rat, cynomolgus monkey, and human IGF-1R. In another embodiment, the antigen binding protein blocks binding of human IGF-1 and IGF-2 with mouse, rat, cynomolgus monkey, and human IGF-1R with similar K. In another embodiment, the antigen binding protein blocks binding of human IGF-1 and IGF-2 with mouse, rat, cynomolgus monkey, and human IGF-1R with a K, of between about 0.57 and about 0.61 nM.

One may determine the selectivity of an antigen binding protein for an IGF-1R using methods well known in the art and following the teachings of the specification. For example, one may determine the selectivity using Western blot, FACS, ELISA or RIA.

In another aspect, the present invention provides an IGF-1R binding antigen binding protein (for example, an anti-IGF-1R antibody), that has one or more of the following characteristics: binds to both human and murine IGF-1R, inhibits the binding of both IGF-1 and IGF-2 to human IGF-1R, inhibits the binding of both IGF-1 and IGF-2 to murine IGF-1R, preferentially inhibits the high affinity binding of IGF-1 and/or of IGF-2 to IGF-1R, binds to the L2 domain of IGF-1R, causes relatively little down-regulation of cell-surface expressed IGF-1R after 17 hours of exposure (as compared to MAB391 (R&D systems, Minneapolis, Minn.); e.g., amount of IGF-1R is reduced by less than 20%), causes a level of down-regulation of cell-surface expressed IGF-1R on Colo-205 or MiaPaCa-2 xenograft tumor cells in mice as MAB391 after four weeks of once weekly doses of 200 micrograms.

Antigen-binding fragments of antigen binding proteins of the invention may be produced by conventional techniques. Examples of such fragments include, but are not limited to, Fab and F(ab′)₂ fragments. Antibody fragments and derivatives produced by genetic engineering techniques also are contemplated.

Additional embodiments include chimeric antibodies, e.g., humanized versions of non-human (e.g., murine) monoclonal antibodies. Such humanized antibodies may be prepared by known techniques, and offer the advantage of reduced immunogenicity when the antibodies are administered to humans. In one embodiment, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of chimeric and further engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. patent application Ser. No. 10/194,975 (published Feb. 27, 2003), U.S. Pat. Nos. 5,869,619, 5,225,539, 5,821,337, 5,859,205, Padlan et al., 1995, FASEB J. 9:133-39, and Tamura et al., 2000, J. Immunol. 164:1432-41.

Procedures have been developed for generating human or partially human antibodies in non-human animals. For example, mice in which one or more endogenous immunoglobulin genes have been inactivated by various means have been prepared. Human immunoglobulin genes have been introduced into the mice to replace the inactivated mouse genes. Antibodies produced in the animal incorporate human immunoglobulin polypeptide chains encoded by the human genetic material introduced into the animal. In one embodiment, a non-human animal, such as a transgenic mouse, is immunized with an IGF-1R polypeptide, such that antibodies directed against the IGF-1R polypeptide are generated in the animal. One example of a suitable immunogen is a soluble human IGF-1R, such as a polypeptide comprising the extracellular domain of the protein of FIG. 10, or other immunogenic fragment of the protein of FIG. 10. Examples of techniques for production and use of transgenic animals for the production of human or partially human antibodies are described in U.S. Pat. Nos. 5,814,318, 5,569,825, and 5,545,806, Davis et al., 2003, Production of human antibodies from transgenic mice in Lo, ed. Antibody Engineering: Methods and Protocols, Humana Press, NJ:191-200, Kellermann et al., 2002, Curr Opin Biotechnol. 13:593-97, Russel et al., 2000, Infect Immun. 68:1820-26, Gallo et al., 2000, Eur J. Immun. 30:534-40, Davis et al., 1999, Cancer Metastasis Rev. 18:421-25, Green, 1999, J Immunol Methods. 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs. 7:607-14, Tsuda et al., 1997, Genomics. 42:413-21, Mendez et al., 1997, Nat. Genet. 15:146-56, Jakobovits, 1994, Curr Biol. 4:761-63, Arbones et al., 1994, Immunity. 1:247-60, Green et al., 1994, Nat. Genet. 7:13-21, Jakobovits et al., 1993, Nature. 362:255-58, Jakobovits et al., 1993, Proc Natl Acad Sci USA. 90:2551-55. Chen, J., M. Trounstine, F. W. Alt, F. Young, C. Kurahara, J. Loring, D. Huszar. “Immunoglobulin gene rearrangement in B cell deficient mice generated by targeted deletion of the JH locus.” International Immunology 5 (1993): 647-656, Choi et al., 1993, Nature Genetics 4: 117-23, Fishwild et al., 1996, Nature Biotechnology 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Internal Review of Immunology 13: 65-93, Neuberger, 1996, Nature Biotechnology 14: 826, Taylor et al., 1992, Nucleic Acids Research 20: 6287-95, Taylor et al., 1994, International Immunology 6: 579-91, Tomizuka et al., 1997, Nature Genetics 16: 133-43, Tomizuka et al., 2000, Proceedings of the National Academy of Sciences USA 97: 722-27, Tuaillon et al., 1993, Proceedings of the National Academy of Sciences USA 90: 3720-24, and Tuaillon et al., 1994, Journal of Immunology 152: 2912-20.

In another aspect, the present invention provides monoclonal antibodies that bind to IGF-1R. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In one embodiment, a hybridoma cell line is produced by immunizing an animal (e.g., a transgenic animal having human immunoglobulin sequences) with an IGF-1R immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds an IGF-1R polypeptide. Such hybridoma cell lines, and anti-IGF-1R monoclonal antibodies produced by them, are encompassed by the present invention.

Monoclonal antibodies secreted by a hybridoma cell line can be purified using any technique known in the art. Hybridomas or mAbs may be further screened to identify mAbs with particular properties, such as the ability to block an IGF-1 and/or IGF-2 induced activity. Examples of such screens are provided in the examples below.

Molecular evolution of the complementarity determining regions (CDRs) in the center of the antibody binding site also has been used to isolate antibodies with increased affinity, for example, antibodies having increased affinity for c-erbB-2, as described by Schier et al., 1996, J. Mol. Biol. 263:551. Accordingly, such techniques are useful in preparing antibodies to IGF-1R.

Antigen binding proteins directed against an IGF-1R can be used, for example, in assays to detect the presence of IGF-1R polypeptides, either in vitro or in vivo. The antigen binding proteins also may be employed in purifying IGF-1R proteins by immunoaffinity chromatography. Those antigen binding proteins that additionally can block binding of IGF-1 and/or IGF-2 to IGF-1R may be used to inhibit a biological activity that results from such binding. Blocking antigen binding proteins can be used in the methods of the present invention. Such antigen binding proteins that function as IGF-1 and/or IGF-2 antagonists may be employed in treating any IGF-1 and/or IGF-2-induced condition, including but not limited to cancer. In one embodiment, a human anti-IGF-1R monoclonal antibody generated by procedures involving immunization of transgenic mice is employed in treating such conditions.

Antigen binding proteins may be employed in an in vitro procedure, or administered in vivo to inhibit an IGF-1 and/or IGF-2-induced biological activity. Disorders caused or exacerbated (directly or indirectly) by the interaction of IGF-1 and/or IGF-2 with cell surface IGF-1R, examples of which are provided above, thus may be treated. In one embodiment, the present invention provides a therapeutic method comprising in vivo administration of an IGF-1 and/or IGF-2 blocking antigen binding protein to a mammal in need thereof in an amount effective for reducing an IGF-1 and/or IGF-2-induced biological activity.

Antigen binding proteins of the invention include partially human and fully human monoclonal antibodies that inhibit a biological activity of IGF-1 and also inhibita biological activity of IGF-2. One embodiment is directed to a human monoclonal antibody that at least partially blocks binding of IGF-1 and of IGF-2 to a cell that expresses human IGF-1R. In one embodiment, the antibodies are generated by immunizing a transgenic mouse with an IGF-1R immunogen. In another embodiment, the immunogen is a human IGF-1R polypeptide (e.g., a soluble fragment comprising all or part of the IGF-1R extracellular domain). Hybridoma cell lines derived from such immunized mice, wherein the hybridoma secretes a monoclonal antibody that binds IGF-1R, also are provided herein.

Although human, partially human, or humanized antibodies will be suitable for many applications, particularly those involving administration of the antibody to a human subject, other types of antigen binding proteins will be suitable for certain applications. The non-human antibodies of the invention can be, for example, derived from any antibody-producing animal, such as mouse, rat, rabbit, goat, donkey, or non-human primate (such as monkey (e.g., cynomologous or rhesus monkey) or ape (e.g., chimpanzee)). Non-human antibodies of the invention can be used, for example, in in vitro and cell-culture based applications, or any other application where an immune response to the antibody of the invention does not occur, is insignificant, can be prevented, is not a concern, or is desired. In one embodiment, a non-human antibody of the invention is administered to a non-human subject. In another embodiment, the non-human antibody does not elicit an immune response in the non-human subject. In another embodiment, the non-human antibody is from the same species as the non-human subject, e.g., a mouse antibody of the invention is administered to a mouse. An antibody from a particular species can be made by, for example, immunizing an animal of that species with the desired immunogen (e.g., a soluble IGF-1R polypeptide) or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

Antigen binding proteins may be prepared by any of a number of conventional techniques. For example, they may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Any expression system known in the art can be used to make the recombinant polypeptides of the invention. In general, host cells are transformed with a recombinant expression vector that comprises DNA encoding a desired polypeptide. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, HeLa cells, BHK (ATCC CRL 10) cell lines, and the CV1/EBNA cell line derived from the African green monkey kidney cell line CV1 (ATCC CCL 70) as described by McMahan et al., 1991, EMBO J. 10: 2821. Appropriate cloning and expression vectors for use with bacterial, fungal, yeast, and mammalian cellular hosts are described by Pouwels et al. (Cloning Vectors: A Laboratory Manual, Elsevier, New York, 1985).

The transformed cells can be cultured under conditions that promote expression of the polypeptide, and the polypeptide recovered by conventional protein purification procedures. One such purification procedure includes the use of affinity chromatography, e.g., over a matrix having all or a portion (e.g., the extracellular domain) of IGF-1R bound thereto. Polypeptides contemplated for use herein include substantially homogeneous recombinant mammalian anti-IGF-1R antibody polypeptides substantially free of contaminating endogenous materials.

Antigen binding proteins may be prepared, and screened for desired properties, by any of a number of known techniques. Certain of the techniques involve isolating a nucleic acid encoding a polypeptide chain (or portion thereof) of an antigen binding protein of interest (e.g., an anti-IGF-1R antibody), and manipulating the nucleic acid through recombinant DNA technology. The nucleic acid may be fused to another nucleic acid of interest, or altered (e.g., by mutagenesis or other conventional techniques) to add, delete, or substitute one or more amino acid residues, for example.

In one aspect, the present invention provides antigen-binding fragments of an anti-IGF-1R antibody of the invention. Such fragments can consist entirely of antibody-derived sequences or can comprise additional sequences. Examples of antigen-binding fragments include Fab, F(ab′)2, single chain antibodies, diabodies, triabodies, tetrabodies, and domain antibodies. Other examples are provided in Lunde et al., 2002, Biochem. Soc. Trans. 30:500-06.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Korff et al., 1997, Prot. Eng. 10:423; Kortt et al., 2001, Biomol. Eng. 18:95-108). By combining different V_{L} V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87. Single chain antibodies derived from antibodies provided herein include, but are not limited to, scFvs comprising the variable domain combinations L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52) are encompassed by the present invention.

Antigen binding proteins (e.g., antibodies, antibody fragments, and antibody derivatives) of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

In one embodiment, an antigen binding protein of the invention comprises the IgG1 heavy chain domain of FIG. 13 or a fragment of the IgG1 heavy chain domain of FIG. 13. In another embodiment, an antigen binding protein of the invention comprises the kappa light chain constant chain region of FIG. 13 or a fragment of the kappa light chain constant region of FIG. 13. In another embodiment, an antigen binding protein of the invention comprises both the IgG 1 heavy chain domain, or a fragment thereof, of FIG. 13 and the kappa light chain domain, or a fragment thereof, of FIG. 13.

Accordingly, the antigen binding proteins of the present invention include those comprising, for example, the variable domain combinations L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52, having a desired isotype (for example, IgA, IgG1, IgG2, IgG3, IgG4, IgM, IgE, and IgD) as well as Fab or F(ab′)₂ fragments thereof. Moreover, if an IgG4 is desired, it may also be desired to introduce a point mutation (CPSCP->CPPCP) in the hinge region as described in Bloom et al., 1997, Protein Science 6:407, incorporated by reference herein) to alleviate a tendency to form intra-H chain disulfide bonds that can lead to heterogeneity in the IgG4 antibodies.

An example of an antigen binding protein of the invention is the IgG1 antibody AMG 479, also known as ganitumab. Ganitumab has the light and heavy chain variable domains of L16H16.

Moreover, techniques for deriving antigen binding proteins having different properties (i.e., varying affinities for the antigen to which they bind) are also known. One such technique, referred to as chain shuffling, involves displaying immunoglobulin variable domain gene repertoires on the surface of filamentous bacteriophage, often referred to as phage display. Chain shuffling has been used to prepare high affinity antibodies to the hapten 2-phenyloxazol-5-one, as described by Marks et al., 1992, BioTechnology, 10:779.

In particular embodiments, antigen binding proteins of the present invention have a binding affinity (Kₐ) for IGF-1R of at least 10⁶, measured as described in the Examples. In other embodiments, the antigen binding proteins exhibit a Kₐ of at least 10⁷, at least 10⁸, at least 10⁹, or at least 10¹⁰.

In another embodiment, the present invention provides an antigen binding protein that has a low dissociation rate from IGF-1R. In one embodiment, the antigen binding protein has a K_{off} of 1×10⁻⁴ s⁻¹ or lower. In another embodiment, the K_{off} is 5×10⁻⁵ s⁻¹ or lower. In another embodiment, the K_{off} is substantially the same as an antibody having a combination of light chain and heavy chain variable domain sequences selected from the group of combinations consisting of L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52. In another embodiment, the antigen binding protein binds to IGF-1R with substantially the same K_{off} as an antibody that comprises one or more CDRs from an antibody having a combination of light chain and heavy chain variable domain sequences selected from the group of combinations consisting of L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52. In another embodiment, the antigen binding protein binds to IGF-1R with substantially the same K_{off} as an antibody that comprises one of the amino acid sequences illustrated in FIGS. 2 through 9. In another embodiment, the antigen binding protein binds to IGF-1R with substantially the same K_{off} as an antibody that comprises one or more CDRs from an antibody that comprises one of the amino acid sequences illustrated in FIGS. 2 through 9.

In another aspect, the present invention provides an antigen binding protein that binds to the L2 domain of human IGF-1R. Antigen binding proteins that bind to the L2 domain can be made using any technique known in the art. For example, such antigen binding proteins can be isolated using the full-length IGF-1R polypeptide (e.g., in a membrane-bound preparation), a soluble extracellular domain fragment of IGF-1R (an example of which is provided in Example 1), or a smaller fragment of the IGF-1R extracellular domain comprising or consisting of the L2 domain (examples of which are provided in Example 10). Antigen binding proteins so isolated can be screened to determine their binding specificity using any method known in the art (an example of which is provided in Example 10).

In another aspect, the present invention provides an antigen binding protein that binds to human IGF-1R expressed on the surface of a cell and, when so bound, inhibits IGF-1R signaling activity in the cell without causing a significant reduction in the amount of IGF-1R on the surface of the cell. Any method for determining or estimating the amount of IGF-1R on the surface and/or in the interior of the cell can be used. In one embodiment, the present invention provides an antigen binding protein that binds to the L2 domain of a human IGF-1R expressed on the surface of a cell and, when so bound, inhibits. IGF-1R signaling activity in the cell without significantly increasing the rate of internalization of the IGF-1R from the surface of the cell. In other embodiments, binding of the antigen binding protein to the IGF-1R-expressing cell causes less than about 75%, 50%, 40%, 30%, 20%, 15%, 10%, 5%, 1%, or 0.1% of the cell-surface IGF-1R to be internalized. In another aspect, binding of the antigen binding protein to the IGF-1R-expressing cell causes a gradual reduction in the amount of IGF-1R on the cell surface such that within a few hours of contacting the cell with the antigen binding protein, little or no decrease in cell surface IGF-1R is detected, but, after several days or weeks of exposure of the cell to the antigen binding protein, a marked decrease in cell surface IGF-1R is detected.

In another aspect, the present invention provides an antigen binding protein having a half-life of at least one day in vitro or in vivo (e.g., when administered to a human subject). In one embodiment, the antigen binding protein has a half-life of at least three days. In another embodiment, the antigen binding protein has a half-life of four days or longer. In another embodiment, the antigen binding protein has a half-life of eight days or longer. In another embodiment, the antigen binding protein is derivatized or modified such that it has a longer half-life as compared to the underivatized or unmodified antigen binding protein. In another embodiment, the antigen binding protein contains one or more point mutations to increase serum half life, such as described in WO 00/09560, published Feb. 24, 2000, incorporated by reference.

The present invention further provides multi-specific antigen binding proteins, for example, bispecific antigen binding protein, e.g., antigen binding protein that bind to two different epitopes of IGF-1R, or to an epitope of IGF-1R and an epitope of another molecule, via two different antigen binding sites or regions. Moreover, bispecific antigen binding protein as disclosed herein can comprise an IGF-1R binding site from one of the herein-described antibodies and a second IGF-1R binding region from another of the herein-described antibodies, including those described herein by reference to other publications. Alternatively, a bispecific antigen binding protein may comprise an antigen binding site from one of the herein described antibodies and a second antigen binding site from another IGF-1R antibody that is known in the art, or from an antibody that is prepared by known methods or the methods described herein.

Numerous methods of preparing bispecific antibodies are known in the art, and discussed in U.S. patent application Ser. No. 09/839,632, filed Apr. 20, 2001 (incorporated by reference herein). Such methods include the use of hybrid-hybridomas as described by Milstein et al., 1983, Nature 305:537, and others (U.S. Pat. No. 4,474,893, U.S. Pat. No. 6,106,833), and chemical coupling of antibody fragments (Brennan et al., 1985, Science 229:81; Glennie et al., 1987, J. Immunol. 139:2367; U.S. Pat. No. 6,010,902). Moreover, bispecific antibodies can be produced via recombinant means, for example by using leucine zipper moieties (i.e., from the Fos and Jun proteins, which preferentially form heterodimers; Kostelny et al., 1992, J. Immunol. 148:1547) or other lock and key interactive domain structures as described in U.S. Pat. No. 5,582,996. Additional useful techniques include those described in Kortt et al., 1997, supra; U.S. Pat. No. 5,959,083; and U.S. Pat. No. 5,807,706.

In another aspect, the antigen binding protein of the present invention comprises a derivative of an antibody. The derivatized antibody can comprise any molecule or substance that imparts a desired property to the antibody, such as increased half-life in a particular use. The derivatized antibody can comprise, for example, a detectable (or labeling) moiety (e.g., a radioactive, colorimetric, antigenic or enzymatic molecule, a detecable bead (such as a magnetic or electrodense (e.g., gold) bead), or a molecule that binds to another molecule (e.g., biotin or streptavidin)), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). Examples of molecules that can be used to derivatize an antibody include albumin (e.g., human serum albumin) and polyethylene glycol (PEG). Albumin-linked and PEGylated derivatives of antibodies can be prepared using techniques well known in the art. In one embodiment, the antibody is conjugated or otherwise linked to transthyretin (TTR) or a TTR variant. The TTR or TTR variant can be chemically modified with, for example, a chemical selected from the group consisting of dextran, poly(n-vinyl pyurrolidone), polyethylene glycols, propropylene glycol homopolymers, polypropylene oxide/ethylene oxide co-polymers, polyoxyethylated polyols and polyvinyl alcohols. US Pat. App. No. 20030195154.

In another aspect, the present invention provides methods of screening for a molecule that binds to IGF-1R using the antigen binding proteins of the present invention. Any suitable screening technique can be used. In one embodiment, an IGF-1R molecule, or a fragment thereof to which an antigen binding protein of the present invention binds, is contacted with the antigen binding protein of the invention and with another molecule, wherein the other molecule binds to IGF-1R if it reduces the binding of the antigen binding protein to IGF-1R. Binding of the antigen binding protein can be detected using any suitable method, e.g., an ELISA. Detection of binding of the antigen binding protein to IGF-1R can be simplified by detectably labeling the antigen binding protein, as discussed above. In another embodiment, the IGF-1R-binding molecule is further analyzed to determine whether it inhibits IGF-1R, IGF-1, and/or IGF-2-mediated signaling.

### Nucleic Acids

In one aspect, the present invention provides isolated nucleic acid molecules. The nucleic acids comprise, for example, polynucleotides that encode all or part of an antigen binding protein, for example, one or both chains of an antibody of the invention, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector. The nucleic acids can be single-stranded or double-stranded and can comprise RNA and/or DNA nucleotides, and artificial variants thereof (e.g., peptide nucleic acids).

Nucleic acids encoding antibody polypeptides (e.g., heavy or light chain, variable domain only, or full length) may be isolated from B-cells of mice that have been immunized with IGF-1R. The nucleic acid may be isolated by conventional procedures such as polymerase chain reaction (PCR).

FIG. 1 provides nucleic acid sequences encoding the variable regions of the heavy and light chain variable regions shown in FIGS. 2 and 3. The skilled artisan will appreciate that, due to the degeneracy of the genetic code, each of the polypeptide sequences in FIGS. 2 through 9 also is encoded by a large number of other nucleic acid sequences. The present invention provides each degenerate nucleotide sequence encoding each antigen binding protein of the invention.

The invention further provides nucleic acids that hybridize to other nucleic acids (e.g., nucleic acids comprising a nucleotide sequence of FIG. 1) under particular hybridization conditions. Methods for hybridizing nucleic acids are well-known in the art. See, e.g., Current Protocols in Molecular Biology, John Wiley & Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98 or 99% identical to each other typically remain hybridized to each other. The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA.

Changes can be introduced by mutation into a nucleic acid, thereby leading to changes in the amino acid sequence of a polypeptide (e.g., an antigen binding protein) that it encodes. Mutations can be introduced using any technique known in the art. In one embodiment, one or more particular amino acid residues are changed using, for example, a site-directed mutagenesis protocol. In another embodiment, one or more randomly selected residues is changed using, for example, a random mutagenesis protocol. However it is made, a mutant polypeptide can be expressed and screened for a desired property (e.g., binding to IGF-1R or blocking the binding of IGF-1 and/or IGF-2 to IGF-1R).

Mutations can be introduced into a nucleic acid without significantly altering the biological activity of a polypeptide that it encodes. For example, one can make nucleotide substitutions leading to amino acid substitutions at non-essential amino acid residues. In one embodiment, a nucleotide sequence provided in FIG. 1, or a desired fragment, variant, or derivative thereof, is mutated such that it encodes an amino acid sequence comprising one or more deletions or substitutions of amino acid residues that are shown in FIGS. 2 through 9 to be residues where two or more sequences differ. In another embodiment, the mutagenesis inserts an amino acid adjacent to one or more amino acid residues shown in FIGS. 2 through 9 to be residues where two or more sequences differ. Alternatively, one or more mutations can be introduced into a nucleic acid that selectively change the biological activity (e.g., binding of IGF-1R, inhibiting IGF-1 and/or IGF-2, etc.) of a polypeptide that it encodes. For example, the mutation can quantitatively or qualitatively change the biological activity. Examples of quantitative changes include increasing, reducing or eliminating the activity. Examples of qualitative changes include changing the antigen specificity of an antigen binding protein.

In another aspect, the present invention provides nucleic acid molecules that are suitable for use as primers or hybridization probes for the detection of nucleic acid sequences of the invention. A nucleic acid molecule of the invention can comprise only a portion of a nucleic acid sequence encoding a full-length polypeptide of the invention, for example, a fragment that can be used as a probe or primer or a fragment encoding an active portion (e.g., an IGF-1R binding portion) of a polypeptide of the invention.

Probes based on the sequence of a nucleic acid of the invention can be used to detect the nucleic acid or similar nucleic acids, for example, transcripts encoding a polypeptide of the invention. The probe can comprise a label group, e.g., a radioisotope, a fluorescent compound, an enzyme, or an enzyme co-factor. Such probes can be used to identify a cell that expresses the polypeptide.

In another aspect, the present invention provides vectors comprising a nucleic acid encoding a polypeptide of the invention or a portion thereof. Examples of vectors include, but are not limited to, plasmids, viral vectors, non-episomal mammalian vectors and expression vectors, for example, recombinant expression vectors.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression; which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

In another aspect, the present invention provides host cells into which a recombinant expression vector of the invention has been introduced. A host cell can be any prokaryotic cell (for example, E. coli) or eukaryotic cell (for example, yeast, insect, or mammalian cells (e.g., CHO cells)). Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

### Indications

In one aspect, the present invention provides methods of treating a subject. The method can, for example, have a generally salubrious effect on the subject, e.g., it can increase the subject's expected longevity. Alternatively, the method can, for example, treat, prevent, cure, relieve, or ameliorate (“treat”) a disease, disorder, condition, or illness (“a condition”). Among the conditions to be treated in accordance with the present invention are conditions characterized by inappropriate expression or activity of IGF-1, IGF-2, and/or IGF-1R. In some such conditions, the expression or activity level is too high, and the treatment comprises administering an IGF-1R antagonist as described herein. In other such conditions, the expression or activity level is too low, and the treatment comprises administering an IGF-1R agonist as described herein.

One example of a type of condition that can be treated using the methods and compositions of the present invention is a condition that involves cell growth, for example, a cancerous condition. Thus, in one embodiment, the present invention provides compositions and methods for treating a cancerous condition. The cancerous condition can be any cancerous condition that can be treated using the compositions comprised herein, for example, IGF-1R antagonizing antigen binding proteins such as anti-IGF-1R antibodies, antibody fragments, or antibody derivatives. Examples of cancerous conditions include, for example, Acute Lymphoblastic Leukemia, Adrenocortical Carcinoma, AIDS-Related Cancers, AIDS-Related Lymphoma, Anal Cancer, Childhood Cerebellar Astrocytoma, Childhood Cerebral Astrocytoma, Basal Cell Carcinoma, Extrahepatic Bile Duct Cancer, Bladder Cancer, Osteosarcoma/Malignant Fibrous Histiocytoma Bone Cancer, Brain Tumors (e.g., Brain Stem Glioma, Cerebellar Astrocytoma, Cerebral Astrocytoma/Malignant Glioma, Ependymoma, Medulloblastoma, Supratentorial Primitive Neuroectodermal Tumors, Visual Pathway and Hypothalamic Glioma), Breast Cancer, Bronchial Adenomas/Carcinoids, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, Childhood Cancers, Chronic Lymphocytic Leukemia, Chronic Myelogenous Leukemia, Chronic Myeloproliferative Disorders, Colon Cancer, Colorectal Cancer, Cutaneous T-Cell Lymphoma, Endometrial Cancer, Ependymoma, Esophageal Cancer, Ewing's Family of Tumors, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, Germ Cell Tumors (e.g., Extracranial, Extragonadal, and Ovarian), Gestational Trophoblastic Tumor, Glioma (e.g., Adult, Childhood Brain Stem, Childhood Cerebral Astrocytoma, Childhood Visual Pathway and Hypothalamic), Hairy Cell Leukemia, Head and Neck Cancer, Hepatocellular (Liver) Cancer, Hodgkin's Lymphoma, Hypopharyngeal Cancer, Hypothalamic and Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocytic, Chronic Myelogenous, and Hairy Cell), Lip and Oral Cavity Cancer, Liver Cancer, Non-Small Cell Lung Cancer, Small Cell Lung Cancer, Lymphoma (e.g., AIDS-Related, Burkitt's, Cutaneous T-Cell, Hodgkin's, Non-Hodgkin's, and Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic Myeloid Leukemia, Multiple Myeloma, Chronic Myeloproliferative Disorders, Nasal Cavity and Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus and Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis and Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-Cell Lymphoma, Testicular Cancer, Thymoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway and Hypothalamic Glioma, Vulvar Cancer, Waldenstrom's Macroglobulinemia, and Wilms' Tumor.

Four different groups have studied a total of 425 breast cancers, mostly ductal in origin, and 48 normal tissues or benign specimens by radioimmunoassay (“RIA”) or immunohistochemistry (“IHC”) (Papa et al., 1993, Cancer Research 53: 3736-40, Happerfield et al., 1997, Journal of Pathology 183: 412-17; Ellis et al., 1998, Breast Cancer Research & Treatment 52: 175-84, Lee et al., 1998, Breast Cancer Research & Treatment 47: 295-302, Schnarr et al., 2000, International Journal of Cancer 89: 506-13). These studies suggest that elevated IGF-1R expression, on the order of 5-10 fold, is associated with favorable prognosis and biomarkers (ER+ PR+), suggesting that estrogen and IGF cooperate in the maintenance or progression of well differentiated tumor. Similarly, estrogen has been shown to be essential for the growth and survival of the ER+ MCF-7 breast cancer cell line, and in this context IGF-1R is up-regulated by estrogen treatment (reviewed in Ellis et al., 1998, Breast Cancer Research & Treatment 52: 175-84). Thus, in one embodiment, the present invention provides a method of treating breast cancer in a subject in need of such treatment, comprising administering to the subject an effective amount of an IGF-1R antagonist as described herein. In another embodiment, the method further comprises administering a hormone inhibitor, e.g., an estrogen inhibitor.

A retrospective IGF-1R IHC analysis has been reported for a collection of 12 colonic adenomas, 36 primary colorectal adenocarcinomas and 27 corresponding metastases, and 34 adjacent normal tissues (Hakam et al., 1999, Human Pathology. 30: 1128-33). The frequency of moderate to strong IHC staining appeared to dramatically increase with higher stage and tumor grade (0% normal vs. 93% metastases). The results are consistent with RNA analysis by RNAse protection assay (“RPA”) (Freier et al., 1999, Gut 44: 704-08). Thus, in one embodiment, the present invention provides a method of treating colon cancer in a subject in need of such treatment, comprising administering to the subject an effective amount of an IGF-1R antagonist as described herein.

High plasma IGF-1 and reduced IGFbp3 in men 40-80 years old is associated with increased prostate cancer risk (Chan et al., 1998, Science 279: 563-6). High IGF-1 is associated with a risk of other cancers including breast (Hankinson et al., 1998, Lancet 351: 1393-96), colon (Ma et al., 1999, Journal of the National Cancer Institute 91: 620-25) and lung (Yu et al., 1999, Journal of the National Cancer Institute 91: 151-56). In transgenic mouse models, tumor incidence is increased by IGF-1 overexpression in diverse locations (Bol et al., 1997, Oncogene 14: 1725-34; DiGiovanni et al., 2000, Cancer Research 60: 1561-70; DiGiovanni et al., 2000, Proceedings of the National Academy of Sciences of the United States of America 97: 3455-60, Hadsell et al., 2000, Oncogene 19: 889-98). These mouse studies point to a role for both serum and stromal produced IGF-1. Thus, in one embodiment, the present invention provides a method of treating a subject in need of such treatment, comprising administering to the subject an effective amount of an antagonist of IGF-1R as described herein, wherein the antagonist inhibits the activation of IGF-1R by IGF-1. In another embodiment, the subject has cancer. In another embodiment, the subject has a tumor. In another embodiment, the cancer is prostate, breast, colon or lung cancer.

It has been observed that bone is the major source of IGF-1 in the body. Thus, in one aspect, the present invention provides compositions and methods for inhibiting IGF-1R in a bone of a subject. In one embodiment, an IGF-1R inhibitor of the present invention is administered to a subject that has, or is at risk for developing, a tumor in a bone. The tumor can be, for example, a primary tumor or a metastatic tumor. The treatment optionally further comprises administering to the subject one or more additional therapeutic and/or palliative treatments, for example, an anti-tumor treatment (e.g., chemotherapy, radiation therapy, or anti-hormone therapy) or a treatment that inhibits bone turnover (e.g., denosumab (Amgen Inc., Thousand Oaks, Calif.)).

IGF-2 is overexpressed in a variety of tumors and stromal tissues. IGF-2 levels appear especially high (as much as 40 fold) in primary liver cancers (Cariani et al., 1988, Cancer Research 48: 6844-49) and adenocarcinoma of the colon (Freier et al., 1999, Gut 44: 704-08). Many of the overgrowth disorders are associated with an increased incidence of childhood tumors. Five to ten percent of individuals with either the prenatal growth disorder Beckwith-Weidmann Syndrome (BWS) or hemihyperplasia develop tumors such as nephroblastoma, adrenal carcinoma, and neuroblastoma (reviewed by Morison et al., 1998, Molecular Medicine Today 4: 110-05). The tumor-predisposing factor in these children appears to be the mosaic loss of maternal IGF-2 gene imprinting, or duplication of the paternal chromosomal arm (11p) that carries IGF-2. Both alterations would increase the level of IGF-2 expression. IGF-2 overexpression as a result of mosaic uniparental disomy or loss of IGF-2 imprinting has also been detected in Wilms tumors. Growth disorders are not observed in these children even though the IGF-2 gene alterations also occur in some normal tissues, perhaps reflecting the tissue distribution of the affected cells. Imprinting of the maternal IGF-2 gene also occurs in mice, and the effects of IGF-2 overexpression are consistent with the human situation (Cariani et al., 1991, Journal of Hepatology 13: 220-26, Schirmacher et al., 1992, Cancer Research 52: 2549-56; Harris et al., 1998, Oncogene 16: 203-09). The incidence of tumors and organomegaly increases in mice that transgenically express excess IGF-2 (Christofori et al., 1994, Nature 369: 414-18, Ward et al., 1994, Proceedings of the National Academy of Sciences of the United States of America 91: 10365-9, Wolf et al., 1994, Endocrinology 135: 1877-86, Bates et al., 1995, British Journal of Cancer 72: 1189-93, Hassan et al., 2000, Cancer Research 60: 1070-76). Local IGF-2 overexpression increases the spontaneous appearance of prostate, mammary, intestinal, liver and epidermal tumors. Plasma specific expression using liver promoters elevate hepatocellular carcinomas and lymphoma. Thus, in one embodiment, the present invention provides a method of treating a subject in need of such treatment, comprising administering to the subject an effective amount of an antagonist of IGF-1R as described herein, wherein the antagonist inhibits the activation of IGF-1R by IGF-2. In another embodiment, the subject has cancer. In another embodiment, the subject has a tumor. In another embodiment, the subject has liver cancer, adenocarcinoma of the colon, Beckwith-Weidmann Syndrome, hemihyperplasia, nephroblastoma, adrenal carcinoma, neuroblastoma, mosaic loss of maternal IGF-2 gene imprinting, duplication of the paternal chromosomal arm (11p), increased IGF-2 expression, a tumor (e.g., a prostate, mammary, intestinal, liver, epidermal, or Wilms tumor), organomegaly, hepatocellular carcinoma, or lymphoma.

In another aspect, the invention provides methods of preventing or inhibiting a cancer from spreading to another part of the body, or of treating a cancer that has spread to another part of the body. In one embodiment, the cancer has spread to a regional lymph node. In another embodiment, the cancer is metastatic. The primary tumor can be any kind of tumor, for example, an adenocarcinoma tumor (e.g., a prostate adenocarcinoma tumor, a breast carcinoma tumor, or a renal cell carcinoma tumor), a non-small cell or small cell lung cancer tumor, a thyroid cancer tumor, etc. The site of the metastatic tumor can be anywhere in the body. It can be, for example, in bone, the lymph system, lung, brain, eye, skin, pancrease, or liver. In one particular embodiment, a subject having a tumor disease is treated with an effective amount of an IGF-1R inhibiting composition of the present invention such that the primary tumor is prevented from metastasizing. In another particular embodiment, a subject having a primary tumor is treated with an effective amount of an IGF-1R inhibiting composition of the present invention such that the primary tumor is inhibited from metastasizing. In another particular embodiment, a subject having a metastatic tumor is treated with an effective amount of an IGF-1R inhibiting composition of the present invention such that growth or spreading of the secondary tumor is inhibited. In another particular embodiment, a subject having a metastatic tumor is treated with an effective amount of an IGF-1R inhibiting composition of the present invention such that the secondary tumor is reduced in size. In a more particular embodiment, the primary tumor is an adenocarcinoma tumor, a non-small cell lung tumor, a small cell lung tumor, or a thyroid cancer. In another more particular embodiment, the metastatic tumor is in a bone. In another more particular embodiment, a metastatic tumor is prevented or inhibited from forming in a bone. In another more particularly defined embodiment, the method comprises treating the subject with an IGF-1R inhibiting composition of the present invention and one or more other treatments (e.g., a treatment that kills or inhibits the growth of cancer cells, such as radiation, hormonal therapy, or chemotherapy, or a treatment that inhibits the turnover of bone, such as denosumab), non-limiting examples of which are provided herein. The one or more other treatments can include, for example the standard of care for the subject's particular condition and/or palliative care.

Without being bound to any particular theory, tumor cells appear to depend on the PI3 Kinase/Akt signaling pathway to resist the apoptosis-inducing activity of chemotherapeutics, radiation, and anti-hormone therapy. Thus, in one embodiment, the present invention provides methods of treating a subject in need of such treatment comprising administering to the subject an IGF-1R antagonist of the present invention and a chemotherapeutic, radiation, and/or an anti-hormone therapy. This concept has been validated experimentally in cell culture models and rodent tumor models by antisense and dominant negative mutations (reviewed by Baserga et al., 1997, Biochimica et Biophysica Acta 1332: F105-26, Baserga, 2000, Oncogene 19: 5574-81). In one embodiment, the chemotherapeutic agents is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti-angiogenesis agents.

One example of a chemotherapeutic agent that can be administered in combination with an IGF-1 receptor inhibitor of the invention is CPT-11. CPT-11 (Irinotecan hydrorchloride trihydrate) is a semi synthetic, water soluble derivative of camptothecin, a plant alkaloid. CPT-11 and an associated metabolite called SN38 inhibit topoisomerase 1 (TOPO1). This enzyme introduces reversible single-strand breaks in DNA that allow unwinding and permit DNA replication to proceed. Inhibition of TOPO1 prevents religation of single-strand breaks after DNA replication resulting in greatly increased chromosomal fragmentation. This DNA damage promotes cell death by apoptosis through the action of p53 and other systems that monitor genome integrity; The cytotoxic effect of CPT-11 is generally limited to cells that are replicating DNA (S-Phase). Quiescent cells are largely unaffected.

In another embodiment, the present invention provides treating a subject in need thereof with an effective amount of an IGF-1R antagonist of the present invention and with an effective amount of an apoptosis-inducing agent.

In another embodiment, an anti-angiogenesis agent, such as an MMP-2 (matrix-metalloproteinase 2) inhibitor, an MMP-9 (matrix-metalloproteinase 9) inhibitor, and/or a COX-II (cyclooxygenase II) inhibitor, is used in conjunction with a compound of the invention. Examples of useful COX-II inhibitors include CELEBREX™ (alecoxib), BEXTRA™ (valdecoxib), and VIOXX™ (rofecoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. In one embodiment, the MMP inhibitor is one that does not demonstrate arthralgia. In another embodiment, the MMP inhibitor selectively inhibits MMP-2 and/or MMP-9 relative to other matrix-metalloproteinases (i.e., MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzene-sulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]-octane-3-carboxylic acid hydroxyamide; (2R, 3R) 1-[4-(2-chloro-4-fluoro-ben-zyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-py-ran-4-carboxylic acid hydroxyamide; 3-[[4-(4-fluoro-phenoxy)-benzenesulfon-yl]-(1-hydroxycarbamoyl-cyclobutyl)-amino]-propionic acid; 4-[4-(4-chloro-phenoxy)-benzenesulfonylamino]-tetrahydro-pyran-4-carboxyl-ic acid hydroxyamide; (R) 3-[4-(4-chloro-phenoxy)-benzenesulfonylamino]-te-trahydro-pyran-3-carboxylic acid hydroxyamide; (2R,3R) 1-[4-(4-fluoro-2-methyl-benzyloxy)-benzenesulfonyl]-3-hydroxy-3-methyl-pi-peridine-2-carboxylic acid hydroxyamide; 3-[[4-(4-fluoro-phenoxy)-benzeneslfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesu-lfonylamino]-8-oxa-icyclo[3.2.1]octane-3-carboxylic acid hydroxyamide; 3-endo-3-[4-(4-fluoro-phenoxy)-benzenesulfonylamino]-8-oxa-icyclo[3.2.1]octane-3-carboxylic acid hydroxyamide; and (R) 3-[4-(4-fluoro-phenoxy)-b-enzenesulfonylamino]-tetrahydro-furan-3-carboxylic acid hydroxyamide; and pharmaceutically acceptable salts, solvates, derivatives, and other preparations of the compounds.

Sporadic mutations that inactivate the PETN gene product occur relatively frequently in most human cancers (Yamada et al., 2001, J Cell Sci 114:2375-82, Hill et al., 2002, Pharmacol Therapeut 93:243-51). Loss of PTEN causes the Akt phosphorylated state to persist through loss of the ability to down-regulate stimulatory signals originating from IGF-1R and other sources. The status of the p53 tumor suppressor also influences the activity of the IGF-1R signaling system. In the ground state, the basal or constitutive transcription of IGF-1R is repressed by p53 via an indirect mechanism. Activation of Akt promotes the phosphorylation of mdm2, which then binds the p53 tumor suppressor and promotes its degradation (Mayo et al., 2002, TIBS 27:462-67), resulting in increased IGF-1R expression. A similar outcome is observed when p53 is inactivated by mutation. When transiently expressed in Saos-2 (a human osteosarcoma cell line) and RD (a rhabdomyosarcoma cell line), wild-type p53 is able to suppress the activity of a cotransfected IGF-1R promoter construct, whereas tumor-derived, mutant versions of p53 have no effect. It has been proposed that the increased level of IGF-1R promotes the resistance to apoptosis associated with p53 loss in malignant cells (Werner et al., 2000, Cell Mol Life Sci 57:932-42). Thus, in one embodiment, the present invention provides a method of treating a cancerous condition in a subject in need of such treatment comprising administering to the subject an effective amount of an IGF-1R antagonist as described herein, wherein the cancerous condition is characterized by cells that have a reduced expression or activity of p53.

The WT1 (Wilms kidney tumor suppressor 1 protein) also has been shown to bind and repress the IGF-1R promoter. Thus, in one embodiment, the present invention provides a method of treating a cancerous condition in a subject in need of such treatment comprising administering to the subject an effective amount of an IGF-1R antagonist as described herein wherein the cancerous condition is characterized by a reduced expression or activity of WT1.

The proliferation of normal fibroblasts has been shown to require, under defined culture conditions, the combined action of IGF and a stromal growth factor (e.g. PDGF, EGF) to ramp-up Ras/Raf/Map Kinase and promote cell cycle entry (the G0 to G1 transition). Fibroblasts derived from IGF-1R (−/−) mice do not respond to growth factor alone, or most oncogenes (e.g. oncogenic Ras) that activate the Ras/Raf/Map Kinase pathway. Thus, in one embodiment, the present invention provides a method of treating a subject in need of such treatment comprising administering to the subject an IGF-1R antagonist as described herein and an agent that targets a growth factor and/or a growth factor receptor, such as a growth factor receptor tyrosine kinase, e.g., the EGFR, HER-2, bcr-abl, VEGFR, Kit, raf, mTOR, CDK1/2, VEGFR2, PKCP, Mek, and/or KDR. Examples of molecules that target such growth factors and/or receptors include panitumumab (Abgenix, Fremont, Calif./Amgen, Thousand Oaks, Calif.), HERCEPTIN™ (Genentech, South San Francisco, Calif.), GLEEVEC™ (Novartis, East Hanover, N.J.), IRESSA™ (AstraZeneca, Wilmington, Del.), ERBITUX™, (ImClone, New York, N.Y.), AVASTIN™, (Genentech), PTK787 (Novartis), SU11248 (Pfizer, New York, N.Y.), TARCEVA™ (OSI Pharmaceuticals, Melville, N.Y.), 43-9006 (Bayer, West Haven, Conn.), CCI-779 (Wyeth, Madison, N.J.), RAD001 (Novartis), BMS-387032 (Bristol-Myers Squibb, New York, N.Y.), IMC-1C11 (ImClone), LY333531 (Eli Lilly, Indianapolis, Ind.), PD 184352 (Pfizer), 2C4 (Genentech), and GW2016 (GlaxoSmithKline, Research Triangle Park, N.C.).

The role of IGF-1R in hematological malignancies has been reviewed by (Novak et al., 2003, Insulin-Like Growth Factors and Hematological Malignancies in Insulin-Like Growth Factors, LeRoith et al., ed.s, Landes Bioscience). A functional role for the IGF-1R in hematopoietic malignancies is demonstrated by, for example, the ability of IGF-1R monoclonal antibodies to block transformed cell growth in culture. IGF-I has been found to enhance growth of freshly isolated human acute myelogenous leukemia and acute lymphoblastic leukemia blasts. With respect to T cell malignancies, IGF-I has been shown to influence the growth of murine lymphoma cells bearing a pre-T cell phenotype and, immature and mature primary human T lineage acute lymphoblastic leukemia cells were found to express high numbers of IGF-1R. Thus, in one embodiment, the present invention provides methods of treating a hematological malignancy in a subject in need thereof comprising administering to the subject an antagonist of IGF-1R as described herein. In another embodiment, the malignancy is an acute myelogenous leukemia, an acute lymphoblastic leukemia, or a T cell malignancy.

In another aspect, the present invention provides methods of identifying subjects who are more likely to benefit from treatment using the compositions and/or methods of treatment of the present invention. Such methods can enable a caregiver to better tailor a therapeutic regimen to a particular subject's needs and reduce the likelihood of an ineffective or counterproductive course of treatment. In one embodiment, the present invention provides a method of determining whether a subject is a candidate for treatment using a composition or method as described herein comprising determining whether a target cell type in the subject expresses IGF-1R, wherein if the target cell type expresses IGF-1R, then the subject is a candidate for treatment. In another embodiment, the method comprises determining the approximate average number of IGF-1R molecules per target cell, wherein 10², 10³, 10⁴, 10⁵, or 10⁶ IGF-1R per cell indicates that the subject is a candidate for treatment. The approximate average number of IGF-1R molecules per target cell can be determined using any technique known in the art, for example, by staining a sample comprising cells of the target cell type with an IGF-1R binding molecule, and detecting the amount of IGF-1R binding molecule bound to the sample, where the amount of IGF-1R binding molecule detected is proportional to the average number of IGF-1R molecules in the sample. In another embodiment, the method comprises comparing the approximate average number of IGF-1R molecules per target cell to a reference standard, wherein if the approximate average number of IGF-1R molecules per target cell is greater than the reference standard, then the subject is more likely to benefit from treatment using the compositions and/or methods of treatment of the present invention. In another embodiment, the target cell type is a cancerous cell type. In another embodiment, the target cell type is a colon cancer cell type, a breast cancer cell type, an NSCLC cell type, or a leukemic cell type.

In another embodiment, a subject who is a candidate for treatment is identified by detecting IGF-1 and/or IGF-2 in the target cell type, or in the stratum of the target cell type. In another embodiment, the target cell type is a cancerous cell type. In another embodiment, the target cell type is a colon cancer cell type, a breast cancer cell type, an NSCLC cell type, or a leukemic cell type.

In another embodiment, a subject who is a candidate for treatment is identified by detecting activity of IGF-1R-mediated signaling in the target cell type (e.g., a tumor or other cancerous tissue), wherein IGF-1R-mediated signaling in the target cell type indicates that the subject is a candidate for treatment. Examples of molecules that can be monitored for IGF-1R-dependent changes are shown in FIG. 10, such as molecules in the PI3/Akt pathway, e.g., IGF-1R, IRS adapters, Akt, etc. Such molecules can be monitored for, for example, a change in phosphorylation status, e.g., an increase in phosphorylation. Phosphospecific antibodies that recognize the activated forms of these protein markers are highly developed, and these reagents have proven to be reliable for immunoblot detection in experimental systems.

In another embodiment, methods and compositions are provided for determining whether a tissue in a subject (for example, a tumor tissue or other cancerous tissue in the subject) has a molecular marker that identifies the subject as being more likely or less likely to respond favorably to treatment using the therapeutic methods and compositions of the present invention. Any such molecular marker can be used. In one embodiment, the molecular marker is a chromosomal abnormality (for example, in tumor-derived tissue), such as a chromosomal abnormality involving the EWS gene and a transcription factor. In one particular embodiment, the molecular marker is a EWS-FLI chromosomal translocation in a tumor or other cancerous tissue. Such translocations can be detected using any method known in the art (see, for example, Giovannini et al., 1994, J Clin Invest. 94:489-96; Delattre et al., 1994, NEJM 331:294-99; and Zoubek et al., 1994, Br J Cancer 70:908-13, each incorporated herein by reference in its entirety and for all purposes). Examples of such detection methods include cytological analysis, fluorescent in situ hybridization (FISH), sequence analysis of a EWS-FLI hybrid gene, detection and/or quantification of a transcriptional product of a EWS-FLI hybrid gene (using, e.g., a PCR-based technique such as RT-PCR, or a hybridization based technique such as in situ hybridization or a northern blot), detection and/or quantification of a polypeptide product of a EWS-FLI hybrid gene (using, e.g., an antibody-based technique such as in situ staining or a western blot), detection and/or quantification of a molecule or an activity associated with a EWS-FLI hybrid gene product, detection and/or quantification of a molecule or an activity dependent upon an activity of a EWS-FLI hybrid gene product, or detection and/or quantification of a molecule or an activity affected by an activity of a EWS-FLI hybrid gene product. In another particular embodiment, detection of a EWS-FLI hybrid gene product (e.g., a product of transcription or of translation) in a tumor or other cancerous tissue indicates that the tumor or cancerous tissue is more likely to respond to treatment using an anti-IGF-1 receptor inhibitor, or another inhibitor of signaling through the IGF-1 receptor signaling pathway, than a tumor or other cancerous tissue in which a EWS-FLI hybrid gene product is not detected. In another particular embodiment, a sample derived from a tumor or other cancerous tissue containing a EWS-FLI chromosomal translocation is tested to determine whether it expresses a EWS-FLI hybrid gene product. Detection of the EWS-FLI hybrid gene product indicates that the tumor or cancerous tissue is more likely to respond to treatment using an anti-IGF-1 receptor treatment or another inhibitor of signaling through the IGF-1 receptor signaling pathway.

In another embodiment, the molecular marker is a mutation in a signaling molecule, for example, in a kinase. The mutation can, for example, increase the activity of the signaling molecule, decrease the activity of the signaling molecule, and/or alter the ligand specificity, substrate specificity, timing, or location of the activity of the signaling molecule. In some embodiments, the signaling molecule is a RAS, and the mutation is an activating mutation. RAS mutations are found in about one third of all human tumors. Examples of activating RAS mutations include mutations to codons 12, 13, and 61. Other examples of activating RAS mutations include mutations in codons 10, 11, 15, 18, and 22. Other types of mutations or other changes can also cause an inappropriate increase in signaling through a RAS molecule. Examples of such other types of changes include gene amplification, overexpression, or upstream activation of a RAS pathway, e.g., approximately 40% of esophageal adenocarcinomas have an amplified KRAS gene, resulting in increased KRAS signaling; high levels of RAS activity are found in about half of all breast cancer tumors and are associated with expression of epidermal growth factor and HER-2, yet RAS mutations are rare in these tumors. Thus, the present invention provides methods for identifying subjects with elevated RAS activity as being more likely to respond favorably to treatment using an inhibitor of IGF-1 receptor signaling, and/or of treating such subjects with an inhibitor of IGF-1 receptor signaling.

In one particular embodiment, it is determined whether a subject has an activating KRAS mutation in at least some cells of at least one tumor, wherein the presence of the activating KRAS mutation indicates that the subject is more likely to respond to treatment of the tumor using an inhibitor of IGF-1 receptor signaling. The activating KRAS mutation can be any known in the art, for example, one affecting codon 10, 11, 12, 13, 15, 18, 22, 59, 61, and 63, such as G12C, G12D, G12E, and G12V. KRAS mutations are the most prevalent type of RAS mutations found in human tumors. Many tumor types are known to comprise activating KRAS mutations, including tumors of the pancreas (72-90% of which have an activating KRAS mutation), colon or rectum (32-57%), lung (15-50%), endometrium (5-50%), gallbladder (14-38%), and testes (9-12%), and multiple myeloma tumors (16-33%). Friday et al., 2005, Biochim Biophys Acta 1756:127-44. Thus, in various embodiments of the invention, methods and compositions are provided for detecting KRAS mutations in at least some cells of a tumor in a subject, and/or treating the subject with an inhibitor of IGF-1 receptor signaling. In particular embodiments, the subject has a tumor of the pancreas, colon, rectum, lung, endometrium, gallbladder, or testes, or a multiple myeloma tumors.

In another embodiment, a tumor that has a wild-type allele of KRAS is treated with an IGF-1 receptor inhibitor. In one particular embodiment, the tumor is also treated with an EGF receptor inhibitor, such as panitumumab or cetuximab. In another particular embodiment, the tumor was previously treated with an EGF receptor inhibitor, such as panitumumab or cetuximab, and is now treated with both an EGF receptor inhibitor (either the same EGF receptor inhibitor previously used, or another) and an IGF-1 receptor inhibitor. In another particular embodiment, the treated tumor is a colorectal tumor.

In another embodiment, it is determined whether some fraction of cells taken from a tumor in a subject has reduced PTEN activity, wherein reduced PTEN activity indicates that the tumor is less likely to respond to inhibition of IGF-1 receptor signaling. The reduction in PTEN activity can be detected using any suitable method. For example, expression levels can be detected using a method that detects PTEN RNA levels (e.g., via a hybridization-based method such as Northern Blot or in situ hybridization), protein levels (e.g., using a detecable PTEN-binding agent, such as a detectably labeled anti-PTEN antibody), or PTEN enzymatic activity (e.g., by measuring PTEN activity directly or indirectly through its effects on other molecules, or by detecting mutations that cause a reduction of PTEN activity, such as partial or complete loss-of-function mutations in PTEN, for example PTEN D331G). See, e.g., Teng et al., 1997, Cancer Res 57:5221-25; Bonneau et al., 2000, Human Mutation 16:109-22, each incorporated herein by reference in its entirety for all purposes.

In another aspect, the present invention provides compositions, kits, and methods for determining whether a patient is a good candidate for treatment with regimen that reduces IGF-1 and/or IGF-2-dependent signaling. It is understood in the art that it is desirable to determine whether a patient is a good candidate to begin a treatment regimen (or to continue with a treatment regimen already begun) as this allows the patient and medical professional to make better informed and more effective treatment decisions. It is particularly desirable to distinguish better from worse candidates for a treatment regimen for diseases that are aggressive or deadly, such as pancreatic cancer. The treatment regimen can target one or more molecules upstream or downstream of IGF-1 and or IGF-2, or target either or both of IGF-1 and IGF-2 themselves. Any such treatment known in the art or described herein can be used. In one embodiment, the treatment targets IGF-1R or IGF-1R/insulin receptor hybrids. Examples of such treatments include small molecules (such as OSI-906, linsitinib, BMS-754807, INSM-18, XL228, AXL1717, BMS-536924, NVP-ADW742, GSK621659A, GSK1838705A, A-928605, AZD4253, TAE226, or AG1024) and anti-IGF-1R antibodies (such as ganitumab, AMG 479, figitumumab, CP-751,871, cixutumumab, IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or fragments or derivatives thereof).

In one embodiment, the invention provides a method of determining whether a cancerous condition in a patient is likely to respond to a treatment that reduces signaling mediated by IGF-1 or IGF-2, comprising determining the concentration of a circulating biomarker in the patient's serum, wherein the amount of the circulating biomarker is predictive of a response to the treatment. Examples of such markers include total IGF-1, free IGF-1, total IGF-2, free IGF-2, IGFBP-1, IGFBP-2, IGFBP-3, IGFBP-4, IGFBP-5, IGFBP-6, and IGFBP-7. In another embodiment, a patient is a better candidate for treatment if the patient's IGFBP-1 and/or IGFBP-2 levels are lower than average for patients with the same condition. In another embodiment, a patient is a better candidate for treatment if the patient's total IGF-1, free IGF-1, total IGF-2, free IGF-2, IGFBP-3, and/or IGFBP-4 is higher than average for patients with the same condition.

In another embodiment, levels of two, three, four, or more of these markers are determined. Taken together, these two or more markers are used as a composite marker. A composite marker can include, for example, the relative quantities of total IGF-1, free IGF-1, total IGF-2, free IGF-2, peptides that include the IGF-2 protein sequence, IGFBP-1, IGFBP-2, IGFBP-3, IGFBP-4, IGFBP-5, IGFBP-6 and/or IGFBP-7. Examples of specific composite markers include, for example, ratios or quantitative differences between IGF-1 and IGF-2, 1GF-1 and IGFBP-1, IGF-1 and IGFBP-2, IGF-1 and IGFBP-3, IGF-1 and IGFBP-4, IGF-1 and IGFBP-5, IGF-1 and IGFBP-6, IGF-1 and IGFBP-7, IGF-2 and IGFBP-1, IGF-2 and IGFBP-2, IGF-2 and IGFBP-3, IGF-2 and IGFBP-4, IGF-2 and IGFBP-5, IGF-2 and IGFBP-6, IGF-2 and IGFBP-7, and between the various binding proteins e.g. IGFBP-2 and IGFBP-3.

Any method of determining the concentration of a circulating biomarker can be used. In one embodiment, a detectable molecule that binds to the circulating biomarker is used. Examples of such molecules include proteins, such as antibodies or fragments of antibodies, optionally coupled to a detectable moiety.

Examples of commercially available kits for detecting IGF-1 include the ELISA kits from Enzo Life Sciences (Farmingdale, N.Y.), Diagnostic Systems Laboratories, Inc. (Webster, Tex.), Abnova Corporation (Taipei City, Taiwan), Antigenix America Inc. (Huntington Station, N.Y.), ARP American Research Products, Inc. (Waltham, Mass.), BioVendor Laboratory Medicine, Inc. (Candler, N.C.), Cell Sciences (Canton, Mass.), DRG International, Inc. (Mountainside, N.J.), ElAab & USCNLIFE (Wuhan EIAab Science Co., Ltd) (Wuhan, China), GenWay Biotech, Inc. (San Diego, Calif.), IBL International GmbH (Hamburg, Germany), R&D Systems (Minneapolis, Minn.), and Raybiotech, Inc. (Norcross, Ga.).

Examples of commercially available kits for detecting IGF-2 include the ELISA kits from Antigenix America Inc. (Huntington Station, N.Y.), BioVendor Laboratory Medicine, Inc. (Candler, N.C.), and Diagnostic Systems Laboratories, Inc. (Webster, Tex.).

Examples of commercially available kits for detecting IGFBP-1 include the ELISA kits from R&D Systems (Minneapolis, Minn.), BioVendor Laboratory Medicine, Inc. (Candler, N.C.), Diagnostic Systems Laboratories, Inc. (Webster, Tex.), Cell Sciences (Canton, Mass.), and Raybiotech, Inc. (Norcross, Ga.).

Examples of commercially available kits for detecting IGFBP-2 include the ELISA kits from R&D Systems (Minneapolis, Minn.), BioVendor Laboratory Medicine, Inc. (Candler, N.C.), Diagnostic Systems Laboratories, Inc. (Webster, Tex.), Cell Sciences (Canton, Mass.), and Raybiotech, Inc. (Norcross, Ga.).

Examples of commercially available kits for detecting IGFBP-3 include the ELISA kits from R&D Systems (Minneapolis, Minn.), BioVendor Laboratory Medicine, Inc. (Candler, N.C.), Diagnostic Systems Laboratories, Inc. (Webster, Tex.), Cell Sciences (Canton, Mass.), and Raybiotech, Inc. (Norcross, Ga.), EIAab & USCNLIFE (Wuhan EIAab Science Co., Ltd) (Wuhan, China), Abnova Corporation (Taipei City, Taiwan), and Antigenix America Inc. (Huntington Station, N.Y.).

Examples of commercially available kits for detecting IGFBP-4 include the ELISA kits from R&D Systems (Minneapolis, Minn.), Cell Sciences (Canton, Mass.), Diagnostic Systems Laboratories, Inc. (Webster, Tex.), and Raybiotech, Inc. (Norcross, Ga.).

Examples of commercially available kits for detecting IGFBP-5 include the ELISA kits from R&D Systems (Minneapolis, Minn.), Antigenix America Inc. (Huntington Station, N.Y.), and Raybiotech, Inc. (Norcross, Ga.).

Examples of commercially available kits for detecting IGFBP-6 include the ELISA kits from R&D Systems (Minneapolis, Minn.) and Raybiotech, Inc. (Norcross, Ga.).

In another aspect, the present invention provides compositions, kits, and methods for determining whether to modulate or discontinue treatment with regimen that reduces IGF-1 and/or IGF-2-dependent signaling. The treatment regimen can target one or more molecules upstream or downstream of IGF-1 and or IGF-2, or target either or both of IGF-1 and IGF-2 themselves. Any such treatment known in the art or described herein can be used. In one embodiment, the treatment targets IGF-1R and/or IGF-1R/insulin receptor hybrids (Soos et al., 1993, Biochem J 290:419-26; Benyoucef et al., 2007, Biochem J 403:603-13). Examples of such treatments include small molecules (such as OSI-906, linsitinib, BMS-754807, INSM-18, XL228, AXL1717, BMS-536924, NVP-ADW742, GSK621659A, GSK1838705A, A-928605, AZD4253, TAE226, or AG1024) and anti-IGF-1R antibodies (such as ganitumab, AMG 479, figitumumab, CP-751,871, cixutumumab, IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or fragments or derivatives thereof).

In one embodiment, the patient's exposure to the treatment is determined. In another embodiment, if the patient's exposure is below the mean exposure for patient's receiving the same treatment regimen for the same cancerous condition, the treatment is discontinued. In another embodiment, if the patient's exposure is below the mean exposure for patient's receiving the same treatment regimen for the same cancerous condition, the dose or frequency of the treatment is increased.

The foregoing methods can be combined with other biomarkers, patient stratification methods, and efficacy-monitoring methods relating to IGF-1 and/or IGF-2-mediated signaling inhibition, for example, those described in US 2010/0184125, US 2010/0316639, US 2009/0092596, US 2009/093488, WO 2010/146059, WO 2009/079587, EP 1 828 249, EP 2 241 634, WO 2010/119126, US 2010/0240665, WO 2010/022268, WO 2010/048123, WO 2010/048123, WO 2010/138908, WO 2007/035744, WO 2008/115470, WO 2008/144345, WO 2011/053779, WO 2012/006681, U.S. Pat. No. 7,129,040, U.S. Pat. No. 7,217,795, WO 2011/087868, WO 2009/106566, WO 2011/133668, US 2009/0258365, WO 2011/066200, EP 1 926 996, US 2011/0217309, US 2011/0275644, U.S. Pat. No. 7,939,272, U.S. Pat. No. 8,048,621, U.S. Pat. No. 8,062,838, WO 2011/109572, WO 2011/109584, WO 2011/083391, US 2011/0060605, WO 2011/031861, EP 1 828 249, EP 2 032 989, EP 2 281 841, EP 2 283 831, WO 2009/079587, WO 2012/000763, US 2011/0052667, US 2011/0262453, and WO 2009/120767.

The compositions and/or methods of the present invention also can be used, for example, in cosmetic treatments, in veterinary treatments, to increase longevity, to treat reproductive defects, and to treat a variety of growth related disorders.

### Therapeutic Methods

Certain methods provided herein comprise administering to a subject an inhibitor of IGF-1R-mediated signaling. Any treatment that results in a reduction of an activity or signal mediated by IGF-1R can be used. Examples of such treatments are provided in Sachdev et al., 2007, Mol Cancer Ther. 6:1-12. In one embodiment, the treatment comprises administering to the subject a substance that reduces an activity mediated by IGF-1R. Examples of such substances include, but are not limited to, antibodies (including fragments and derivatives thereof), peptibodies, and AVIMERS™ (Amgen, Inc., Thousand Oaks, Calif.) that bind to IGF-1R, IGF-1, or IGF-2, soluble, IGF-1- and/or IGF-2-binding derivatives of IGF-1R, small molecules that bind to IGF-1R, IGF-1, IGF-2, IRS1, SHC, GRB2, SOS1, PI3K, SHP2, or any other molecule that acts in the IGF-1R signaling cascade, IGF-1 or IGF-2 binding proteins (and derivatives thereof), inhibitory nucleic acids (such as siRNA) and derivatives thereof (including peptide nucleic acids). Non-limiting examples of such molecules can be found in, for example, U.S. Pat. No. 7,329,7347 (published Feb. 12, 2008), 173,005 (issued Feb. 6, 2007), 7,071,300 (issued Jul. 4, 2006), 7,020,563 (issued Mar. 28, 2006), 6875741 (issued Apr. 5, 2005); U.S. Pat. App. Pub. No. 07/029,9010 (published Dec. 27, 2007), 07/026,5189 (published Nov. 15, 2007), 07/013,5340 (published Jun. 14, 2007), 07/012,9399 (published Jun. 7, 2007), 07/000,4634 A1 (published Jan. 4, 2007), 05/0282761 A1 (published Dec. 22, 2005), 05/0054638 A1 (published Mar. 10, 2005), 04/0023887 A1 (published Feb. 5, 2004), 03/0236190 A1 (published Dec. 25, 2003), 03/0195147 A1 (published Oct. 16, 2003); PCT Pub. No. WO 07/099,171 (published Sep. 7, 2007), WO 07/099,166 (published Sep. 7, 2007), 07/031,745 (published Mar. 22, 2007), WO 07/029,106 (published Mar. 15, 2007), WO 07/029,107 (published Mar. 15, 2007), WO 07/004,060 (published Jan. 11, 2007), WO 06/074057 A2 (published Jul. 13, 2006), WO 06/069202 A2 (published Jun. 29, 2006), WO 06/017443 A2 (published Feb. 16, 2006), WO 06/012422 A1 (published Feb. 2, 2006), WO 06/009962 A2 (published Jan. 26, 2006), WO 06/009950 A2 (published Jan. 26, 2006), WO 06/009947 A2 (published Jan. 26, 2006), WO 06/009933 A2 (published Jan. 26, 2006), WO 05/097800 A1 (Oct. 20, 2005), WO 05/082415 A2 (published Sep. 9, 2005), WO 05/037836 A2 (published Apr. 28, 2005), WO 03/070911 A2 (published Aug. 28, 2003), WO 99/28347 A2 (published Jun. 10, 1999); European Pat. No. EP 1 732 898 B1 (published Jan. 23, 2008), EP 0 737 248 B1 (published Nov. 14, 2007), European Pat. App. No. EP 1 496 935 A2 (published Jan. 19, 2005) and EP 1 432 433 A2 (published Jun. 30, 2004), and D'ambrosio et al., 1996, Cancer Res. 56:4013-20, each of which is incorporated herein by reference in its entirety. Specific examples of such molecules include OSI-906 (OSI Pharmaceuticals, Melvilee, N.Y.), BMS 536924 (Wittman et al., 2005, J Med. Chem. 48:5639-43; Bristol Myers Squibb, New York, N.Y.), XL228 (Exelexis, South San Francisco, Calif.), INSM-18, NDGA, and rhIGFBP-3 (Insmed, Inc., Richmond, Va.; Breuhahn et al, 2002006, Curr Cancer Ther Rev. 2:157-67; Youngren et al., 2005, Breast Cancer Res Treatment 94:37-46; U.S. Pat. No. 6,608,108), each of which reference is incorporated herein by reference in its entirety.

In one aspect, any suitable anti-IGF-1R antibody, antibody fragment, or antibody derivative can be used in the methods of the present invention. In one embodiment, the antibody, antibody fragment, or antibody derivative binds to the extracellular domain of IGF-1R. In another embodiment, the antibody, antibody fragment, or antibody derivative competes for binding to IGF-R with IGF-1 and/or IGF-2. In another embodiment, the antibody, antibody fragment, or antibody derivative, when bound to IGF-1R, reduces the amount of IGF-1 and/or IGF-2 that binds to the IGF-1R. In another embodiment, the antibody, antibody fragment, or antibody derivative binds to the L1 subdomain of the IGF-1R extracellular domain. In another embodiment, the antibody, antibody fragment, or antibody derivative binds to the CR subdomain of the IGF-1R extracellular domain. In another embodiment, the antibody, antibody fragment, or antibody derivative binds to the L2 subdomain of the IGF-1R extracellular domain. In another embodiment, the antibody, antibody fragment, or antibody derivative binds to the FnIIII subdomain of the IGF-1R extracellular domain. In another embodiment, the antibody, antibody fragment, or antibody derivative binds to the FnIII2-ID subdomain of the IGF-1R extracellular domain. In another embodiment, the antibody, antibody fragment, or antibody derivative binds to the FnIII subdomain of the IGF-1R extracellular domain. (The IGF-1R extracellular subdomains are defined in Example 12, below.) In another embodiment, the antibody, antibody fragment, or antibody derivative binds to more than one IGF-1R extracellular domain. Non-limiting examples of anti-IGF-1R antibodies that can be used in the methods of the present invention include each of the antibodies identified herein as L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20, H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52, and IGF-1R-binding fragments and derivatives thereof. Other non-limiting examples of anti-IGF-1R antibodies for use in the methods of the present invention include those described in US Pat. App. Pub. No. 06/0040358 (published Feb. 23, 2006), 05/0008642 (published Jan. 13, 2005), 04/0228859 (published Nov. 18, 2004), e.g., antibody 1A (DSMZ Deposit No. DSM ACC 2586), antibody 8 (DSMZ Deposit No. DSM ACC 2589), antibody 23 (DSMZ Deposit No. DSM ACC 2588) and antibody 18 as described therein; PCT Pub. No. WO 06/138729 (published Dec. 28, 2006), WO 05/016970 (published Feb. 24, 2005), and Lu et al., 2004, J Biol. Chem. 279:2856-65, e.g., antibodies 2F8, A12, and IMC-A12 as described therein; PCT Pub. No. WO 07/012,614 (published Feb. 1, 2007), WO 07/000,328 (published Jan. 4, 2007), WO 06/013472 (published Feb. 9, 2006), 05/058967 (published Jun. 30, 2005), 03/059951 (published Jul. 24, 2003), US Pat. App. Pub. No. 05/0084906 (published Apr. 21, 2005), e.g., antibody 7C10, chimaeric antibody C7C10, antibody h7C10, antibody 7H2M, chimaeric antibody *7C10, antibody GM 607, humanized antibody 7C10 version 1, humanized antibody 7C10 version 2, humanized antibody 7C10 version 3, and antibody 7H2HM, as described therein; US Pat. App. Pub. No. 05/0249728 (published Nov. 10, 2005), 05/0186203 (published Aug. 25, 2005), 04/0265307 (published Dec. 30, 2004), 03/0235582 (published Dec. 25, 2003), Maloney et al., 2003, Cancer Res. 63:5073-83, e.g., antibody EM 164, resurfaced EM 164, humanized EM164, huEM164 v1.0, huEM164 v1.1, huEM164 v1.2, and huEM164 v1.3, as described therein; U.S. Pat. No. 7,037,498 (issued May 2, 2006), US Pat. App. No. 05/0244408 (published Nov. 30, 2005), 04/0086503 (published May 6, 2004), Cohen, et al., 2005, Clinical Cancer Res. 11:2063-73, e.g., antibody CP-751,871, each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein; US Pat. App. No. 05/0136063 (published Jun. 23, 2005), 04/0018191 (published Jan. 29, 2004), e.g. antibody 19D12 and an antibody comprising a heavy chain encoded by a polynucleotide in plasmid 15H12/19D12 HCA (γ4), deposited at the ATCC under number PTA-5214, and a light chain encoded by a polynucleotide in plasmid 15H12/19D12 LCF (κ), deposited at the ATCC under number PTA-5220, as described therein; US Pat. App. No. 04/0202655 (published Oct. 14, 2004), e.g., antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, as described therein; U.S. patent application Ser. No. 07/024,3194 (published Oct. 18, 2007), e.g., antibodies M13-C06, M14-G11, M14-C03, M14-B01, M12-E01, and M12-G04, and antibodies produced by hybridomas P2A7.3E11, 20C8.3B8, P1A2.2B11, 20D8.24B11, P1E2.3B12, and P1G10.2B8. Each of the foregoing references is incorporated herein by reference in its entirety. Also suitable for use are antibodies, antibody fragments, or antibody derivatives that compete for binding to IGF-1 receptor with one of the aforementioned antibodies. In one embodiment, the antibody, antibody fragment, or antibody derivative binds to the same epitope as one of the aforementioned antibodies, or to an epitope that overlaps with the epitope of one of the aforementioned antibodies.

In particular embodiments, methods of the invention involve contacting endogenous IGF-1R with an IGF-1R binding antigen binding protein, e.g., via administration to a subject or in an ex vivo procedure.

The term “treatment” encompasses alleviation or prevention of at least one symptom or other aspect of a disorder, or reduction of disease severity, and the like. A treatment need not effect a complete cure, or eradicate every symptom or manifestation of a disease, to constitute a viable therapy. As is recognized in the pertinent field, drugs or other treatments employed as therapeutic agents may reduce the severity of a given disease state, but need not abolish every manifestation of the disease to be regarded as therapeutically useful. Similarly, a prophylactically administered treatment need not be completely effective in preventing the onset of a condition in order to constitute a viable prophylactic agent. Simply reducing the impact of a disease (for example, by reducing the number or severity of its symptoms, by delaying the onset of the condition, by accelerating the reduction of symptoms, by increasing the effectiveness of another treatment, or by producing another beneficial effect), or reducing the likelihood that the disease will occur or worsen in a subject, is sufficient. Therapeutically useful treatments also include treatments that are effective in some patients, but not in others. One embodiment of the invention is directed to a method comprising administering to a patient an IGF-1R antagonist in an amount and for a time sufficient to induce a sustained improvement over baseline of an indicator that reflects the severity of the particular disorder.

The progress of a course of treatment can be monitored or measured using any suitable technique. For treating a tumor, such techniques include detecting the size, or change in size, of the tumor. The size of the tumor can be measured by its length, circumference, volume, etc., as determined or estimated using any suitable technique, including direct observation, radiological techniques, and the like. In certain embodiments, progress of the treatment is monitored using the RECIST techniques and criteria (Therasse et al. 2000, J Natl Cancer Inst. 92:205-16, incorporated herein by reference in its entirety for all purposes). Progress of the treatment can also be monitored in other ways, for example, by determining the relative health or vigor of the tumor tissue, e.g., by measuring the tumor's uptake of glucose using a PET scan, or by monitoring an aspect of the tumor that is correlated with the health or vigor of the tumor tissue, or with the effectiveness of the treatment. Examples of such aspects of the tumor include expression levels of particular genes or proteins, phosphorylation states or other post-translational modifications of particular proteins, and the like.

As is understood in the pertinent field, pharmaceutical compositions comprising the molecules of the invention are administered to a subject in a manner appropriate to the indication. Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Localized administration, e.g. at a site of disease or injury is contemplated, as are transdermal delivery and sustained release from implants. Delivery by inhalation includes, for example, nasal or oral inhalation, use of a nebulizer, inhalation of the antagonist in aerosol form, and the like. Other alternatives include eyedrops; oral preparations including pills, syrups, lozenges or chewing gum; and topical preparations such as lotions, gels, sprays, and ointments.

Use of pharmaceutical compositions in ex vivo procedures also is contemplated. For example, a patient's blood or other bodily fluid may be contacted with an inhibitor of IGF-1R signaling ex vivo. The inhibitor may be bound to a suitable insoluble matrix or solid support material.

IGF-1R signaling inhibitors of the instant invention can be administered in the form of a composition comprising one or more additional components such as a physiologically acceptable carrier, excipient or diluent. Optionally, the composition additionally comprises one or more physiologically active agents, for example, a second IGF-1R signaling inhibitor, an anti-angiogenic substance, a chemotherapeutic substance, an analgesic substance, etc., non-exclusive examples of which are provided herein. In various particular embodiments, the composition comprises one, two, three, four, five, or six physiologically active agents in addition to an IGF-1R binding antigen binding protein

In one embodiment, the pharmaceutical composition comprise an inhibitor of IGF-1R signaling together with one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, Pa.

Kits for use by medical practitioners include an IGF-1 receptor-inhibiting substance of the invention and a label or other instructions for use in treating any of the conditions discussed herein. In one embodiment, the kit includes a sterile preparation of one or more inhibitors of IGF-1R signaling, which may be in the form of a composition as disclosed above, and may be in one or more vials.

Dosages and the frequency of administration may vary according to such factors as the route of administration, the particular antigen binding proteins employed, the nature and severity of the disease to be treated, whether the condition is acute or chronic, and the size and general condition of the subject. Appropriate dosages can be determined by procedures known in the pertinent art, e.g. in clinical trials that may involve dose escalation studies. “Intermittent dosing” refers to methods of administering to a subject a therapeutic compound (for example, an inhibitor of IGF-1R signaling) in multiple doses, wherein there is an interval of time between administration of a particular dose and any subsequent dose. Any schedule of dosing can be used so long as it is therapeutically effective or otherwise medically justified. The interval between consecutive doses can be very short, on the order of seconds or minutes, or longer, on the order of hours, days, weeks, months, or even years. The interval can be the same between every dose, for example, one dose per week or month, or it can vary from dose to dose. Likewise, the amount of the therapeutically active compound (e.g., an inhibitor of IGF-1R signaling or chemotherapeutic agent) can vary from dose to dose. In one embodiment, the period between consecutive doses and the amount of a therapeutically active substance in each dose are selected to keep a pharmacodynamic or pharmacokinetic parameter of interest (for example, serum concentration of said substance or percent reduction in IGF-1R signaling activity) within a desired range. In another embodiment, the interval between doses and the amount of therapeutically active substance vary according to other criteria (for example, subject's objective or subjective response to the course of treatment).

In other embodiments, the IGF-1R signal inhibiting substance of the invention is administered over a period of at least a month or more, e.g., for one, two, or three months, six months, a year, for several years, or even indefinitely. For treating chronic conditions, long-term treatment is generally most effective. However, for treating acute conditions, administration for shorter periods, e.g. from one to six weeks, may be sufficient. In general, the IGF-1R signal inhibiting substance of the invention is administered until the patient manifests a medically relevant or desirable degree of improvement over baseline for the chosen indicator or indicators.

Particular embodiments of the present invention involve administering an IGF-1R inhibiting substance at a dosage of from about 1 ng of antigen binding protein per kg of subject's mass per dose (“1 ng/kg/dose”) to about 50 mg/kg/dose, more preferably from about 1 mg/kg/dose to about 30 mg/kg/dose, and most preferably from about 10 mg/kg/dose to about 20 mg/kg/dose, to a subject. In additional embodiments, the IGF-1R inhibiting substance is administered to adults one time per month, once every two weeks, once per week, two times per week, or three or more times per week, to treat an IGF-1 and/or IGF-2 mediated disease, condition or disorder, e.g., a medical disorder disclosed herein. If injected, the effective amount of IGF-1R inhibiting substance per adult dose may range from 1-20 mg/m², and preferably is about 5-12 mg/m². Alternatively, a flat dose may be administered; the amount may range from 5-100 mg/dose. One range for a flat dose is about 20-30 mg per dose. In one embodiment of the invention, a flat dose of 25 mg/dose is repeatedly administered by injection. If a route of administration other than injection is used, the dose is appropriately adjusted in accordance with standard medical practices. One example of a therapeutic regimen involves injecting a dose of about 20-30 mg of IGF-1R inhibiting substance from one to three times per week over a period of at least three weeks, though treatment for longer periods may be necessary to induce the desired degree of improvement. For pediatric subjects (age 4-17), one exemplary suitable regimen involves the subcutaneous injection of 0.4 mg/kg, up to a maximum dose of 25 mg of IGF-1R inhibiting substance administered two or three times per week.

Particular embodiments of the methods provided herein involve subcutaneous injection of from 0.5 mg to 500 mg, preferably from 50 to 300 mg, of an antigen binding protein, once or twice per week. Another embodiment is directed to pulmonary administration (e.g., by nebulizer) of 3 or more mg of IGF-1R inhibiting substance.

Other examples of therapeutic regimens provided herein comprise subcutaneous or intravenous administration of a dose of 1, 3, 5, 6, 7, 8, 9, 10, 11, 12, 15, 20, 25, 30, 35, 40, 45, 50, 75, 100, 125, 150, 175, 200, 250, 300, 400, or 500 milligrams of an IGF-1R inhibitor of the present invention per kilogram body mass of the subject (mg/kg). The dose can be administered once to the subject, or more than once at a certain interval, for example, once a day, three times a week, twice a week, once a week, three times a month, twice a month, once a month, once every two months, once every three months, once every six months, or once a year. The duration of the treatment, and any changes to the dose and/or frequency of treatment, can be altered or varied during the course of treatment in order to meet the particular needs of the subject.

In another embodiment, an antigen binding protein is administered to the subject in an amount and for a time sufficient to induce an improvement, preferably a sustained improvement, in at least one indicator that reflects the severity of the disorder that is being treated. Various indicators that reflect the extent of the subject's illness, disease or condition may be assessed for determining Whether the amount and time of the treatment is sufficient. Such indicators include, for example, clinically recognized indicators of disease severity, symptoms, or manifestations of the disorder in question. In one embodiment, an improvement is considered to be sustained if the subject exhibits the improvement on at least two occasions separated by two to four weeks. The degree of improvement generally is determined by a physician, who may make this determination based on signs, symptoms, biopsies, or other test results, and who may also employ questionnaires that are administered to the subject, such as quality-of-life questionnaires developed for a given disease. An improvement in a subject's condition can be one that is, for example, detected, measured, or quantified by a physician or other health care provider using any appropriate technique. Such techniques include, but are not limited to, observing the subject, testing the subject or a sample taken from the subject, and collecting from the subject, directly or indirectly, the subject's impressions of the subject's condition. Such impressions can relate to any aspect of the subject's health or well-being, particularly those aspects that are affected directly or indirectly by subject's tumor disease. Examples of such aspects include, but are not limited to, pain, discomfort, sleep, appetite, thirst, mobility, strength, flexibility, and mental state.

Elevated levels of IGF-1 and/or IGF-2 are associated with a number of disorders, including, for example, cancer (e.g., lung, prostate, breast and colon cancers), and acromegaly and other overgrowth disorders (e.g., constitutionally tall children). Subjects with a given disorder may be screened, to identify those individuals who have elevated IGF-1 and/or IGF-2 levels, thereby identifying the subjects who may benefit most from treatment with an IGF-1R signaling inhibitor. Thus, treatment methods provided herein optionally comprise a first step of measuring a subject's IGF-1 and/or IGF-2 levels. An antigen binding protein may be administered to a subject in whom IGF-1 and/or IGF-2 levels are elevated above a normal or a desirable level.

A subject's levels of IGF-1 and/or IGF-2 may be monitored before, during and/or after treatment with an antigen binding protein, to detect changes, if any, in their levels. For some disorders, the incidence of elevated IGF-1 and/or IGF-2 levels may vary according to such factors as the stage of the disease or the particular form of the disease. Known techniques may be employed for measuring IGF-1 and/or IGF-2 levels, e.g., in a subject's serum. IGF-1 and/or IGF-2 levels in blood samples may be measured using any suitable technique, for example, ELISA.

Particular embodiments of methods and compositions of the invention involve the use of an antigen binding protein and one or more additional IGF-1R antagonists, for example, two or more antigen binding proteins of the invention, or an antigen binding protein of the invention and one or more other IGF-1R antagonists. In further embodiments, antigen binding protein are administered alone or in combination with other agents useful for treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which an antigen binding protein is administered at least once during a course of treatment that involves administering at least one other therapeutic agent to the patient.

Examples of other agents that may be co-administered with an antigen binding protein are other antigen binding proteins or therapeutic polypeptides that are chosen according to the particular condition to be treated. Alternatively, non-proteinaceous drugs that are useful in treating one of the particular conditions discussed above may be co-administered with an IGF-1R antagonist.

Combination Therapy

In another aspect, the present invention provides a method of treating a subject with an IGF-1R inhibiting antigen binding protein and one or more other treatments. In one embodiment, such a combination therapy achieves synergy or an additive effect by, for example, attacking multiple sites or molecular targets in a tumor. Types of combination therapies that can be used in connection with the present invention include inhibiting or activating (as appropriate) multiple nodes in a single disease-related pathway, multiple pathways in a target cell, and multiple cell types within a target tissue (e.g., within a tumor). For example, an IGF-1R inhibitor of the present invention can be combined with a treatment that inhibits IGF-1, promotes apoptosis, inhibits angiogenesis, or inhibits macrophage. In another embodiment, a targeted agent, that, when used by itself, fails to elicit a therapeutically desired effect, could be used to, for example, sensitize cancer cells or augment treatment effect of other agents. In another embodiment, an IGF-1R inhibitor according to the invention is used in combination with a cytotoxic drug or other targeted agent that induces apoptosis. In another embodiment, an IGF-1R inhibitor is used in combination with one or more agents that inhibit different targets that are involved in cell survival (e.g., PKB, mTOR), different receptor tyrosine kinases (e.g., ErbB1, ErbB2, c-Met, c-kit), or different cell types (e.g., KDR inhibitors, c-fms). In another embodiment, an IGF-1R inhibitor of the invention is added to the existing standard of care for a particular condition. Examples of therapeutic agents include, but are not limited to, gemcitabine, taxol, taxotere, and CPT-11.

In another embodiment, a combination therapy method comprises administering to the subject two, three, four, five, six, or more of the IGF-1R agonists or antagonists described herein. In another embodiment, the method comprises administering to the subject two or more treatments that together inhibit or activate (directly or indirectly) IGF-1R-mediated signal transduction. Examples of such methods include using combinations of two or more IGF-1R inhibiting antigen binding proteins, of an IGF-1R inhibiting antigen binding protein and one or more other IGF-1, IGF-2, and/or IGF-1R agonists or antagonists (e.g., IGF-1 and/or IGF-2 binding polypeptides, IGF-1R binding polypeptides, IGF-1 and/or IGF-2 derivatives, anti-IGF-1 and/or IGF-2 antibodies, anti-sense nucleic acids against IGF-1, IGF-2, and/or IGF-1R, or other molecules that bind to IGF-1, IGF-2, and/or IGF-1R polypeptides or nucleic acids), or of an IGF-1R inhibiting antigen binding protein and one or more other treatments (e.g., surgery, ultrasound, radiotherapy, chemotherapy, or treatment with another anti-cancer agent), as described, for example, in U.S. Pat. No. 5,473,054 (issued Dec. 5, 1995), 6,051,593 (issued Apr. 18, 2000), 6,084,085 (issued Jul. 4, 2000), 6,506,763 (issued Jan. 14, 2003), US Pat. App. Pub. No.s 03/0092631 (published May 15, 2003), 03/0165502 (published Sep. 4, 2003), 03/0235582 (published Dec. 25, 2003), 04/0886503 (published May 6, 2004), 05/0272637 (published Dec. 8, 2005), PCT Pub. Ser. No.s WO 99/60023 (published Nov. 25, 1999), WO 02/053596 (published Jul. 11, 2002), WO 02/072780 (published Sep. 19, 2002), WO 03/027246 (published Mar. 3, 2003), WO 03/020698 (published Mar. 13, 2003), WO 03/059951 (published Jul. 24, 2003), WO 03/100008 (published Dec. 4, 2003), WO 03/106621 (published Dec. 24, 2003), WO 04/071529 (published Aug. 26, 2004), WO 04/083248 (published Sep. 30, 2004), WO 04/087756 (published Oct. 14, 2004), WO 05/112969 (published Dec. 1, 2005), Kull et al., 1983, J Biol Chem 258:6561-66, Flier et al., 1986, Proc Natl Acad Sci USA 83:664-668, Conover et al., 1987, J Cell Physiol 133:560-66, Rohlik et al., 1987, Biochem Biophys Res Comm 149:276-81, Arteaga et al., 1989, J Clinical Investigation 84:1418-23, Arteaga et al., 1989, Cancer Res 49:6237-41, Gansler et al., 1989, American J Pathol 135:961-66, Gustafson et al., 1990, J Biol Chem 265:18663-67, Steele-Perkins et al., 1990, Biochem Biophys Res Comm 171:1244-51, Cullen et al., 1992, Mol Endocrinol 6:91-100, Soos et al., 1992, J Biol Chem 267:12955-63, Xiong et al., 1992, Proc Natl Acad Sci USA 89:5356-60, Brunner et al., 1993, Euro J Cancer 29A:562-69, Furlanetto et al., 1993, Cancer Res 53:2522-26, Li et al., 1993, Biochem Biophys Res Comm 196:92-98, Kalebic et al., 1994, Cancer Res 54:5531-34, Lahm et al., 1994, Intl J Cancer 58:452-59, Zia et al., 1996, J Cell Biochem Supp 24:269-75, Jansson et al., 1997, J Biol Chem 272:8189-97, Scotlandi et al., 1998, Cancer Res 58:4127-31, Logie et al., 1999, Li et al., 2000, Cancer Immunol Immunotherapy 49:243-52, J Mol Endocrinol 23:23-32, De Meyts et al., 2002, Nature Reviews 1:769-83, Hailey et al., 2002, Mol Cancer Therapeutics 1:1349-53, Maloney et al., 2003, Cancer Research 63:5073-83, Burtrum et al., 2003, Cancer Research 63:8912-21, and Karavitaki et al., 2004, Hormones 3:27-36, (each incorporated herein by reference in its entirety) may be employed in methods and compositions of the present invention. Furthermore, one or more anti-IGF-1R antibodies or antibody derivatives can be used in combination with one or more molecules or other treatments, wherein the other molecule(s) and/or treatment(s) do not directly bind to or affect IGF-1R, IGF-1, or IGF-2, but which combination is effective for treating or preventing a condition, such as cancer or an overgrowth disorder (e.g., acromegaly). In one embodiment, one or more of the molecule(s) and/or treatment(s) treats or prevents a condition that is caused by one or more of the other molecule(s) or treatment(s) in the course of therapy, e.g., nausea, fatigue, alopecia, cachexia, insomnia, etc. In every case where a combination of molecules and/or other treatments is used, the individual molecule(s) and/or treatment(s) can be administered in any order, over any length of time, which is effective, e.g., simultaneously, consecutively, or alternately. In one embodiment, the method of treatment comprises completing a first course of treatment with one molecule or other treatment before beginning a second course of treatment. The length of time between the end of the first course of treatment and beginning of the second course of treatment can be any length of time that allows the total course of therapy to be effective, e.g., seconds, minutes, hours, days, weeks, months, or even years.

In another embodiment, the method comprises administering one or more of the IGF-1R antagonists described herein and one or more other treatments (e.g., a therapeutic or palliative treatment), for example, anti-cancer treatments (such as surgery, ultrasound, radiotherapy, chemotherapy, or treatment with another anti-cancer agent). Where a method comprises administering more than one treatment to a subject, it is to be understood that the order, timing, number, concentration, and volume of the administrations is limited only by the medical requirements and limitations of the treatment, i.e., two treatments can be administered to the subject, e.g., simultaneously, consecutively, alternately, or according to any other regimen. Examples of agents that can be administered in combination with the IGF-1R antagonists described herein include, but are not limited to, neutrophil-boosting agents, irinothecan, SN-38, gemcitabine, herstatin, or an IGF-1R-binding herstatin derivative (as described, for example, in US Pat. App. No. 05/0272637), AVASTIN® (Genentech, South San Francisco, Calif.), HERCEPTIN® (Genentech), RITUXAN® (Genentech), ARIMIDEX® (AstraZeneca, Wilmington, Del.), IRESSA® (AstraZeneca), BEXXAR®(Corixa, Seattle, Wash.), ZEVALIN® (Biogen Ides, Cambridge, Mass.), ERBITUX® (Imclone Systems Inc., New York, N.Y.), GEMZAR® (Eli Lilly and Co., Indianapolis, Ind.), CAMPTOSAR® (Pfizer, New York, N.Y.), GLEEVEC® (Novartis), SU-11248 (Pfizer), BMS-354825 (Bristol-Myers Squibb), VECTIBIX™ (Abgenix, Fremont, Calif./Amgen Inc., Thousand Oaks, Calif.), and denosumab (Amgen Inc., Thousand Oaks, Calif.).

In another embodiment, the present invention provides a combination therapy for treating a tumor disease comprising administering to a subject an inhibitor of IGF-1 receptor signaling before, during, or after treatment of the subject with an inhibitor of RAS signaling, e.g., an inhibitor of KRAS, NRAS, or FIRAS. Any inhibitor of RAS activity can be used. Examples of types of RAS inhibitors include antisense oligonucleotides, RNA interference, inhibition of RAS post-translational modification or processing (e.g., farnesyltransferase inhibitors (FTIs), such as CAAX peptidomimetics like FTI-276 and FTI-277, and non-peptidomimetics like tipifarnib (R115777), lonafarnib (SCH663366), and BMS-214662)), geranylgeranyltransferase inhibitors (GGTIs), combination FTI/GGTIs, inhibitors of RAS proteolytic cleavage, methylation, or palmitoylation, immunological approaches (e.g., vaccination against an activated RAS mutant), mutant RAS peptide inhibitors, and inhibitors of downstream RAS effectors such as Raf kinase (e.g., BAY 43-9006), MEK (e.g., CI-1040, PD0325901, and ARRY-142886), and mTOR (e.g., rapamycin, CCI-779, RAD001, and AP23573). See Friday et al., 2005, Biochim Biophys Acta 1756:127-44, incorporated herein by reference in its entirety for all purposes.

The following examples, both actual and prophetic, are provided for the purpose of illustrating specific embodiments or features of the instant invention and do not limit its scope.

### Example 1

### Preparation of Antibodies

This example demonstrates a method of preparing antibodies recognizing the IGF-1 receptor.
   IGF-1 receptor polypeptides may be employed as immunogens in generating monoclonal antibodies by conventional techniques. It is recognized that polypeptides in various forms may be employed as immunogens, e.g., full length proteins, fragments thereof, fusion proteins thereof such as Fc fusions, cells expressing the recombinant protein on the cell surface, etc.

To summarize an example of such a procedure, an IGF-1R immunogen emulsified in complete Freund's adjuvant is injected subcutaneously into Lewis rats, in amounts ranging from 10-100 μl. Three weeks later, the immunized animals are boosted with additional immunogen emulsified in incomplete Freund's adjuvant and boosted every three weeks thereafter. Serum samples are periodically taken by retro-orbital bleeding or tail-tip excision for testing by dot-blot assay, ELISA (enzyme-linked immunosorbent assay), or inhibition of binding of ¹²⁵I-IGF-1 or ¹²⁵I-IGF-2 to extracts of IGF-1R-expressing cells. Following detection of an appropriate antibody titer, positive animals are given a final intravenous injection of antigen in saline. Three to four days later, the animals are sacrificed, splenocytes harvested, and fused to the murine myeloma cell line AG8653. The resulting hybridoma cell lines are plated in multiple microtiter plates in a HAT selective medium (hypoxanthine, aminopterin, and thymidine) to inhibit proliferation of non-fused cells, myeloma hybrids, and spleen cell hybrids.

Hybridoma clones thus generated are screened for reactivity with IGF-1R. Initial screening of hybridoma supernatants utilizes an antibody capture and binding of partially purified ¹²⁵I-IGF-1 receptor. Hybridomas that are positive in this screening method are tested by a modified antibody capture to detect hybridoma cells lines that are producing blocking antibody. Hybridomas that secrete a monoclonal antibody capable of inhibiting ¹²⁵I-IGF-1 binding to cells expressing IGF-1R are thus detected. Such hydridomas then are injected into the peritoneal cavities of nude mice to produce ascites containing high concentrations (>1 mg/ml) of anti-IGF-1R monoclonal antibody. The resulting monoclonal antibodies may be purified by ammonium sulfate precipitation followed by gel exclusion chromatography, and/or affinity chromatography based on binding of antibody to Protein G.

Similar methods can be used to generate human antibodies in transgenic mice. See, e.g., Chen et al., 1993, internat. Immunol. 5: 647-56; Chen et al., 1993, EMBO J. 12: 821-30; Choi et al., 1993, Nature Genetics 4: 117-23; Fishwild et al., 1996, Nature Biotech. 14: 845-51; Harding et al., 1995, Annals New York Acad. Sci.; Lonberg et al., 1994, Nature 368: 856-59; Lonberg, 1994, Handbook Exper.l Pharmacol. 113: 49-101; Lonberg et al., 1995, Internal Rev. Immunol. 13: 65-93; Morrison, 1994, Nature 368: 812-13; Neuberger, 1996, Nature Biotech. 14: 826; Taylor et al., 1992, Nuc. Acids Res. 20: 6287-95; Taylor et al., 1994, Internat. Immunol. 6: 579-91; Tomizuka et al., 1997, Nature Genetics 16: 133-43; Tomizuka et al., 2000, Proc. Nat. Acad. Sci. USA 97: 722-27; Tuaillon et al., 1993, Proc. Nat. Acad. Sci. USA 90: 3720-24; Tuaillon et al., 1994, J. Immunol. 152: 2912-20; Russel et al., 2000, Infection and Immunity April 2000: 1820-26; Gallo et al., 2000, Eur. J. Immunol. 30: 534-40; Davis et al., 1999, Cancer Metastasis Rev. 18:421-25; Green, 1999, J. Immunol. Methods 231:11-23; Jakobovits, 1998, Advanced Drug Delivery Rev. 31:33-42; Green et al., 1998, J. Exp. Med. 188: 483-95; Jakobovits, 1998, Exp. Opin. Invest. Drugs 7: 607-14; Tsuda et al., 1997, Genomics 42: 413-21; Mendez et al., 1997, Nature Genetics 15: 146-56; Jakobovits, 1996, Weir's Handbook of Experimental Immunology, The Integrated Immune System Vol. IV, 194.1-194.7; Mendez et al., 1995, Genomics 26: 294-307; Jakobovits, 1994, Current Biol. 4: 761-63; Arbones, 1994, Immunity 1: 247-60; Green et al., 1994, Nature Genetics 7: 13-21; Jakobovits et al., 1993, Nature 362: 255-58; Jakobovits et al., 1993, Proc. Nat. Acad. Sci. USA 90: 2551-55.

### Example 2

### Isolation of Human IGF-1R(ECD)-C3-muIgG1

This example provides a method of making a soluble fragment of IGF-1R useful for raising antibodies.

Cloning of pDSRα:huIGF-1R(ECD)-C3-muIgG1Fc

Primers 2830-36:
5′ AGCAAGCTTCCACCATGAAGTCTGGCTCCGGAGGAGG 3′ SEQ ID NO:256) and 2830-38:
5′ ATTTGTCGACTTCGTCCAGATGGATGAAGTTTTCAT 3′, SEQ ID NO:257)
   were used to amplify the human IGF-1R extracellular domain (1-906) cDNA sequence. The primers included a Kozak translation initiation sequence (underlined above) preceding the start codon, restriction sites for subsequent subcloning, and a caspace-3 site, which is inserted next to the extracellular domain C-terminus. PCR was performed on a PerkinElmer 2400 (PerkinElmer, Torrance, Calif.) under the following conditions: 1 cycle at 95° C. for 2 min, 23 cycles at 95° C. for 30 sec, 58.5° C. for 30 sec, and 72° C. for 3 min, and 1 cycle at 72° C. for 10 min. Final reaction conditions were 1× pfu TURBO® buffer (Stratagene, La Jolla, Calif.), 200 μM dNTPs, 2 μM each primer, 5 U pfu TURBO® (Stratagene) and 1 ng template DNA. The PCR product was purified using a Clontech Nucleospin Column (Clontech, Palo Alto, Calif.) according to the manufacturers instructions, digested with Hind III and Sal I (Roche, Indianapolis, Ind.) and gel purified. The human IGF-1R insert was ligated into Hind III/Sal I digested pDSRα-muIgG1. Integrity of the insert was confirmed by DNA sequencing. The sequence of the protein encoded by the resulting open reading frame (IGF-1R-C3-muFc) is shown in FIG. 10. The final expression vector, pDSRα:huIGF1R(ECD)-C3-muIgG1Fc, is described in Table 1.

TABLE 1
pDSRα:huIGF1R(ECD)-C3-muIgG1Fc

| Plasmid Base | |
|---|---|
| Pair Number: | |
| 11-3496 | atgaagtctggctccggaggagggtccccgacctcgctgtgggggctcctgtttctctccgccgcgct |
| | ctcgctctggccgacgagtggagaaatctgcgggccaggcatcgacatccgcaacgactatcagca |
| | gctgaagcgcctggagaactgcacggtgatcgagggctacctccacatcctgctcatctccaaggcc |
| | gaggactaccgcagctaccgcttccccaagctcacggtcattaccgagtacttggctgctgttccgagtg |
| | gctggcctcgagagcctcggagacctcttccccaacctcacggtcatccgcggctggaaactcttcta |
| | caactacgccctggtcatcttcgagatgaccaatctcaaggatattgggctttacaacctgaggaacatt |
| | actcggggggccatcaggattgagaaaaatgctgacctctgttacctctccactgtggactggtccctg |
| | atcctggatgcggtgtccaataactacattgtggggaataagcccccaaaggaatgtggggacctgtgt |
| | ccagggaccatggaggagaagccgatgtgtgagaagaccaccatcaacaatgagtacaactaccgc |
| | tgctggaccacaaaccgctgccagaaaatgtgcccaagcacgtgtgggaagcgggcgtgcaccga |
| | gaacaatgagtgctgccaccccgagtgcctgggcagctgcagcgcgcctgacaacgacacggcctg |
| | tgtagccttgccgccactactactatgccggtgtctgtgtgcctgcctgcccgcccaacacctacaggttt |
| | gagggctggcgctgtgtggaccgtgacttctgcgccaacatcctcagcgccgagagcagcgactcc |
| | gaggggtttgtgatccacgacggcgagtgcatgcaggagtgcccctcgggcttcatccgcaacggca |
| | gccagagcatgtactgcatcccttgtgaaggtccttgcccgaaggtctgtgaggaagaaaagaaaaca |
| | aagaccattgattctgttacttctgctcagatgctccaaggatgcaccatcttcaagggcaatttgctcatt |
| | aacatccgacgggggaataacattgcttcagagctggagaacttcatggggctcatcgaggtggtgac |
| | gggctacgtgaagatccgccattctcatgccttggtctccttgtccttcctaaaaaaccttcgcctcatcct |
| | aggagaggagcagctagaagggaattactccttctacgtcctcgacaaccagaacttgcagcaactgt |
| | gggactgggaccaccgcaacctgaccatcaaagcagggaaaatgtactttgctttcaatcccaaattat |
| | gtgtttccgaaatttaccgcatggaggaagtgacggggactaaagggcgccaaagcaaaggggaca |
| | taaacaccaggaacaacggggagagagcctcctgtgaaagtgacgtcctgcatttcacctccaccac |
| | cacgtcgaagaatcgcatcatcataacctggcaccggtaccggccccctgactacagggatctcatca |
| | gcttcaccgtttactacaaggaagcaccctttaagaatgtcacagagtatgatgggcaggatgcctgcg |
| | gctccaacagctggaacatggtggacgtggacctcccgcccaacaaggacgtggagcccggcatct |
| | tactacatgggctgaagccctggactcagtacgccgtttacgtcaaggctgtgaccctcaccatggtgg |
| | agaacgaccatatccgtggggccaagagtgagatcttgtacattcgcaccaatgcttcagttccttccat |
| | tcccttggacgttctttcagcatcgaactcctcttctcagttaatcgtgaagtggaaccctccctctctgcc |
| | caacggcaacctgagttactacattgtgcgctggcagcggcagcctcaggacggctacctttaccggc |
| | acaattactgctccaaagacaaaatccccatcaggaagtatgccgacggcaccatcgacattgaggag |
| | gtcacagagaaccccaagactgaggtgtgtggtggggagaaagggccttgctgcgcctgccccaaa |
| | actgaagccgagaagcaggccgagaaggaggaggctgaataccgcaaagtctttgagaatttcctgc |
| | acaactccatcttcgtgcccagacctgaaaggaagcggagagatgtcatgcaagtggccaacaccac |
| | catgtccagccgaagcaggaacaccacggccgcagacacctacaacatcactgacccggaagagct |
| | ggagacagagtaccctttcttgagagcagagtggataacaaggagagaactgtcatttctaaccttcg |
| | gcctttcacattgtaccgcatcgatatccacagctgcaaccacgaggctgagaagctgggctgcagcg |
| | cctccaacttcgtctttgcaaggactatgcccgcagaaggagcagatgacattcctgggccagtgacct |
| | gggagccaaggcctgaaaactccatctttttaaagtggccggaacctgagaatcccaatggattgattc |
| | taatgtatgaaataaaatacggatcacaagttgaggatcagcgagaatgtgtgtccagacaggaataca |
| | ggaagtatggaggggccaagctaaaccggctaaacccggggaactacacagcccggattcaggcc |
| | acatctctctctgggaatgggtcgtggacagatcctgtgttcttctatgtccaggccaaaacaggatatg |
| | aaaacttcatccatctggacgaagtcgacggttgtaagccttgcatatgtacagtcccagaagtatcatct |
| | gtcttcatcttccccccaaagcccaaggatgtgctcaccattactctgactcctaaggtcacgtgtgttgt |
| | ggtagacatcagcaaggatgatcccgaggtccagttcagctggtttgtagatgatgtggaggtgcaca |
| | cagctcagacgcaaccccgggaggagcagttcaacagcactttccgctcagtcagtgaacttcccatc |
| | atgcaccaggactggctcaatggcaaggagttcaaatgcagggtaaacagtgcagctttccctgcccc |
| | catcgagaaaaccatctccaaaaccaaaggcagaccgaaggctccacaggtgtacaccattccacct |
| | cccaaggagcagatggccaaggataaagtcagtctgacctgcatgataacagacttcttccctgaaga |
| | cattactgtggagtggcagtggaatgggcagccagcggagaactacaagaacactcagcccatcatg |
| | gacacagatggctcttacttcgtctacagcaagctcaatgtgcagaagagcaactgggaggcaggaa |
| | atactttcacctgctctgtgttacatgagggcctgcacaaccaccatactgagaagagcctctcccactc |
| | tcctggtaaa (SEQ ID NO: 258) |
| 3507 to 4391 | A transcription termination/polyadenylation signal from the α-subunit |
| | of the bovine pituitary glycoprotein hormone (α-FSH) (Goodwin et al., |
| | 1983, Nucleic Acids Res. 11:6873-82; Genbank Accession Number X00004) |
| 4600 to 5163 | A mouse dihydrofolate reductase (DHFR) minigene containing the |
| | endogenous mouse DHFR promoter, the cDNA coding signals (Gasser et al., |
| | 1982, Proc. Natl. Acad. Sci. U.S.A. 79:6522-6; Nunberg et al., 1980, |
| | Cell 19:355-64; Setzer et al., 1982, J. Biol. Chem. 257:5143-7; McGrogan |
| | et al., 1985, J. Biol. Chem. 260:2307-14) |
| 6389 to 7246 | pBR322 sequences containing the ampicillin resistance marker gene and |
| | the origin for replication of the plasmid in E. coli (Genbank Accession |
| | Number J01749) |
| 7459 to 7802 | An SV40 early promoter, enhancer and origin of replication (Takebe et |
| | al., 1988, Mol. Cell Biol. 8:466-72, Genbank Accession Number J02400) |
| 7809 to 8065 | A translational enhancer element from the HTLV-l LTR domain (Seiki et |
| | al., 1983, Proc. Natl. Acad. Sci. U.S.A. 80:3618-22, Genbank Accession |
| | Number J02029) |
| 8109 to 8205 | An intron from the SV40 16S, 19S splice donor/acceptor signals |
| | (Okayama and berg, 1983. Mol. Cell Biol. 3:280-9, Genbank Accession |
| | Number J02400) |

Expression of hu IGF-1R(ECD)-C3-muIgG1Fc

Fifteen micrograms of linearized expression vector pDSRα:huIGF1R(ECD)-C3-muIgG1Fc was transfected into AM-1/D CHOd-cells using LT1 lipofection reagent (PanVera Corp., Madison, Wis.), and cells cultured under conditions to allow expression and secretion of protein into the cell media. Twenty-four colonies were selected after 10-14 days on DHFR selection medium (Dulbecco's Modified Eagles Medium (Invitrogen) supplemented with 10% dialyzed fetal bovine serum, 1× penicillin-streptomycin (Invitrogen)) and expression levels evaluated by western blot. To perform this assay, 0.5 ml of serum free medium was added to a single well confluent cells cultured in a 24 well plate (Falcon). The conditioned medium was recovered after 48 hr. Samples for western blotting were run in 10% Tris-glycine gel (Novex), and blotted on 0.45 μm Nitrocellulose membrane (Invitrogen), using the Mini Trans-Blot cell (Biorad). The blotted membranes were incubated with rabbit anti-mouse IgG Fc antibody, conjugated with Horseradish Peroxidase (Pierce). The clone expressing the highest level of IGF-1R(ECD)-C3-muIgG1Fc was expanded in DHFR selection medium and 2×10⁷ cells were inoculated into 50 roller bottles each (Corning) in 250 ml of high-glucose DMEM (Invitrogen), 10% dialyzed FBS (Invitrogen), 1× glutamine (Invitrogen), 1× Non essential amino acids (Invitrogen), 1× sodium pyruvate (Invitrogen). Medium was gassed with 10% CO₂/balance air for 5 seconds before capping the roller bottle. Roller bottles were kept at 37° C. on roller racks spinning at 0.75 rpm.

When cells reached approximately 85-90% confluency (after approximately 5-6 days in culture), growth medium was discarded, cells washed with 100 ml PBS and 200 ml production medium was added (50 DMEM (Invitrogen)/50% F12 (Invitrogen), 1× glutamine (Invitrogen), 1× non-essential amino acids (Invitrogen), 1× sodium pyruvate (Invitrogen), 1.5% DMSO (Sigma)). The conditioned medium was harvested and replaced at one week intervals. The resulting 30 liters of conditioned medium were filtered through a 0.45 μm cellulose acetate filter (Corning, Acton, Mass.).

Purification of hu IGF-1R(ECD)-C3-muIgG1Fc

The resulting filtrate from the conditioned medium was concentrated 20-fold using a spiral-wound cartridge (molecular weight cut-off=10 kDa), then diluted 1:1 with 3 M KCl, 1 M glycine, pH 9.0 to bring the final salt concentration to 1.5 M KCl, 0.5 M glycine, pH 9.0. This sample was applied to a rProtein A-Sepharose column (Amersham Pharmacia Biotech, Uppsala, Sweden) which had been equilibrated in 1.5 M KCl, 0.5 M glycine, pH 9.0. The column was washed with 40 column volumes of the same buffer, then eluted with 20 column volumes of 0.1 M glycine-HCl, pH 2.8. Five-mL fractions were collected and immediately neutralized with 1 mL of 1 M Tris-HCl, pH 7.5. Fractions containing huIGF1R(ECD)-C3-muIgGFc were identified by SDS-PAGE, pooled, and dialyzed against phosphate-buffered saline. The yield was 2.4 mg/L of conditioned medium. The major protein species detected were the mature α and β chains and murine Fc, each of which appeared to be properly glycosylated based on their elevated and heterogeneous molecular weights. Unprocessed IGF-1R(ECD), as well as glycosylated but not proteolytically cleaved IGF-1R(CED), was also present in the preparation. The shift in bands to higher molecular weights under non-reducing conditions indicates that disulfide linkages joined the α □ and β chains. Amino-terminal sequencing of the final product indicated that 60% of the protein was correctly processed between the α- and β-chains of IGF-1R(ECD), while 40% remained unprocessed.

### Example 3

### Isolation of Human INSR(ECD)-muIgG1

This example presents a method of cloning and expressing a soluble fragment of the human insulin receptor.

Cloning of pDSRα:huINSR(ECD)-muIgG1Fc

Primers 2830-40:
5′ AGCAAGCTTCCACCATGGGCACCGGGGGCCGG 3′ SEQ ID NO:259

(Hind III site underlined) and 2830-41:
5′ ATTTGTCGACTTTTGCAATATTTGACGGGACGTCTAA 3′ SEQ ID NO:260
   (Sal I site underlined) were used to amplify the human INSR extracellular domain (1-929) from and INSR parental plasmid encoding the B form of the INSR splice variant (Ullrich et al., 1985, Nature 313:756-61; Ebina et al., 1985, Cell 40:747-58). The primers included a Kozak translation initiation sequence preceding the start codon and restriction sites for subsequent sub-cloning. PCR was performed on a PerkinElmer 2400 under the following conditions: 1 cycle at 95° C. for 2 min, 32 cycles at 95° C. for 30 sec, 58.5° C. for 30 sec, and 72° C. for 3 min, and 1 cycle at 72° C. for 10 min. Final reaction conditions were 1× pfu TURBO® buffer, 200 μM dNTPs, 2 μM each primer, 5 U pfu TURBO® (Stratagene) and 10 ng template DNA. The PCR product was purified using a NUCLEOSPIN® Column (BD Biosciences Clontech, Palo Alto, Calif.) according to the manufacturer's instructions, digested with Hind III and Sal I (Roche), and gel purified prior to ligation into Hind III/Sal I digested pDSRα-muIgG1. The integrity of the insert was confirmed by DNA sequencing. The protein sequence of the INSR-muFc is shown in FIG. 11. The final expression vector is described in Table 2.

TABLE 2

| Plasmid Base | |
|---|---|
| Pair Number: | |
| 11-3550 | HuINSR-muIgG1Fc |
| | atgggcaccgggggccggcggggggcggcggccgcgccgctgctggtggcggtggccgcgctg |
| | ctactgggcgccgcgggccacctgtaccccggagaggtgtgtcccggcatggatatccggaacaac |
| | ctcactaggttgcatgagctggagaattgctctgtcatcgaaggacacttgcagatactcttgatgttcaa |
| | aacgaggcccgaagatttccgagacctcagtttccccaaactcatcatgatcactgattacttgctgctct |
| | tccgggtctatgggctcgagagcctgaaggacctgttccccaacctcacggtcatccggggatcacga |
| | ctgttctttaactacgcgctggtcatcttcgagatggttcacctcaaggaactcggcctctacaacctgat |
| | gaacatcacccggggttctgtccgcatcgagaagaacaatgagctctgttacttggccactatcgactg |
| | gtcccgtatcctggattccgtggaggataatcacatcgtgttgaacaaagatgacaacgaggagtgtgg |
| | agacatctgtccgggtaccgcgaagggcaagaccaactgccccgccaccgtcatcaacgggcagtt |
| | gtcgaacgatgttggactcatagtcactgccagaaagtttgcccgaccatctgtaagtcacacggctgc |
| | accgccgaaggcctctgttgccacagcgagtgcctgggcaactgttctcagcccgacgaccccacca |
| | agtgcgtggcctgccgcaacttctacctggacggcaggtgtgtggagacctgcccgcccccgtacta |
| | ccacttccaggactggcgctgtgtgaacttcagcttctgccaggacctgcaccacaaatgcaagaactc |
| | gcggaggcagggctgccaccagtacgtcattcacaacaacaagtgcatccctgagtgtccctccggg |
| | tacacgatgaattccagcaacttgctgtgcaccccatgcctgggtccctgtcccaaggtgtgccacctc |
| | ctagaaggcgagaagaccatcgactcggtgacgtctgcccaggagctccgaggatgcaccgtcatc |
| | aacgggagtctgatcatcaacattcgaggaggcaacaatctggcagctgagctagaagccaacctcg |
| | gcctcattgaagaaatttcagggtatctaaaaatccgccgatcctacgctctggtgtcactttccttcttcc |
| | ggaagttacgtctgattcgagagagaccttggaaattgggaactactccttctatgccttggacaacca |
| | gaacctaaggcagctctgggactggagcaaacacaacctcaccaccactcaggggaaactcttcttcc |
| | actataaccccaaactctgcttgtcagaaatccacaagatggaagaagtttcaggaaccaaggggcgc |
| | caggagagaaacgacattgccctgaagaccaatggggacaaggcatcctgtgaaaatgagttactta |
| | aattttcttacattcggacatcttttgacaagatcttgctgagatgggagccgtactggccccccgacttcc |
| | gagacctcttggggttcatgctgttctacaaagaggccccttatcagaatgtgacggagttcgatgggc |
| | aggatgcgtgtggttccaacagttggacggtggtagacattgacccacccctgaggtccaacgacccc |
| | aaatcacagaaccacccagggtggctgatgcggggtctcaagccctggacccagtatgccatctttgt |
| | gaagaccctggtcaccttttcggatgaacgccggacctatggggccaagagtgacatcatttatgtcca |
| | gacagatgccaccaacccctctgtgcccctggatccaatctcagtgtctaactcatcatcccagattattc |
| | tgaagtggaaaccaccctccgaccccaatggcaacatcacccactacctggttttctgggagaggcag |
| | gcggaagacagtgagctgttcgagctggattattgcctcaaagggctgaagctgccctcgaggacctg |
| | gtctccaccattcgagtctgaagattctcagaagcacaaccagagtgagtatgaggattcggccggcg |
| | aatgctgctcctgtccaaagacagactctcagatcctgaaggagctggaggagtcctcgtttaggaag |
| | acgtttgaggattacctgcacaacgtggttttcgtccccagaaaaacctcttcaggcactggtgccgag |
| | gaccctaggccatctcggaaacgcaggtcccttggcgatgttgggaatgtgacggtggccgtgccca |
| | cggtggcagctttccccaacacttcctcgaccagcgtgcccacgagtccggaggagcacaggcctttt |
| | gagaaggtggtgaacaaggagtcgctggtcatctccggcttgcgacacttcacgggctatcgcatcga |
| | gctgcaggcttgcaaccaggacacccctgaggaacggtgcagtgtggcagcctacgtcagtgcgag |
| | gaccatgcctgaagccaaggctgatgacattgttggccctgtgacgcatgaaatctttgagaacaacgt |
| | cgtccacttgatgtggcaggagccgaaggagcccaatggtctgatcgtgctgtatgaagtgagttatcg |
| | gcgatatggtgatgaggagctgcatctctgcgtctcccgcaagcacttcgctctggaacggggctgca |
| | ggctgcgtgggctgtcaccggggaactacagcgtgcgaatccgggccacctcccttgcgggcaacg |
| | gctcttggacggaacccacctatttctacgtgacagactatttagacgtcccgtcaaatattgcaaaagtc |
| | gacggttgtaagccttgcatatgtacagtcccagaagtatcatctgtcttcatcttccccccaaagcccaa |
| | ggatgtgctcaccattactctgactcctaaggtcacgtgttgtggtagacatcagcaaggatgatccc |
| | gaggtccagttcagctggtttgtagatgatgtggaggtgcacacagctcagacgcaaccccgggagg |
| | agcagttcaacagcactttccgctcagtcagtgaacttcccatcatgcaccaggactggctcaatggca |
| | aggagttcaaatgcagggtaaacagtgcagctttccctgcccccatcgagaaaaccatctccaaaacc |
| | aaaggcagaccgaaggctccacaggtgtacaccattccacctcccaaggagcagatggccaaggat |
| | aaagtcagtctgacctgcatgataacagacttcttccctgaagacattactgtggagtggcagtggaatg |
| | ggcagccagcggagaactacaagaacactcagcccatcatggacacagatggctcttacttcgtctac |
| | agcaagctcaatgtgcagaagagcaactgggaggcaggaaatactttcacctgctctgtgttacatga |
| | gggcctgcacaaccaccatactgagaagagcctctcccactctcctggtaaa (SEQ ID NO: 261) |
| 3557 to 4441 | A transcription termination/polyadenylation signal from the α-subunit of |
| | the bovine pituitary glycoprotein hormone (α-FSH) (Goodwin et al., 1983, |
| | Nucleic Acids Res. 11:6873-82; Genbank Accession Number X00004) |
| 4446 to 5586 | A mouse dihydrofolate reductase (DHFR) minigene containing the endogenous |
| | mouse DHFR promoter, the cDNA coding sequences, and the DHFR transcription |
| | termination/polyadenylation signals (Gasser et al., 1982, Proc. Natl. |
| | Acad. Sci. U.S.A. 79:6522-6; Nunberg et al., 1980, Cell 19:355-64; |
| | Setzer et al., 1982, J. Biol. Chem. 257:5143-7; McGrogan et al., |
| | 1985, J. Biol. Chem. 260:2307-14) |
| 5594 to 6241 | pBR322 sequences containing the ampicillin resistance marker gene and |
| | the origin for replication of the plasmid in E. coli (Genbank Accession |
| | Number J01749) |
| 7513 to 7856 | An SV40 early promoter, enhancer and origin of replication (Takebe et |
| | al., 1988, Mol. Cell Biol. 8:466-72, Genbank Accession Number J02400) |
| 7863 to 8119 | A translational enhancer element from the HTLV-1 LTR domain |
| | (Seiki et al., 1983, Proc. Natl. Acad. Sci. U.S.A. 80:3618-22, Genbank |
| | Accession Number J02029) |
| 8163 to 8259 | An intron from the SV40 16S, 19S splice donor/acceptor signals |
| | (Okayama and Berg, 1983. Mol. Cell Biol. 3:280-9, Genbank Accession |
| | Number J02400) |

Expression of hu INSR(ECD)-C3-muIgG1Fc

AM-1/D CHOd-cells were transfected with 15 μm of linearized expression vector pDSRa:huINSR(ECD)-muIgG1Fc using FUGENE™ 6 lipofection reagent (Roche Diagnostics Corp., Indianapolis, Ind.), then cultured under conditions to allow expression and secretion of protein into the cell medium. Colonies were selected and analyzed as described above.

Purification of hu INSR(ECD)-C3-muIgG1Fc

The filtered conditioned medium containing huINSR(ECD)-muIgGFc was concentrated 17-fold using a spiral-wound cartridge (molecular weight cut-off=10 kDa), then diluted 1:1 with 3 M KCl, 1 M glycine, pH 9.0 to bring the final salt concentration to 1.5 M KCl, 0.5 M glycine, pH 9.0. This sample was applied to a rProtein A-Sepharose column (Pharmacia) which had been equilibrated in 1.5 M KCl, 0.5 M glycine, pH 9.0. The column was washed with 40 column volumes of the same buffer, then eluted with 20 column volumes of 0.1 M glycine-HCl, pH 2.8. Five-mL fractions were collected and immediately neutralized with 1-mL of 1 M Tris-HCl, pH 7.5. Fractions containing huINSR(ECD)-muIgGFc were identified by SDS-PAGE, pooled, and dialyzed against phosphate-buffered saline. The yield was 0.9 mg/L of conditioned medium. The major protein species were the mature α and β chains and murine Fc. Each of these species appeared to be properly glycosylated based on its elevated and heterogeneous molecular weight. Unprocessed INSR (ECD) as well as glycosylated but not proteolytically cleaved INSR (CED) also was present in the preparation. The shift in bands to higher molecular weights under non-reducing conditions indicated that disulfide linkages joined the α □ and β chains. Amino-terminal sequencing of the final product indicated that 87% of the protein was correctly processed between the α- and β-chains of INSR(ECD), while 13% remained unprocessed.

### Example 3

### Initial Screen for Anti-IGF-1R Phage Fab

This example provides a method of identifying anti-IGF-1R antibodies.

A Target Quest Q Fab library (“the TQ library”; Target Quest, Maastricht, the Netherlands), which was constructed using peripheral blood lymphocytes from four healthy donors and splenic lymphocytes from one patient with gastric carcinoma, was obtained. The library diversity was 3.7×10¹⁰ clones, containing 3×10⁹ heavy chains. The source, screening methods, and characterization of the library have been published (de Haard et al, 1999, J Biol Chem 274:18218-30). Dynabeads (200 μl) M-450 Uncoated (catalog #140.02, Dynal, Lake Success, N.Y.) were washed 3 times with PBS, resuspended in 200 μl of IGF1R(ECD)-C3-mFc to a concentration of 0.5 μM in PBS, and incubated at 4° C. on a rotator overnight. The IGF-1R(ECD)-C3-mFc coated beads were washed 3× with 1 ml of 2% non-fat dry milk (M) in PBS (2% MPBS), and then blocked with 1 ml of 2% MPBS at room temperature for 1 hour. In parallel, 750 μA of the TQ library (4×10¹² pfu) was preblocked by mixing with 250 μl 8% MPBS at room temperature for 30 minutes to 1 hour. 500 μl of blocked beads were transferred into another microfuge tube and separated from the blocking solution on a magnetic separator. The preblocked phage mixture was added to the blocked beads and incubated for 90 minutes on a rotator at room temperature. Bead-bound phage were separated from the unbound phage, and then washed 6× with 1 ml 2% MPBS/0.1% Tween 20, 6× with 1 ml PBS/0.1% Tween 20, 2× with PBS with a change of tubes between different wash solutions. Bound phage was eluted with 1 ml of 0.1M TEA (pH11) for 10 minutes, then immediately separated from the beads and neutralized with 0.5 ml of 1 M Tris.HCl. The eluted phage pool was mixed with 4 ml 2×YT broth (10 g yeast extract, 16 g bacto-tryptone, 5 g NaCl per liter of water) and 5 ml of TG1 bacterial culture (O.D.₅₉₀ about 0.5) in a 50-ml conical tube. The infection mixture was incubate at 37° C. in an incubator for 30 min., then centrifuged at 3500 rpm for 20 min. The cell pellet was resuspended in 1500 μl 2xYT-CG broth and 300 μl were spread on each of five 2xYT-CG (2×YT broth containing 100 μg/ml carbenicillin and 2% glucose) plates. After 20 hours of incubation at 30° C., 4 ml of 2×YT-AG were added to each plate and the cells were recovered with cell scraper from the plates. This step was repeated three times. A small portion of the recovered cells was used for phage rescue (see below). The remaining cell suspension was centrifuged at 3500 rpm for 20 min. The cell pellet was suspended into an amount of 50% glycerol roughly half the volume of the pellet size and stored at −80° C.

In order to rescue phage, the plated-amplified cell suspension was used to inoculate

ml of 2×YT-CG to an OD₅₉₀ of about 0.05. The culture was incubated at 37° C. on a shaker to OD₅₉₀ 0.5. The log phase culture was infected with M13KO7 helper phage (GIBCO BRL, Gaithersburg, Md., catalog #18311-019, 1.1×10¹¹ pfu/ml) at M.O.I. 20 followed by incubation at 37° C. for 30 min. The infected cells were centrifuged at 4000 rpm for 20 min. The cell pellet was re-suspended in 200 ml of 2xYT-CK (100 μg/ml carbenicillin and 40 μg/ml kanamycin) and transferred to two 250-ml flasks and incubated at 30° C. with shaking at 270 rpm for 20 hours. The over-night culture was centrifuged at 4000 rpm for 20 min to removal cell debris. The centrifugation was repeated to ensure the removal of cell debris. About ⅕ volume of PEG solution (20% PEG 8000, 2.5 M NaCl) was added to the supernatant 63 precipitate the phage particles. The mixture was incubated on ice for at least 1 hour, followed by centrifugation at 4000 rpm for 20 min to collect the precipitated phage particles. The phage pellet was re-suspended into 1 ml of PBS and transferred to a microfuge tube. The phage suspension was left on ice for 1 hour to allow complete suspension of phage particles, and clarified by centrifugation at 14,000 rpm for 2 min to remove the residual cell debris. Phage precipitation step was repeated. The final phage pellet was suspended into PBS after clarification. The rescued phage suspension was used in the next round of selection.

Four rounds of selection were performed that included alterations of various standard binding parameters. The second round of selection was identical to the first round of selection. Variations in input phage number and elution reagent were introduced in rounds three and four. For the round three selection, 5×10¹¹ pfu of phages were selected and bound phages were eluted either with 1 μM IGF-1 (catalog #I3769, Sigma, St. Louis, Mo.) or with a 1 μM concentration of a chimeric αIR3-huFc antibody to yield two round-three pools, TQ4-3IS and TQ4-3CA. Round four selection was carried out on rescued phage pools from both round three pools. Two rounds of negative selection with mouse IgG Fc-coated DYNABEADS® (Dynal Biotech, Oslo, Norway) were included to remove mouse Fc binders prior to actual IGF-1R selection. The incubation time for negative selection was 30 minutes each. 3.78×10¹¹ pfu of TQ4-3IS pool and 3.75×10¹² pfu of TQ4-3CA pool were selected separately. Bound phage were eluted with 1 μM IGF-2 (catalog # I2526, Sigma, St. Louis, Mo.) to yield two round-4 pools, TQ4-4ISI2 and TQ4-4CAI2. The sequence of about 96-192 phage DNA inserts was determined at each elution step.

In some cases, a secondary screen was done. Phagemid DNA mixtures of the total TQ library, and the selected phage amplified after several rounds of selection against IGF-1R, were prepared using a DNA Maxiprep kit according to the manufacturer's instructions (Qiagen, Valencia, Calif.). All four DNA preparations were digested with Asc I and EcoR (New England Biolab, Beverly, Mass.). The resulting two Asc I/EcoR I fragments were separated on preparative 0.5% agarose gels. The 2.1 kb fragments containing heavy chains were gel purified from the IGF-1R selected phage. The 3.9 kb fragments containing the light chains and pCES1 vector portion were gel purified from the total TQ library DNA. The 2.1 kb fragments were ligated to the 3.9 kb fragments from the DNA sample of TQ library in 3:1 ratio. The ligated DNA was precipitated and used to transform TG1 cells by electroporation. The library size of the resulted light chain shuffled secondary library was 8.8×10⁸. After sequencing 96 randomly picked clones, 76 unique light chain sequences were obtained, indicating that the attempt to shuffle light chains was successful.

The binding, washing and elution condition for screening the light chain shuffle library were essentially the same as described for the initial screen. However, several variations were included to increase selection pressure for amplification of IGF-1R binders with higher affinities, especially those with significantly slower off-rates. These parameters were: higher number of input phage (2−2.7×10¹³ pfu), smaller bead volume (100 μl for round one, 50 μl for round two, and 25 μl for round three), and extended specific elution time up to 20 hours. Elution buffers were 0.1 M TEA for round one (RD1), 1 μM IGF-1 in 0.4% MPBS for RD2 and 1 μM IGF-1 or IGF-2 in 0.4% MPBS for RD3. In RD2 and RD3, binders that were eluted in 15 min or 2 hours were discarded. Elution was continued and eluted phages were collected after 8-10 hours and again after 20 hours.

### Phage Fab ELISA Screen

In 96-well 2-ml deep-well blocks, 480 μl/well 2xYT-CG broth was inoculated with 20 μl of overnight cultures of the individual clones, then incubated at 37° C., 300 rpm for 3 hours. To each well, 50 μl of 1:3 diluted M13KO7 helper phage were added to infect the cells. The block was incubated at 37° C. without shaking for 30 minutes, and then shaken gently for another 30 minutes at 150 rpm. The block was centrifuged at 3600 rpm for 20 minutes to pellet the infected cells. The cell pellet in each well was suspended into 480 μl of 2xYT-CK (2xYT broth containing 100 μg/ml carbenicillin and 40 μg/ml kanamycin), and incubated at 30° C. overnight for about 20 hours. The cell debris was separated by centrifugation at 3600 rpm for 20 minutes. The rescued phage supernatant was used in the phage ELISA to check for IGF-1R-specific, INSR-cross reactive, or mouse Fc binding of individual clones.

Three sets of Nunc MaxiSorb Immunoplates were coated with 100 μl/well of IGF-1R-C3-mFc at 5 μg/ml, INSR-mFc at 5 μg/ml, or mouse IgG1 (catalog #010-0103, Rockland, Gilbertsville, Pa.) at 2 μg/ml in PBS, respectively, at 4° C. overnight. The coated plates were washed 3× with 300 μl/well of PBS. The washed plates were blocked with 300 μl/well 2% MPBS at room temperature for one hour. Meanwhile, rescued phages of individual clones were pre-blocked by mixing 170 μl of rescued phage with 170 μl of 4% MPBS. The blocked plates were washed 5× with 300 μl/well TBST (TBS: 10 mM Tris-HCl, pH 7.5, 1 mM EDTA, 150 mM NaCl; Tween-20. 0.1%). 100 μl/well of pre-blocked phage dilutions were distributed to each set of coated plate, which were incubated at room temperature on a rocker for 90 minutes. The plates were washed 5× with 300 μl/well TBST. 100 μl/well of anti-M13-HRP in 2% MPBS (1:3000 dilution, catalog number 27-9421-01, Amersham Pharmacia Biotech) were distributed, and plates were incubated at room temperature on rocker for one hour. The plates were washed 5× with 300 μl/well TBST. 100 μl/well of the substrate 1-Step™ ABTS (Pierce Biotechnology, Rockford, Ill., catalog number 37615) were added. Plates were incubated for one hour. OD₄₀₅ was measured for signal detection.

The phage displayed antibodies exhibited essentially no crossreactivity with the insulin receptor and murine Fc domain. The signal observed in the IGF-1R ELISA is therefore specific for the IGF-1R extracellular domain. Results from similar assays for four of the phage-displayed antibodies are shown in FIG. 14.

The DNA inserts of IGF-1R positive, INSR and mu IgG1 negative, clones were sequenced. Fifty-two unique Fab sequences were identified, having the following combinations of light chain and heavy chain variable domain sequences: L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, and L52H52, wherein “Lx” indicates light chain variable domain number “x” and “Hx” indicates heavy chain variable domain number “x.” FIG. 1 presents the polynucleotide sequences of each of these light and heavy variable domains. FIGS. 2 and 3 present the corresponding amino acid sequences.

### Example 4

### Subcloning of V_{H} and V_{L} into IgG1 Expression Vectors

This example presents a method of subcloning the previously identified variable domain sequences into an IgG 1 expression vector.

Construction of pDSRα20 and pDSRα20:hIgG1C_{H}

The pDSRα20:hIgG1C_{H} expression vector (WO 90/14363) was a derivative of pDSR19:hIgG1C_{H} (see U.S. Provisional Patent Application No. 60/370,407, filed Apr. 5, 2002, “Human Anti-OPGL Neutralizing Antibodies As Selective OPGL Pathway Inhibitors,” incorporated herein by reference in its entirety). The pDSRα19:hIgG1C_{H} plasmid encoded a rat variable region/human constant region IgG1 (rVh/hCh1). The plasmid was constructed by the three-piece ligation of Xba I and BsmB I terminated rat antibody variable region PCR product, the human IgG1 constant region (C_{H1}, hinge, C_{H2} and C_{H3} domains) derived by Sal I cleavage and gel isolation of the BsmB I and Sal I fragment from the linear plasmid pDSRα19:hIgG1C_{H} (Hind III and BsmB I ends) and a linearized pDSRα19 with Xba I and Sal I ends. pDSRα20 was produced by changing nucleotide 2563 in pDSRα19 from a guanosine to an adenosine by site directed mutagenesis. The heavy chain expression vector, pDSRα20:hIgG1C_{H} rat variable region/human constant region IgG1 (rVh/hCh1), is 6163 base pairs and contains the 7 functional regions described in Table 3.

TABLE 3

| Plasmid Base | |
|---|---|
| Pair Number: | |
| 2 to 881 | A transcription termination/polyadenylation signal from the α-subunit |
| | of the bovine pituitary glycoprotein hormone (α-FSH) (Goodwin et al., |
| | 1983, Nucleic Acids Res. 11:6873-82; Genbank Accession Number |
| | X00004) |
| 882 to 2027 | A mouse dihydrofolate reductase (DHFR) minigene containing the |
| | endogenous mouse DHFR promoter, the cDNA coding sequences, and |
| | the DHFR transcription termination/polyadenylation signals (Gasser et |
| | al., 1982, Proc. Natl. Acad. Sci. U.S.A. 79:6522-6; Nunberg et al., |
| | 1980, Cell 19:355-64; Setzer et al., 1982, J. Biol. Chem. 257:5143-7; |
| | McGrogan et al., 1985, J. Biol. Chem. 260:2307-14) |
| 2031 to 3947 | pBR322 sequences containing the ampicillin resistance marker gene and |
| | the origin for replication of the plasmid in E. coli (Genbank |
| | Accession Number J01749) |
| 3949 to 4292 | An SV40 early promoter, enhancer and origin of replication |
| | (Takebe et al., 1988, Mol. Cell Biol. 8:466-72, Genbank Accession |
| | Number J02400) |
| 4299 to 4565 | A translational enhancer element from the HTLV-l LTR domain |
| | (Seiki et al., 1983, Proc. Natl. Acad. Sci. U.S.A. 80:3618-22, |
| | Genbank Accession Number J02029) |
| 4574 to 4730 | An intron from the SV40 16S, 19S splice donor/acceptor signals |
| | (Okayama and Berg, 1983. Mol. Cell Biol. 3:280-9, Genbank Accession |
| | Number J02400) |
| 4755 to 6158 | The rVh/hCh1 heavy chain cDNA between the Xbal and Sall sites. This |
| | heavy chain fragment sequence is shown below (SEQ ID NO: 262) with |
| | the sequences of the restriction sites underlined: |
| | XbaI |
| | TCTAG ACCACCATGG ACATCAGGCT CAGCTTAGTT |
| | TTCCTTGTCC TTTTCATAAA AGGTGTCCAG TGTGAGGTAG |
| | AACTGGTGGA GTCTGGGGGC GGCTTAGTAC AACCTGGAAG |
| | GTCCATGACA CTCTCCTGTG CAGCCTCGGG ATTCACTTTC |
| | AGAACCTATG GCATGGCCTG GGTCCGCCAG GCCCCAACGA |
| | AGGGTCTGGA GTGGGTCTCA TCAATTACTG CTAGTGGTGG |
| | TACCACCTAC TATCGAGACT CCGTGAAGGG CCGCTTCACT |
| | ATTTTTAGGG ATAATGCAAA AAGTACCCTA TACCTGCAGA |
| | TGGACAGTCC GAGGTCTGAG GACACGGCCA CTTATTTCTG |
| | TACATCAATT TCGGAATACT GGGGCCACGG AGTCATGGTC |
| | BsmB1 |
| | ACCGTCTCTA GTGCCTCCAC CAAGGGCCCA TCGGTCTTCC |
| | CCCTGGCACC CTCCTCCAAG AGCACCTCTG GGGGCACAGC |
| | GGCCCTGGGC TGCCTGGTCA AGGACTACTT CCCCGAACCG |
| | GTGACGGTGT CGTGGAACTC AGGCGCCCTG ACCAGCGGCG |
| | TGCACACCTT CCCGGCTGTC CTACAGTCCT CAGGACTCTA |
| | CTCCCTCAGC AGCGTGGTGA CCGTGCCCTC CAGCAGCTTG |
| | GGCACCCAGA CCTACATCTG CAACGTGAAT CACAAGCCCA |
| | GCAACACCAA GGTGGACAAG AAAGTTGAGC CCAAATCTTG |
| | TGACAAAACT CACACATGCC CACCGTGCCC AGCACCTGAA |
| | CTCCTGGGGG GACCGTCAGT CTTCCTCTTC CCCCCAAAAC |
| | CCAAGGACAC CCTCATGATC TCCCGGACCC CTGAGGTCAC |
| | ATGCGTGGTG GTGGACGTGA GCCACGAAGA CCCTGAGGTC |
| | AAGTTCAACT GGTACGTGGA CGGCGTGGAG GTGCATAATG |
| | CCAAGACAAA GCCGCGGGAG GAGCAGTACA ACAGCACGTA |
| | CCGTGTGGTC AGCGTCCTCA CCGTCCTGCA CCAGGACTGG |
| | CTGAATGGCA AGGAGTACAA GTGCAAGGTC TCCAACAAAG |
| | CCCTCCCAGC CCCCATCGAG AAAACCATCT CCAAAGCCAA |
| | AGGGCAGCCC CGAGAACCAC AGGTGTACAC CCTGCCCCCA |
| | TCCCGGGATG AGCTGACCAA GAACCAGGTC AGCCTGACCT |
| | GCCTGGTCAA AGGCTTCTAT CCCAGCGACA TCGCCGTGGA |
| | GTGGGAGAGC AATGGGCAGC CGGAGAACAA CTACAAGACC |
| | ACGCCTCCCG TGCTGGACTC CGACGGCTCC TTCTTCCTCT |
| | ATAGCAAGCT CACCGTGGAC AAGAGCAGGT GGCAGCAGGG |
| | GAACGTCTTC TCATGCTCCG TGATGCATGA GGCTCTGCAC |
| | AACCACTACA CGCAGAAGAG CCTCTCCCTG TCTCCGGGTA |
| | SalI |
| | AATGATAAGT CGAC |

The linear plasmid pDSRα20:hIgG1C_{H} was prepared by digesting the pDSR20: rat variable region/human constant region IgG1 plasmid with the restriction enzymes Xba I and BsmB I to remove the rat variable region and purified using a QIAquick Gel Extraction kit. The linear plasmid pDSRα20:hIgG1C_{H} containing the 1.0 kbp human IgG1 constant region domain was used to accept anti-IGF-1R variable heavy chain coding sequences.

Construction of the anti-IGF-1R IgG1 Heavy Chain Expression Clones

The sequence coding for the anti-IGF-1R variable region of the heavy chains was amplified from phagemid DNA with complementary oligonucleotide primers. Primers for polymerase chain reaction (PCR) were designed to incorporate a Hind III site, Xba I site, Kozak sequence (CCACC) and signal sequence (translated peptide is MDMRVPAQLLGLLLLWLRGARC; SEQ ID NO:263) onto the 5′ end of the variable region, while a BsmB I site was added onto the 3′ end of the PCR product. The PCR products were digested with Xba I and BsmB I, and then cloned into the Xba I-BsmB I linear pDSRα20:hIgG1C_{H} expression vector containing the human IgG1 constant region (FIG. 13). The final expression vectors contained the seven functional regions described in Table 4.

TABLE 4
Plasmid Base

| Pair Number: | |
|---|---|
| 2 to 881 | A transcription termination/polyadenylation signal from the α-subunit of |
| | the bovine pituitary glycoprotein hormone (α-FSH) (Goodwin et al., |
| | 1983, Nucleic Acids Res. 11: 6873-82; Genbank Accession Number |
| | X00004) |
| 882 to 2027 | A mouse dihydrofolate reductase (DHFR) minigene containing the |
| | endogenous mouse DHFR promoter, the cDNA coding sequences, and |
| | the DHFR transcription termination/polyadenylation signals (Gasser et |
| | al., 1982, Proc. Natl. Acad. Sci. U.S.A. 79: 6522-6; Nunberg et al., |
| | 1980, Cell 19: 355-64; Setzer et al., 1982, J. Biol. Chem. 257: 5143-7; |
| | McGrogan et al., 1985, J. Biol. Chem. 260: 2307-14) |
| 2031 to 3947 | pBR322 sequences containing the ampicillin resistance marker gene and |
| | the origin for replication of the plasmid in E. coli (Genbank Accession |
| | Number J01749) |
| 3949 to 4292 | An SV40 early promoter, enhancer and origin of replication (Takebe et |
| | al., 1988, Mol. Cell Biol. 8: 466-72, Genbank Accession Number |
| | J02400) |
| 4299 to 4565 | A translational enhancer element from the HTLV-1 LTR domain |
| | (Seiki et al., 1983, Proc. Natl. Acad. Sci. U.S.A. 80: 3618-22, Genbank |
| | Accession Number J02029) |
| 4574 to 4730 | An intron from the SV40 16S, 19S splice donor/acceptor signals |
| | (Okayama and Berg, 1983. Mol. Cell Biol. 3: 280-9, Genbank Accession |
| | Number J02400) |
| 4755 to 6185 | The heavy chain IgG1 cDNA between the Xbal and Sall sites |

### Construction of the Anti-IGF-1R IgG1 Variable Chain Expression Clones.

The light chains encoded in anti-IGF-1R phage were either kappa or lambda class. They were cloned using one of two approaches. Complementary primers were designed to add a Hind III site, an Xba I site, Kozak sequence (CCACC) and signal sequence (translated peptide is MDMRVPAQLLGLLLLWLRGARC, SEQ ID NO:264) were added to the 5′ end of the coding region. Those chains that had error-free coding regions were cloned as full-length products. The full-length light chains were cloned as Xba I and Sal I fragments into the expression vector pDSRα20. The final expression vectors contained the seven functional regions described in Table 5.

TABLE 5
Plasmid Base

| Pair Number: | |
|---|---|
| 2 to 881 | A transcription termination/polyadenylation signal from the α-subunit of |
| | the bovine pituitary glycoprotein hormone (α-FSH) (Goodwin et al., |
| | 1983, Nucleic Acids Res. 11: 6873-82; Genbank Accession Number |
| | X00004) |
| 882 to 2027 | A mouse dihydrofolate reductase (DHFR) minigene containing the |
| | endogenous mouse DHFR promoter, the cDNA coding sequences, and |
| | the DHFR transcription termination/polyadenylation signals (Gasser et |
| | al, 1982, Proc. Natl. Acad. Sci. U.S.A. 79: 6522-6; Nunberg et al., 1980, |
| | Cell 19: 355-64; Setzer et al., 1982, J. Biol. Chem. 257: 5143-7; |
| | McGrogan et al., 1985, J. Biol. Chem. 260: 2307-14) |
| 2031 to 3947 | pBR322 sequences containing the ampicillin resistance marker gene and |
| | the origin for replication of the plasmid in E. coli (Genbank Accession |
| | Number J01749) |
| 3949 to 4292 | An SV40 early promoter, enhancer and origin of replication (Takebe et |
| | al., 1988, Mol. Cell Biol. 8: 466-72, Genbank Accession Number |
| | J02400) |
| 4299 to 4565 | A translational enhancer element from the HTLV-1 LTR domain |
| | (Seiki et al., 1983, Proc. Natl. Acad. Sci. U.S.A. 80: 3618-22, Genbank |
| | Accession Number J02029) |
| 4574 to 4730 | An intron from the SV40 16S, 19S splice donor/acceptor signals |
| | (Okayama and Berg, 1983, Mol. Cell Biol. 3: 280-9, Genbank Accession |
| | Number J02400) |
| 4755 to 5485 | The kappa light chain cDNA between the Xbal and Sall sites |

Some kappa clones had errors in their constant regions when compared to natural human constant region sequence. To eliminate these discrepancies, the kappa variable region was amplified with a primer that would introduce an Xba I site into the 5′ end and a BsmB I site into the 3′ end. This fragment was then ligated along with a human kappa constant region (FIG. 13) with a compatible BsmB I on the 5′ end and a 3′Sal I ends into pDSRα20 with Xba I and Sal I ends.

### Example 5

### Transient Expression of Antibodies

This example provides a method of transiently expressing anti-IGF-1R antibodies.

The antibodies were expressed transiently in serum-free suspension adapted 293T cells. All transfections were performed as 250 mL cultures. Briefly, 1.25×10⁸ cells (5.0×10⁵ cells/mL×250 mL) were centrifuged at 2,500 RPM for 10 minutes at 4° C. to remove the conditioned medium. The cells were resuspended in serum-free DMEM and centrifuged again at 2,500 RPM for 10 minutes at 4° C. After aspirating the wash solution, the cells were resuspended in growth medium [DMEM/F12 (3:1)+1× Insulin-Transferrin-Selenium Supplement+1× Pen Strep Glut+2 mM L-Glutamine+20 mM HEPES+0.01% Pluronic F68] in a 500 mL spinner flask culture. The spinner flask culture was maintained on magnetic stir plate at 125 RPM which was placed in a humidified incubator maintained at 37° C. and 5% CO₂. The plasmid DNA was incubated with the transfection reagent in a 50 mL conical tube. The DNA-transfection reagent complex was prepared in 5% of the final culture volume in serum-free DMEM. One microgram of plasmid DNA per milliliter of culture was first added to serum-free DMEM, followed by 1 μl X-TremeGene RO-1539/mL culture. The complexes were incubated at room temperature for approximately 30 minutes and then added to the cells in the spinner flask. The transfection/expression was performed for 7 days, after which the conditioned medium was harvested by centrifugation at 4,000 RPM for 60 minutes at 4° C.

If the initial transfection failed to yield the required 100 μg purified antibody, those clones were re-expressed in roller bottles. These transfections used 293T adherent cells grown and maintained in DMEM supplemented with 5% FBS+1× Non-Essential Amino Acids+1× Pen Strep Glut+1× Sodium Pyruvate. Approximately, 4−5×10⁷ 293T cells were seeded in a 850 cm² roller bottles overnight. The previously seeded cells were then transfected the following day using FUGENE™ 6 transfection reagent. The DNA-transfection reagent mixture was prepared in approximately in 6.75 mL serum-free DMEM. 675 μl FUGENE™ 6 transfection reagent was first added, followed by 112.5 μg plasmid DNA. The complex was incubated at room temperature for 30 minutes. The entire mixture was then added to a roller bottle. The roller bottle was infused with a 5% CO₂ gas mixture, capped tightly and placed in a 37° C. incubator on a roller rack rotating at 0.35 RPM. The transfection was performed for 24 hours after which the medium was replaced with 100 mL DMEM+1× Insulin-Transferrin-Selenium Supplement+1× Pen Strep Glu+1× Non-Essential Amino Acids+1× Sodium Pyruvate. Typically, 2-3 harvests (100 ml) were obtained from each roller bottle at a 48 hr interval. The harvested serum-free conditioned medium was pooled together and centrifuged at 4,000 RPM for 30 minutes at 4° C.

### Example 6

### Anti-IGF-1R Antibody Small-Scale Purification

This example provides a method of purifying anti-IGF-1R antibodies on a small scale. Conditioned medium was filtered through a 0.45 μm cellulose acetate filter and concentrated approximately 8-fold using a Vivaflow 200 50 K tangential flow membrane (Vivascience, Goettingen, Germany). rProtein A SEPHAROSE™ Fast Flow resin (Amersham Biosciences, Piscataway, N.J.) was washed with phosphate buffered saline (2.7 mM potassium chloride, 138 mM sodium chloride, 1.5 mM potassium phosphate, and 8.1 mM sodium phosphate, pH 7.4) (PBS) four times then directly applied to the concentrated media. The amount of resin used was based on antibody concentration determined by ELISA where 1 μl of resin was used per 5 μg antibody. The medium was incubated overnight at 4° C. with gentle agitation. The resin was centrifuged at 500 g for 10 min. at 4° C. The supernatant was decanted as the unbound fraction. The resin was washed with PBS four times for one minute at room temperature with gentle agitation, each time collecting the resin by centrifugation at 500 g for 10 min. at 4° C. The antibody was eluted by incubating the resin with 1.5 volumes of 0.1 M glycine pH 3.0 for 10 min. at room temperature. The resin was centrifuged at 500 g for 10 min. at 4° C. and the supernatant decanted as eluted antibody. The elution step described above was repeated for a total of three elutions; each time the eluted material was neutralized with 0.04 volumes of 1.0 M tris-HCl, pH 9.2. The sample was filtered through a 0.2 μM cellulose acetate filter. Protein concentration was determined by the Bradford method using the Bio-Rad Protein Assay (Bio-Rad Laboratories, Hercules, Calif.) as per the supplied instructions using Human IgG (Sigma-Aldrich, St. Louis, Mo.) as a standard. The sample was compared to a Human IgG1, K standard (Sigma-Aldrich, St. Louis, Mo.) using a 4-20% tris-glycine SDS polyacrylamide gel (SDS-PAGE) gel stained with Coomassie brilliant blue dye. No contaminating protein was visible in these preparations.

### Example 7

### Isolation of Stable CHO Clones Expressing Antibodies

This example provides a method for isolating stable CHO cell lines expressing anti-IGF-1R antibodies.

Stable expression of TQ11C, TQ25, TQ 58 and TQ59 IgG1 was achieved by co-transfection of AM1-D CHO cells (U.S. Pat. No. 6,210,924, incorporated herein by reference in its entirety) with pDSRα20 heavy and light chain IgG1 expression constructs. The plasmid transfections were performed using LF2000 (Invitrogen, Carlsbad, Calif.) according to the manufacturer's instructions. Briefly, 4×106AM1-D CHO cells were plated 24 hours prior to transfection, in 100 mm diameter FALCON™ plastic petri dishes (BD Falcon, Franklin Lakes, N.J.) in 10 ml of Dulbecco's Modified Eagles Medium (Invitrogen) supplemented with 5% fetal bovine serum, 1× penicillin-streptomycin and glutamine (Invitrogen), non-essential amino acids (Invitrogen), sodium pyruvate, and HT (0.1 mM sodiumhypoxanthine, 16 nM thymidine; Invitrogen). Approximately 15 mg of each pDSRα21-light chain and heavy chain plasmid DNA were linearized using Pvu I (New England Biolabs) and diluted in 2 ml of OPTI-MEM® (Invitrogen). The diluted plasmids were mixed with 75 μl of LIPOFECTAMINE™ 2000 (LF2000; GIBCO/BRL) diluted in 2 ml of OPTI-MEM® and the mixture was incubated for 20 min at room temperature. The following day fresh growth medium was added. The cells were cultured in complete growth medium for 48 hours, then plated in HT-selection medium in 1:20 and 1:50 dilutions. Approximately 2 weeks after transfection, 12-24 visible colonies were picked into 24-well plates, using the sterile cloning discs (RPI). The clones expressing the highest level of TQ11C, TQ25, TQ58 and TQ59 IgG1 were identified by western immunoblot analysis. To perform this assay, 0.5 ml of serum free medium was added to a single-well confluent cells cultured in a 24 well plate (BD Falcon). The conditioned medium was recovered after 24 hr, and 10 μl of CM was mixed with an equal volume of loading buffer to run a 10% Tris-Glycine polyacrylamide protein gel (Invitrogen). The gel was transferred to a 0.45 μm pore size nitrocellulose membrane (Invitrogen), and western blot analysis was done using 1:1000 dilution of goat anti-human IgG Fc ImmunoPure antibody (Pierce Biotechnology, Inc., Rockford, Ill.) and ECL as detection agent.

### Example 8

### Mid-Scale Expression of Antibodies

This example provides a method of expressing anti IGF-1R antibodies expressed by stable CHO cell lines.

The CHO cell lines made according to Example 7 were expanded to T-175 tissue culture flasks (Falcon) for scale-up expression. A confluent TI 75 flask (approximately 2-3×107 cells) was used to seed 3-850 cm2 roller bottles (Corning Life Sciences, Acton, Mass.), and three confluent roller bottles (approximately 1−2×108 cells per roller bottle) were used to seed 30 rollers in 250 ml of high-glucose DMEM (Invitrogen), 10% dialyzed FBS (Invitrogen), 1× glutamine (Invitrogen), 1× non-essential amino acids (Invitrogen), 1× sodium pyruvate (Invitrogen). Medium was infused with 10% CO₂/balance air for 5 seconds before capping the roller bottle. Roller bottles were incubated at 37° C. on roller racks spinning at 0.75 rpm.

When cells reached approximately 85-90% confluency (approximately 5-6 days in culture), the growth medium was discarded, the cells were washed with 100 ml PBS, and 200 ml production medium was added (50% DMEM (Invitrogen)/50% F12 (Invitrogen), 1× glutamine (Invitrogen), 1× non-essential amino acids (Invitrogen), 1× sodium pyruvate (Invitrogen), 1.5% DMSO (Sigma). Conditioned medium was harvested every seven days for a total of four harvests.

Conditioned medium was filtered through a 0.45 μm cellulose acetate filter and concentrated approximately 10-fold using a Sartorius Sartocon Slice Disposable 30 K tangential flow membrane (Sartorius AG, Goettingen, Germany). The concentrated material was applied to a 10 ml rProtein A Sepharose column at 4° C. and the flowthrough was collected as the unbound fraction. The column was washed with four column volumes of PBS. The bound sample was eluted with approximately four column volumes of 0.1 M glycine pH 3.0. The eluate peak was collected and neutralized with 0.04 volumes of 1.0 M tris-HCl, pH 9.2. The eluate was dialyzed against 150 volumes of PBS overnight at 4° C. The sample was filtered through a 0.2 μm cellulose acetate filter and protein concentration was measured by determining the absorbance at 280 nm using an extinction coefficient of 14,000 M-1. The sample was compared to a Human IgG1, K standard (Sigma-Aldrich, St. Louis, Mo., USA) using a 4-20% tris-glycine SDS-PAGE gel stained with Coomassie brilliant blue stain. Endotoxin levels in each antibody preparation was determined using the Pyrotell Limulus Amebocyte Lysate Assay (Associates of Cape Cod, Inc., Falmouth, Ma) as per the supplied instructions.

### Example 9

### ORIGEN® Dose Response Competition Assays

This example provides methods for testing the ability of an antibody to block ligand binding to IGF-1R.

An ORIGEN® binding assay was used to determine whether TQ11C, TQ25, TQ 58 and TQ59 IgG1 antibodies could block ligand binding to IGF-1R using procedures provided by the manufacturer (Igen, Inc., Gaithersburg, Md.). To label IGF-1 and IGF-2 with ruthenium, lyophilized proteins were dissolved into PBS to give a 1.0 mg/ml solution. Label (ORI-TAG-NHS ester from Igen, Cat #110034) was added to the protein at a molar ratio of 5:1 (label:protein) from a label stock of 5 mg/ml in DMSO. The mixture was incubated at room temperature (20-22°□ C.) for 1 hr in the dark then treated with 20 μl 2M glycine for 10 min at room temperature. The labeled protein was separated from the free label by application to an Amersham Biosciences NAP-5 column (Amersham Biosciences, Piscataway, N.J.) equilibrated in PBS and 0.33 ml fractions collected. The protein concentration of the fractions was determined by Micro BCA Protein Assay (Pierce Biotechnology, Inc., Rockford, Ill.). Fractions two and three contained significant protein and were combined. The amount of incorporated ruthenium label was assessed using the following formula: ruthenium tris-bipyridyl compound (Ru(bpy)₃²⁺) labeling of IGF-1 and IGF-2.

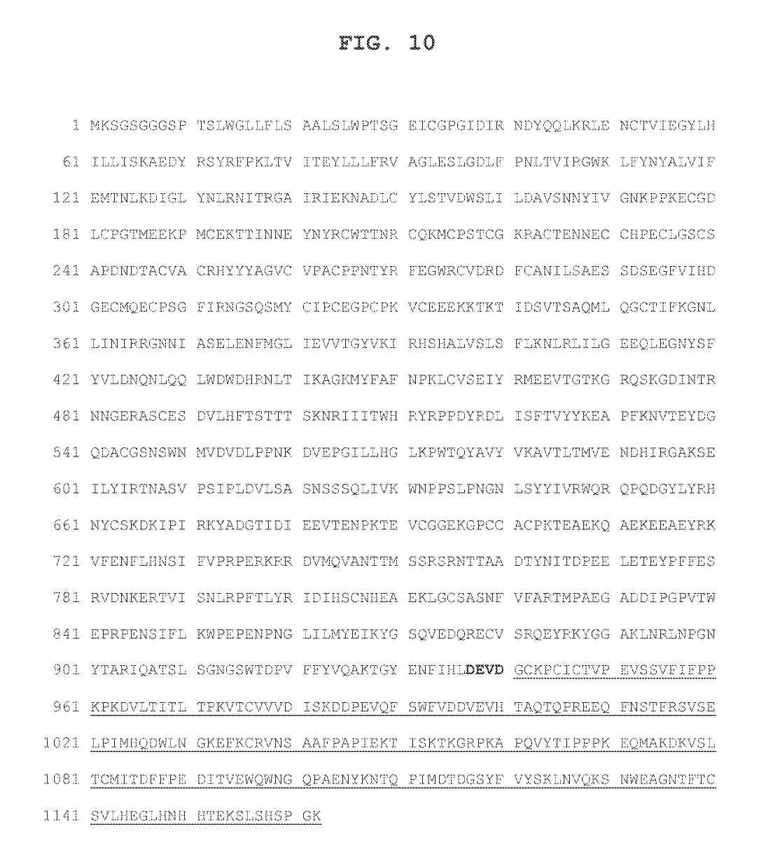

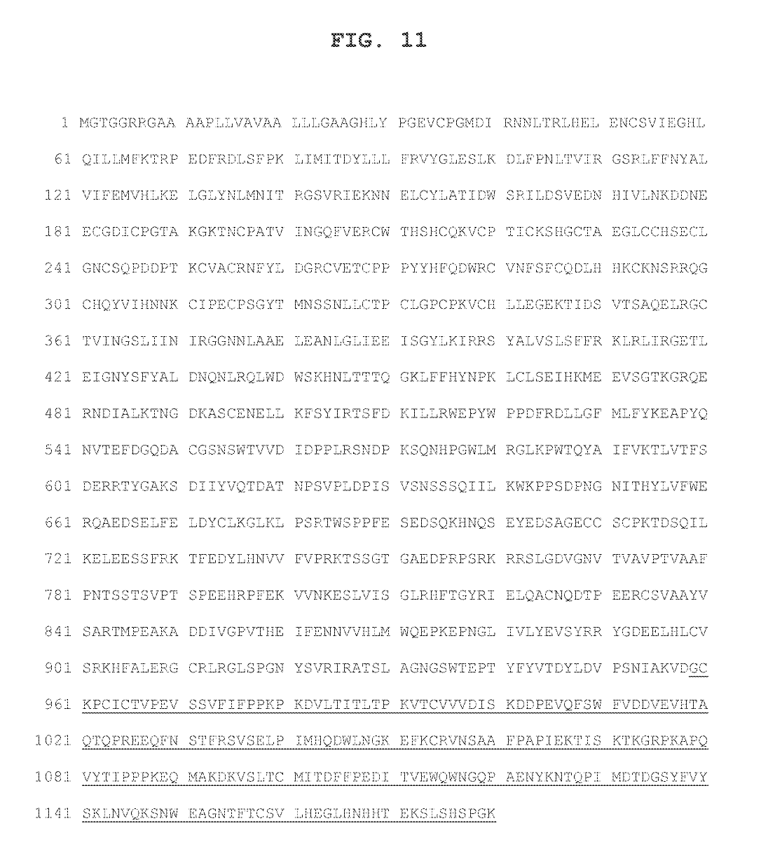

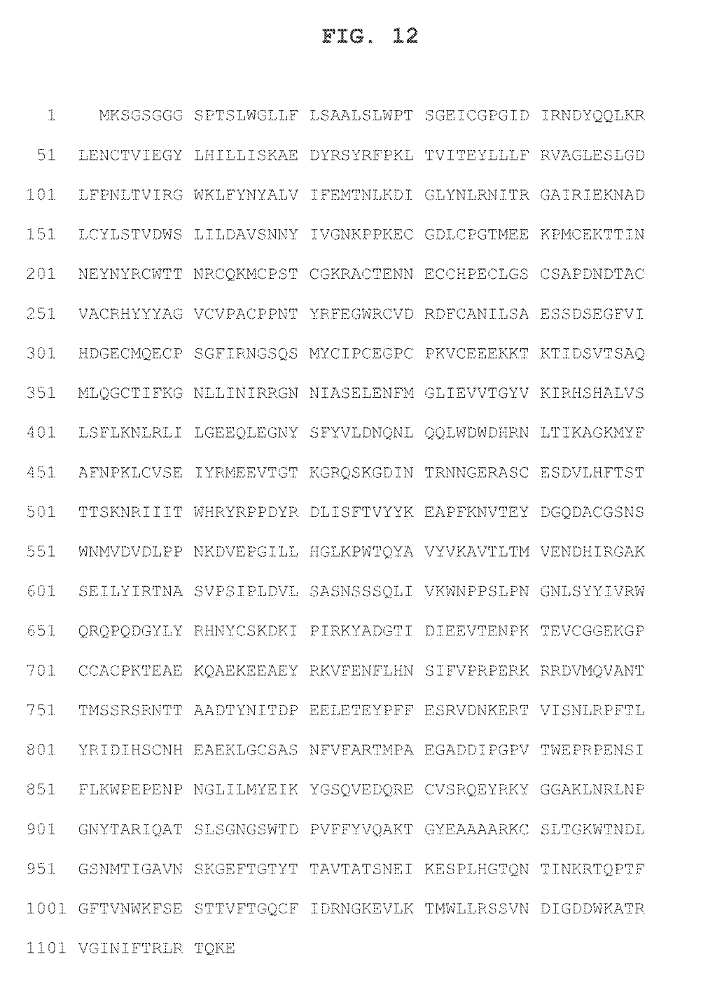

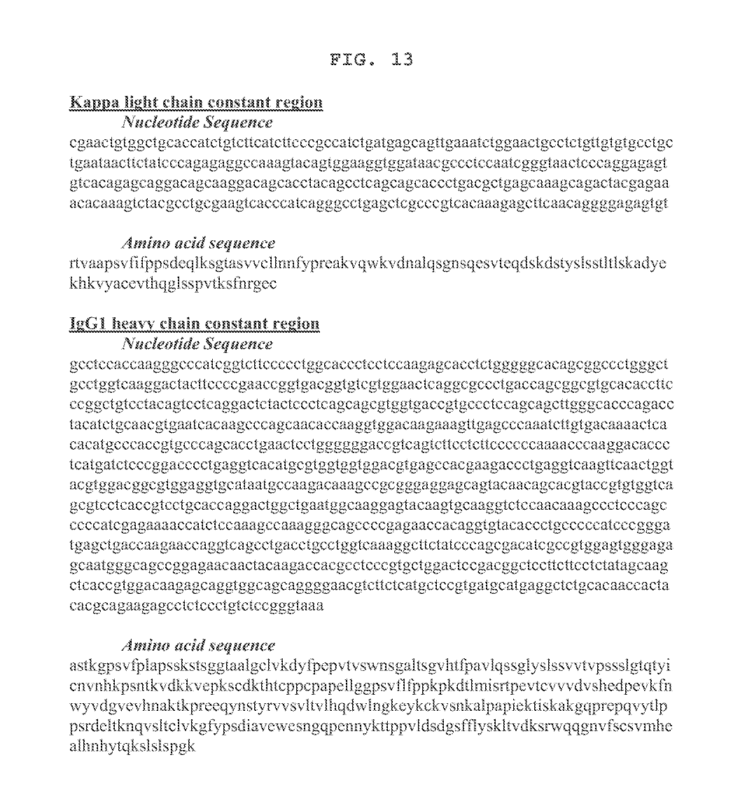

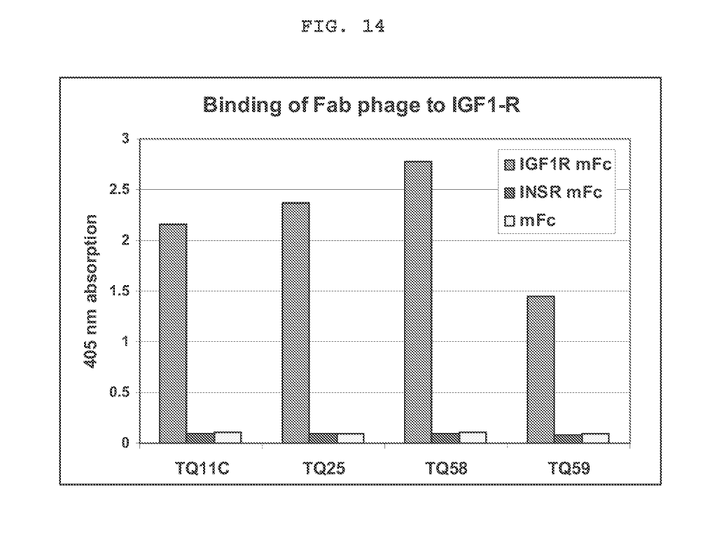

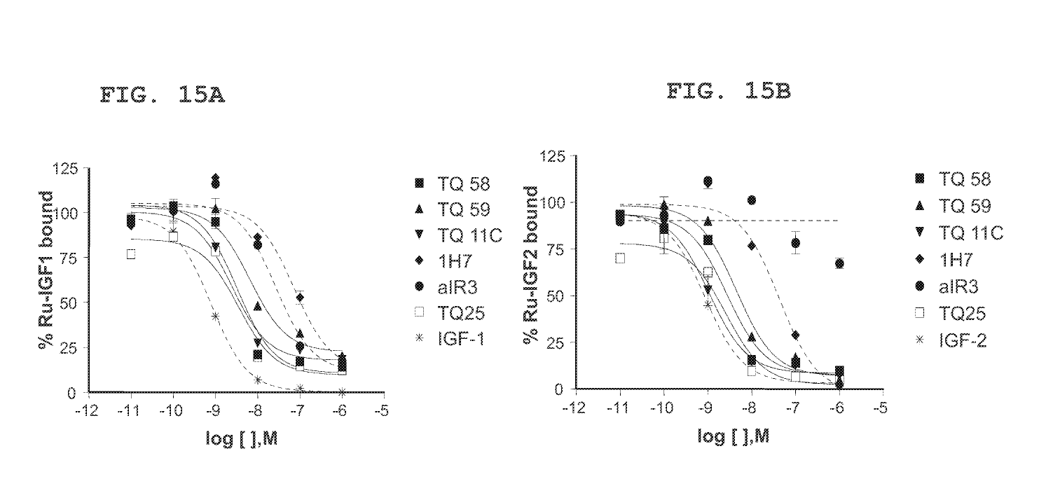

Dynal M450 paramagnetic beads coated with sheep anti-mouse IgG was used as the solid support phase for the IGF-1R(ECD)-C3-muFc. The M450 beads were prepared for receptor loading by washing three times with assay buffer containing 1×PBS, 0.05% TWEEN™ 20 (ICI Americas, Inc., Wilmington Del.) 0.1% BSA, 0.01% sodium azide. The IGF-1R(ECD)-C3-muFc was bound for 1 hr at a ratio of 50 ng receptor per 1×10⁶ M450 beads in a volume of 25 μl assay buffer. To generate dose response data, the antibodies or unlabeled IGF-1 and IGF-2 factors were added at increasing concentrations (10⁻¹¹M to 10⁻⁶M) simultaneously with 1 nM Ru-IGF-1 or 2 nM Ru-IGF-2. The final reaction volume was 100 μl. After incubation at room temperature in the dark for 2 hr, an M8 Analyzer (Igen) was used to remove free ruthenium labeled ligand and determine the amount of ligand bound to receptor. The data were expressed as the percent of total ligand bound minus background remaining after competition with excess unlabeled growth IGF1 or IGF-2. Competition curves were generated with GraphPad Prism software (GraphPad Software, San Diego, Calif.) using a single component equilibrium model. Essentially all (>98%) binding was competed with excess unlabeled growth factors. The positive control antibodies in the binding analysis were the murine anti-IGF-1R antibodies αIR3 (Calbiochem, San Diego, Calif.) or MAB391 (R&D systems, Minneapolis, Minn.), 24-57 (Biocarta, San Diego, Calif.) and 1H7 (Santa Cruz Biotechnology, Inc., Santa Cruz, Calif.). The negative control antibody was an anti-CD20 antibody. Ligand competition data are shown in FIG. 15. The Ki and maximum inhibition values observed for IGF-1 and IGF-2 binding reactions are listed in Table 6.

TABLE 6

| | |
|---|---|
| IGF-1 | IGF-2 |

| Antibody | Ki (nM)¹ | Max (%)² | Ki (nM)¹ | Max (%)² |
|---|---|---|---|---|
| TQ11C | 0.6 | 84 | 0.3 | 91 |
| TQ25 | 0.8 | 88 | 0.8 | 94 |
| TQ58 | 0.8 | 91 | 0.8 | 91 |
| TQ59 | 1.5 | 79 | 1.4 | 91 |
| 1H7 | 16.0 | 89 | 13.1 | 99 |

| | | | |
|---|---|---|---|
| αIR3 | 5.3 | 91 | No Inhibition |

¹Ki of inhibition.
²Maximum level of inhibition at 1 μM antibody concentration.

### Example 10

### SPA Dose Response Competition Assay

This example presents a scintillation proximity assay (SPA) for assessing the effect of antibodies on the interaction of insulin (INS) with the insulin receptor (INSR) and of IGF-1 and IGF-2 to IGF-1R.

IGF-1R binding reactions for TQ11C, TQ25, TQ 58 and TQ59 IgG1 antibodies contained 1×PBS, 0.05% TWEEN® 20 (Mallinkrodt), 0.1% BSA (EM Science, Gibbstown, N.J.), 50 ng IGF-1R(ECD)-C3-muFc, 500 ug SPA PVT anti-mouse IgG fluoromicrospheres (Amersham) and ¹²⁵I-labeled IGF-1 or IGF-2 obtained from Amersham at a final concentration of 0.64 nM. The total reaction volume was 100 μl. The INSR binding reactions were identical except they contained 50 ng INSR(ECD)-muFc and 0.64 nM ¹²⁵I-INS (Amersham). Receptor was loaded onto SPA PVT microspheres for 1 h at room temperature prior to assembly of the binding reactions. To generate dose response data, antibodies or unlabeled growth factors were added at increasing concentrations (10⁻¹¹ M to 10⁻⁶M) simultaneously with ¹²⁵I-labeled growth factors. Essentially all binding was competed with excess unlabeled growth factors. The receptor-independent background, caused by random γ stimulation of the SPT PVT microspheres, was less than 0.5% of the input ¹²⁵I cpm. The data were expressed as the percent of total ligand bound minus background remaining after competition with excess unlabeled growth IGF1 or IGF-2. Competition curves were generated with GraphPad Prism software using a single component equilibrium model.

### Example 11

### Antibody Binding to IGF-1R

This example provides a method of detecting the binding of an anti-IGF-1R antibody to IGF-1R.

BIACORE® 2000, sensor chip CM5, surfactant P20, HBS-EP (10 mM HEPES, 0.15M NaCl, 3.4 mM EDTA, 0.005% P20, pH 7.4), amine coupling kit, 10 mM acetate pH 4.5 and 10 mM glycine pH 1.5 all were purchased from BIACore, Inc. (Piscataway, N.J.). Phosphate-buffered saline (PBS, 1×, no calcium chloride, no magnesium chloride) was from Gibco. Bovine serum albumin (BSA, fraction V, IgG free) was from Sigma. Recombinant Protein G (“rProtein G”) was from Pierce Biotechnology.

Immobilization of rProtein G and IGF-1R-C3-muFc to the sensor chip surface was performed according to manufacturer's instructions, using a continuous flow of 10 mM HEPES, 0.15M NaCl, 3.4 mM EDTA, 0.005% P20, pH 7.4 (HBS-EP buffer). Briefly, carboxyl groups on the sensor chips's surfaces were activated by injecting 60 μl of a mixture containing 0.2 M N-ethyl-N′-(dimethylaminopropyl)carbodiimide (EDC) and 0.05 M N-hydroxysuccinimide (NHS). Specific surfaces were obtained by injecting rProtein A (Pierce) or IGF-1R-C3-mFc diluted in 10 mM acetate, pH 4.5 at concentrations between 20 and 50 μg/ml. Excess reactive groups on the surfaces were deactivated by injecting 60 μl of 1 M ethanolamine. Final immobilized levels were 5,000-6,000 resonance units (RU) for the Protein G surfaces, and ˜7,800 RU for the IGF-1R-mFc surfaces. A blank, mock-coupled reference surface was also prepared on the IGF-1R-mFc sensor chip.

The kinetic analysis of the interaction between IGF-1R-mFc and antibodies was performed as follows. Antibodies as well as a positive control antibody (anti-IR3-CDR-human-mouse chimera) were diluted in PBS+0.005% P20+0.1 mg/ml BSA and injected over the Protein G surfaces to capture the antibodies. IGF-1R-mFc was diluted in PBS+0.005% P20+0.1 mg/ml BSA from 500 nM to 3.9 nM, and each concentration was injected over the captured antibody surfaces, as well as over a blank Protein G surface for background subtraction. After a 10 minute dissociation, each surface was regenerated by injecting 10 mM glycine, pH 1.5. Kinetic analysis of the resulting sensorgrams was performed using BIAEvaluation, v. 3.2 (BIACore, Inc.).

A solution affinity analysis was done by incubating two different concentrations (0.2 nM and 1 nM) of antibody with varying concentrations (0.01 nM to 50 nM) of IGF-1R-mFc in PBS+0.005% P-20+0.1 mg/ml BSA. Incubations were done at room temperature for at least five hours to allow samples to reach equilibrium. Samples were then injected over the immobilized IGF-1R-mFc surface. After the sample injection, the surfaces were regenerated by injecting 25 μl 8 mM glycine, pH 1.5. The binding signal obtained is proportional to the free antibody in solution at equilibrium. The dissociation equilibrium constant (K_{D}) was obtained from nonlinear regression analysis of the competition curves using a dual-curve one-site homogeneous binding model (KinExA software v. 2.3, Sapidyne Instruments Inc., Boise Id.). The data are shown in Table 7

TABLE 7

| | | | K_{d} (kₐ/k_{d}) | |
|---|---|---|---|---|
| | | | Kinetic | K_{d} |
| Antibody | kₒₐ (1/Ms) | K_{d} (1/s) | Method | Equilibrium Method |
| TQ11C | 6.0 × 10⁴ | 6.7 × 10⁻⁵ | 1.1 nM | 0.3 nM |
| TQ25 | 4.4 × 10⁴ | <<5 × 10⁻⁵ | | 0.10 nM |
| TQ58 | 1.1 × 10⁵ | 2.8 × 10⁻⁵ | 0.25 nM | 0.25 nM |
| TQ59 | 6.9 × 10⁴ | 2.1 × 10⁻⁴ | 3.0 nM | 0.30 nM |

### Example 12

### Epitope Mapping Avidin-Fusion Proteins

This example provides a method of determining the epitope of IGF-1R bound by an anti-IGF-1R antibody.

The subdomains of IGF-1R bound by antibodies TQ11C, TQ25, TQ58, and TQ59 were determined using avidin-IGF-1R fusion proteins. To express each protein the coding DNA sequences of the complete IGF-1R(ECD) was cloned into the expression vector pCep4-avidin-C such that chicken avidin sequence is joined to the C-terminus of the expressed IGF-1R protein. The ECD coding sequence (1-932) was PCR amplified from a parental IGF-1R plasmid using PCR primers 2804-25:

SEQ ID NO: 265
5′ GCAAGCTTGGGAGAAATCTGCGGGCCAG 3′
and
2825-68:
SEQ ID NO: 266
5′ ATTGCGGCCGCTTCATATCCTGTTTTGGCCTG 3′

The primers include a 5′ Hind III site and a 3′ Not I site for cloning into pCep4avidin-C. The amino acid sequence of the avidin-human IGF-1R(ECD) fusion protein is shown in FIG. 12. The IGF-1R subdomains constructs used for epitope mapping included: L1 (1-151), CR (152-298), L2 (299-461), FnIII-1 (461-579), FnIII-2/ID (580-798), FnIII-3 (799-901), L1+CR+L2 (1-461), and L1+CR (1-298). The amino acid coordinates of the IGF-1R subdomain represented in each expression plasmid are given in parenthesis. The coding sequence of each domain was PCR amplified from a parental IGF1R cDNA clone using the following primer pairs:

L1:
2804-25:
(SEQ ID NO: 265)
2804-19:
SEQ ID NO: 267
5′ ATTGCGGCCGCCCCACATTCCTTTGGGGGC 3′
CR:
2804-38:
SEQ ID NO: 268
5′ AGCAAGCTTGGACCTGTGTCCAGGGACC 3′
2804-20:
SEQ ID NO: 269
5′ ATTGCGGCCGCGCAAGGACCTTCACAAGGG 3′
L2:
2804-39:
SEQ ID NO: 270
5′ AGCAAGCTTGCCGAAGGTCTGTGAGGAAG 3′
2804-23:
SEQ ID NO: 271
5′ ATTGCGGCCGCACTTTCACAGGAGGCTCTC 3′
FnIII-1:
2808-08:
SEQ ID NO: 272
5′ AGCAAGCTTGGACGTCCTGCATTTCACCTC 3′
2804-52:
SEQ ID NO: 273
5′ ATTGCGGCCGCGGTGCGAATGTACAAGATCTC 3′
FnIII-2+ID:
2804-41:
SEQ ID NO: 274
5′ AGCAAGCTTGAATGCTTCAGTTCCTTCCATTC 3′
2804-51:
SEQ ID NO: 275
5′ ATTGCGGCCGCAGTCCTTGCAAAGACGAAGTTG 3′
FnIII-3:
2804-42:
SEQ ID NO: 276
5′ AGCAAGCTTGATGCCCGCAGAAGGAGCAG 3′
2804-50:
SEQ ID NO: 277
5′ ATTGCGGCCGCTTTAATGGCCACTCTGGTTTC 3′
L1 + CR + L2:
2804-25:
SEQ ID NO: 278
5′ AGCAAGCTTGGGAGAAATCTGCGGGCCAG 3′
2804-23
(SEQ ID NO: 272)
L1 + CR:
2804-25:
(SEQ ID NO: 279)
AGC AAG CTT GGG AGA AAT CTG CGG GCC AG
2804-20
(SEQ ID NO: 270)

The primers included Hind III and Not I site for cloning as described for the IGF-1R (ECD). The IGF-1R subdomains were cloned into the expression vector pCep4avidin-N such that chicken avidin sequence (with endogenous signal sequence) is joined to the N-terminus of the expressed IGF-1R proteins.

Expression of each avidin-fusion protein was achieved by transient transfection of human 293-EBNA cells (Invitrogen) in roller bottles cultures. The cells were grown and maintained in DMEM supplemented with 5% FBS+1× Non-Essential Amino Acids+1× Pen Strep Glut+1× Sodium Pyruvate. Approximately 4−5×10⁷ 293-EBNA cells were seeded in 850 cm² roller bottles overnight. The previously seeded cells were then transfected with pCep4-avidin plasmid DNA the following day using FUGENE™ 6 transfection reagent. The DNA-transfection reagent mixture was prepared in approximately in 6.75 mL serum-free DMEM. 675 μl FUGENE™ 6 transfection reagent was first added, followed by 112.5 μg plasmid DNA. The complex was incubated at room temperature for 30 minutes. The entire mixture was then added to a roller bottle. The roller bottle was gassed with a 5% CO₂ gas mixture, capped tightly and placed in a 37° C. incubator on a roller rack rotating at 0.35 RPM. The transfection was performed for 24 hours after which the medium was replaced with 100 mL DMEM+1× Insulin-Transferrin-Selenium Supplement+1× Pen Strep Glu+1× Non-Essential Amino Acids+1× Sodium Pyruvate. Harvest of the condition medium and replacement with fresh medium occurred 48 hr intervals (2-3 cycles). The harvested serum-free conditioned medium was pooled together and clarified by centrifugation at 10,000×g for 30 minutes at 4° C.

The concentration of avidin-fusion in each conditioned medium was determined using a quantitative FACS based method. The avidin fusion protein in 200 μl of conditioned medium was captured by incubation for 2 hr at room temperature with 5 μl (˜3.5×10⁵) of biotin coated polystyrene beads (Spherotech, Inc., Libertyville, Ill.). The conditioned medium was removed by three cycles of centrifugation and resuspension of the avidin-coated beads in PBS containing 0.5% BSA (BPBS). The avidin-beads were stained with 1 μg/ml of goat FITC-labeled anti-avidin antibody (Vector Lab Burlingame, Calif.) in 1 ml BPBS. After 0.5 hr incubation antibody-beads complexes were collected by centrifugation at 1800 rpm for 5 min and the pellet was washed three times. The FITC fluorescence was detected with a FACSCAN (Beckton Dickson Bioscience, Franklin Lakes, N.J.). The signal was converted to protein mass using a standard curve derived with recombinant avidin. For epitope mapping the biotin-beads were loaded with 50-100 ng avidin-fusion protein per ˜3.5×10⁵ beads of beads by incubation with the appropriate amount (1-20 ml) of conditioned medium. The loaded beads were washed extensively and resuspended in 1 ml BPBS. For all experiment the biotin-beads were blocked with 10% BSA in PBS prior to loading fusion protein.

Method 1, One Color Assay:

Biotin-coated polystyrene beads loaded with IGF-1R (ECD) and IGF-1R subdomain fusion proteins were mixed with 1 μg of anti-IGF-1R antibody in 1 ml of BPBS. After incubation for 1 hr at room temperature, 4 ml washing buffer was added and the antibody-beads complexes were collected by centrifugation for 5 min at 750 g. The pellet was washed 3 times by resuspension in 4 ml of BPBS. The antibody bound to avidin-bead complexes was detected by treatment with 0.5 μg/ml Phycoerythrin-(PE) labeled goat anti-human F(ab′)2 (Southern Biotech Associates, Inc., Birmingham, Ala.) in 1 ml BPBS. Tested antibodies were found to bind to the avidin-fusion protein containing the complete IGF-1R ECD and the L2 domain. Binding to L1, CR or FnIII-1 was not detected in this experiment. A relatively weak reaction was also observed with the L1 domain.

Method 2, Two Color Assay:

To simultaneously monitor the amounts of anti-IGF-1R monoclonal antibody and avidin-fusion bound to biotin-beads, FITC-labeled anti-avidin antibody was included (1 μg/ml) was included in the binding reaction in combination with 0.5 μg/ml PE-labeled goat anti-human IgG1. The beads were prepared for FACSCAN analysis as described for the one color assay.

Method 3, Antibody Competition:

To prepare for labeling with fluorescein the antibodies were dialyzed or resuspended at a concentration of 1 mg/ml in PBS (pH 8.5). Label ([6-fluorescein-5-(and -6)-carboxamido]hexanoic acid, succinimidyl ester 5(6)-SFX] mixed isomers from Molecular Probes (Eugene, Oreg., Cat. No. F2181) was added to the protein at a molar ratio 9.5:1 (label:protein) from a label stock of 5 mg/ml in DMSO. The mixture was incubated at 4°□ C. overnight in the dark. The labeled antibody was separated from the free label by dialysis in PBS. The FITC/antibody ratios obtained ranged from 3 to 8. For each competition experiment, a binding reaction was assembled that contained a 50 fold excess (10-50 μg/ml) of unlabeled competitor antibody, 3.5×10⁵ biotin beads coated with avidin fusion protein in BPBS. The FITC-labeled antibody (1 μg/ml) was added after a 30 min preincubation. The process followed the one color method from this point forward.

Each of the four tested antibodies binds to the IGF-1R L2 domain, as shown in Table 8. However, the precise amino acid contacts of each antibody in the IGF-1R L2 domain may differ.

TABLE 8

| Antibody | L1¹ | CR¹ | L2¹ | FnIII-1¹ | ECD^{1,2} |
|---|---|---|---|---|---|
| TQ11C | No | No | Yes | No | Yes |
| TQ25 | No | No | Yes | No | Yes |
| TQ58 | Yes | No | Yes | No | Yes |
| TQ59 | No | No | Yes | No | Yes |

¹Epitope mapping was performed with avidin-IGF-1R fusion proteins containing the indicated human IGF-1R regions.
²The ECD fusion contains L1 + CR + L2 + FnIII-1 + FnIII-2 + ID + FnIII-3.

### Example 13

### Antibody Binding to Cell-Surface IGF-1R

This example provides a method for detecting the binding of an anti-IGF-1R antibody to cell-surface expressed IGF-1R.

The ability of antibodies TQ11C, TQ25, TQ58, and TQ59 to bind to human IGF-1R displayed on the cell surface was evaluated using Balb/C 3T3 fibroblasts and MCF-7 human breast cancer cells engineered to overexpress the human IGF-1R receptor at a level of ˜3−4×10⁵ molecules per cell. A Balb/C 3T3 cell line that stably overexpresses the human IGF-1R (˜3×10⁵ receptors per cell) was derived using with a retroviral vector essentially as described by Pietrzkowski et al., 1992, Cell Growth Differentiation 3:199-205. MCF-7 breast cancer cells that overproduce huIGF-1R were transfected with a pcDNA3.1 expression vector (Invitrogen Corp.). Zeocin resistant cells that express a high level of hu IGF-1R (˜4×10⁵ receptors per cell) were expanded after selection by FACS using anti-IGF-1R monoclonal antibody αIR3 and an PE-labeled goat anti murine IgG antibody (Caltag Laboratories, Burlingame, Calif.). The process of selection and expansion was repeated four times.

IGF-1R Receptor antibody staining and receptor expression was monitored by FACS as follows: the cells were released from T175 flasks (Corning) by washing 2 times with excess PBS (Ca/Mg free) followed by treatment with 5 ml of Cell Dissociation Buffer (Sigma) for 10 min at room temperature. The cells were collected by centrifugation and washed two times by resuspending them in PBS and centrifugation. For primary antibody staining, 1 μg of antibody was added to 10⁶ cells resuspended in 100 μl PBS plus 0.5% BSA (BPBS) and the cells were incubated at 4°^{□} C. for 1.5 hr. The cells were collected by centrifugation and washed twice with BPBS to remove unbound primary antibody. The cells were resuspended in 100 μl of BPBS and incubated with 1 μg of FITC-labeled goat anti-human F(ab′)2 (Southern Biotechnology Associates, Inc., Birmingham, Ala.) at 4°□ C. for 30 minutes. After washing to remove unbound FITC secondary antibody, the cells were resuspended in 1 ml of PBS+0.5% BSA and FITC cell fluorescence was detected with a FACSCAN (Beckton Dickson Bioscience, Franklin Lakes, N.J.). The fluorescence levels were converted to absolute receptor levels using Quantum microbead (Bangs Laboratories, Inc., Fishers, Ind.) with predetermined IgG1 binding capacity to generate a standard curve. Data reduction was performed with QuickCal v2.1 software (Verity Software House, Topsham, Me.) provided by the manufacturer.

The peak fluorescent intensity of anti-IGF-1R antibody labeling of the IGF-1R overexpressors was increased 10-20 fold relative to parental Balb/C 3T3 and MCF-7 cells for each of the tested antibodies. This is the result predicted for an antibody that specifically binds IGF-1R. Background fluorescence of cells treated with no antibodies or FITC-labeled secondary alone were insignificant.

### Example 14

### Inhibition of IGF-1R

This example presents methods of detecting inhibition of IGF-1R by anti-IGF-1R antibodies.

### 32D hu IGF-1R+IRS-1 Cell Inhibition

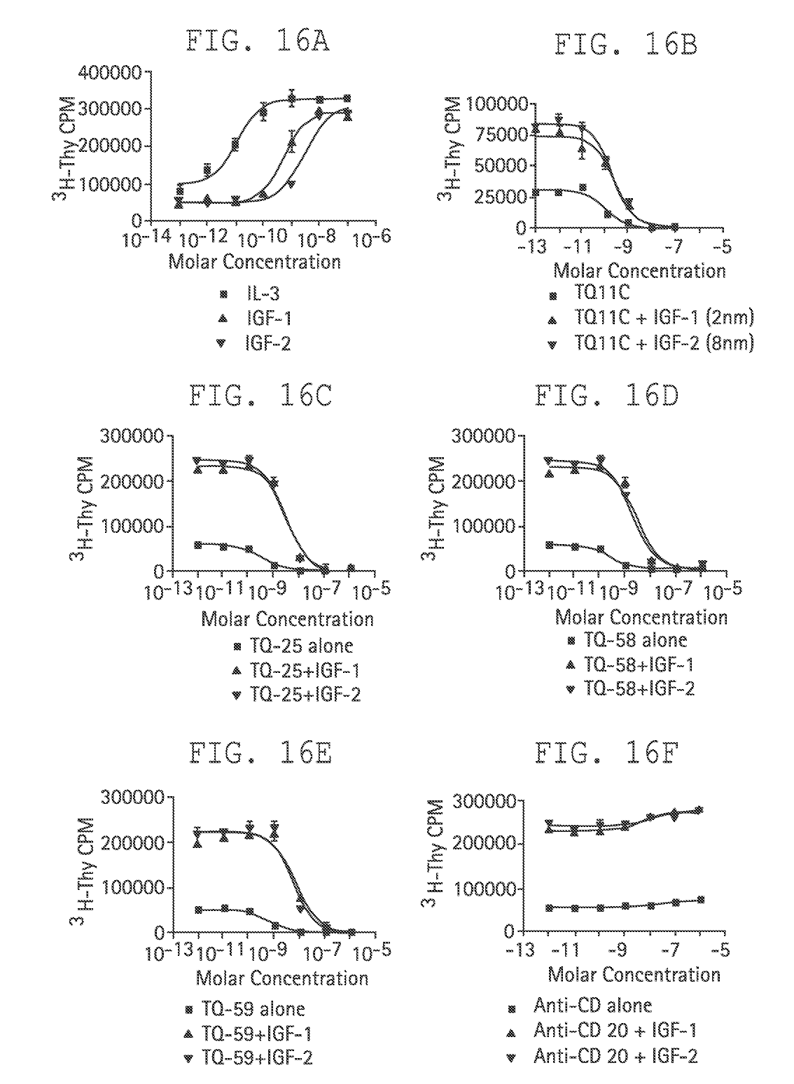

Murine 32D cells that coexpress the human IGF-1R receptor (20K per cell) and human IRS-1 have proven to be a effective system to examine the molecular components IGF-1R signaling Valentinis et al., 1999, J Biol Chem 274:12423-30. Normal 32D cells express relatively low levels of the murine orthologs of these two gene products. 32D cell normally required IL3 for growth and survival. IGF-1 or IGF-2 can replace IL3 in 32D huIGF-1R+IRS-1 cells as shown in FIG. 16, panel A. The EC₅₀ to the IGF-1 dose response curve was about 0.5 nM, whereas the IGF-2 EC₅₀ (2.8 nM) is about six fold higher reflecting weaker affinity of IGF-2 for IGF-1R. To assess the ability of the antibodies TQ11C, TQ25, TQ58, and TQ59 to block IGF-1 or IGF-2 stimulation, 96-well microtitre plates were seeded with 30,000 32D hu IGF-1R+IRS-1 cells per well in a volume of 200 μl of RPMI (Gibco/BRL) containing 5% fetal bovine serum (Gibco/BRL) and 1× penicillin, streptomycin, glutamine (Giboco/BRL) and increasing concentrations of antibody (10⁻¹²M to 10⁻⁶M) or no antibody. IGF-1 (2 nM), IGF-2 (8 nM) or nothing was added after 1 hr preincubation with antibody. ³H-thymidine (1 μCi per well) was added at 27 hr post-antibody addition. The cells were harvested 21 hr later, and incorporation of ³H-thymidine into DNA was determined for each sample. The assays were performed in triplicate. An anti-CD20 antibody was used as a negative control. Each of antibodies TQ11C, TQ25, TQ58, and TQ59 was able to completely block the IGF-1 and IGF-2 mediated stimulation of the 32D cells. The reduction of background proliferation in the absence of added IGF-1 and IGF-2 is due to the inhibition of serum IGF-1 and IGF-2. The binding data were analyzed using GraphPad PRIZM™ software. The data are shown in FIG. 16.

### Balb/C 3T3 hu IGF-1R Cell Inhibition

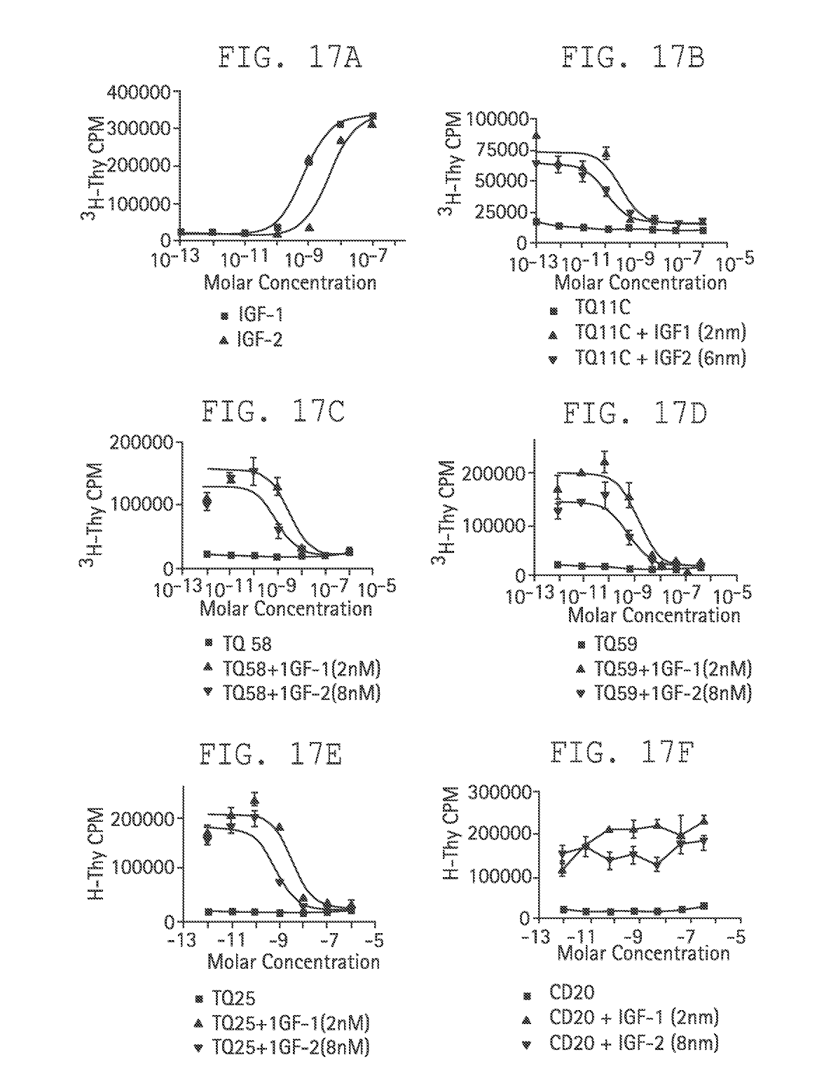

IGF-1 greatly stimulates the incorporation of ³H-thymidine by serum-starved cultures of mouse embryonic fibroblasts (Balb/C 3T3 or NIH 3T3) that overexpress IGF-1R (˜1×10⁶ IGF1R per cell). Kato et al., 1993, J Biol Chem 268:2655-61; Pietrzkowski et al., 1992, Cell Growth Differentiation 3:199-205. This phenomenon is recapitulated with both IGF-1 and IGF-2 in a Balb/C 3T3 cell line hu IGF-1R overexpressor. Both growth factors stimulated ³H-thymidine incorporation by about 20-fold. The EC₅₀ of the IGF-1 dose response curve was about 0.7 nM, whereas the IGF-2 EC₅₀ (4.4 nM) is sevenfold higher, indicating a weaker affinity of IGF-2 for IGF-1R. To assess the ability of a given antibody to block IGF-1 or IGF-2 stimulation, 96-well microtitre plates were seeded with 10,000 cells per well in a volume of 200 μl of DMEM (Gibco/BRL) containing 10% calf serum (Gibco/BRL) and 1× penicillin, streptomycin, glutamine (Giboco/BRL). After overnight incubation when the cells were about 80% confluent the growth medium was replaced with 100 μl DMEM containing 0.1% BSA after washing once with 200 μl PBS. Antibodies at increasing concentrations (10⁻¹² M to 10⁻⁶M), or no antibody, were added at 24 hr post-serum starvation. IGF-1 (2 nM), IGF-2 (8 nM) and ³H-thymindine (1 μCi per well) were added after a 1 hr preincubation with antibody. The cells were harvested 24 hr later, and incorporation of ³H-thymidine into DNA was determined for each sample. The assays were performed in triplicate. Each tested antibody was able to completely block the IGF-1 and IGF-2 mediated stimulation of Balb/C 3T3 cells, as shown in FIG. 17. An anti-CD20 antibody was used as a negative control (“CD20” in FIG. 17).

### Example 15

### Treatment of Cancer in Humans with an Anti-IGF-1R Antibody

This example demonstrates that inhibition of the IGF-1R pathway is effective for treating a variety of types of tumors in human subjects.

Human subjects were selected for treatment in a First in Human Phase 1 clinical trial with a fully-human anti-human IGF-1 receptor IgG1 monoclonal antibody comprising the light chain variable domain identified herein as L16 and the heavy chain variable domain identified herein as H16 (“Study Drug”), as shown in Table 9.

TABLE 9
Cohort #1 (1 mg/Kg)

| Subject #5 | | |
|---|---|---|
| Diagnosis | Thymus | |
| Baseline TM (cm) | 10 | |
| Antibody per Dose (mg) | 92.5 | |
| Dosed at Days | 1, 15, 29 | |
| Day 50 Tumor (cm) | 10.4 | (+4%) |
| Subject #8 | | |
| Diagnosis | Unknown | |
| Baseline TM (cm) | 18.5 | |
| Antibody per Dose (mg) | 84.1 | |
| Dosed at Days | 1, 15, 29, 57, 71, 85, 99, 113, 127, 141, 155 | |
| Day 50 Tumor (cm) | 18.2 | (−2%) |
| Day 106 Tumor (cm) | 18.9 | (+2%) |
| Day 162 Tumor (cm) | 23.2 | (+25%) |
| Subject #7 | | |
| Diagnosis | Adenoid | |
| Baseline TM (cm) | 31.1 | |
| Antibody per Dose (mg) | 60 | |
| Dosed at Days | 1, 15, 29, 57, 71, 85 | |
| Day 50 Tumor (cm) | 30.9 | (−1%) |

Cohort #2 (3 mg/Kg)

| Subject #1 | | |
|---|---|---|
| Diagnosis | Nerve Sheath | |
| Baseline TM (cm) | 1.1 | |
| Antibody per Dose (mg) | 208 | |
| Dosed at Days | 1, 15, 29 | |
| Day 50 Tumor (cm) | 1.4 | (+27%) |
| Subject #11 | | |
| Diagnosis | Carcinoid | |
| Baseline TM (cm) | 13.1 | |
| Antibody per Dose (mg) | Week 1-35: 207 | |
| | Week 39 and on: 828 | |
| Dosed at Days | 1, 15, 29, 57, 85, 99, 120, 134, 148, 162, 176, | |
| | 190, 204, 218, 232, 246, 260, 274, 296, 308, | |
| | 331 | |
| Day 50 Tumor (cm) | 14 | (+7%) |
| Day 106 Tumor (cm) | 11 | (−16%) |
| Day 169 Tumor (cm) | 10.6 | (−19%) |
| Day 225 Tumor (cm) | 8.4 | (−36%) |
| Day 281 Tumor (cm) | 8.2 | (−37%) |
| Day 338 Tumor (cm) | 6.8 | (−48%) |

Cohort #3 (10 mg/Kg)

| Subject #2 | | |
|---|---|---|
| Diagnosis | Prostate | |
| Baseline TM (cm) | 15.6 | |
| Antibody per Dose (mg) | 790 | |
| Dosed at Days | 1, 15, 29 | |
| Day 50 Tumor (cm) | 18.8 | (+21%) |
| Subject #6 | | |
| Diagnosis | Melanoma | |
| Baseline TM (cm) | 28.1 | |
| Antibody per Dose (mg) | 854.5 | |
| Dosed at Days | 1, 15, 29, 57, 71 | |
| Day 50 Tumor (cm) | 28.4 | (+1%) |
| Subject #4 | | |
| Diagnosis | Colorectal | |
| Baseline TM (cm) | 42.2 | |
| Antibody per Dose (mg) | 895 | |
| Dosed at Days | 1, 15, 29 | |
| Day 50 Tumor (cm) | 45.3 | (+7%) |

Cohort #4 (20 mg/Kg)

| Subject #3 | | |
|---|---|---|
| Diagnosis | Ovarian | |
| Baseline TM (cm) | 15.9 | |
| Antibody per Dose (mg) | 2118 | |
| Dosed at Days | 1, 15, 29 | |
| Day 50 Tumor (cm) | 18.6 | (+17%) |
| Subject #9 | | |
| Diagnosis | Breast | |
| Baseline TM (cm) | 4.8 | |
| Antibody per Dose (mg) | 1570 | |
| Dosed at Days | 1, 15, 29, 57, 71 | |
| Day 50 Tumor (cm) | 4.7 | (−2%) |

Cohort #5 (12 mg/Kg)

| Subject #12 | | |
|---|---|---|
| Diagnosis | Ewing's | |
| Baseline TM (cm) | 9.8 | |
| Antibody per Dose (mg) | 1190 | |
| Dosed at Days | 1, 15, 29, 57, 71 | |
| Day 50 Tumor (cm) | 2.2 | (−78%) |
| Day 85 Tumor (cm) | 0.0 | (−100%) |

Cohort #6 (20 mg/Kg)

| Subject #10 | | |
|---|---|---|
| Diagnosis | Adenoid R eye | |
| Baseline TM (cm) | 38.7 | |
| Antibody per Dose (mg) | 1763.6 | |
| Dosed at Days | 1, 15, 29, 57, 71 | |
| Day 50 Tumor (cm) | 35.2 | (−9%) |

Prior to being selected for the study, each subject had failed available conventional treatments for his or her particular tumor disease, if such treatments were available, and was receiving only supportive care.

Each subject was assigned to one of six dosing cohorts. Subjects in any given cohort each received the same dose of the Study Drug intravenously. Dosing between cohorts ranged from 1 to 20 milligrams of Study Drug per kilogram of subject's body mass (mg/kg), as shown in Table 9. The Study Drug was formulated at 30 mg/ml in 10 mM acetate, pH 5.2, 5.0% w/v sorbitol, and 0.004% w/v Polysorbate 20. During the course of treatment, the subjects received the Study Drug as their only anti-tumor treatment. The subjects also received individualized palliative care, as appropriate, to reduce the severity of their symptoms.

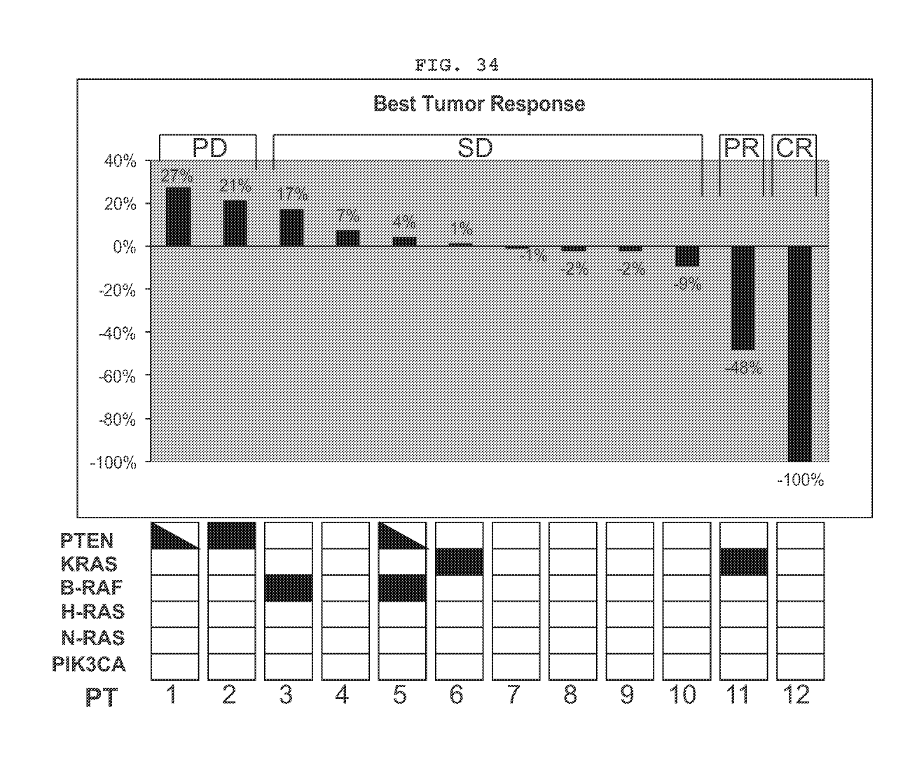

Response to treatment was assessed using the Response Evaluation Criteria in Solid Tumors (RECIST) criteria as described in Therasse et al. 2000, J Natl Cancer Inst. 92:205-16, incorporated herein by reference in its entirety for all purposes. Briefly, prior to administration of the first dose, each subject was given a computerized tomography (CT) scan to determine the length of the largest measurable tumor along its longest diameter (“Baseline TM (cm)” in Table 9). CT scans were used to measure the same tumors along the same diameter at certain points after initiation of treatment (“Day X Tumor (cm)” in Table 9). Each such measurement was compared to the baseline tumor measurement for the same subject to calculate the percent increase or decrease in tumor size. As shown in FIG. 34 and in Table 9, two of the subjects showed a reduction in tumor size of at least 30%. One of these subjects was classified as a partial responder (PR) according to RECIST. The other had a 100% reduction in tumor dimension and so was classified as a complete responder (CR) according to RECIST. Eight other subjects had as a best response either a reduction of tumor size of less than 30% or an increase of less than 20%, and so are classified as having stable disease (SD) using RECIST criteria (note that one of these subject's had as a best response an initial 2% reduction in tumor size, but that subsequently the tumor showed and overall increase in size of 25%). Each of these subjects (except the CR subject, discussed below) eventually showed disease progression and was taken off of study. The remaining two subjects had RECIST tumor measurements that increased by more than 20%, indicating a best response of progressive disease (PD).

The CR subject had classical Ewing's sarcoma (characterized by a EWS-FLI genetic translocation; see, e.g., Dagher et al., 2001, J Pediatr Hematol Oncol. 23:221-24; Morishita et al., 2001, Mol. Biotechnol. 18:97-104, each incorporated herein by reference in its entirety for all purposes) that had formed large metastatic tumors in the lungs, making breathing difficult, particularly while lying prone. The subject was resistant to multiple prior chemotherapy regimens, including 1) adriamycin and cytoxan, 2) ifosphamide and vincristine, 3) topotecan and vincristine, 4) taxotere, and 5) gemcitabine. The subject received a first dose of 12 mg/kg of anti-IGF-1R antibody. The subject experienced significant symptomatic relief within two days of receiving the first dose of the Study Drug, allowing him to comfortably sleep in a prone position for the first time in several months. The subject subsequently received three doses of 12 mg/kg at 14 day intervals. Fifty days after the first injection, a CT scan of the subject showed a decrease in tumor size from the baseline measurement of 9.8 cm to 2.2 cm, or 78%, using RECIST. At day 50, the subject was also given a PET scan, which showed no detectable uptake of labeled glucose, indicating that most or all of the remaining tumor tissue was dead. At day 85, the subject underwent a CT scan that showed a complete resolution of tumor from the pre-treatment diameter of 9.8 cm to 0 cm. The subject continued to receive 12 mg/kg of the Study Drug at 14 day intervals and at day 434 still had a CR according to RECIST.

The PR subject had a mid gut carcinoid tumor and achieved a partial response after 33 weeks in the trial with a RECIST tumor dimension decrease from 13.1 to 6.8 cm, or 48%. The subject continued to receive 3 mg/kg of the Study Drug at 14 day intervals and showed a maximum RECIST tumor dimension reduction of 63%. At day 655, the subject was discovered to have new bone metastases and was taken off of the study.

Some subjects exhibited grade 3 or 4 thrombocytopenia. In every case where thrombocytopenia was detected, it resolved spontaneously with cessation or interruption of dosing. There were no cases of spontaneous bleeding noted in these subjects.

Additional patients were treated on this study who also had diagnoses of either Ewing's sarcoma or Desmoplastic Small Round Cell Tumors. Each of these subjects had had multiple prior cytotoxic chemotherapy regimens and had subsequently shown progression. Twelve such subjects received either 12 mg/kg (n=6) or 20 mg/kg (n=6) of the Study Drug at two week intervals. Table 10 shows the results for the study.

TABLE 10

| Subject | Study | | | PET | Best |
|---|---|---|---|---|---|
| Number | Drug Dose | Translocation | Study Status | D8 | Response |
| 1 | 20 mg/kg | N/A | Off at day 127 | −32% | SD |
| 2 | 12 mg/kg | N/A | Off at day 114 | −10% | SD |
| 3 | 20 mg/kg | N/A | Off at day 79 | −57% | N/A |
| 4 | 20 mg/kg | N/A | Off at day 58 | −60% | PD |
| 5 | 12 mg/kg | N/A | Off at day 57 | +16% | PD |
| 6 | 12 mg/kg | N/A | Off at day 48 | +10% | PD |
| 7 | 20 mg/kg | Negative | Off at day 43 | +11% | PD |
| 8 | 12 mg/kg | N/A | Off at day 39 | +25% | PD |
| 9 | 20 mg/kg | “EWS-FLI” | Off at day 37 | −11% | PD |
| 10 | 12 mg/kg | Negative | Off at day 35 | +1% | PD |
| 11 | 20 mg/kg | N/A | Off at day 34 | −35% | PD |
| 12 | 12 mg/kg | “EWS-FLI” | Off at day 23 | −12% | PD |

Two subjects were classified as having a best response of SD using RECIST criteria. One of them showed a reduction in tumor metabolic activity of 32%, the other of 10%, on day 8 according to a PET scan. A third subject achieved a PR according to RECIST and a 57% reduction in metabolic activity on day 8. The tumors in all three subjects subsequently progressed, and so the subjects were taken off of the study. The remaining subjects all showed progressive disease as a best response and were taken off of the study, although several of them showed reductions in metabolic activity on day 8 of between 11% and 35%.

The tumor genotypes of the three best responders were not available. However, two of the subjects who showed a reduction in metabolic activity on day 8 (but whose best RECIST response was PD) were found to contain the EWS-FLI translocation. Two other subjects who showed a best RECIST response of PD, and who showed no change or a slight increase in tumor metabolic activity on day 8, were found to not have the translocation.

Another study was done in subjects with carcinoid tumors. Five subjects were given either 6 (n=1) or 20 mg/kg (n=4) of the Study Drug at two week intervals. The results are shown in Table 11.

TABLE 11

| Subject | Study Drug Dose | | | Best |
|---|---|---|---|---|
| Number | (mg/kg) | Study Status | RECIST | Response |
| 1 | 20 | Off at day 288 | −32% | PR |
| 2 | 20 | Continued past | −20% | SD |
| | | day 378 | | |
| 3 | 20 | Continued past | −2% | SD |
| | | day 282 | | |
| 4 | 6 | Off at day 112 | N/A | SD |
| 5 | 20 | Off at day 191 | −5% | SD |

Each of the subjects was enrolled in the study after having tried and failed other treatments. Subject 1 showed a best response of PR (32% reduction in tumor size according to RECIST criteria). The remaining subjects showed best responses of SD, with between a 2% and 20% reduction in tumor size according to RECIST criteria.

Subjects 2 and 3 remained on the study past day 378 and day 282, respectively. Subject 1 was removed from the study on day 288 after showing progressive disease. Subject 4 was removed from the study on day 112 for noncompliance. Subject 5 was removed from the study on day 191 after developing a pulmonary embolus.

Another study was done in subjects with colorectal cancer (CRC). Seven subjects were each given 6 mg/kg of panitumumab (a human anti-EGF receptor antibody) and either 6 (n=3) or 12 mg/kg (n=4) of the Study Drug at two week intervals. The results are shown in Table 12.

TABLE 12

| | Panitumumab | Study | | | | Wk 8 | Wk 8 |
|---|---|---|---|---|---|---|---|
| Subject | Dose | Drug Dose | Study | Prior | Best WHO | change | change |
| Number | (mg/kg) | (mg/kg) | Status | EGFR | Response | (WHO) | (RECIST) |
| 1 | 6 | 6 | Off at | Yes | SD | N/A | N/A |
| | | | day 99 | | | | |
| 2 | 6 | 6 | Off at | Yes | SD | −39% | −27% |
| | | | day 113 | | | | |
| 3 | 6 | 6 | Off at | — | PD | N/A | N/A |
| | | | day 58 | | | | |
| 4 | 6 | 12 | Off at | Yes | SD | −19% | −5% |
| | | | day 168 | | | | |
| 5 | 6 | 12 | Continued | Yes | PR | −54% | −36% |
| | | | Past Day | | | | |
| | | | 191 | | | | |
| 6 | 6 | 12 | Off at | Yes | PD** | | |
| | | | day 7 | | | | |
| 7 | 6 | 12 | Off at | No | PD*** | −7% | +1% |
| | | | day 57 | | | | |

* “Yes” indicates subject previously treated with EGF receptor inhibitor
**Newly discovered brain metastases at day 7
***Progression of non-index lesions at day 57

All of the subjects had advanced solid malignancies refractory to standard therapy. In table 12, “Yes” in the “Prior EGFR” column means that the subject had previously been treated with an anti-EGF receptor antibody (either panitumumab or cetuximab). “Best WHO Response” and “Wk 8 CT change (WHO)” refer to tumor assessments done using WHO criteria (Miller et al., 1981, Cancer 47:207-14, incorporated herein by reference in its entirety for all purposes).

Subject 5 showed a best WHO response of PR. The tumors of subject 5, who experienced a best WHO response of PR and who continued on the study past day 191, were found to have a wild-type allele of KRAS. Before beginning the study, subject 5 had failed four prior chemotherapy regimens and five cycles of irinotecan and cetuximab.

Three subjects with non-CRC tumors also received 6 mg/kg panitumumab and 6 mg/kg of Study Drug and had their best responses evaluated according to WHO criteria. None of these subjects had previously been treated with an EGF receptor inhibitor. A first subject with a thyroid tumor showed a best response of progressive disease and was removed from the study on day 55. This subject was prediabetic prior to participation in the study, with a fasting glucose level of 113 mg/dL, and experienced a dose limiting toxicity of Grade 3 hyperglycemia. A second subject with a GE Junction tumor had a best response of stable disease and was removed from the study on day 114. A third subject with a pancreatic tumor had a best response of stable disease and was removed from the study on day 106.

Another study was done using Study Drug in combination with gemcitabine treatment in subjects with a variety of tumor types. Eleven subjects were each given three doses of gemcitabine at 1000 mg/kg every four weeks and were also given Study Drug at either 6 (n=6) or 12 mg/kg (n=5) every 2 weeks. The results are shown in Table 13.

TABLE 13

| | Study | | | | Best |
|---|---|---|---|---|---|
| Subject | Drug Dose | | | | WHO |
| Number | (mg/kg) | Diagnosis | Study Status | DLT | Response |
| 1 | 6 | Ovarian | Off at day 157 | No | SD |
| 2 | 6 | Ovarian | Off at day 126 | No | SD |
| 3 | 6 | Lung | Off at day 53 | Yes* | PD |
| 4 | 6 | Carcinoid | Off at day 112 | No | SD |
| 5 | 6 | Lung | Off at day 56 | No | SD |
| 6 | 6 | Head and Neck | Off at day 123 | No | SD |
| 7 | 12 | Colon | Off at day 106 | No | SD |
| 8 | 12 | Breast | Off at day 184 | No | SD |
| 9 | 12 | Colon | Continued past | No | SD |
| | | | day 116 | | |
| 10 | 12 | Prostate | Continued past | No | SD |
| | | | day 114 | | |
| 11 | 12 | Gallbladder | Continued past | No | N/A |
| | | | day 47 | | |

*Grade 4 Neutropenia on day 8

All but one evaluated subject had a best response according to WHO criteria of stable disease. Subject 3 had a best response of progressive disease, and also showed a dose limiting toxicity (“DLT” in Table 13) of Grade 4 neutropenia on day 8.

### Example 16

### Correlation of Molecular Markers with Response to Inhibition of IGF-1Receptor Signaling

This example demonstrates that molecular markers can be used to determine whether a subject is more likely or less likely to respond to an anti-tumor treatment comprising an inhibitor of IGF-1 receptor signaling.

The presence or absence of certain biomarkers was found to correlate with the response of subjects to treatment with an inhibitor of IGF-1 receptor signaling. Of the subjects listed in Table 9, both of the subjects with disease progression (PD) after eight weeks of treatment exhibited a reduction of PTEN expression (complete loss of PTEN expression in 10% of the tumor cells observed in one subject, complete loss of PTEN in 5% of tumor cells in the other subject) as assessed by immunohistochemical staining of archival formalin fixed paraffin embedded tumor sections by a contract laboratory (Ventana Medical Systems, Tucson, Ariz.), as shown in FIG. 34. PTEN expression was completely eliminated (absent in 100% of tumor cells) in one subject with stable disease (this subject exhibited a 4% increase in his tumor RECIST measurement). PTEN loss was not observed in either subject who had a PR or a CR to treatment with the anti-IGF-1R antibody.

The subject showing a complete loss of PTEN expression in 5% of tumor cells also was found to have a PTEN loss of function mutation (D331 G).

An activating mutation of the gene encoding KRAS that changed the glycine normally found at codon twelve to a cysteine (i.e., KRAS G12C) was observed in the PR subject with the mid gut carcinoid tumor and in another subject with metastatic melanoma who had stable disease after eight weeks of treatment (RECIST 1% increased).

To further define the relationship between PTEN genotype and responsiveness to treatment with an anti-IGF-1 receptor inhibitor, six human tumor cell lines were identified that display negative PTEN status. Their sensitivity to an anti-IGF-1R antibody was tested in vivo in a mouse xenograft model. The cell lines used were PC-3 and LnCap (prostate), U-87MG (Glioblastoma), Cal-51 (Breast), 786-0 (Kidney), and Colo-320 (Colon/carcinoid). Five million cells of each of these cell lines were injected subcutaneously in the left flank of 4-6 week old female athymic nude mice. When the average tumor size reached approximately 200-220 mm³, mice were randomly assigned into groups (10 mice/group). Therapy with anti-IGF-1R antibody (“Antibody”) at three doses (30, 100, or 300 μg/dose), or human IgG1 control (“Control”; 300 μg/dose) started on randomization day and continued until the end of each study. Administration of Antibody or Control occurred twice per week, intraperitoneally. Tumor volume and body weight of each animal were measured twice per week using calipers and an analytical scale, respectively. Data were gathered as mean+/−standard error. Cell lines were considered responsive to Antibody if a statistically significant decrease in tumor volume was measured between any dose group and the Control group. For the statistical analysis, repeated measures ANOVA (RMANOVA), post-hoc Scheffe, was employed. Results are shown in Table 14. Xenograft data showed that none of the six PTEN null models studied was sensitive to Antibody. In contrast, all sensitive xenograft models displayed wild-type PTEN status. These data support the clinical observations and support the use of PTEN status as a negative stratification marker for treatment with IGF-1R inhibitors.

TABLE 14

| | | | | Antibody |
|---|---|---|---|---|
| Cell Line | PTEN Status | p53 Status | Tumor Type | p < 0.05 TGI |
| Colo 205 | WT | Mut | Colon | Yes |
| DLD-1 | WT | Mut | Colon | Yes |
| BT-474 | WT | Mut | Breast | Yes |
| BxPC-3 | WT | Mut | Pancreas | Yes |
| MiaPaCa | WT | Mut | Pancreas | Yes |
| SJSA-1 | WT | mdm2 | Osteosarcoma | Yes |
| U-87MG | Null | Wt | GMB | No |
| Cal-51 | Null | Wt | BBC | No |
| PC-3 | Null | Mut | Prostate | No |
| LnCap | Null | Wt | Prostate | No |
| Cal-51 | Null | Mut | Breast | No |
| 786-O | Null | Mut | Kidney | No |
| Colo-320 | Null | Mut | Colon/Carcinoid | No |

### Example 17

### A Placebo-Controlled, Randomized Phase 2 Study of Conatumumab (AMG 655) or Ganitumab (AMG 479) or Placebo Plus Gemcitabine in Patients with Metastatic Pancreatic Cancer

This example demonstrates that ganitumab increased median overall survival and progress free survival in a Phase 2 clinical study in metastatic pancreatic cancer patients.

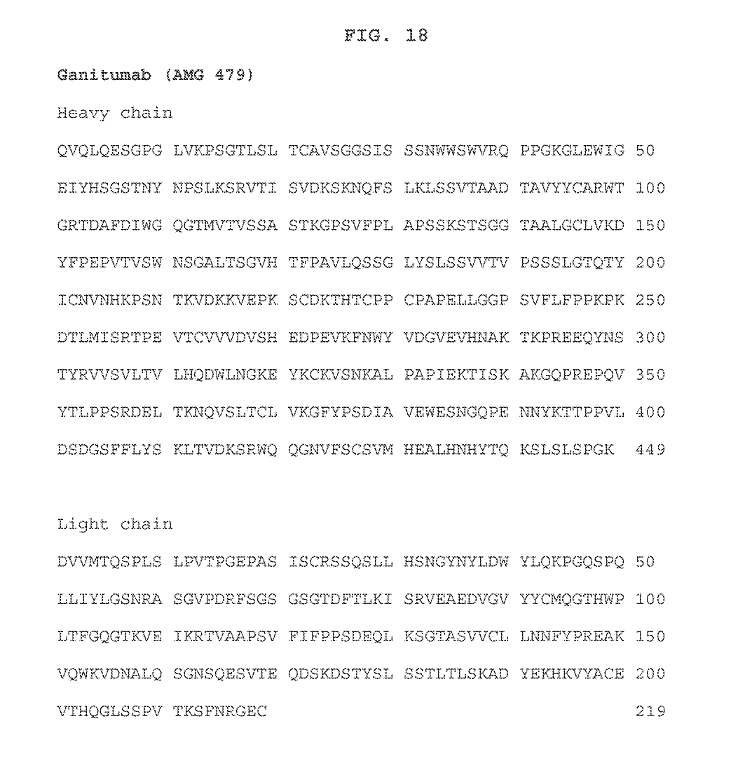

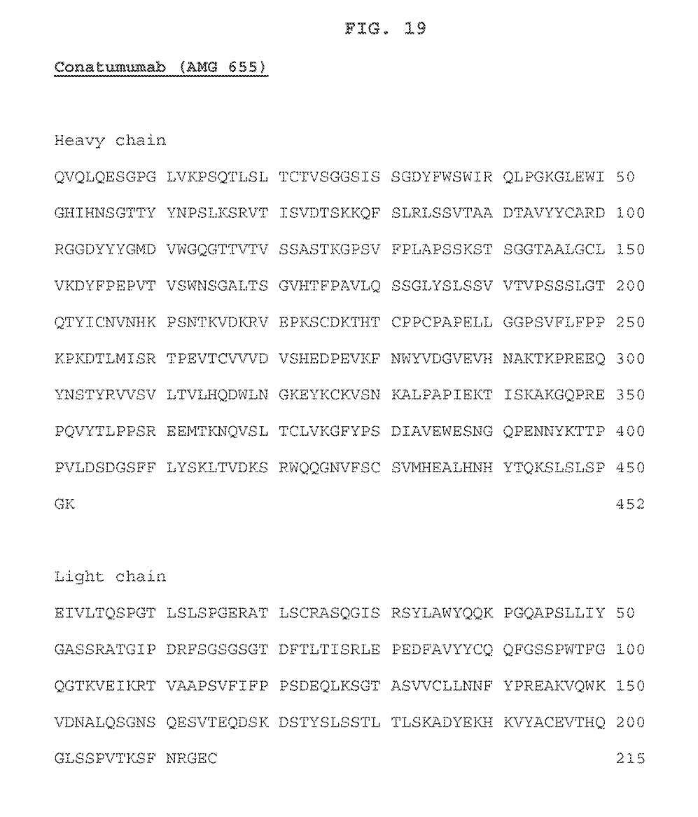

A three-arm, placebo-controlled, randomized phase 2 study was conducted to evaluate ganitumab (FIG. 18) plus gemcitabine or conatumumab (FIG. 19) plus gemcitabine versus placebo plus gemcitabine in metastatic pancreatic cancer patients.

The primary endpoint was six month overall survival rate. Secondary endpoints were response rate (by RECIST; Investigator defined, no central review), progression-free survival (PFS, investigator defined), overall survival (OS), incidence of adverse events and laboratory abnormalities, incidence of anti-ganitumab or anti-conatumumab antibodies, pharmacokinetics of ganitumab and conatumumab, and dose intensity of gemcitabine when combined with ganitumab, conatumumab, or placebo.

Key inclusion criteria were histologically or cytologically documented metastatic adenocarcinoma of the pancreas, age eighteen years or older, ECOG PS 0 or 1, adequate hematologic, hepatic, renal, and coagulation function, amylase and lipase≦2.0×ULN, adequately controlled type 1 or 2 diabetes (amended during protocol to fasting blood glucose<160 mg/mL only), fasting blood glucose≦160 mg/mL, HbAlc<8%, and written informed consent.

Key exclusion criteria were uncontrolled cardiac disease and prior chemotherapy or radiation in the adjuvant or metastatic setting.

The study was designed as an estimation study with a planned sample size of 120 patients (40 patients/arm); the actual number of patients was greater as shown in Table 15. As designed (estimation, not hypothesis-testing), the trial had 80% power to detect a difference between a 6-month OS rate of 45% for placebo, 69% for conatumumab or ganitumab (target improvement, 10%).

The study schema is shown in FIG. 20.

Baseline Demographics are shown in Table 15:

| | | |
|---|---|---|
| Conatumumab | Ganitumab | |
| 10 mg/kg (Q2W) | 12 mg/kg (Q2W) | Placebo |
| (N = 41) | (N = 42) | (N = 42) |

Sex - n (%)

| | | | |
|---|---|---|---|
| Male | 24 (59) | 25 (60) | 26 (62) |
| Female | 17 (41) | 17 (40) | 16 (38) |

Race/ethnicity - n (%)

| | | | |
|---|---|---|---|
| White or Caucasian | 32 (78) | 35 (83) | 37 (88) |
| Black or African | 3 (7) | 3 (7) | 3 (7) |
| American | | | |
| Hispanic or Latino | 5 (12) | 4 (10) | 2 (5) |
| Asian | 1 (2) | 0 (0) | 0 (0) |

Age (years) at Randomization

| | | | |
|---|---|---|---|
| Mean | 62.3 | 62.1 | 62.6 |
| Median | 61.0 | 66.0 | 61.0 |
| Min, Max | 45, 80 | 37, 82 | 43, 82 |

ECOG performance status at enrollment - n (%)

| | | | |
|---|---|---|---|
| 0 | 17 (41) | 19 (45) | 16 (38) |
| 1 | 24 (59) | 23 (55) | 26 (62) |

Lesion sites at baseline are described in Table 16:

| | | | |
|---|---|---|---|
| Conatumumab | AMG 479 | | |
| 10 mg/kg | 12 mg/kg | | |
| (Q2W) + | (Q2W) + | Placebo + | All |
| gemcitabine | gemcitabine | gemcitabine | Subjects |
| (N = 41) | (N = 42) | (N = 42) | (N = 125) |

Lesions in the Pancreas - n (%)

| | | | | |
|---|---|---|---|---|
| Head | 11(27) | 12(29) | 12(29) | 35(28) |
| Body/Tail | 7(17) | 9(21) | 6(14) | 22(18) |
| Neck | 1(2) | 2(5) | 3(7) | 6(5) |
| NOS | 13(32) | 12(29) | 15(36) | 40(32) |

Lesions outside the Pancreas - n (%)

| | | | | |
|---|---|---|---|---|
| Liver | 27(66) | 29(69) | 33(79) | 89(71) |
| Lung | 19(46) | 14(33) | 13(31) | 46(37) |
| Lymph nodes | 21(51) | 17(40) | 18(43) | 56(45) |
| Abdomen | 3(7) | 4(10) | 8(19) | 15(12) |
| Other | 2(5) | 1(2) | 0(0) | 3(2) |

Lesion sizes at baseline are described in Table 17:

| | | |
|---|---|---|
| Conatumumab | | |
| 10 mg/kg | Ganitumab | |
| (Q2W) + | 12 mg/kg (Q2W) + | Placebo + |
| gemcitabine | gemcitabine | gemcitabine |
| (N = 41) | (N = 42) | (N = 42) |

Sum of the longest diameters (SLD) of target lesions at baseline (mm)

| | | | |
|---|---|---|---|
| n | 38 | 39 | 40 |
| Mean | 98.83 | 85.26 | 108.14 |
| SD | 61.87 | 45.71 | 69.81 |
| Median | 73.00 | 77.00 | 97.00 |
| Q1, Q3 | 59.00, 122.00 | 51.20, 97.00 | 58.00, 144.50 |
| Min, Max | 30.0, 284.0 | 21.0, 218.0 | 24.0, 395.0 |
| Number of | 13 (32) | 12 (29) | 17 (40) |
| patients with at | | | |
| least one lesion | | | |
| >5 cm, n(%) | | | |
| Number of | 27 (66) | 29 (69) | 33 (79) |
| patients with | | | |
| liver metastases - | | | |
| n (%) | | | |

Efficacy was determined as overall survival (OS) and progression-free survival (PFS). The OS and PFS results suggested improvements in both metrics that were robust after adjusting for baseline covariates (age, gender, ECOG PS status, liver metastases, and tumor sum of longest diameter). Results are summarized in Table 18:

| | Conatumumab + | Ganitumab + | Placebo + |
|---|---|---|---|
| | Gemcitabine | Gemcitabline | Gemcitabine |
| 6-Month OS Rate | 59% (42, 73) | 57% (41, 70) | 50% (33, 64) |
| (95% CI)^{a} | | | |
| 12-Month OS Rate | 20% (9, 34) | 39% (25, 54) | 23% (12, 38) |
| (95% CI) | | | |
| Median PFS, | 4.0 (3.3, 5.0) | 5.1 (2.8, 5.8) | 2.1 (1.9, 3.3) |
| Months (95% CI) | | | |
| Stratified HR | 0.65 (0.41, 1.05) | 0.65 (0.41, 1.04) | |
| (95% CI) | | | |
| P-value | 0.08 | 0.07 | |
| Median OS, Months | 7.5 (4.8, 10.0) | 8.7 (5.3, 12.2) | 5.9 (4.1, 9.7) |
| (95% CI) | | | |
| Stratified HR | 0.87 (0.53, 1.43) | 0.67 (0.41, 1.12) | |
| (95% CI) | | | |
| P-value | 0.59 | 0.12 | |

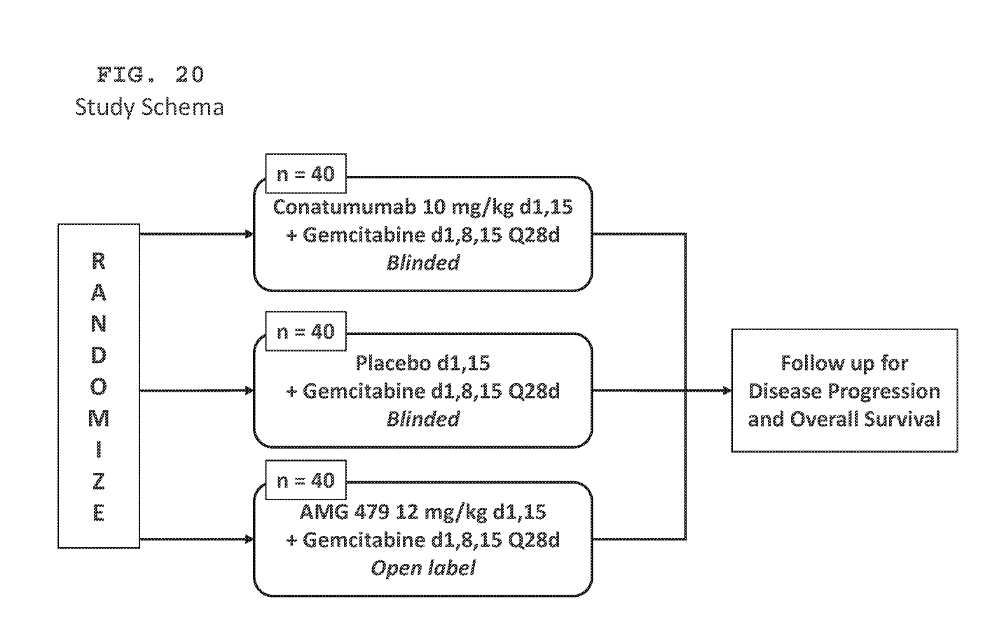

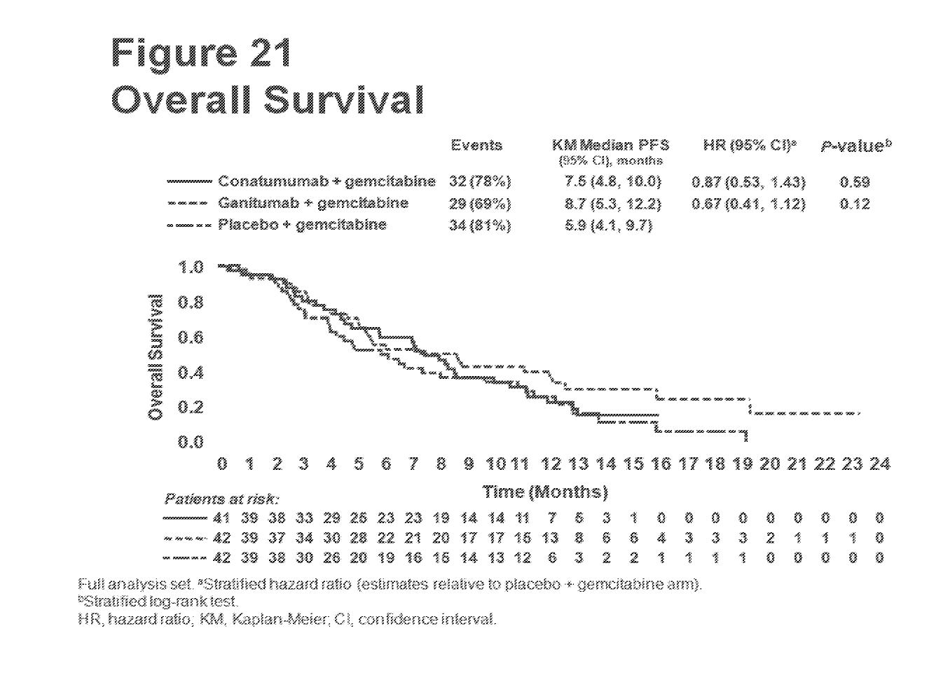

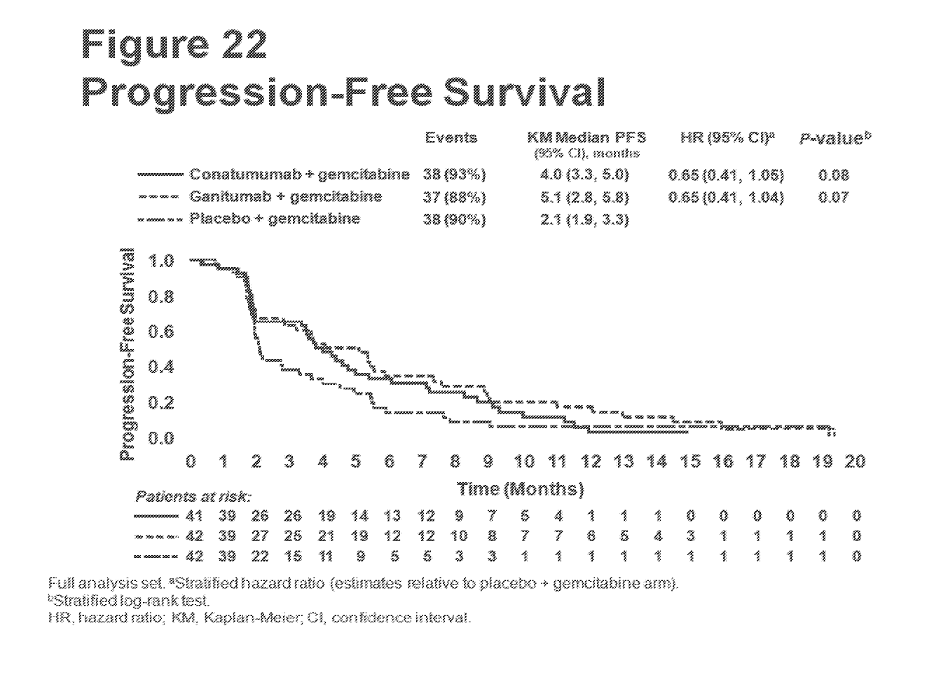

Overall survival data are presented graphically in FIG. 21. Progression free survival data are presented graphically in FIG. 22.

The best overall tumor response data are presented in Table 19:

| | | | |
|---|---|---|---|
| Conatumumab + | Ganitumab + | Placebo + | All |
| Gemcitabine | Gemcitabine | Gemcitabine | Patients |
| (N = 41) | (N = 42) | (N = 42) | (N = 125) |

Number of Patients with Measurable

| | | | | |
|---|---|---|---|---|
| Disease at | 38 | 39 | 40 | 117 |
| Baseline | | | | |

Best Overall Response Assessment - n (%)

| | | | | |
|---|---|---|---|---|
| Confirmed | 0 (0) | 0 (0) | 0 (0) | 0 (0) |
| CR | | | | |
| Confirmed | 1 (3) | 4 (10) | 1 (3) | 6 (5) |
| PR | | | | |
| Unconfirmed | 2 (5) | 0 (0) | 3 (8) | 5 (4) |
| PR | | | | |
| SD (Includes | 22 (58) | 16 (41) | 15 (38) | 53 (45) |
| Unconfirmed | | | | |
| PR) | | | | |
| PD | 12 (32) | 13 (33) | 18 (45) | 43 (37) |
| Unknown | 3 (8) | 6 (15) | 6 (15) | 15 (13) |

Grade 3-5 adverse events occurring in at least 5% of patients are presented in Table 20:

| | Conatumumab + | Ganitumab + | Placebo + | |
|---|---|---|---|---|
| | Gemcitabine | Gemcitabine | Gemcitabine | All Pts |
| n (%) | (N = 41) | (N = 40) | (N = 40) | (N = 121) |
| Number of Patients | | | | |
| Reporting ≧1 AE | 36 (88) | 34 (85) | 27 (68) | 97 (80) |
| Hematologic | | | | |
| Neutropenia | 9 (22) | 7 (18) | 5 (13) | 21 (17) |
| Thrombocytopenia | 7 (17) | 6 (15) | 3 (8) | 16 (13) |
| Anemia | 4 (10) | 4 (10) | 1 (3) | 9 (7) |
| Non-Hematologic | | | | |
| Abdominal pain | 7 (17) | 3 (8) | 5 (13) | 15 (12) |
| Fatigue | 5 (12) | 5 (13) | 2 (5) | 12 (10) |
| Alanine Aminotransferase | | | | |
| Increased | 2 (5) | 6 (15) | 3 (8) | 11 (9) |
| Aspartate Aminotransferase | | | | |
| Increased | 3 (7) | 4 (10) | 2 (5) | 9 (7) |
| Hyperglycemia | 1 (2) | 7 (18) | 1 (3) | 9 (7) |
| Deep Vein | | | | |
| Thrombosis | 4 (10) | 2 (5) | 1 (3) | 7 (6) |
| Hyperbilirubinemia | 2 (5) | 3 (8) | 2 (5) | 7 (6) |
| Hypotension | 3 (7) | 3 (8) | 0 (0) | 6 (5) |
| Pulmonary Embolism | 1 (2) | 2 (5) | 3 (8) | 6 (5) |
| Grade 5 AE | 7 (17)^{a} | 4 (10)^{b} | 1 (3)^{c} | 12 (10) |

^{a}One GI perforation, one GI hemorrhage, one hepatic failure in a patient with liver metastases, 4 PD;
^{b}One acute renal failure, one hemorrhage, two PD;
^{c}One GI hemorrhage

Drug exposure data are presented in Table 21:

| | Conatumumab + | Ganitumab + | Placebo + |
|---|---|---|---|
| Median | Gemcitabine | Gemcitabine | Gemcitabine |
| (Range) | (N = 41) | (N = 40) | (N = 40) |
| Number of | 4 (1 to 17) | 4 (1 to 17) | 2 (1 to 11) |
| Cycles | | | |
| Number of | 7 (1 to 31) | 6 (1 to 32) | 4 (1 to 21) |
| Infusions of | | | |
| IP Per Patient | | | |
| Number of | 10 (1 to 39) | 9 (2 to 47) | 6 (2 to 31) |
| Infusions of | | | |
| Gemcitabine | | | |
| Per Patient | | | |
| Average Dose | 10.1 (8.2 to 10.7) | 12.1 (9.2 to 12.7) | 0 |
| of IP | | | |
| Delivered, | | | |
| mg/kg/infusion | | | |
| Relative Dose | 0.70 (0.3 to 1.0) | 0.80 (0.4 to 1.0) | 0.78 (0.1 to 1.0) |
| Intensity of | | | |
| Gemcitabine^{a} | | | |
| Time on IP, | 3.38 (0.3 to 14.8) | 3.56 (0.3 to 15.4) | 1.94 (0.5, 10.6) |
| Months | | | |

^{a}Ratio of cumulative dose of drug:protocol specified cumulative dose over the specified period.
IP, investigational product (conatumumab or ganitumab)

The results show evidence of activity for ganitumab with trends across several efficacy parameters in metastatic pancreatic cancer, including higher 6-month OS rate (the study's primary endpoint; 57% vs 50%), higher 12-month OS (39% vs 24%), longer PFS (5.1 vs. 2.1 months, HR 0.65), longer OS (8.7 vs. 5.9 months, HR 0.67), and higher rates of SD+PR (51% vs 41%). The study also shows evidence of activity for conatumumab, including higher 6-month OS rate (the study's primary endpoint; 59% vs. 50%), longer PFS (4.0 vs. 2.1 months, I-ER 0.65), and higher rates of SD+PR (61% vs 41%). Both drug combinations were well-tolerated.

### Example 18

### Exposure-Response Analysis for Ganitumab (AMG 479) in Combination with Gemcitabine to Treat Metastatic Pancreatic Cancer

Pooled data from 37 patients with metastatic pancreatic cancer (mPC; 35 from the Phase 2 trial described in Example 17 and two from a first-in-human study) and 62 patients with advanced non-pancreatic solid tumors were used to construct a population PK model for ganitumab.

TABLE 22

| | | | | | |
|---|---|---|---|---|---|
| | Ganitumab Dose | | | | |
| | of PK Samples | | | PK Sampling | |
| Study | (No. of Patients) | Tumor type | Study Drug(s) | Frequency | No. |

Phase 1

| | | | | | |
|---|---|---|---|---|---|
| Ganitumab | 1 mg/kg (n = 3) | Non-pancreatic, n = 51 | Ganitumab | PK profiles (0-336 hour) for 1st | 914 |
| monotherapy IV | 3 mg/kg (n = 3) | Pancreatic, n = 2 | alone | and 3rd doses; sampled pre- and | |
| Q2W2 | 10 mg/kg (n = 3) | (1 received 12 mg/kg; | | end of infusion in specified | |
| | 12 mg/kg (n = 21) | 1 received 20 mg/kg) | | cycles | |
| | 20 mg/kg (n = 23) | | | | |
| Ganitumab + | 6 mg/kg (n = 8) | Non-pancreatic, n = 11 | Ganitumab + | PK profiles (0-336 hour) for 3rd | 117 |
| gemcitabine | 12 mg/kg (n = 3) | | gemcitabine | dose; sampled pre- and end of | |
| IV Q2W3 | | | | infusion in specified cycles | |

Phase 2

| | | | | | |
|---|---|---|---|---|---|
| Ganitumab + | 12 mg/kg (n = 35)^{a} | Pancreatic, n = 35 | Ganitumab + | PK profiles (0-336 hour) for 1st | 196 |
| gemcitabine IV | | | gemcitabine | dose; sampled pre- and end of | |
| Q2W1 | | | | infusion in specified cycles | |

^{a}35 out of 40 patients who received ganitumab in the Phase 2 study had ganitumab concentration data.

Eighty patients with mPC who received either ganitumab plus gemcitabine or placebo plus gemcitabine were included in the exposure-response analysis.

A population PK model was constructed with a two-compartment disposition structural model with non-linear mixed effects modeling (NONMEM).

Univariate analysis was performed to illustrate the effect of exposure of patients to ganitumab which was calculated as the integral of the exposure curve over time (AUC_{SS}). When patients receiving ganitumab were stratified based on exposure (into a high exposure group where AUC_{SS} was greater than or equal to the median value (19.2 mg·h/mL) and a low exposure group where AUC_{SS} was less than the median value), Kaplan-Meier plots for OS and PFS for the high and low exposure groups were used to evaluate the effect of exposure on OS and PFS using Cox proportional hazard models. Multivariate analysis was performed to evaluate the effect of exposure on OS and PFS adjusting for potential confounding factors using Cox proportional hazard models with a forward selection technique. All baseline factors were initial candidates in the forward selection model. Only the most statistically significant variables were added to the final model after the forward selection process.

Baseline factors for the multivariate analysis are listed in Table 23:

Demographics
Height, Weight*, Gender, Age, BSA
Baseline Lab Values/Vital Signs
Bilirubin, SGOT, SGPT, alkaline phosphatase, serum creatinine,
* creatinine clearance, BUN, albumin,* fasting glucose, platelets, ANC,
WBC, lymphocytes, systolic/diastolic BP
Baseline Prognostic Factors
ECOG PS, Liver metastasis, Years of diagnosis, Baseline tumor size,
CA 19-9
*Covariate affecting PK
Abbreviations:
BSA, body surface area;
SGOT, serum glutamic-oxaloacetic transaminase;
SGPT, serum glutamic pyruvic transaminase;
BUN, blood urea nitrogen;
ANC, absolute neutrophil count;
WBC, white blood cell;
ECOG PS, Eastern Cooperative Oncology Group Performance Status.

The effect of exposure on the incidence of selected adverse events (hearing and vestibular disorders, hepatic disorders, hyperglycemia, infusion reaction, neutropenia, rash and skin-related toxicity, thrombocytopenia, and venous embolic and thrombotic events) was summarized with descriptive statistics.

The effect of exposure on changes in selected laboratory values (aspartate transaminase, alanine transaminase, fasting glucose, total neutrophils, and platelets) was assessed by linear regression.

The AUC_{SS} distribution and PFS profiles for 12 and 20 mg/kg ganitumab Q2W were evaluated with Monte Carlo simulations.

Ganitumab PK parameters from the final model are shown in Table 24:

| Population PK | | | Inter- |
|---|---|---|---|
| Individual | | | variability |
| Parameters | Tumor Type | Typical Values | (%) |
| CL (L/h) | Pancreatic | 0.0481 · (WT/74.55)^{0.984} · (ALB/38^{)-0.859} · (CR/0.8)^{-0.394} | 26.19% |
| | Non-Pancreatic | 0.0283 · (WT/74.55)^{0.984} · (ALB/38)^{-0.859} · (CR/0.8)^{-0.394} | |
| Vc (L) | Pancreatic | 5.13 · (WT/74.55)^{0.559} | 21.66% |
| | Non-Pancreatic | 3.85 · (WT/74.55)^{0.559} | |
| Q (L/h) | | 0.0261 | 43.70% |
| Vp (L) | | 3.41 | 60.25% |
| Error Model | | | |
| Proportional Error (%) | | 16.43% | NA |

ALB, albumin (g/L);
CL, clearance;
CR, serum creatinine (mg/dL);
NA, not applicable;
Q, inter-compartmental clearance;
Vc, central volume of distribution;
Vp, peripheral volume of distribution;
WT, weight (kg).

Faster clearance (CL) and larger central volume of distribution (Vc) were observed in patients with mPC resulting in lower ganitumab exposure. Tumor type (pancreatic versus non-pancreatic) and baseline body weight, albumin, and serum creatinine were identified as statistically significant covariates of ganitumab PK parameters. Gemcitabine co-administration was not a significant covariate of ganitumab PK parameters.

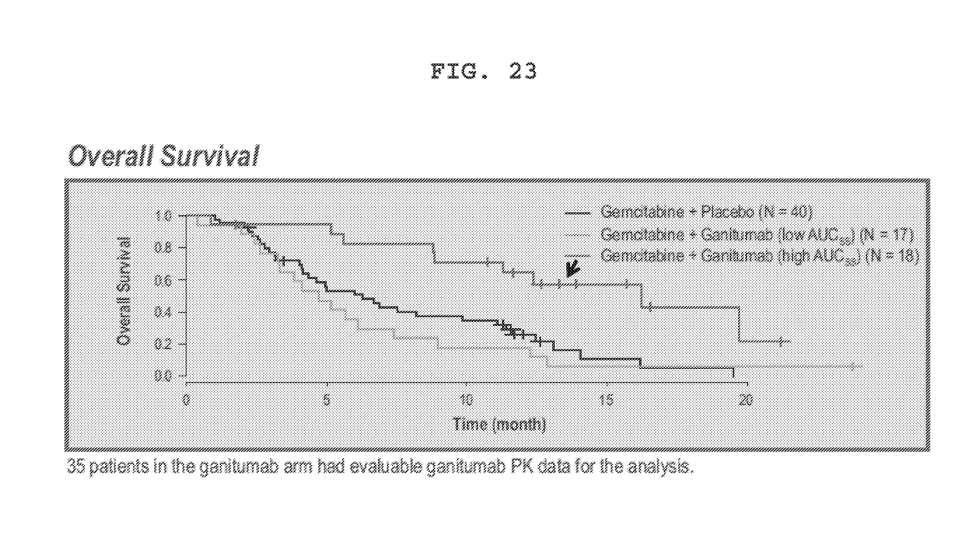

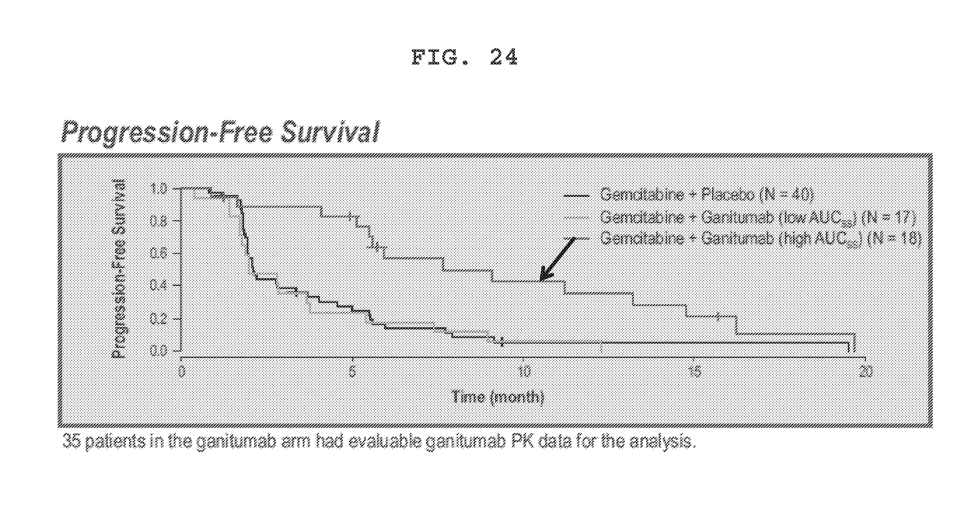

The effect of ganitumab exposure on overall survival and progression free survival is shown in FIG. 23, FIG. 24 and in Table 25:

| Clinical | Exposure | Ganitumab Only | | Ganitumab and Placebo | |
|---|---|---|---|---|---|
| Endpoints | Parameter | *HR (95% CI) | P-value | HR (95% CI) | P-value |
| PFS | logAUCₛₛ | 0.0611 (0.0160, 0.2327) | <.0001 | 0.8322 (0.7111, 0.9741) | 0.0222 |
| OS | logAUCₛₛ | 0.0421 (0.0089, 0.1983) | <.0001 | 0.8288 (0.6978, 0.9845) | 0.0325 |

*HR = Cox proportional hazard ratio for a survival event (PFS or OS) for one unit increment in AUCₛₛ (natural log scale). Results from the ganitumab arm and the placebo (logAUCₛₛ = 0) plus ganitumab arms are presented.

The association between exposure and OS and PFS was significant whether or not data from the placebo arm (assigned an AUCss of zero) were included. Imbalances in baseline prognostic factors between the high and low exposure groups may confound the exposure-PFS and exposure-OS relationships. Therefore, multivariate analyses and sensitivity analyses were performed to confirm these relationships.

The effect of baseline factors on progression free survival and overall survival is shown in Table 26:

| | | | | | | | |
|---|---|---|---|---|---|---|---|
| | | Parameter | | Chi- | | | |
| Variable | DF | Estimate | SE | Square | p-value | HR | 95% CI |

Effect on Progression Free Survival

| | | | | | | | |
|---|---|---|---|---|---|---|---|
| Log AUCss | 1 | −1.9986 | 0.7762 | 6.6302 | 0.0100 | 0.1355 | 0.0296, 0.6204 |
| Baseline ALP | 1 | 0.0035 | 0.0018 | 3.9088 | 0.0480 | 1.0035 | 1.0000, 1.0071 |
| Baseline CrCl | 1 | 0.0130 | 0.0051 | 6.5334 | 0.0106 | 1.0130 | 1.0030, 1.0232 |

Effect on Overall Survival

| | | | | | | | |
|---|---|---|---|---|---|---|---|
| Log AUCₛₛ | 1 | −2.4742 | 0.8840 | 7.8335 | 0.0051 | 0.0842 | 0.0149, 0.4764 |
| Baseline ALP | 1 | 0.0064 | 0.0019 | 11.8663 | 0.0006 | 1.0064 | 1.0028, 1.0101 |

Data are from the ganitumab-treated arm.
ALP, alkaline phosphatase; C, confidence interval; CrCl, creatine clearance; AUCss, steady state AUC; HR, hazard ratio; SE, standard error. DF, degree of freedom.

Higher exposure and lower baseline ALP were associated with longer OS and PFS; lower baseline creatine clearance (CrCl) was associated with longer PFS. The effect of exposure on efficacy remained after adjusting for potential baseline factors.

In general, the incidence of AEs was not dependent on exposure to ganitumab. The incidences of grade 3/4 hyperglycemia and thrombocytopenia were higher in the high-exposure group; the incidence of grade 3/4 hepatic disorders was higher in the low exposure group. Grade 5 AEs included three patients in the low-exposure group (2 disease progression, 1 hemorrhage), one in the high-exposure group (acute renal failure; patient had prior history of IV contrast-induced renal insufficiency), and one in the placebo arm (gastrointestinal hemorrhage). There was no strong evidence of an association between exposure and selected laboratory values.

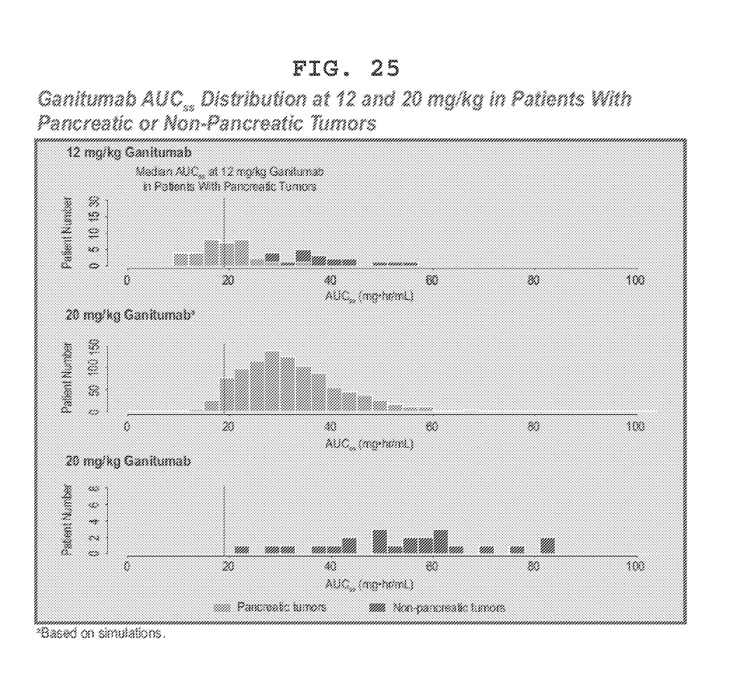

Ganitumab AUC_{SS} distributions at 12 and 20 mg/kg in patients with pancreatic or non-pancreatic tumors (observed and predicted by simulation) are shown in FIG. 25.

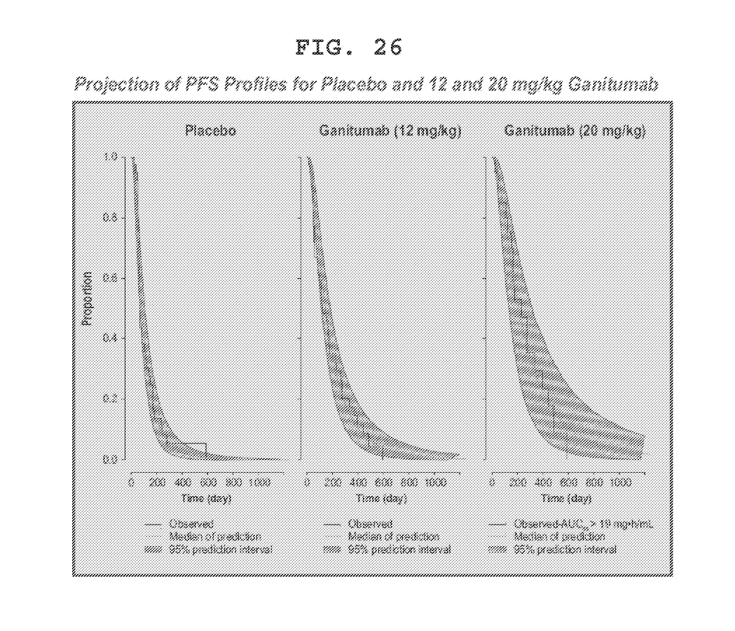

Projections of PFS profiles for placebo and 12 and 20 mg/kg ganitumab are shown in FIG. 26. The projected median PFS times in the placebo and 12 and 20 mg/kg ganitumab dose groups were 2.6, 4.7, and 6.7 months, respectively, indicating that a higher dose of ganitumab may further improve PFS.

In summary, ganitumab exposure among patients with mPC was ˜40% lower than that observed among patients with other tumor types. An effect of gemcitabine on ganitumab PK was not evident. PFS and OS were longer among patients in the high exposure group. The relationship between exposure and survival remained after adjusting for baseline prognostic factors. There was no strong association between exposure and the incidence of most adverse events.

### Example 19

### Identification of Biomarkers

To ascertain whether bioavailable ligand concentrations predict response to ganitumab treatment, the relationship between baseline levels of IGF-1 (free and total), IGF-1 (total), and the major binding proteins (IGFBP-1, -2, -3, -4) and overall survival was carried out for the clinical study described in Example 17.

Descriptive statistics on the baseline levels of circulating biomarkers (free IGF-1, total IGF-1, IGF-II and IGFBP1-4) are summarized in Table 27:

TABLE 27
Distribution of baseline levels of IGF-1R ligands and binding proteins in patients treated
with (a) ganitumab in combination with gemcitabine or (b) placebo in combination with
gemcitabine. Free/total IGF-1, Total IGF-2 and IGFBP-1, -2, -3, and -4 were analyzed by
immunoassay. Median, Mean, Min., and Max. values aregiven in ng/mL, except for
IGFBP-3, which are given in μg/mL.

| Circulating | (a) ganitumab (AMG 479; N = 34) | | | | |
|---|---|---|---|---|---|
| Biomarker | N | Median | Mean (SD) | Q1, Q3 | Min, Max |
| Total IGF-1 | 31 | 103 | 111.48 | 75.00, 137.00 | 41.00, 208.00 |
| | | | (46.96) | | |
| IGF-2 | 33 | 2034.78 | 2072.10 | 1587.47, 2463.27 | 1110.67, 3480.08 |
| | | | (624.58) | | |
| IGF-BP1 | 31 | 28.29 | 36.74 | 13.01, 50.95 | 3.47, 116.82 |
| | | | (31.56) | | |
| IGF-BP2 | 30 | 155.77 | 194.13 | 135.70, 227.80 | 68.84, 577.11 |
| | | | (113.72) | | |
| IGF-BP3 | 31 | 1.9 | 1.85 | 1.30, 2.30 | 0.70, 3.50 |
| | | | (0.69) | | |
| IGF-BP4 | 33 | 46.1 | 49.27 | 36.40, 57.20 | 24.20, 101.90 |
| | | | (19.08) | | |
| Free IGF-1 | 31 | 0.06 | 0.23 | 0.06, 0.37 | 0.06, 1.22 |
| | | | (0.28) | | |
| Circulating | (b) Placebo (N = 33) | | | | |
| Biomarker | N (%) | Median | Mean (SD) | Q1, Q3 | Min, Max |
| Total IGF-1 | 29 | 118 | 129.79 | 90.00, 152.00 | 43.00, 383.00 |
| | | | (66.08) | | |
| IGF-2 | 33 | 1734.16 | 1827.53 | 1448.53, 2224.83 | 838.38, 2994.14 |
| | | | (524.59) | | |
| IGF-BP1 | 26 | 30.27 | 37.67 | 11.39, 47.73 | 1.75, 167.84 |
| | | | (35.81) | | |
| IGF-BP2 | 28 | 169.59 | 179.47 | 125.23, 228.39 | 33.57, 490.12 |
| | | | (91.51) | | |
| IGF-BP3 | 29 | 1.9 | 1.95 | 1.60, 2.30 | 0.80, 3.00 |
| | | | (0.51) | | |
| IGF-BP4 | 33 | 39.9 | 41.74 | 30.60, 49.00 | 23.60, 96.80 |
| | | | (15.39) | | |
| Free IGF-1 | 27 | 0.06 | 0.12 | 0.06, 0.06 | 0.06, 0.67 |
| | | | (0.14) | | |

To establish the relationship between levels of these baseline markers and overall survival, a Cox proportional hazard model was used to calculate hazard ratios (HR) using normalized log transformed marker concentrations. This analysis tests the risk of death associated with a unit increase in the normalized log baseline biomarker and this risk is expressed as a hazard ratio. Because of the small sample size, it is difficult to dissect prognostic associations from relationships that are modified by treatment with ganitumab relative to placebo, i.e., predictive. This is because the addition of treatment effect adds a variable to the analysis that compromises the power of the test masking significant predictive relationships that may exist. However even a prognostic relationship identified in this analysis is important because it informs the hypothesized relationship between circulating bioavailable ligands and the adaptation of the tumor to IGF-1R dependent progression, and in turn sensitivity to inhibitors that block IGF-1R or downstream signaling pathways.

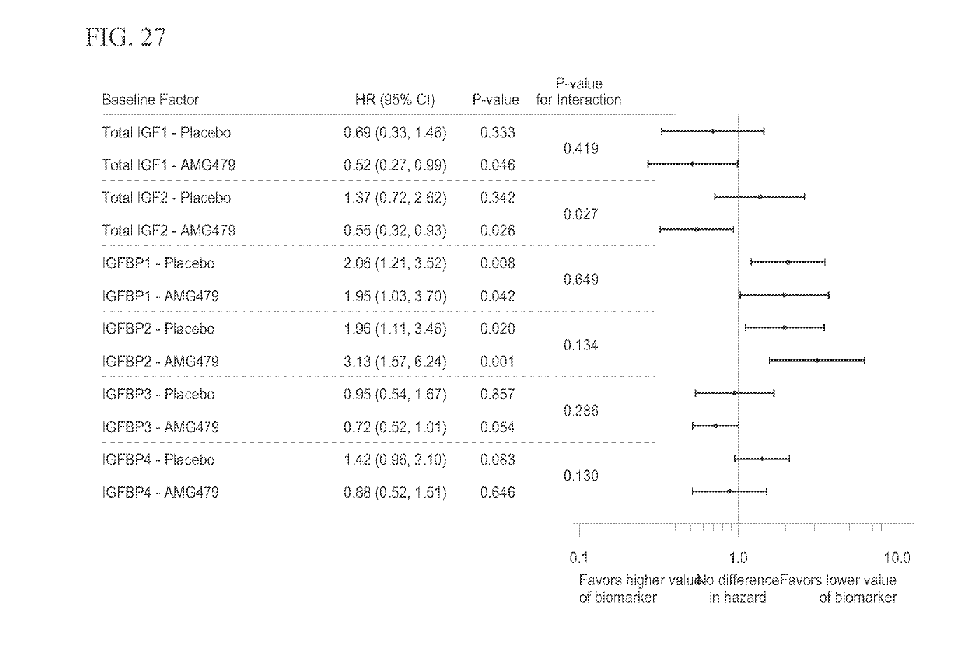

Cox model data are tabulated and represented graphically as a Forest plot in FIG. 27. With the caveat described above, the hazard ratio (HR), with its corresponding 95% CI and P-value, was estimated independently for the ganitumab and placebo arms and the significance modification of biomarker effect with treatment status was tested using both ganitumab and placebo arms. The significance is expressed as a P-value for interaction. Although this interaction analysis may be overly stringent, any marker that is associated with a significant P-value for interaction is likely to be highly predictive of ganitumab response.

As shown in FIG. 27, in the ganitumab (AMG 479) arm significant relationships between overall survival and baseline levels of total IGF-1, IGF-2, IGFBP-1, IGFBP-2 and IGFBP-3 were observed suggesting that circulating components of the IGF system are associated with tumor progression in pancreatic cancer. This relationship can be either prognostic or predictive of ganitumab treatment. To test the latter, these relationships were tested in the placebo treatment arm. In this case, significant relationships were observed for IGFBP-1 and IGFBP-2. The significance of the difference between arms, i.e., the specificity for ganitumab treatment (or predictive value) was calculated by testing the interaction with treatment status. Using the conventional threshold p-value of 0.05, the treatment effect is significant for IGF-2 suggesting that baseline levels of this marker are predictive of response to ganitumab. However, given the small sample size, a less stringent p-value threshold can be used. If a more reasonable p-value cut-off 0.15 is used, IGFBP-2 and IGFBP-4 each reaches significance as well. Without being bound to any particular theory, the association of these markers with OS in pancreatic cancer supports the hypothesis that pancreatic tumors are adapted to using IGF-1R for growth and survival and that circulating levels of IGF-1R ligand and the binding proteins that regulate bio-availability of these ligands are important determinants of tumor progression and survival. The interaction term between baseline biomarker and treatment is significant for some, but not all, of these markers; however, the small sample size of the study and minor differences of markers, which may have a prognostic relationship as well as a predictive relationship, between arms means that this test may miss predictive relationships.

To further test this hypothesis, median values of each of the markers from the overall population (i.e., both treatment and placebo arms) were used as a threshold and the predictive value for OS between the resulting high and low biomarker defined subgroups was established by comparing each sub-group in the ganitumab arm with the corresponding group from the placebo arm. This allows a direct test of the predictive value of using these markers in a dichotomized way to predict OS in pancreatic cancer patients that are treated with inhibitors of IGF-1R.

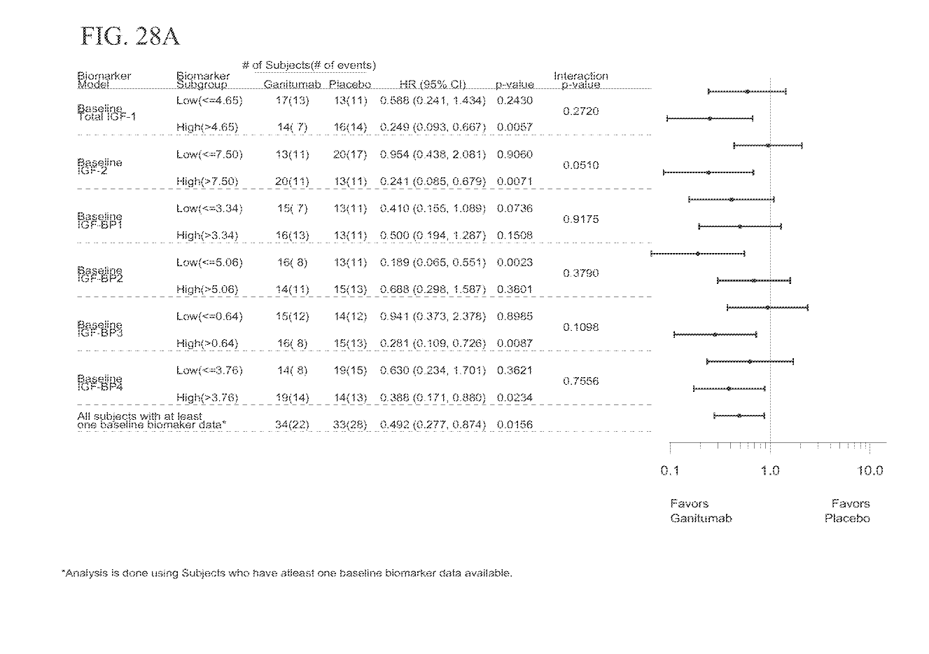

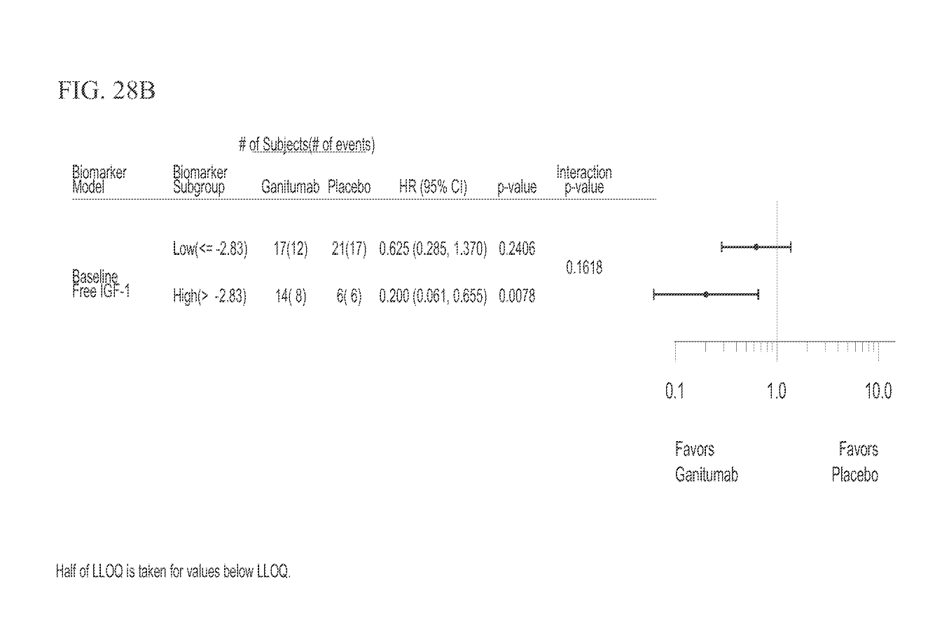

The biomarker defined sub-group analysis is shown in Table 28 and FIG. 28 with the HR and associated P-values for comparisons of ganitumab treatment effect on OS in the biomarker defined low and high sub-groups. Low and significant HR for ganitumab versus placebo was observed in low sub-groups, but not in high sub-groups, for baseline IGFBP-2, suggesting that levels below median may be predictive for response to ganitumab. In contrast, low and significant HR were observed in high sub-groups, but not low sub-groups, for total IGF-1, IGF-2 and IGFBP-3, suggesting that levels above median may be predictive for response to ganitumab.

TABLE 28
Analysis of the relationship between OS in ganitumab and placebo arms in baseline biomarker
subgroups defined by median levels of each marker in the overall population. The sample
size is shown together with the number of OS events in that group in parentheses.

| | Treatment effect (ganitumab vs placebo) | | | | | | | | |
|---|---|---|---|---|---|---|---|---|---|
| | Low (<median) | | | | High (>=median) | | | | |
| Circulating | N (N of | HR | P | Median OS | N (N of | HR | P | Median OS | Interaction |
| Biomarker | events) | (95% CI) | value | (95% CI) | events) | (95% CI) | value | (95% CI) | P value |
| Total IGF-1 | 30(24) | 0.59 | 0.243 | 142 | 30(21) | 0.25 | 0.006 | 487 | 0.272 |
| | | (0.24, 1.43) | | (79, 371) | | (0.09, 0.67) | | (339, —) | |
| IGF-2 | 33(28) | 0.95 | 0.906 | 217.5 | 33(22) | 0.24 | 0.007 | 368 | 0.051 |
| | | (0.44, 2.08) | | (99, 371) | | (0.09, 0.68) | | (184, —) | |
| IGF-BP1 | 28(18) | 0.41 | 0.074 | — | 29(24) | 0.5 | 0.151 | 170 | 0.918 |
| | | (0.15, 1.09) | | (184, —) | | (0.19, 1.29) | | (115, 371) | |
| IGF-BP2 | 29(19) | 0.19 | 0.002 | 487 | 29(24) | 0.69 | 0.38 | 165 | 0.379 |
| | | (0.06, 0.55) | | (371, —) | | (0.3, 1.59) | | (99, 184) | |
| IGF-BP3 | 29(24) | 0.94 | 0.899 | 168 | 31(21) | 0.28 | 0.009 | 487 | 0.11 |
| | | (0.37, 2.38) | | (79, 371) | | (0.11, 0.73) | | (264, —) | |
| IGF-BP4 | 33(23) | 0.63 | 0.362 | 174.5 | 33(27) | 0.39 | 0.023 | 368 | 0.756 |
| | | (0.23, 1.7) | | (123, —) | | (0.17, 0.88) | | (264, 591) | |
| | Treatment effect (conatumumab vs placebo) | | | | | | | | |
| | Low (<median) | | | | High (>=median) | | | | |
| Circulating | N (N of | HR | P | Median OS | N (N of | HR | P | Median OS | Interaction |
| Biomarker | events) | (95% CI) | value | (95% CI) | events) | (95% CI) | value | (95% CI) | P-value |
| Total IGF-1 | 31(26) | 0.53 | 0.183 | 178 | 31(26) | 0.56 | 0.214 | 231 | 0.91 |
| | | (0.21, 1.35) | | (129, 323) | | (0.23, 1.39) | | (139, 305) | |
| IGF-2 | 34(30) | 1.08 | 0.857 | 263 | 35(27) | 0.56 | 0.188 | 214 | 0.15 |
| | | (0.47, 2.46) | | (116, —) | | (0.23, 1.33) | | (135, 261) | |
| IGF-BP1 | 29(22) | 0.68 | 0.375 | 254 | 29(26) | 0.77 | 0.529 | 178 | 0.785 |
| | | (0.29, 1.6) | | (139, 393) | | (0.35, 1.72) | | (82, 261) | |
| IGF-BP2 | 29(23) | 0.91 | 0.835 | 244 | 30(26) | 0.69 | 0.361 | 178 | 0.562 |
| | | (0.39, 2.14) | | (147, 365) | | (0.31, 1.53) | | (92, 323) | |
| IGF-BP3 | 24(20) | 0.86 | 0.76 | 129 | 38(32) | 0.77 | 0.483 | 244 | 0.939 |
| | | (0.32, 2.3) | | (71, 323) | | (0.38, 1.58) | | (147, 305) | |
| IGF-BP4 | 34(29) | 0.86 | 0.701 | 216 | 35(28) | 0.97 | 0.935 | 216 | 0.478 |
| | | (0.4, 1.86) | | (139, 305) | | (0.42, 2.22) | | (84, 343) | |

These relationships suggest that these markers can be used to predict OS in pancreatic cancer patients that are treated with an inhibitor of IGF-1R. These data also suggest that markers that predict response to drugs that inhibit IGF-1R will also be predictive of OS for drugs that act anywhere on the pathway upstream (i.e., agents that regulate the ligands or binding proteins) or downstream (i.e., agents that inhibit intracellular targets that are downstream of IGF-1R which would include, but are not limited to, constituent signaling proteins of the Ras/Raf and PI3K signaling pathways).

Having shown that predictive relationships exist between these circulating proteins and OS for an IGF-1R inhibitor, it was next demonstrated that these predictive relationships are clinically meaningful, i.e., that the differences in OS between high and low biomarker defined subgroups are substantial. To represent these relationships Kaplan-Maier plots were generated that show the OS for the high and low subgroups by plotting the fraction of patients in each sub-group that survive against time. These plots are shown in FIGS. 29 through 33.

In the case of total IGF-1 (FIG. 29; Table 29), there is a clear separation between the curves for high and low sub-groups in the ganitumab arm, while the curves for high and low sub-groups are indistinguishable from each other in the placebo arm.

TABLE 29

| | | Median KM OS Time, Months (95% CI) | | | |
|---|---|---|---|---|---|
| Baseline | Biomarker | Ganitumab + | | | |
| Biomarker | Subgroup | Gemcitabine | | Placebo + Gemcitabine | |
| Total IGF-1 | Low | 4.67 | (2.60-12.19) | 4.27 | (2.73-5.91) |
| | High | 16 | (8.84-NE) | 6.77 | (3.98-11.24) |
| Free IGF-1 | Low | 8.84 | (3.25-16) | 6.57 | (4.07, 11.24) |
| | High | 11.14 | (5.42-NE) | 3.55 | (1.15, 10.91) |
| IGF-2 | Low | 5.59 | (2.60-12.19) | 4.86 | (2.73-12.29) |
| | High | 16 | (6.05-NE) | 5.91 | (3.15-11.24) |
| IGFBP-1 | Low | NE | (6.05-NE) | 7.43 | (3.98-12.29) |
| | High | 5.59 | (3.78-12.19) | 4.07 | (2.73-4.93) |
| IGFBP-2 | Low | 12.65 | (11.14-NE) | 6.57 | (2.73-12.29) |
| | High | 5.52 | (3.25-8.84) | 4.86 | (2.89-6.77) |
| IGFBP-3 | Low | 5.52 | (2.60-12.19) | 4.27 | (2.60-7.43) |
| | High | 16 | (8.67-NE) | 6.77 | (3.98-11.24) |
| IGFBP-4 | Low | 8.59 | (4.04-NE) | 6.77 | (4.07-11.24) |
| | High | 12.09 | (5.59-16.00) | 3.71 | (2.30-11.50) |

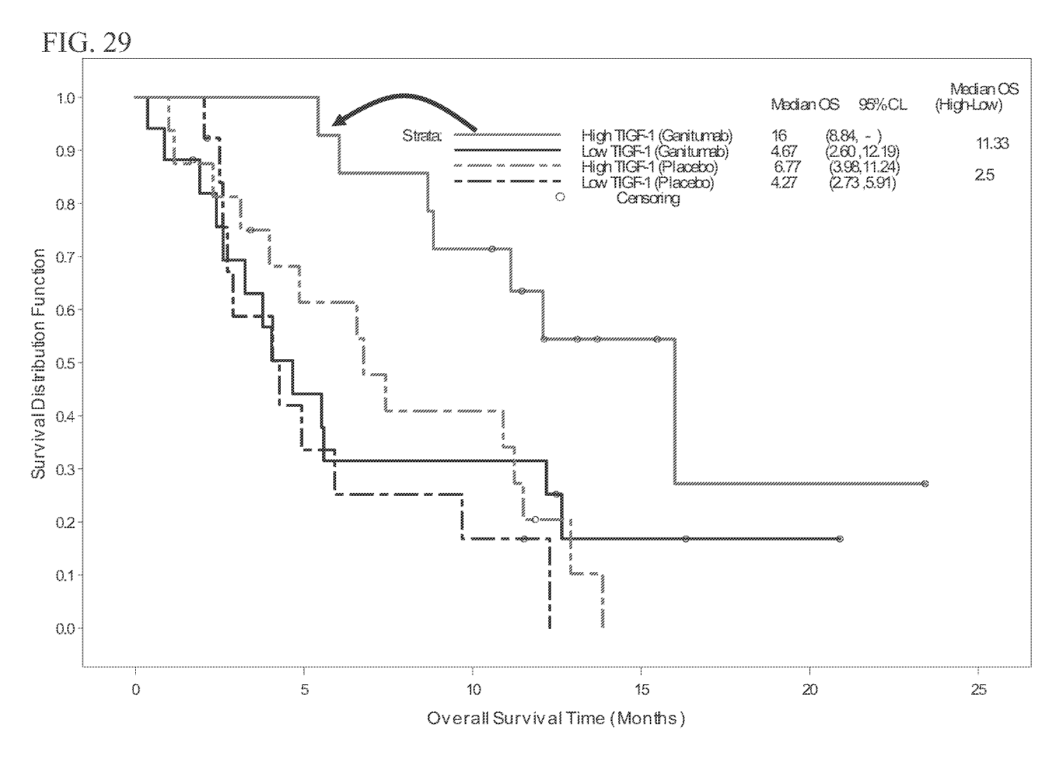

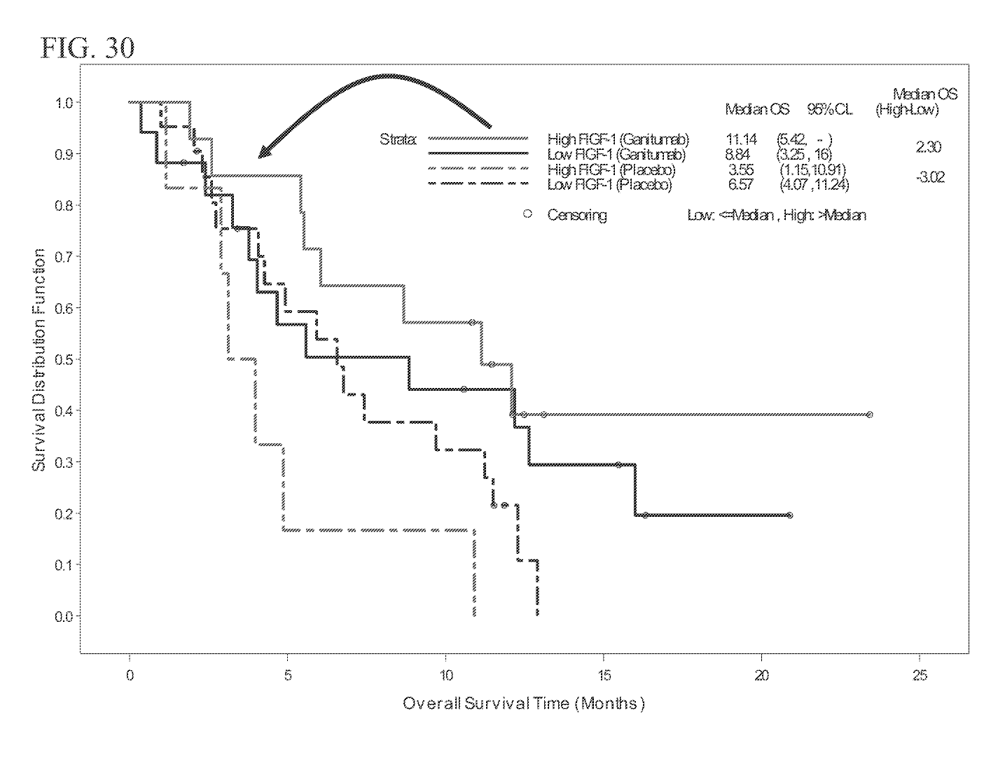

For free IGF-1 (FIG. 30; Table 29), in the high subgroup the median OS in the ganitumab treatment arm was 11.14 months compared to 3.55 months in the placebo arm, a difference of 7.59 months.

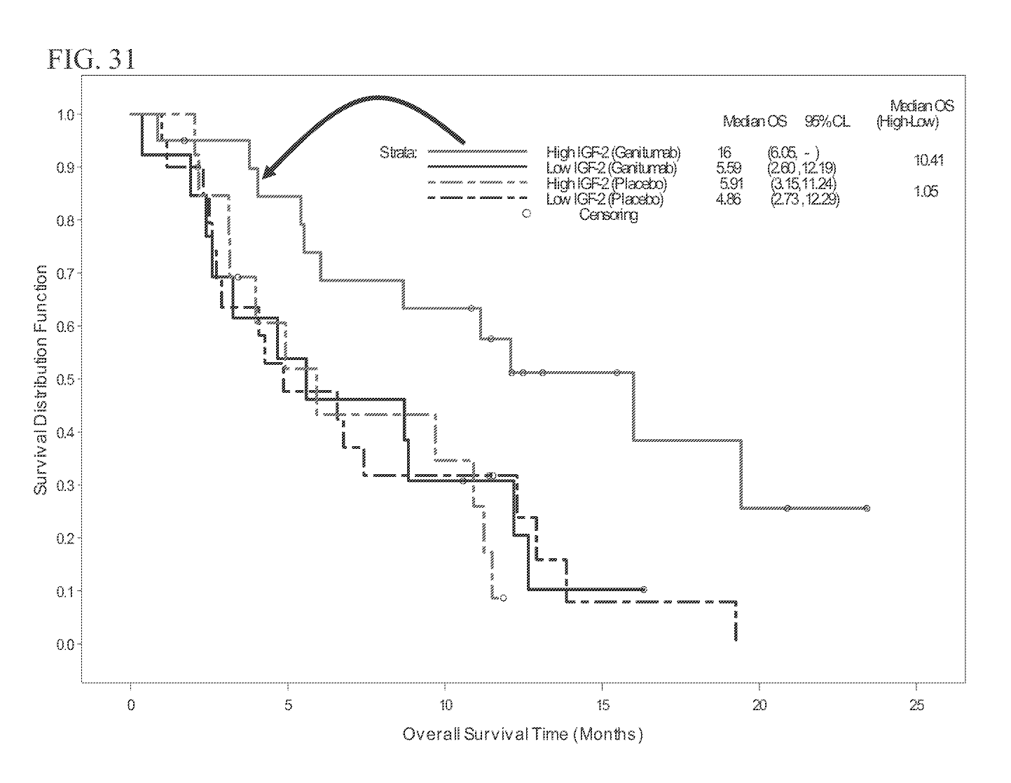

For IGF-2 (FIG. 31; Table 29), again the difference between high and low subgroup OS profiles is apparent in ganitumab arm, with the high subgroup showing improved OS in comparison to the low subgroup and both subgroups in the placebo arms.

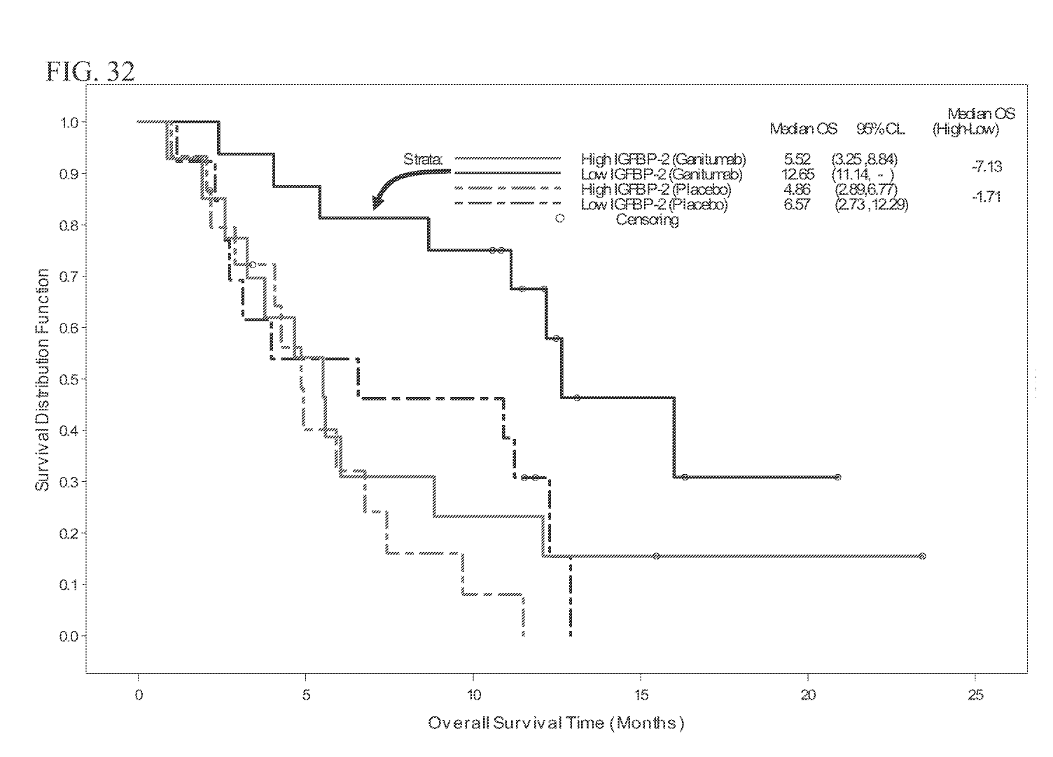

For IGFBP-2 (FIG. 32; Table 29), again a difference between high and low subgroup OS profiles in the ganitumab arm is apparent, however in this case the low subgroup in the ganitumab arm shows improved OS in comparison to the high subgroup in the ganitumab arm and to both subgroups in the placebo arm.

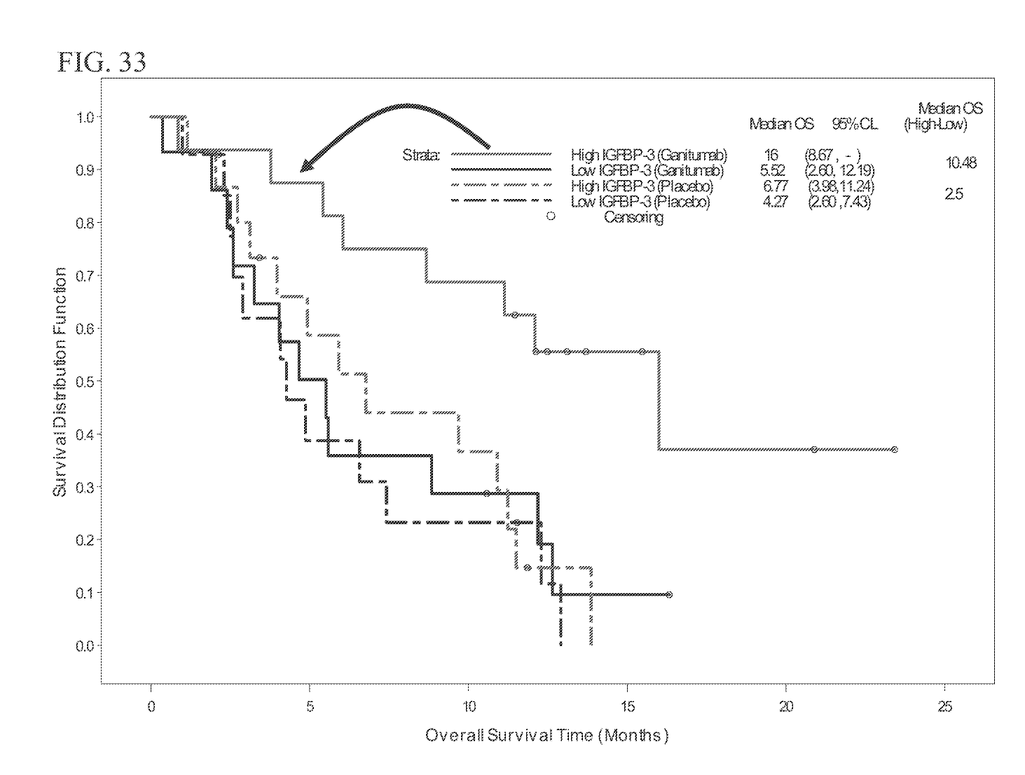

For IGFBP-3 (FIG. 33; Table 29), again the difference between high and low subgroup OS profiles is apparent, with the high subgroup showing improved OS in comparison to the low subgroup and both subgroups in the placebo arms.

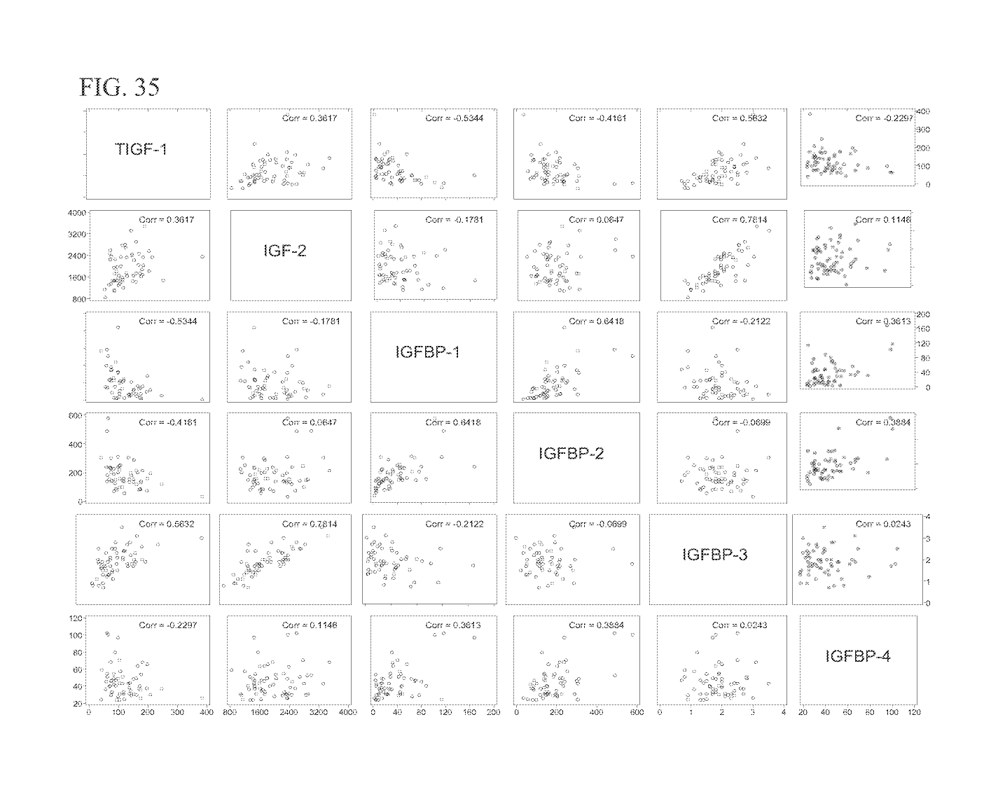

Analysis of the correlations between these markers (non-parametric Spearman correlation) reveals that IGF-1 and IGF-2 levels both significantly correlate with IGFBP-3 levels. No relationship was observed between IGFBP-2 and IGFBP-3 or IGFBP-2 and IGF-2 suggesting that these markers predict different patient populations and that the combination of these markers as a composite panel would be more predictive than any of these alone, as shown in FIG. 35.

As shown in FIG. 35, the correlations between many of the circulating biomarkers were not strong suggesting that they may operate as independent predictors of efficacy. This suggests that ratios of these markers are predictive. In order to compare the predictive potential for biomarker ratios with the individual biomarkers alone, sensitivity and specificity for prediction of nine month survival status were determined through analysis of receiver operator characteristic (ROC) with predictive values presented as the area under the ROC curve (AUC_{ROC}). The strongest effects were observed for the ratios IGF-2/IGFBP-2 and IGFBP-2/IGFBP-3. AUC_{ROC} was higher with the ratio IGF-2/IGFBP-2 (AUC_{ROC}=0.92) than with the individual factors (IGF-2: AUC_{ROC}=0.727; IGFBP-2: AUC_{ROC}=0.772). Likewise, the ratio IGFBP-2/IGFBP-3 had a higher AUC_{ROC} (0.906) than either factor alone (IGFBP-2: AUC_{ROC}=0.772; IGFBP-3 AUC_{ROC}=0.715).

The effect of treatment (ganitumab versus placebo) on OS was analyzed in patients who had higher- or lower-than-median values of these ratios. The median ratio of IGF-2/IGFBP-2 was 11.1509. The median ratio of IGFBP-2/IGFBP-3 was 0.0834. Patients with either a higher-than-median IGF-2/IGFBP-2 ratio or a lower-than-median IGFBP-2/IGFBP-3 ratio were more likely to show a ganitumab-dependent increase in OS. Patients with a lower-than-median IGF-2/IGFBP-2 ratio and a higher-than-median IGFBP-2/IGFBP-3 ratio were less likely to show a ganitumab-dependent increase in OS. These observations are consistent with the subgroup analysis based on single biomarkers (i.e., patients with lower than median IGFBP-2, or higher than median IGF-2, or higher than median IGFBP-3, were more likely to show ganitumab-dependent increases in OS).

Each reference cited herein is incorporated by reference in its entirety for all that it teaches and for all purposes.

1-148. (canceled)

149. A method for assessing the prognosis for a patient comprising determining the amount of a circulating biomarker, wherein said patient has a cancerous condition and the amount of said circulating biomarker indicates whether said patient has a better prognosis.

150. The method of claim 149, wherein said cancerous condition is characterized by IGF-1R signaling activity.

151. The method of claim 149, wherein said cancerous condition is of a type that responds to treatment using an inhibitor of IGF-1R signaling.

152. The method of claim 149, wherein said patient has a tumor.

153. The method of claim 152, wherein said tumor is a solid tumor.

154. The method of claim 152, wherein said tumor is a primary tumor.

155. The method of claim 152, wherein said tumor is a metastatic tumor.

156. The method of claim 152, wherein said tumor is a pancreatic cancer tumor, a sarcoma tumor, a Ewing's sarcoma tumor, an ovarian tumor, a breast cancer tumor, a small cell lung cancer tumor, a non-small cell lung cancer tumor, a colorectal cancer tumor with an activating KRAS mutation, a colorectal cancer tumor with a wild-type KRAS allele, a prostate cancer tumor, a hepatic cancer tumor, a head and neck cancer tumor, a carcinoid tumor, a gastric cancer tumor, a multiple myeloma tumor, a neuroendocrine cancer tumor, an adrenal cell carcinoma tumor, or a hepatocellular carcinoma tumor.

157. The method of claim 156, wherein said pancreatic cancer tumor is a metastatic pancreatic cancer tumor.

158. The method of claim 156, wherein said pancreatic cancer tumor is a locally advanced pancreatic cancer tumor.

159. The method of claim 149, wherein said cancerous condition is Acute Lymphoblastic Leukemia, Adrenocortical Carcinoma, an AIDS-Related Cancer, AIDS-Related Lymphoma, Anal Cancer, Childhood Cerebellar Astrocytoma, Childhood Cerebral Astrocytoma, Basal Cell Carcinoma, Extrahepatic Bile Duct Cancer, Bladder Cancer, Osteosarcoma/Malignant Fibrous Histiocytoma Bone Cancer, a Brain Tumor (e.g., Brain Stem Glioma, Cerebellar Astrocytoma, Cerebral Astrocytoma/Malignant Glioma, Ependymoma, Medulloblastoma, a Supratentorial Primitive Neuroectodermal Tumor, Visual Pathway or Hypothalamic Glioma), Breast Cancer, a Bronchial Adenoma/Carcinoid, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, a Childhood Cancer, Chronic Lymphocytic Leukemia, Chronic Myelogenous Leukemia, a Chronic Myeloproliferative Disorder, Colon Cancer, Colorectal Cancer, Cutaneous T-Cell Lymphoma, Endometrial Cancer, Ependymoma, Esophageal Cancer, a Ewing's Family Tumor, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, a Germ Cell Tumor (e.g., Extracranial, Extragonadal, or Ovarian), Gestational Trophoblastic Tumor, Glioma (e.g., Adult, Childhood Brain Stem, Childhood Cerebral Astrocytoma, Childhood Visual Pathway or Hypothalamic), Hairy Cell Leukemia, Head and Neck Cancer, Hepatocellular (Liver) Cancer, Hodgkin's Lymphoma, Hypopharyngeal Cancer, Hypothalamic or Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocytic, Chronic Myelogenous, or Hairy Cell), Lip or Oral Cavity Cancer, Liver Cancer, Non-Small Cell Lung Cancer, Small Cell Lung Cancer, Lymphoma (e.g., AIDS-Related, Burkitt's, Cutaneous T-Cell, Hodgkin's, Non-Hodgkin's, or Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, a Myelodysplastic Syndrome, a Myelodysplastic/Myeloproliferative Disease, Myelogenous Leukemia, Chronic Myeloid Leukemia, Multiple Myeloma, a Chronic Myeloproliferative Disorder, Nasal Cavity or Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus or Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis or Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-Cell Lymphoma, Testicular Cancer, Thymoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway or Hypothalamic Glioma, Vulvar Cancer, Waldenstrom's Macroglobulinemia, or Wilms' Tumor.

160. The method of claim 149, wherein said circulating biomarker is total IGF-1, IGFBP-2, IGFBP-3, the ratio of IGF-2/IGFBP-2, or the ratio of IGFBP-2/IGFBP-3.

161. The method of claim 160, wherein said circulating biomarker is total IGF-1, and wherein a higher total IGF-1 concentration indicates that said patient has a better prognosis.

162. The method of claim 161, wherein said patient's total IGF-1 concentration is higher if it is greater than about 118 ng/mL and lower if it is less than about 118 ng/mL.

163-166. (canceled)

167. The method of claim 160, wherein said circulating biomarker is IGFBP-2, and wherein a lower IGFBP-2 concentration indicates that said patient has a better prognosis.

168. The method of claim 167, wherein said patient's total IGFBP-2 concentration is higher if it is greater than about 170 ng/mL and lower if it is less than about 170 ng/mL.

169. The method of claim 160, wherein said circulating biomarker is IGFBP-3, and wherein a higher IGFBP-3 concentration indicates that said patient has a better prognosis.

170. The method of claim 169, wherein said patient's total IGFBP-3 concentration is higher if it is greater than about 1.9 μg/mL and lower if it is less than about 1.9 μg/mL.

171-172. (canceled)

173. The method of claim 160, further comprising determining the concentration of a second circulating biomarker listed in claim 160.

174. The method of claim 173, further comprising determining the concentration of a third circulating biomarker listed in claim 160.

175. The method of claim 174, further comprising determining the concentration of a fourth circulating biomarker listed in claim 160.

176. The method of claim 173, wherein said biomarkers are IGFBP-2 and total IGF-1.

177. (canceled)

178. The method of claim 173, wherein said biomarkers are IGFBP-2 and IGFBP-3.

179. (canceled)

180. The method of claim 173, wherein said biomarkers are total IGF-1 and IGFBP-3.

181. (canceled)

182. (canceled)

183. The method of claim 149, further comprising treating said patient with an anti-tumor treatment if said patient has a better prognosis.

184. The method of claim 183, wherein said treatment is a chemotherapeutic agent, radiation therapy, or anti-hormone therapy.

185. The method of claim 183, wherein said chemotherapeutic agent is a mitotic inhibitor, alkylating agent, anti-metabolite, intercalating antibiotic, growth factor inhibitor, cell cycle inhibitor, enzyme, topoisomerase inhibitor, anti-survival agent, biological response modifier, anti-hormone, anti-androgen, anti-angiogenesis agent, CPT-11, camptothecin, SN38, adriamycin, cytotoxan, ifosphamide, vincristine, topotecan, vincristine, taxotere, and gemcitabine.

186. The method of claim 149, further comprising further comprising providing said patient with palliative care if said patient does not have a better prognosis.

187. A kit for practicing the method of claim 149.
